SEQUENCE LISTING (238 sequences)

SEQ ID NO: 1 - protein, 17 aa, unidentified
MKCLLYLAFLFIGVNCK

SEQ ID NO: 2 - protein, 16 aa, unidentified
MKCLLYLAFLFIGVNC

SEQ ID NO: 3 - DNA, 20 bp, synthetic construct
  source 1..20: mol_type = other DNA
gagcagcgtcttcgagagtg

SEQ ID NO: 4 - protein, 4 aa, unidentified
RNKR

SEQ ID NO: 5 - protein, 19 aa, synthetic construct
CKNEKKNKIERNNKLKQPP

SEQ ID NO: 6 - protein, 19 aa, synthetic construct
DNEKLRKPKHKKLKQPADG

SEQ ID NO: 7 - protein, 9 aa, synthetic construct
RLTRKRGLK

SEQ ID NO: 8 - protein, 8 aa, synthetic construct
CGRGDSPC

SEQ ID NO: 9 - protein, 5 aa, synthetic construct
RGDYK

SEQ ID NO: 10 - protein, 5 aa, synthetic construct
RGDFK

SEQ ID NO: 11 - protein, 6 aa, synthetic construct
PHSCNK

SEQ ID NO: 12 - protein, 8 aa, synthetic construct
CSRNLIDC

SEQ ID NO: 13 - protein, 12 aa, synthetic construct
VHWDFRQWWQPS

SEQ ID NO: 14 - protein, 17 aa, synthetic construct
CHPREVDVELYSTVFGH

SEQ ID NO: 15 - protein, 40 aa, synthetic construct
CEPEAEADAEAGPAGIGAVLKVLTTGLPALISWIKRKRQQ

SEQ ID NO: 16 - protein, 7 aa, synthetic construct
RPARPAR

SEQ ID NO: 17 - protein, 9 aa, synthetic construct
CRGDKGPDC

SEQ ID NO: 18 - protein, 9 aa, synthetic construct
AKRGARSTA

SEQ ID NO: 19 - protein, 9 aa, synthetic construct
CKRGARSTC

SEQ ID NO: 20 - protein, 9 aa, synthetic construct
CGNKRTRGC

SEQ ID NO: 21 - protein, 31 aa, synthetic construct
HAEGTFTSDVSSYLEGQAAKEFIAWLVRGRG

SEQ ID NO: 22 - protein, 21 aa, synthetic construct
KNSRSLGENDDGNNEDNEKLR

SEQ ID NO: 23 - protein, 12 aa, synthetic construct
AQQAANVAATLK

SEQ ID NO: 24 - protein, 33 aa, synthetic construct
AQQAANVAATLKKNSRSLGENDDGNNEDNEKLR

SEQ ID NO: 25 - protein, 9 aa, synthetic construct
CDGRPDRAC

SEQ ID NO: 26 - protein, 14 aa, synthetic construct
GNSTMCDGRPDRAC

SEQ ID NO: 27 - protein, 25 aa, Homo sapiens
MGVLLTQRTLLSLVLALLFPSMASM

SEQ ID NO: 28 - protein, 19 aa, Homo sapiens
MGWSCIILFLVATATGVHS

SEQ ID NO: 29 - protein, 21 aa, Homo sapiens
MWWRLWWLLLLLLLLWPMVWA

SEQ ID NO: 30 - protein, 22 aa, Homo sapiens
MDMRVPAQLLGLLLLWLRGARC

SEQ ID NO: 31 - protein, 16 aa, Homo sapiens
MPLLLLLPLLWAGALA

SEQ ID NO: 32 - protein, 23 aa, Homo sapiens
MDAMKRGLCCVLLLCGAVFVSPS

SEQ ID NO: 33 - protein, 18 aa, Homo sapiens
MAFLWLLSCWALLGTTFG

SEQ ID NO: 34 - protein, 15 aa, Homo sapiens
MNLLLILTFVAAAVA

SEQ ID NO: 35 - protein, 20 aa, Homo sapiens
MYRMQLLSCIALSLALVTNS

SEQ ID NO: 36 - protein, 16 aa, Homo sapiens
MKWVTFISLLFSSAYS

SEQ ID NO: 37 - protein, 24 aa, Homo sapiens
MALWMRLLPLLALLALWGPDPAAA

SEQ ID NO: 38 - protein, 24 aa, Homo sapiens
MPSSVSWGILLLAGLCCLVPVSLA

SEQ ID NO: 39 - protein, 1936 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLSGGSKRTADGSEPKKKRKVGS

SEQ ID NO: 40 - protein, 1710 aa, synthetic construct
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 41 - protein, 198 aa, Homo sapiens [sequence omitted]

SEQ ID NO: 42 - protein, 191 aa, Homo sapiens
LMDPHIFTSNFNNGIGRHKTYLCYEVERLDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGL

SEQ ID NO: 43 - protein, 175 aa, Homo sapiens
MDPHIFTSNFNNGIGRHKTYLCYEVERLDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPL

SEQ ID NO: 44 - protein, 229 aa, Rattus norvegicus [sequence omitted]

SEQ ID NO: 45 - protein, 397 aa, Mus musculus
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFRPWKGLEIISRRTQRRLRRIKESWGLQDLVNDFGNLQLGPPMSN

SEQ ID NO: 46 - protein, 199 aa, Mus musculus
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRR

SEQ ID NO: 47 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 48 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 49 - protein, 186 aa, Homo sapiens
PPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 50 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 51 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 52 - protein, 189 aa, Homo sapiens
KEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 53 - protein, 383 aa, Escherichia coli
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 54 - protein, 1226 aa, Homo sapiens
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLGSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 55 - protein, 701 aa, Homo sapiens
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 56 - protein, 1419 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSGGGGSGKRTADGSEFEPKKKRKVSSGGDYKDHDGDYKDHDIDYKDDDDK

SEQ ID NO: 57 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 58 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 59 - protein, 1082 aa, Neisseria meningitidis
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 60 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 61 - protein, 1228 aa, unidentified
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 62 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 63 - protein, 1152 aa, Leptotrichia wadei
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENGITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 64 - protein, 118 aa, Homo sapiens
NPDREGWLLKLGGGRVKTWKRRWFILTDNCLYYFEYTTDKEPRGIIPLENLSIREVDDPRKPNCFELYIPNNKGQLIKACKTEADGRVVEGNHMVYRISAPTQEEKDEWIKSIQAAVS

SEQ ID NO: 65 - protein, 118 aa, Homo sapiens
NPDREGWLLKLGGGRVKTWKCRWFILTDNCLYYFEYTTDKEPRGIIPLENLSIREVDDPRKPNCFELYIPNNKGQLIKACKTEADGRVVEGNHMVYRISAPTQEEKDEWIKSIQAAVS

SEQ ID NO: 66 - protein, 59 aa, Homo sapiens
DNEVQNCMACGKGFSVTVRRHHCRQCGNIFCAECSAKNALTPSSKKPVRVCDACFNDLQ

SEQ ID NO: 67 - protein, 59 aa, Homo sapiens
DNEVQNCMACGKGFSVTVRRHHCLQCGNIFCAECSAKNALTPSSKKPVRVCDACFNDLQ

SEQ ID NO: 68 - protein, 122 aa, unidentified
DVAISANIADIEEKRGFTSHFVFVIEVKTKGGSKYLIYRRYRQFHALQSKLEERFGPDSKSSALACTLPTLPAKVYVGVKQEIAEMRIPALNAYMKSLLSLPVWVLMDEDVRIFFYQSPYDS

SEQ ID NO: 69 - protein, 122 aa, unidentified
DVAISANIADIEEKRGFTSHFVFVIEVKTKGGSKYLIYLRYRQFHALQSKLEERFGPDSKSSALACTLPTLPAKVYVGVKQEIAEMRIPALNAYMKSLLSLPVWVLMDEDVRIFFYQSPYDS

SEQ ID NO: 70 - protein, 126 aa, Homo sapiens
MQTGRTEDDLVPTAPSLGTKEGYLTKQGGLVKTWKTRWFTLHRNELKYFKDQMSPEPIRILDLTECSAVQFDYSQERVNCFCLVFPFRTFYLCAKTGVEADEWIKILRWKLSQIRKQLNQGEGTIR

SEQ ID NO: 71 - protein, 153 aa, Homo sapiens
PFKIPEDDGNDLTHTFFNPDREGWLLKLGGRVKTWKRRWFILTDNCLYYFEYTTDKEPRGIIPLENLSIREVEDPRKPNCFELYNPSHKGQVIKACKTEADGRVVEGNHVVYRISAPSPEEKEEWMKSIKASISRDPFYDMLATRKRRIANKK

SEQ ID NO: 72 - protein, 154 aa, Homo sapiens
MPFKIPEDDGNDLTHTFFNPDREGWLLKLGGRVKTWKCRWFILTDNCLYYFEYTTDKEPRGIIPLENLSIREVEDPRKPNCFELYNPSHKGQVIKACKTEADGRVVEGNHVVYRISAPSPEEKEEWMKSIKASISRDPFYDMLATRKRRIANKK

SEQ ID NO: 73 - protein, 101 aa, Homo sapiens
MGSGSAREGWLFKWTNYIKGYQRRWFVLSNGLLSYYRSKAEMRHTCRGTINLATANITVEDSCNFIISNGGAQTYHLKASSEVERQRWVTALELAKAKAVK

SEQ ID NO: 74 - protein, 101 aa, Homo sapiens
MGSGSAREGWLFKWTNYIKGYQERWFVLSNGLLSYYRSKAEMRHTCRGTINLATANITVEDSCNFIISNGGAQTYHLKASSEVERQRWVTALELAKAKAVK

SEQ ID NO: 75 - protein, 177 aa, Homo sapiens
MAAVILESIFLKRSQQKKKTSPLNFKKRLFLLTVHKLSYYEYDFERGRRGSKKGSIDVEKITCVETVVPEKNPPPERQIPRRGEESSEMEQISIIERFPYPFQVVYDEGPLYVFSPTEELRKRWIHQLKNVIRYNSDLVQKYHPCFWIDGQYLCCSQTAKNAMGCQILENRNGSLKP

SEQ ID NO: 76 - protein, 177 aa, Homo sapiens
MAAVILESIFLKRSQQKKKTSPLNFKKCLFLLTVHKLSYYEYDFERGRRGSKKGSIDVEKITCVETVVPEKNPPPERQIPRRGEESSEMEQISIIERFPYPFQVVYDEGPLYVFSPTEELRKRWIHQLKNVIRYNSDLVQKYHPCFWIDGQYLCCSQTAKNAMGCQILENRNGSLKP

SEQ ID NO: 77 - protein, 98 aa, Homo sapiens
MEGVLYKWTNYLTGWQPRWFVLDNGILSYYDSQDDVCKGSKGSIKMAVCEIKVHSADNTRMELIIPGEQHFYMKAVNAAERQRWLVALGSSKACLTDT

SEQ ID NO: 78 - protein, 95 aa, Homo sapiens
PVERCGVLSKWTNYIHGWQDRWVVLKNNALSYYKSEDETEYGCRGSICLSKAVITPHDFDECRFDISVNDSVWYLRAQDPDHRQQWIDAIEQHKT

SEQ ID NO: 79 - protein, 102 aa, Homo sapiens
MRIQLSGMYNVRKGKMQLPVNRWTRRQVILCGTCLIVSSVKDSLTGKMHVLPLIGGKVEEVKKHQHCLAFSSSGPQSQTYYICFDTFTEYLRWLRQVSKVAS

SEQ ID NO: 80 - protein, 98 aa, Homo sapiens
MDVLKQGYMMKKGHRRKNWTERWFVLKPNIISYYVSEDLKDKKGDILLDENCCVESLPDKDGKKCLFLVKCFDKTFEISASDKKKKQEWIQAIHSTIH

SEQ ID NO: 81 - protein, 98 aa, Homo sapiens
MDVLKQGYMMKKGHEEKNWTERWFVLKPNIISYYVSEDLKDKKGDILLDENCCVESLPDKDGKKCLFLVKCFDKTFEISASDKKKKQEWIQAIHSTIH

SEQ ID NO: 82 - protein, 114 aa, Homo sapiens
MDMLSSHHYKSFKVSMIHRLRFTTDVQLGISGDKVEIDPVTNQKASTKFWIKQKPISIDSDLLCACDLAEEKSPSHAIFKLTYLSNHDYKHLYFESDAATVNEIVLKVNYILES

SEQ ID NO: 83 - protein, 122 aa, Homo sapiens
MGTVMKEGWMVHYTSKDTLRKRHYWRLDSKCITLFQNDTGSRYYKEIPLSEILSLEPVKTSALIPNGANPHCFEITTANVVYYVGENVVNPSSPSPNNSVLTSGVGADVARMWEIAIQHALM

SEQ ID NO: 84 - protein, 105 aa, Homo sapiens
FIMEGPLTRIGAKHERHIFLFDGLMISCKPNHGQTRLPGYSSAEYRLKEKFVMRKIQICDKEDTCEHKHAFELVSKDENSIIFAAKSAEEKNNWMAALISLHYRS

SEQ ID NO: 85 - protein, 107 aa, Homo sapiens
QGTNLPPSRQIVIRKGWLTISNIGIMKGGSKGYWFVLTAESLSWYKDDEEKEKKYMLPLDNLKVRDVEKSFMSSKHIFALFNTEQRNVYKDYRFLELACDSQEDVDS

SEQ ID NO: 86 - protein, 159 aa, Homo sapiens
QLLKDSFMVELVEGARKLRHVFLFTDLLLCTKLKKQSGGKTQQYDCKWYIPLTDLSFQMVDELEAVPNIPLVPDEELDALKIKISQIKNDIQREKRANKGSKATERLKKKLSEQESLLLLMSPSMAFRVHSRNGKSYTFLISSDYERAEWRENIREQQK

SEQ ID NO: 87 - protein, 117 aa, Homo sapiens
KLLMQGSFSVWTDHKRGHTKVKELARFKPMQRHLFLHEKAVLFCKKREENGEGYEKAPSYSYKQSLNMAAVGITENVKGDAKKFEIWYNAREEVYIVQAPTPEIKAAWVNEIRKVLT

SEQ ID NO: 88 - protein, 175 aa, Homo sapiens
MDSGRDFLTLHGLQDDEDLQALLKGSQLLKVKSSSWRRERFYKLQEDCKTIWQESRKVMRTPESQLFSIEDIQEVRMGHRTEGLEKFARDVPEDRCFSIVFKDQRNTLDLIAPSPADAQHWVLGLHKIIHHSGSMDQRQKLQHWIHSCLRKADKNKDNKMSFKELQNFLKELNIQ

SEQ ID NO: 89 - protein, 170 aa, Homo sapiens
MDSGRDFLTLHGLQDDEDLQALLKGSQLLKVKSTSWRRELFYKLQEDCKTIWQESRKVMRTPESQLFSIEDIQEVRMGHRTEGLEKFARDVPEDRCFSIVFKDQRNTLDLIAPSPADAQHWVLGLHKIIHHSGSMDQRQKLQHWIHSCLRKADKNKDNKMSFKELQNFLK

SEQ ID NO: 90 - protein, 168 aa, Homo sapiens
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 91 - protein, 168 aa, Homo sapiens
MSDVAIVKEGWLHKRGKYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 92 - protein, 168 aa, Homo sapiens
MSDVAIVKEGWLHRRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 93 - protein, 168 aa, Homo sapiens
MSDVAIVREGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 94 - protein, 168 aa, Homo sapiens
MSDVAIVKEGWLHKRGEYIKTWRPRYFLLKNDGTFIGYKERPQDVDQREAPLNNFSVAQCQLMKTERPRPNAFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 95 - protein, 168 aa, Homo sapiens
MSDVAIVKEGWLHKRGEYIKTWRPRYFLLKNDGTFIGYKERPQDVDQREAPLNNFSVAQCQLMKTERPRPNTFIIRCLQWTTVIERTFHVEAPEEREEWTTAIQTVADGLKKQEEEEMDFRSGSPSDNSGAEEMEVSLAKPKHRVTMNEFEYLKLLGKGTFGKVDPPV

SEQ ID NO: 96 - protein, 168 aa, Homo sapiens
MSDVAIVKEGWLHKRGEYIKTWRPCYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 97 - protein, 168 aa, Homo sapiens
MSDVAIVKEGWLHKRGEYIKTWRPRYFLLKNDGDFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 98 - protein, 168 aa, Homo sapiens
MSDVAIVKEGWLHKRGEYIKTWRPRYFLLKNDGFFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 99 - protein, 168 aa, Homo sapiens
MSDVAIVKEGWLHKRGEYIKTWRPRYFLLKNDGLFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 100 - protein, 168 aa, Homo sapiens
MSDVAIVKEGWLHKRGEYIKTWRPRYFLLKNDGTFIGYKERPQDVDQREAPLNNFSVAQCQLMKTERPRPNTFIIRCLQWYTVIERTFHVETPEEREEWTTAIQTVADGLKKQEEEEMDFRSGSPSDNSGAEEMEVSLAKPKHRVTMNEFEYLKLLGKGTFGKVDPPV

SEQ ID NO: 101 - protein, 168 aa, Homo sapiens
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPAAAVTMNEFEYLKLLGKGTFGKVDPPV

SEQ ID NO: 102 - protein, 168 aa, Homo sapiens
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTCAIQTVADGLKKQEEEEMDFRSGSPSDNSGAEEMEVSLAKPKHRVTMNEFEYLKLLGKGTFGKVDPPV

SEQ ID NO: 103 - protein, 92 aa, Homo sapiens
KMGPVDKRKGLFARRRQLLLTEGPHLYYVDPVNKVLKGEIPWSQELRPEAKNFKTFFVHTPNRTYYLMDPSGNAHKWCRKIQEVWRQRYQSH

SEQ ID NO: 104 - protein, 130 aa, synthetic construct
MASNFTQFVLVDNGGTGDVTVAPSNFANGIAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 105 - protein, 62 aa, synthetic construct
MKSIRCKNCNKLLFKADSFDHIEIRCPRCKRHIIMLNACEHPTEKHCGKREKITHSDETVRY

SEQ ID NO: 106 - protein, 131 aa, synthetic construct
MAKTIVLAVGEATRTLTEIQSTADRQIFEEKVGPLVGRLRLTASLRQNGAKTAYRVNLKLDQADVVDASTSVAGELPKVRYTQVWSHDVTIVANSTEASRKSLYDLTKSLVATSQVEDLVVNLVPLGRSLE

SEQ ID NO: 107 - protein, 102 aa, synthetic construct
MAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFV

SEQ ID NO: 108 - protein, 74 aa, Homo sapiens
MADFETDESVLMRRQKQINYGKNTIAYDRYIKEVPRHLRQPGIHPKTPNKFKKYSRRSWDQQIKLWKVALHFWD

SEQ ID NO: 109 - protein, 38 aa, Human alphaherpesvirus 1
PTDALDDFDLDMLPADALDDFDLDMLPADALDDFDLDM

SEQ ID NO: 110 - protein, 53 aa, synthetic construct
GRADALDDFDLDMLGSDALDDFDLDMLGSDALDDFDLDMLGSDALDDFDLDML

SEQ ID NO: 111 - protein, 261 aa, Homo sapiens
SQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALL

SEQ ID NO: 112 - protein, 190 aa, Human gammaherpesvirus 8
RDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 113 - protein, 72 aa, Homo sapiens
MDAKSLTAWSRTLVTFKDVFVDFTREEWKLLDTAQQIVYRNVMLENYKNLVSLGYQLTKPDVILRLEKGEEP

SEQ ID NO: 114 - protein, 289 aa, Homo sapiens
EASVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVS

SEQ ID NO: 115 - protein, 718 aa, Homo sapiens
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 116 - protein, 912 aa, Homo sapiens
MPAMPSSGPGDTSSSAAEREEDRKDGEEQEEPRGKEERQEPSTTARKVGRPGRKRKHPPVESGDTPKDPAVISKSPSMAQDSGASELLPNGDLEKRSEPQPEEGSPAGGQKGGAPAEGEGAAETLPEASRAVENGCCTPKEGRGAPAEAGKEQKETNIESMKMEGSRGRLRGGLGWESSLRQRPMPRLTFQAGDPYYISKRKRDEWLARWKREAEKKAKVIAGMNAVEENQGPGESQKVEEASPPAVQQPTDPASPTVATTPEPVGSDAGDKNATKAGDDEPEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPVCHDSDESDTAKAVEVQNKPMIEWALGGFQPSGPKGLEPPEEEKNPYKEVYTDMWVEPEAAAYAPPPPAKKPRKSTAEKPKVKEIIDERTRERLVYEVRQKCRNIEDICISCGSLNVTLEHPLFVGGMCQNCKNCFLECAYQYDDDGYQSYCTICCGGREVLMCGNNNCCRCFCVECVDLLVGPGAAQAAIKEDPWNCYMCGHKGTYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 117 - protein, 546 aa, unidentified
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAM

SEQ ID NO: 118 - protein, 584 aa, Homo sapiens
MIFAGKAPSNTSTLMKFYSLLLYSLLFSFPFLCHPLPLPSYLHHTINLTHSLLAASNPSLVNNCWLCISLSSSAYTAVPAVQTDWATSPISLHLRTSFNSPHLYPPEELIYFLDRSSKTSPDISHQQAAALLRTYLKNLSPYINSTPPIFGPLTTQTTIPVAAPLCISWQRPTGIPLGNLSPSRCSFTLHLRSPTTNINETIGAFQLHITDKPSINTDKLKNISSNYCLGRHLPCISLHPWLSSPCSSDSPPRPSSCLLIPSPENNSERLLVDTRRFLIHHENRTFPSTQLPHQSPLQPLTAAALAGSLGVWVQDTPFSTPSHLFTLHLQFCLAQGLFFLCGSSTYMCLPANWTGTCTLVFLTPKIQFANGTEELPVPLMTPTQQKRVIPLIPLMVGLGLSASTVALGTGIAGISTSVMTFRSLSNDFSASITDISQTLSVLQAQVDSLAAVVLQNRRGLDLLTAEKGGLCIFLNEECCFYLNQSGLVYDNIKKLKDRAQKLANQASNYAEPPWALSNWMSWVLPIVSPLIPIFLLLLFGPCIFRLVSQFIQNRIQAITNHSIRQMFLLTSPQYHPLPQDLPSA

SEQ ID NO: 119 - protein, 563 aa, Homo sapiens
MIFAGRASSNTSTLMKFYSLLLYSLLFSFPILCHPLPLPSYLHHTINLTHSLLAVSNPSLAKNCWLCISLPSSAYPAVPALQTDWGTSPVSPHLRTSFNSPHLYPPEKLIYFLDRSSKTSPDISHQQAAALLCTYLKNLSPYINSTPPTFGPLTTQTTIPVAAPLCISRQRPTGIPLGNLSPSRCSFTLHLRSPTTHITETNGAFQLHITDKPSINTDKLKNVSSNYCLGRHLSCISLHPWLFSPCSSDSPPRPSSCLLIPSPKNNSESLLVDAQRFLIYHENRTSPSTQLPHQSPLQPLTAAPLGGSLRVWVQDTPFSTPSHLFTLHLQFCLVQSLFFLCGSSTYMCLPANWTGTCTLVFLTSKIQFANGTEELPVPLMTPTRQKRVIPLIPLMVGLGLSASTVALGTGIAGISTSVTTFRILSNDFSASITDISQTLSGLQAQVDSSAAVVLQNRQGLDLLTAEKGGLCIFLNEESYFYLNQSGLVYDNIKKLKDKAQNLANQASNYAEPPWPLSNWMSWVLPILSPLIPIFLLLFFRPCIFHLVSQFIQNHIQAITDHSI

SEQ ID NO: 120 - protein, 555 aa, Homo sapiens
MILAGRAPSNTSTLMKFYSLLLYSLLFSFPFLYHPLPLPSYLHHTINLTHSLPAASNPSLANNCWLCISLSSSAYIAVPTLQTDRATSPVSLHLRTSFNSPHLYPPEELIYFLDRSSKTSPDISHQPAAALLHIYLKNLSPYINSTPPIFGPLTTQTTIPVAAPLCISRQRPTGIPLGNISPSRCSFTLHLQSPTTHVTETIGVFQLHIIDKPSINTDKLKNVSSNYCLGRHLPYISLHPWLPSPCSSDSPPRPSSCLLTPSPQNNSERLLVDTQRFLIHHENRTSSSMQLAHQSPLQPLTAAALAGSLGVWVQDTPFSTPSHPFSLHLQFCLTQGLFFLCGSSTYMCLPANWTGTCTLVFLTPKIQFANGTKELPVPLMTLTPQKRVIPLIPLMVGLGLSASTIALSTGIAGISTSVTTFRSPSNDFSASITDISQTLSVLQAQVDSLAAVVLQNRRGLGLSILLNEECCFYLNQSGLVYENIKKLKDRAQKLANQASNYAESPWALSNWMSWVLPILSPLIPIFLLLLFGPCIFHLVSQFIQNRIQAITNHSI

SEQ ID NO: 121 - protein, 698 aa, Homo sapiens
MHPSEMQRKAPPRRRRHRNRAPLTHKMNKMVTSEQMKLPSTKKAEPPTWAQLKKLTQLATKYLENTKVTQTPESMLLAALMIVSMVVSLPMPAGAAAANYTNWAYVPFPPLIRAVTWMDNPIEVYVNDSVWVHGPIDDRCPAKPEEEGMMINISIGYHYPPICLGRAPGCLMPAVQNWLVEVPTVSPISRFTYNMVSGMSLRPRVNYLQDFSYQRSLKFRPKGKPCPKEIPKESKNTEVLVWEECVANSVVILQNNEFGTIIDWAPRGQFYHNCSGQTQSCPSAQVSPAVDSDLTESLDKHKHKKLQSFYPWEWGEKGISTPRPKIISPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSIHILTEVLKGVLNRSKRFIFTLIAVIMGLIAVTAMAAVAGVALHSFVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRFTQQLRRDSYHRERAMMTMVVLSKRKGGNVGKSKRDQIVTVSV

SEQ ID NO: 122 - protein, 699 aa, Homo sapiens
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPTEVYVNDSVWVPGPIDDRCPAKPEEEGMMINISIGYHYPPICLGRAPGCLMPAVQNWLVEVPTVSPICRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTIDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 123 - protein, 699 aa, Homo sapiens
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPTDDHCPAKPEEEGMMINISIGYRYPPICLGRAPGCLMPAVQNWLVEVPTVSPISRFTYHMVSGMSLRPRVNYLQDFSYQRSFKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIISPVSGPEHPELWRLTVASHHIRIWSGNQTLETRDRKPFYTVDLNSSVTVPLQSCIKPPYMLVVGNIVIKPDSQTITCENCRLLTCIDSTFNWQHRILLVRAREGVWIPVSMDRPWETSPSIHTLTEVLKGVLNRSKRFIFTLIAVIMGLIAVTATAAVAGVALHSSVQSVNFVNDWQKNSTRLWNSQSSIDQKLANQINDLRQTVIWMGDRLMSLEHRFQLQCDWNTSDFSITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 124 - protein, 698 aa, Homo sapiens
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTNWAYVPFPPLIRAVTWMDNPIEVYVNDSVWVPGPIDDRCPAKPEEEGMMINISIGYRYPICLGRAPGCLMPAVQNWLVEVPIVSPICRFTYHMVSGMSLRPRVNYLQDFSYQRSLKFRPKGKPCPKEIPKESKNTEVLVWEECVANSAVILQNNEFGTIIDWTPQGQFYHNCSGQTQSCPSAQVSPAVDSDLTESLDKHKHKKLQSFYPWEWGEKGISTPRPKIISPVSGPEHPELWRLTVASHHIRIWSGNQTLETRDRKPFYTVDLNSSLTLPLQSCVKPPYMLVVGNIVIKPDSQTITCENCRLLTCIDSTFNWQHRILLVRAREGVWIPVSMDRPWEASPSIHILTEVLKGVLNRSKRFIFTLIAVIMGLIAVTATAAVAGVALHSSVQSVNFVNDGQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 125 - protein, 699 aa, Homo sapiens
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEIYVNDSVWVPGPTDDCCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTLIDWAPRGQFYHNCSGQTQSCPSAQVSPAVDSDLTESLDKHKHKKLQSFYPWEWGEKGISTARPKIISPVSGPEHPELWRLTVASHHIRIWSGNQTLETRDRKPFYTIDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQNNSTRLWNSQSSIDQKLANQINDLRQTVIWMGDRLMSLEHRFQLQCDWNTSDFCITPQIYNESEHHWDMVRCHLQGREDNLTLDISKLKEQIFEASKAHLNLVPGTEAIAGVADGLANLNTVTWVKTIGSTTIINLILILVCLFCLLLVYRCTQQLRRDSDHRERAMMTMVVLSKRKGGNVGKSKRDQIVTVSV

SEQ ID NO: 126 - protein, 699 aa, Homo sapiens
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAVANYTNWAYVPFPPLIRAVTWMDNPIEVYVNDSVWVPGPIDDRCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKRISTPRPKIVSPVSGPEHPELWRLTVASHHIRIWSGNQTLETRDRKPFYTVDLNSSLTLPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDGQKNSTRLWNSQSSIDQKLANQINDLRQTVIWMGDRLMSLEHRFQLQCDWNTSDFCITPQIYNDSEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 127 - protein, 626 aa, Homo sapiens
MGPEAWVRPLKTAPKPGEAIRLILFIYLSCFFLPVMSSEPSYSFLLTSFTTGRVFANTTWRAGTSKEVSFAVDLCVLFPEPARTHEEQHNLPVIGAGSVDLAAGFGHSGSQTGCGSSKGAEKGLQNVDFYLCPGNHPDASCRDTYQFFCPDWTCVTLATYSGGSTRSSTLSISRVPHPKLCTRKNCNPLTITVHDPNAAQWYYGMSWGLRLYIPGFDVGTMFTIQKKILVSWSSPKPIGPLTDLGDPIFQKHPDKVDLTVPLPFLVPRPQLQQQHLQPSLMSILGGVHHLLNLTQPKLAQDCWLCLKAKPPYYVGLGVEATLKRGPLSCHTRPRALTIGDVSGNASCLISTGYNLSASPFQATCNQSLLTSISTSVSYQAPNNTWLACTSGLTRCINGTEPGPLLCVLVHVLPQVYVYSGPEGRQLIAPPELHPRLHQAVPLLVPLLAGLSIAGSAAIGTAALVQGETGLISLSQQVDADFSNLQSAIDILHSQVESLAEVVLQNCRCLDLLFLSQGGLCAALGESCCFYANQSGVIKGTVKKVRENLDRHQQERENNIPWYQSMFNWNPWLTTLITGLAGPLLILLLSLIFGPCILNSFLNFIKQRIASVKLTYLKTQYDTLVNN

SEQ ID NO: 128 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 129 - protein, 538 aa, Homo sapiens
MGLLLLVLILTPSLAAYRHPDFPLLEKAQQLLQSTGSPYSTNCWLCTSSSTETPGTAYPASPREWTSIEAELHISYRWDPNLKGLMRPANSLLSTVKQDFPDIRQKPPIFGPIFTNINLMGIAPICVMAKRKNGTNVGTLPSTVCNVTFTVDSNQQTYQTYTHNQFRHQPRFPKPPNITFPQGTLLDKSSRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAPGNLSLPIPIYGNSPLPRVRRAIHFIPLLAGLGILAGTGTGIAGITKASLTYSQLSKEIANNIDTMAKALTTMQEQIDSLAAVVLQNRRGLDMLTAAQGGICLALDEKCCFWVNQSGKVQDNIRQLLNQASSLRERATQGWLNWEGTWKWFSWVLPLTGPLVSLLLLLLFGPCLLNLITQFVSSRLQAIKLQTNLSAGRHPRNIQESPF

SEQ ID NO: 130 - protein, 604 aa, Homo sapiens
MLGMNMLLITLFLLLPLSMLKGEPWEGCLHCTHTTWSGNIMTKTLLYHTYYECAGTCLGTCTHNQTTYSVCDPGRGQPYVCYDPKSSPGTWFEIHVGSKEGDLLNQTKVFPSGKDVVSLYFDVCQIVSMGSLFPVIFSSMEYYSSCHKNRYAHPACSTDSPVTTCWDCTTWSTNQQSLGPIMLTKIPLEPDCKTSTCNSVNLTILEPDQPIWTTGLKAPLGARVSGEEIGPGAYVYLYIIKKTRTRSTQQFRVFESFYEHVNQKLPEPPPLASNLFAQLAENIASSLHVASCYVCGGMNMGDQWPWEARELMPQDNFTLTASSLEPAPSSQSIWFLKTSIIGKFCIARWGKAFTDPVGELTCLGQQYYNETLGKTLWRGKSNNSESPHPSPFSRFPSLNHSWYQLEAPNTWQAPSGLYWICGPQAYRQLPAKWSGACVLGTIRPSFFLMPLKQGEALGYPIYDETKRKSKRGITIGDWKDNEWPPERIIQYYGPATWAEDGMWGYRTPVYMLNRIIRLQAVLEIITNETAGALNLLAQQATKMRNVIYQNRLALDYLLAQEEGVCGKFNLTNCCLELDDEGKVIKEITAKIQKLAHIPVQTWKG

SEQ ID NO: 131 - protein, 514 aa, Homo sapiens
MDPLHTIEKVPARRNIHDRGHQGHRMGDGTPGRPKISVQQMTRFSLIIFFLSAPFVVNASTSNVFLQWAHSYADGLQQGDPCWVCGSLPVTNTMELPWWVSPLQGKDWVFFQSFIGDLKQWTGAQMTGVTRKNISEWPINKTLNEPGHDKPFSVNETRDKVIAFAIPLLDTKVFVQTSRPQNTQYRNGFLQIWDGFIWLTATKGHLSQIAPLCWEQRNHSLDNWPNTTRVMGWIPPGQCRHTILLQQRDLFATDWSQQPGLNWYAPNGTQWLCSPNLWPWLPSGWLGCCTLGIPWAQGRWVKTMEVYPYLPHVVNQGTRAIVHRNDHLPTIFMPSVGLGTVIQHIEALANFTQRALNDSLQSISLMNAEVYYMHEDILQNRMALDILTAAEGGTCALIKTECCVYIPNNSRNISLALEDTCRQIQVISSSALSLHDWIASQFSGRPSWWQKILIVLATLWSVGIALCCGLYFCRMFSQHIPQTHSIIFQQELPLSPPSQEHYQSQRDIFHSNAP

SEQ ID NO: 132 - protein, 527 aa, Homo sapiens
MNSPCDRLQQFIQVLLEESWSFPSFANTLHWPENLLSYIDELVWQGSLQNFHQHEVRFDKPPLRLPLTGFSSLTENWSSRQAVSSRLVATAASPPAGCQAPIAFLGLKFSSLGPARKNPALCFLYDQSNSKCNTSWVKENVGCPWHWCNIHEALIRTEKGSDPMFYVNTSTGGRDGFNGFNLQISDPWDPRWASGVDGGLYEHKTFMYPVAKIRIARTLKTTVTGLSDLASSIQSAEKELTSQLQPAADQAKSSRFSWLTLISEGAQLLQSTGVQNLSHCFLCAALRRPPLVAVPLPTPFNYTINSSTPIPPVPKGQVPLFSDPIRHKFPFCYSTPNASWCNQTRMLTSTPAPPRGYFWCNSTLTKVLNSTGNHTLCLPISLIPGLTLYSQDELSHLLAWTEPRPQNKSKWAIFLPLVLGISLASSLVASGLGKGALTHSIQTSQDLSTHLQLAIEASAESLDSLQRQITTVAQVAAQNRQALDLLMAEKGRTCLFLQEECCYYLNESGVVENSLQTLKKKKSSKRS

SEQ ID NO: 133 - protein, 584 aa, Homo sapiens
MARPSPLCLLLLLTLLTPIVPSNSLLTEPPFRWRFYLHETWTQGNRLSTVTLATVDCQPHGCQAQVTFNFTSFKSVLRGWSNPTICFVYDQTHSNCRDYWVDTNGGCPYAYCRMHVTQLHTAKKLQHTYRLTSDGRTTYFLTIPDPWDSRWVSGVTGRLYRWPTDSYPVGKLRIFLTYIRVIPQVLSNLKDQADNIKHQEEVINTLVQSHPKADMVTYDDKAEAGPFSWITLVRHGARLVNMAGLVNLSHCFLCTALSQPPLVAVPLPQAFNTSGNHTAHPSGVFSEQVPLFRDPLQPQFPFCYTTPNSSWCNQTYSGSLSNLSAPAGGYFWCNFTLTKHLNISSNNTLSRNLCLPISLVPRLTLYSEAELSSLVNPPMRQKRAVFPPLVIGVSLTSSLVASGLGTGAIVHFISSSQDLSIKLQMAIEASAESLASLQRQITSVAKVAMQNRRALDLLTADKGGTCMFLGEECCYYINESGLVETSLLTLDKIRDGLHRPSSTPNYGGGWWQSPLTTWIIPFISPILIICLLLLIAPCVLKFIKNRISEVSRVTVNQMLLHPYSRLPTSEDHYDDALTQQEAAR

SEQ ID NO: 134 - protein, 699 aa, Homo sapiens
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 135 - protein, 631 aa, Homo sapiens
MGPQAWARPLKTAPKSSEAIKLILFIYLFCLFPPITPSAPSYSFLLTSFTTGRVFANTTWKAGTSKEVSFAVDLCALFPEPARTHEEQCNLPVMGAGNVDLAAGFGHTGSRTGCGSSKGAEKGLQSVDFYLCPGNHPDSSCRDSYQFFCPHWSCVTLATYSGGSTRSSTLSITRTSRPRPCTIRNCNPLTITVRNPNSAQWYYGMSWGLRLYISGFDVGTMFTIQKKVLVPWSPPKPIGPLTDLGDPMFQKHPDRVDLTVPPPLLVPKSQLQRQHLQPSLMSILDGVHHLLNLTQPKLAQDCWLCLKAKPPYYVGLGVEAMFKVNSLSCHTRPHALTLGDVSGNASCLISAGYNLSASPFQATCNQSLLTSLNASVSYQAPNNTWLACTSGLTRCINGTEPGPLLCVLVHVLPQVYVYSGPEGQLLIAPPELHSRFRRAAPLLVPLLAGLSIAGSAAIGTAALVQGETGLMSLSQQVDADLSNLQSAINMLHTQVESLAEVVLQNRRGLDLLFLSQGGLCAALGESCCFYANQSGVIKDTLQRVRENLDRRQQERENNTPWYQSMFNWNPWLTTLITGLAGPLLLLLLGLVFGPCILNWFLNFVKQRIASVKLTYLRTQYNPLVITEESMI

SEQ ID NO: 136 - protein, 665 aa, Homo sapiens
MDPSHPSQESTAPSSVMGHSPRGKSYQTKAKESLILFHLFCYSFFFPCALASHLIINVTRSDSPQTITFDACLVIPCGDLQSQRQLAAAEKYLCPSEADASTLFSFPFCHTWEYVVWTTQRQDWVPSQDFPLAVLKPYIHFTKGIAPPNCRYNQCNPVQISITIPTLQDSSPTLNRFYGMGADVRGKDPIGFFELHLSTSPSLISPRLSSSTPANQTIVSSSNDKSKVAIVEVKNLKQTLTIETGYKETNAWMEWIEYSVRSLNKSDCYACAQGRPEAQVVPFPLGWSSDQPGMGCMVALFQHPTAWDSEFCRTLSVLFPETQHLEGEPPRAIQPPSPDAKFTSCLSRQGKNLEFLGDLKGCSELKSFQELTNQSALVHARADVWWYCGGHLLDTLPSNWSGTCALIQLAIPFTLAFQQPEKKKPQRRKTREAPQGSFDSHVYVDEIGVPQGVPDRFKARDPVAAGFESLFPMVAINKNVAWINYIYYNQQRFINYTRDAIQGIAEQLGPTSQMAWENRMALDMILAEKGGVCVMIGTQCCTYIPNNTAPDGTITKALQGLTSLSDELATNSGITDPFTGWLGQWFGKWKGLMASIVTSLAIAIAVLILVGCCIMPCIRGLVQRLIETASNKTFPSSSQSYSNKFFPVNEHEIRIILDRFKAEHV

SEQ ID NO: 137 - protein, 477 aa, Homo sapiens
MTEKFLFLYLSLLPMPLLSQAQWNENSLVSFSKIIASGNHLSNCWICHNFITRSSSYQYILVRNFSLNLTFGSGIPEGQHKSVPLQVSLANSAHQVPCLDLTPPFNQSSKTSFYFYNCSSLNQTCCPCPEGHCDRKNTSEEGFPSPTIHPMSFSPAGCHPNLTHWCPAKQMNDYRDKSPQNRCAAWEGKELITWRVLYLLPKAHTVPTWPKSTVPLGGPLSPACNQTIPAGWKSQLHKWFDSHIPRWACTPPGYVFLCGPQKNKLPFDGSPKITYSTPPVANLYTCINNIQHTGECAVGLLGPRGIGVTIYNTTQPRQKRALGLILAGMGAAIGMIAPWGGFTYHDVTLRNLSRQIDNIAKSTRDSISKLKASIDSLANVVMNNRLALDYLLAEQGGVCAVISKSCCIYVNNSGAIEEDIKKIYDEVTWLHNFGKGDSAGSIWEAVKSALPSLTWFVPLLGPAALNSLLSPLWPLSL

SEQ ID NO: 138 - protein, 563 aa, Homo sapiens
MGSLSNYALLQLTLTAFLTILVQPQHLLAPVFRTLSILTNQSNCWLCEHLDNAEQPELVFVPASASTWWTYSGQWMYERVWYPQAEVQNHSTSSYRKVTWHWEASMEAQGLSFAQVRLLEGNFSLCVENKNGSGPFLGNIPKQYCNQILWFDSTDGTFMPSIDVTNESRNDDDDTSVCLGTRQCSWFAGCTNRTWNSSAVPLIGLPNTQDYKWVDRNSGLTWSGNDTCLYSCQNQTKGLLYQLFRNLFCSYGLTEAHGKWRCADASITNDKGHDGHRTPTWWLTGSNLTLSVNNSGLFFLCGNGVYKGFPPKWSGRCGLGYLVPSLTRYLTLNASQITNLRSFIHKVTPHRCTQGDTDNPPLYCNPKDNSTIRALFPSLGTYDLEKAILNISKAMEQEFSATKQTLEAHQSKVSSLASASRKDHVLDIPTTQRQTACGTVGKQCCLYINYSEEIKSNIQRLHEASENLKNVPLLDWQGIFAKVGDWFRSWGYVLLIVLFCLFIFVLIYVRVFRKSRRSLNSQPLNLALSPQQSAQLLVSETSCQVSNRAMKGLTTHQYDTSLL

SEQ ID NO: 139 - protein, 658 aa, Homo sapiens
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCT

SEQ ID NO: 140 - protein, 611 aa, Homo sapiens
MGPQAWARPLKTAPKSSEAIKLILFIYLFCLFPPITPSAPSYSFLLTSFTTGRVFANTTWKAGTSKEVSFAVDLCALFPEPARTHEEQCNLPVMGAGNVDLAAGFGHTGSRTGCGSSKGAEKGLQSVDFYLCPGNHPDSSCRDSYQFFCPHWSCVTLATYSGGSTRSSTLSITRTSRPRPCTIRNCNPLTITVRNPNSAQWYYGMSWGLRLYISGFDVGTMFTIQKKVLVPWSPPKPIGPLTDLGDPMFQKHPDRVDLTVPPPLLVPKSQLQRQHLQPSLMSILDGVHHLLNLTQPKLAQDCWLCLKAKPPYYVGLGVEAMFKVNSLSCHTRPHALTLGDVSGNASCLISAGYNLSASPFQATCNQSLLTSLNASVSYQAPNNTWLACTSGLTRCINGTEPGPLLCVLVHVLPQVYVYSGPEGQLLIAPPELHSRFRRAAPLLVPLLAGLSIAGSAAIGTAALVQGETGLMSLSQQVDADLSNLQSAINMLHTQVESLAEVVLQNRRGLDLLFLSQGGLCAALGESCCFYANQSGVIKDTLQRVRENLDRRQQERENNTPWYQSMFNWNPWLTTLITGLAGPLLLLLLGLVFGPCILNWFLNFVKQRIASV

SEQ ID NO: 141 - protein, 480 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRI

SEQ ID NO: 142 - protein, 483 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAV

SEQ ID NO: 143 - protein, 518 aa, Homo sapiens
MGSLSNYALLQLTLTAFLTILVQPQHLLAPVFRTLSILTNQSNCWLCEHLDNAEQPELVFVPASASTWWTYSGQWMYERVWYPQAEVQNHSTSSYRKVTWHWEASMEAQGLSFAQVRLLEGNFSLCVENKNGSGPFLGNIPKQYCNQILWFDSTDGTFMPSIDVTNESRNDDDDTSVCLGTRQCSWFAGCTNRTWNSSAVPLIGLPNTQDYKWVDRNSGLTWSGNDTCLYSCQNQTKGLLYQLFRNLFCSYGLTEAHGKWRCADASITNDKGHDGHRTPTWWLTGSNLTLSVNNSGLFFLCGNGVYKGFPPKWSGRCGLGYLVPSLTRYLTLNASQITNLRSFIHKVTPHRCTQGDTDNPPLYCNPKDNSTIRALFPSLGTYDLEKAILNISKAMEQEFSATKQTLEAHQSKVSSLASASRKDHVLDIPTTQRQTACGTVGKQCCLYINYSEEIKSNIQRLHEASENLKNVPLLDWQGIFAKVGDWFRSWGYVLLIVLFCLFIFVLIYVRVFRKSRRS

SEQ ID NO: 144 - protein, 521 aa, Homo sapiens
MGSLSNYALLQLTLTAFLTILVQPQHLLAPVFRTLSILTNQSNCWLCEHLDNAEQPELVFVPASASTWWTYSGQWMYERVWYPQAEVQNHSTSSYRKVTWHWEASMEAQGLSFAQVRLLEGNFSLCVENKNGSGPFLGNIPKQYCNQILWFDSTDGTFMPSIDVTNESRNDDDDTSVCLGTRQCSWFAGCTNRTWNSSAVPLIGLPNTQDYKWVDRNSGLTWSGNDTCLYSCQNQTKGLLYQLFRNLFCSYGLTEAHGKWRCADASITNDKGHDGHRTPTWWLTGSNLTLSVNNSGLFFLCGNGVYKGFPPKWSGRCGLGYLVPSLTRYLTLNASQITNLRSFIHKVTPHRCTQGDTDNPPLYCNPKDNSTIRALFPSLGTYDLEKAILNISKAMEQEFSATKQTLEAHQSKVSSLASASRKDHVLDIPTTQRQTACGTVGKQCCLYINYSEEIKSNIQRLHEASENLKNVPLLDWQGIFAKVGDWFRSWGYVLLIVLFCLFIFVLIYVRVFRKSRRSLNS

SEQ ID NO: 145 - protein, 626 aa, Homo sapiens
MDPSHPSQESTAPSSVMGHSPRGKSYQTKAKESLILFHLFCYSFFFPCALASHLIINVTRSDSPQTITFDACLVIPCGDLQSQRQLAAAEKYLCPSEADASTLFSFPFCHTWEYVVWTTQRQDWVPSQDFPLAVLKPYIHFTKGIAPPNCRYNQCNPVQISITIPTLQDSSPTLNRFYGMGADVRGKDPIGFFELHLSTSPSLISPRLSSSTPANQTIVSSSNDKSKVAIVEVKNLKQTLTIETGYKETNAWMEWIEYSVRSLNKSDCYACAQGRPEAQVVPFPLGWSSDQPGMGCMVALFQHPTAWDSEFCRTLSVLFPETQHLEGEPPRAIQPPSPDAKFTSCLSRQGKNLEFLGDLKGCSELKSFQELTNQSALVHARADVWWYCGGHLLDTLPSNWSGTCALIQLAIPFTLAFQQPEKKKPQRRKTREAPQGSFDSHVYVDEIGVPQGVPDRFKARDPVAAGFESLFPMVAINKNVAWINYIYYNQQRFINYTRDAIQGIAEQLGPTSQMAWENRMALDMILAEKGGVCVMIGTQCCTYIPNNTAPDGTITKALQGLTSLSDELATNSGITDPFTGWLGQWFGKWKGLMASIVTSLAIAIAVLILVGCCIMPCIRGLVQRLI

SEQ ID NO: 146 - protein, 515 aa, Homo sapiens
MGLLLLVLILTPSLAAYRHPDFPLLEKAQQLLQSTGSPYSTNCWLCTSSSTETPGTAYPASPREWTSIEAELHISYRWDPNLKGLMRPANSLLSTVKQDFPDIRQKPPIFGPIFTNINLMGIAPICVMAKRKNGTNVGTLPSTVCNVTFTVDSNQQTYQTYTHNQFRHQPRFPKPPNITFPQGTLLDKSSRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAPGNLSLPIPIYGNSPLPRVRRAIHFIPLLAGLGILAGTGTGIAGITKASLTYSQLSKEIANNIDTMAKALTTMQEQIDSLAAVVLQNRRGLDMLTAAQGGICLALDEKCCFWVNQSGKVQDNIRQLLNQASSLRERATQGWLNWEGTWKWFSWVLPLTGPLVSLLLLLLFGPCLLNLITQFVSSRL

SEQ ID NO: 147 - protein, 555 aa, Homo sapiens
MIFAGKAPSNTSTLMKFYSLLLYSLLFSFPFLCHPLPLPSYLHHTINLTHSLLAASNPSLVNNCWLCISLSSSAYTAVPAVQTDWATSPISLHLRTSFNSPHLYPPEELIYFLDRSSKTSPDISHQQAAALLRTYLKNLSPYINSTPPIFGPLTTQTTIPVAAPLCISWQRPTGIPLGNLSPSRCSFTLHLRSPTTNINETIGAFQLHITDKPSINTDKLKNISSNYCLGRHLPCISLHPWLSSPCSSDSPPRPSSCLLIPSPENNSERLLVDTRRFLIHHENRTFPSTQLPHQSPLQPLTAAALAGSLGVWVQDTPFSTPSHLFTLHLQFCLAQGLFFLCGSSTYMCLPANWTGTCTLVFLTPKIQFANGTEELPVPLMTPTQQKRVIPLIPLMVGLGLSASTVALGTGIAGISTSVMTFRSLSNDFSASITDISQTLSVLQAQVDSLAAVVLQNRRGLDLLTAEKGGLCIFLNEECCFYLNQSGLVYDNIKKLKDRAQKLANQASNYAEPPWALSNWMSWVLPIVSPLIPIFLLLLFGPCIFRLVSQFIQNRI

SEQ ID NO: 148 - protein, 476 aa, Homo sapiens
MDPLHTIEKVPARRNIHDRGHQGHRMGDGTPGRPKISVQQMTRFSLIIFFLSAPFVVNASTSNVFLQWAHSYADGLQQGDPCWVCGSLPVTNTMELPWWVSPLQGKDWVFFQSFIGDLKQWTGAQMTGVTRKNISEWPINKTLNEPGHDKPFSVNETRDKVIAFAIPLLDTKVFVQTSRPQNTQYRNGFLQIWDGFIWLTATKGHLSQIAPLCWEQRNHSLDNWPNTTRVMGWIPPGQCRHTILLQQRDLFATDWSQQPGLNWYAPNGTQWLCSPNLWPWLPSGWLGCCTLGIPWAQGRWVKTMEVYPYLPHVVNQGTRAIVHRNDHLPTIFMPSVGLGTVIQHIEALANFTQRALNDSLQSISLMNAEVYYMHEDILQNRMALDILTAAEGGTCALIKTECCVYIPNNSRNISLALEDTCRQIQVISSSALSLHDWIASQFSGRPSWWQKILIVLATLWSVGIALCCGLYFCRMF

SEQ ID NO: 149 - protein, 585 aa, Homo sapiens
MLGMNMLLITLFLLLPLSMLKGEPWEGCLHCTHTTWSGNIMTKTLLYHTYYECAGTCLGTCTHNQTTYSVCDPGRGQPYVCYDPKSSPGTWFEIHVGSKEGDLLNQTKVFPSGKDVVSLYFDVCQIVSMGSLFPVIFSSMEYYSSCHKNRYAHPACSTDSPVTTCWDCTTWSTNQQSLGPIMLTKIPLEPDCKTSTCNSVNLTILEPDQPIWTTGLKAPLGARVSGEEIGPGAYVYLYIIKKTRTRSTQQFRVFESFYEHVNQKLPEPPPLASNLFAQLAENIASSLHVASCYVCGGMNMGDQWPWEARELMPQDNFTLTASSLEPAPSSQSIWFLKTSIIGKFCIARWGKAFTDPVGELTCLGQQYYNETLGKTLWRGKSNNSESPHPSPFSRFPSLNHSWYQLEAPNTWQAPSGLYWICGPQAYRQLPAKWSGACVLGTIRPSFFLMPLKQGEALGYPIYDETKRKSKRGITIGDWKDNEWPPERIIQYYGPATWAEDGMWGYRTPVYMLNRIIRLQAVLEIITNETAGALNLLAQQATKMRNVIYQNRLALDYLLAQEEGVCGKFNLTNCCLELDDEGKVIK

SEQ ID NO: 150 - protein, 448 aa, Homo sapiens
MTEKFLFLYLSLLPMPLLSQAQWNENSLVSFSKIIASGNHLSNCWICHNFITRSSSYQYILVRNFSLNLTFGSGIPEGQHKSVPLQVSLANSAHQVPCLDLTPPFNQSSKTSFYFYNCSSLNQTCCPCPEGHCDRKNTSEEGFPSPTIHPMSFSPAGCHPNLTHWCPAKQMNDYRDKSPQNRCAAWEGKELITWRVLYLLPKAHTVPTWPKSTVPLGGPLSPACNQTIPAGWKSQLHKWFDSHIPRWACTPPGYVFLCGPQKNKLPFDGSPKITYSTPPVANLYTCINNIQHTGECAVGLLGPRGIGVTIYNTTQPRQKRALGLILAGMGAAIGMIAPWGGFTYHDVTLRNLSRQIDNIAKSTRDSISKLKASIDSLANVVMNNRLALDYLLAEQGGVCAVISKSCCIYVNNSGAIEEDIKKIYDEVTWLHNFGKGDSAGSIWEAVKS

SEQ ID NO: 151 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVADQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 152 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVADAAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 153 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVADAAAEKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 154 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQAQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 155 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVAAQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 156 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVAAAAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 157 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVAAAAGEKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 158 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDAAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 159 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGGQDAAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 160 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGGQDAAAEKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 161 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAAEKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 162 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGGQDQAAEKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 163 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGGQAQAAEKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 164 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGGQAAAAEKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 165 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGGQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 166 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQGREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 167 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQGAEKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 168 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVADQGAEKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 169 - protein, 50 aa, synthetic construct
NAVGQDTQEVIVVPHSLPFKVVVISAILALVVLTIISLIILIMLWQKKPR

SEQ ID NO: 170 - protein, 178 aa, synthetic construct
NNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 171 - protein, 34 aa, synthetic construct
TGKLFWALVVVAGVLFCYGLLVTVALCVIWVRSG

SEQ ID NO: 172 - protein, 53 aa, synthetic construct
IYIWAPLAGTCGVLLLSLVITLYCNHRNRRRVCKCPRPVVKSGDKPSLSARYV

SEQ ID NO: 173 - protein, 62 aa, synthetic construct
MALIVLGGVAGLLLFIGLGIFFCVRCRHRRRQAERMSQIKRLLSEKKTCQCPHRFQKTCSPI

SEQ ID NO: 174 - protein, 202 aa, synthetic construct
LVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 175 - protein, 187 aa, synthetic construct
GDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 176 - protein, 37 aa, synthetic construct
PPDHIPWITAVLPTVIICVMVFCLILWKWKKKKRPRS

SEQ ID NO: 177 - protein, 333 aa, synthetic construct
ILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKL

SEQ ID NO: 178 - protein, 394 aa, synthetic construct
PYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 179 - protein, 413 aa, synthetic construct
EKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 180 - protein, 353 aa, synthetic construct
CWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 181 - DNA, 1896 bp, synthetic construct
  source 1..1896: mol_type = other DNA
atgggcccacaggcttgggccagaccccttaaaaccgcccctaagtccagcgaggccatcaagctgatcctgttcatctacctgttttgcctgtttcctccaatcacacctagcgctccatcctacagctttctgctgacaagcttcacaaccggcagagtgtttgccaataccacctggaaggccggcacctccaaggaagtgtccttcgccgtggacctgtgcgccctgttccccgagcctgccagaacacacgaggaacagtgcaacctgcctgtgatgggagccggcaacgtggatctggccgctggcttcggccacaccggctcacggaccggttgtggcagctctaagggcgccgagaagggcctgcaaagcgtcgacttctacctgtgccccggcaatcaccccgacagctcttgtagagatagctaccagttcttctgccctcattggagctgtgtgaccctggctacatacagcggcggcagcaccagaagcagcaccctgagcatcaccagaaccagcagacctagaccttgcactatcagaaactgcaaccctctgacaatcaccgtgcggaatccgaattctgcccagtggtactacggcatgagctggggcctgagactgtacatcagcggcttcgacgtgggcaccatgttcacaatccagaaaaaggtgctggtgccatggagccctcctaaacccatcggccccctgaccgacctcggagatcctatgttccagaagcaccctgacagagtggacctgaccgtgcctcccccactgctggtccctaagagccagctgcagagacagcacctgcagcctagcctcatgagcatcctggatggcgttcatcacctgctcaacctgacccaacctaagctggctcaggactgctggctgtgcctgaaggctaagcccccctactacgtcggcctgggcgtggaagccatgttcaaggtgaacagcctgagctgccacacccgcccccacgccctgaccctgggcgacgtgtccggcaacgcctcttgtctgattagcgccggttataacctgagcgcctctcctttccaagccacatgcaatcaatctctcctgacaagcctgaatgcctctgtgtcttatcaggcccctaacaacacctggctggcctgcacaagcggactgacccggtgcatcaacggcacagaacctggccctctgctgtgtgtgctggtgcacgtgctgcctcaggtatacgtgtactctggacctgagggccagctgctgattgcccctcctgagctgcacagcagattcagacgggccgctccactgctggtgcccttgctcgccggactgagcatcgccggatcagctgctatcggcaccgccgccctggtgcaaggcgagaccggcctgatgagcctgagccagcaggtggatgccgacctgtccaacctgcagagcgccatcaacatgctgcacacacaggtggaaagtctggccgaggttgtgctgcagaaccggcggggcctggatctgctgtttctgtctcagggaggactgtgtgccgccctgggggagagctgctgcttctacgccaaccagagcggagttatcaaggacaccctgcaaagggtgcgggaaaacctggaccggagacagcaggagagagagaacaacacaccctggtaccagagcatgtttaactggaacccctggctgaccacactgatcaccggcctcgcaggccccctcctgctgctgctgctgggcctcgtgttcggcccttgtatcctgaactggttcctgaacttcgtgaagcagcggatcgccagcgtaaaacttacatacctgagaactcagtacaaccctctggtcatcaccgaggaatccatgatctga

SEQ ID NO: 182 - DNA, 1755 bp, synthetic construct
  source 1..1755: mol_type = other DNA
atgatctttgccggcaaggcccctagtaataccagcaccctgatgaaattctacagcctgttgctgtacagcctgctgtttagcttccccttcctgtgccacccccttccgctgccctcctacctgcatcacaccatcaacctgacacacagcctgctggctgctagcaaccctagcctcgttaacaactgctggctgtgcataagcctgagctctagcgcctatacagccgtgcctgccgtgcagacagactgggccacatctcccatcagcctgcacctcagaacttctttcaacagcccacacctgtatcctccagaagagctgatctacttcctcgacagatctagcaagacctcccctgacatctctcaccagcaggccgctgccctgctgagaacctacctgaaaaacctgagcccctacatcaacagcacccctccaatcttcggacctctgaccacccagaccaccatccctgtggccgctcctctgtgcatcagctggcagagacctaccggcatccctctgggaaacctgtctcccagcagatgcagctttacgctgcacctgcggagccccacaaccaatatcaacgagacaatcggcgccttccagctgcacatcacagataagccttctatcaacaccgataagctgaagaacatctcaagcaactactgcctgggaagacacctgccttgtatcagcctgcatccttggctgtcttctccatgtagcagtgatagcccacctagaccctctagctgcctgctgattcctagccctgaaaacaacagcgagcggctgctggtcgacacaaggagattcctgatccaccacgagaaccggacattccctagcacccagctgccacaccagagccctctgcaacctctgacagccgctgccctggccggaagcctgggagtgtgggtccaggacacccctttcagcacacctagccacctgttcaccctgcacctccagttttgcctggcccagggcctgttcttcctgtgtggcagctccacgtacatgtgcctgcctgctaattggaccggcacctgcaccctggtgttcctgacccccaagatccagttcgccaacggcacagaggaactgcccgtgcccctgatgaccccaacccaacagaagcgggtgatccccctgatccctctgatggtgggcctgggcctgtctgcctctacagtggccttaggcaccggcatcgccggcatcagcacaagcgtgatgaccttccggagcctgtctaacgacttcagcgccagcatcaccgacatctctcagactctgtccgtgctgcaggctcaggtggactctctggccgccgtggtgctgcagaaccgcagaggcctcgatctgctgaccgccgagaagggcggcctctgtatttttctgaacgaagagtgctgcttctacctgaaccagtcaggcctggtgtacgacaacatcaagaagctgaaggaccgggcccagaaactggccaatcaagcctccaattacgccgaacctccctgggctctgtccaattggatgagctgggtgctccctatcgtgtcccccctgatccccatcttcctcctgctgctgtttggcccttgtatcttcagactggtgtcccagttcatccaaaacagaattcaggccatcaccaaccacagcatcagacagatgttcctgctgaccagccctcagtaccaccctcttcctcaggatttgcctagcgcctga

SEQ ID NO: 183 - DNA, 1998 bp, synthetic construct
  source 1..1998: mol_type = other DNA
atggacccgagccacccctcccaggagtctacagctcccagcagcgtgatgggccacagccccagaggaaagagttatcagactaaggctaaggaaagcctgattctgttccacctgttctgctacagcttcttcttcccatgtgccctggcttcccatctgatcatcaacgtgaccagaagcgactctcctcaaaccatcaccttcgatgcatgcctggtcatcccctgtggcgacctgcagtctcagagacagctggccgctgctgaaaagtacctgtgcccttccgaagctgatgccagcacactgttcagcttccccttctgtcacacctgggagtacgtggtttggaccacccagagacaggactgggtccccagccaggacttcccactggccgtgctgaagccctacatccacttcaccaagggcatcgccccacctaactgcagatataaccagtgtaatcctgtgcagatctctatcacaattcctaccctgcaagatagcagccccaccctgaatagattctacggcatgggcgccgacgtgcggggcaaggaccctatcggcttcttcgagctgcacctgagcacaagcccttcactgatcagccctagactgagcagcagtacccctgccaaccagaccatcgtgtcctctagcaacgacaagtcaaaggtggcgatcgtggaagtgaagaacctgaaacagacactcacaatcgagacaggctacaaggaaacaaacgcctggatggagtggatcgaatactccgtgcggagcctgaacaaaagcgactgctacgcctgcgcccagggcagacctgaggcccaggtggtgcctttcccactgggctggtcatctgatcagcccggcatgggctgcatggtggccctctttcagcaccctacagcctgggacagcgagttctgtagaaccctgagcgtgctgttccctgagacccagcaccttgagggcgagcctcctcgggccattcaacctcctagccccgacgccaagtttaccagctgcctgagtagacagggaaagaatctggaattcctgggagatctgaagggctgcagcgaactcaagagcttccaggagctgaccaaccagagcgccctggtgcacgcccgggccgacgtgtggtggtactgcggcggacacctgctggacaccctgccttccaattggagcggaacctgcgccctgatccagctggccatccccttcaccctggccttccaacaacctgaaaagaagaaacctcagaggcggaagaccagagaggcccctcagggcagctttgactcccatgtgtacgtggacgagatcggcgtgccccagggagtgcctgaccggtttaaggccagagatcccgtggccgccggatttgagagcctgtttcccatggtcgctatcaacaagaacgtggcctggatcaactacatctactacaaccaacagagattcatcaactacacccgggacgccatccagggcatcgccgaacagctgggccctacaagccagatggcctgggaaaaccggatggctctggacatgatcctggcagagaaaggcggcgtgtgcgtgatgatcggaacacagtgctgcacctacatccctaacaataccgcccctgatggcacaataaccaaggccctgcagggcctgacctctctgtctgatgagctggctaccaacagcggcatcaccgacccctttaccggctggctgggccagtggttcggcaaatggaagggcctgatggcttctatcgttacaagcctggccattgccatcgccgtgctgatcctggttggttgttgtatcatgccatgcatcagagggctggtgcagcgactgatcgagacagcctctaacaagacattccctagctctagccagtcctactccaacaagttctttccagtgaacgagcacgagatccggatcatcctggatagattcaaggccgagcacgtgtga

SEQ ID NO: 184 - DNA, 1545 bp, synthetic construct
  source 1..1545: mol_type = other DNA
atggaccccctgcataccatcgaaaaggtgcccgccagaagaaacatccacgatagaggccaccagggacatagaatgggcgacggcacccctggcaggcctaagatcagcgtccagcagatgaccaggttcagcctgatcatcttcttcctgtctgcccctttcgtggtgaatgcctctacaagcaacgtgttcctgcagtgggcccactcctacgccgatggcctgcagcaaggcgatccttgttgggtgtgcggcagcctgcccgtgaccaacaccatggaactgccctggtgggtgtctccactgcagggcaaggactgggtctttttccagagctttatcggagatctgaagcagtggaccggtgcccagatgacaggcgtgacaagaaagaacatctccgagtggcctatcaacaagaccctgaacgagcctggccacgacaaaccttttagcgtgaacgagacacgggacaaggtgatcgccttcgccatccctctgctggacaccaaggtgttcgtgcagaccagcagacctcagaacacccagtaccggaatggcttcctgcagatctgggacggattcatctggctgaccgccacaaagggccacctgagccagattgccccactgtgttgggaacagagaaaccacagcctggacaactggcctaacaccacaagagtgatgggctggatcccgccaggacagtgcagacacaccatcctgctgcagcagcgggacctgttcgccaccgactggtctcagcagcctggcctgaactggtacgcccctaacggcacacagtggctgtgcagccctaacctgtggccctggctccccagcggctggctgggctgctgcacactgggaataccttgggctcaaggaagatgggttaaaacaatggaagtgtatccttacctgccccacgtggtcaaccagggaacgcgggccatagtgcaccggaacgaccacctgcccaccatctttatgcctagcgtgggcctgggcaccgtgatccagcacatcgaggccctggctaatttcacccagagagccctgaatgactccctgcaatctatttctcttatgaacgccgaggtgtactacatgcacgaggacatcctgcaaaaccggatggccctggatattctgacagccgctgaaggcggcacctgcgccctgatcaagacagagtgctgcgtgtacatccctaacaacagccggaatatcagcctggccctggaagatacctgtcgacagatccaggtgatctccagcagcgccctcagccttcacgactggatcgccagccaattctctggcagaccaagctggtggcagaaaatcctgatcgtgctggcaaccctgtggtccgtgggcatcgctctctgttgcggcctgtacttctgcagaatgttcagccaacacatcccccagacccacagtatcatctttcagcaggagctgcctctgagccccccttctcaggagcactaccagagccaaagagatatcttccacagcaatgccccttga

SEQ ID NO: 185 - DNA, 1815 bp, synthetic construct
  source 1..1815: mol_type = other DNA
atgctgggcatgaacatgctcctgatcaccctgttcctgctgctgcctctgagcatgctgaaaggcgaaccttgggagggctgcctgcactgcacccacaccacctggagcggcaacatcatgaccaagacactgttgtaccacacctactacgagtgcgccggtacatgtctgggcacctgtacacacaaccagacaacatactctgtctgcgaccctggcagaggccaaccttacgtgtgctacgaccccaagagcagccccggcacctggttcgagatccacgtcggcagcaaggaaggcgatctgctgaatcagaccaaggtgttcccctccggcaaagatgtggtgtctctgtacttcgacgtgtgccagatcgtgtccatgggctctctgtttccagtgatcttcagcagcatggaatactatagcagctgccacaagaacagatacgcccatcctgcctgcagcacagacagccccgtgaccacctgttgggactgcaccacatggtccacaaatcagcaatctctgggccctatcatgctgaccaagatccccctggagcctgattgcaagaccagcacctgcaacagcgtgaacctgaccatcctggagcctgaccagcctatctggaccacaggcctgaaggcccctctgggcgcccgggtgtccggagaagaaatcggcccaggagcctacgtgtacctgtatatcatcaagaagactagaaccagaagcacccagcaatttagagtgttcgagtctttctatgagcacgttaaccagaagctgcctgagcccccccccctggcctccaatctgttcgcccagctggcagaaaacatcgctagctctctgcacgtggccagttgttacgtgtgtggcggcatgaacatgggagatcagtggccttgggaagctagagaactgatgccccaggacaacttcaccctcaccgcctcctctctggagcctgctcctagcagccagagcatctggtttctgaagaccagcatcattggcaagttctgcatcgccagatggggcaaggcctttaccgatcctgtgggcgaactgacatgtctgggccagcagtactacaacgagacactgggaaaaacactttggcggggaaaaagcaacaacagcgagagcccccatccgagccctttttcaagattccccagcctgaaccactcttggtaccagctcgaggccccaaacacctggcaggccccaagcggactgtactggatctgcggccctcaggcctacagacagctgcccgccaagtggagcggcgcttgtgtgctgggaacgatccggcctagcttcttcctgatgcctctgaagcagggcgaggccctgggctaccctatctacgatgagacaaagaggaagagtaagcggggaatcacgattggcgactggaaggacaatgagtggcctcctgaaagaatcatccaatactacggcccagccacctgggccgaggacggcatgtggggctaccggacccctgtctacatgctgaaccggatcatcagactgcaggccgtgctggaaatcatcaccaacgagacagccggggccctgaacctgctggctcagcaggccacaaagatgcggaacgtaatctatcagaacagactggctctggactacctgctggcccaggaggaaggcgtgtgcggcaagttcaatctgaccaattgctgcctcgaactggacgacgagggcaaagtgatcaaagagattaccgctaagatccagaaactggcccacatccctgtgcaaacctggaagggctga

SEQ ID NO: 186 - DNA, 1434 bp, synthetic construct
  source 1..1434: mol_type = other DNA
atgaccgagaagttcctgttcctgtacctctctctcctgcctatgcctctgctgtctcaggcccagtggaacgagaacagcctggtttctttttccaaaatcatcgccagcggcaaccacctgtctaattgctggatctgccacaactttatcaccagaagcagcagctaccagtacatcctggtcagaaatttcagcctgaatctgaccttcggctctggaatccctgagggccagcacaaaagcgtgcccctgcaagtgtccctggctaatagcgcccaccaggtgccgtgcctggacctgacccctcctttcaaccagagctccaaaaccagcttttacttctacaactgcagctccctgaaccagacctgttgtccttgtcctgaaggacactgcgacagaaagaacacaagcgaggaaggcttcccctcccctaccatccaccctatgagcttcagccccgccggttgtcaccccaacctgacccactggtgccccgccaaacagatgaatgactacagagataagtccccacagaacagatgcgccgcttgggagggaaaggaactgatcacatggcgcgtgctgtatctgcttcctaaggcccatacagtgcctacatggcccaagtctaccgtgccactgggaggacctctgagccccgcctgcaaccaaacaatccctgccggctggaagagccagctgcacaagtggttcgacagccacatccccagatgggcctgtaccccaccaggctacgtgttcctgtgcggccctcagaaaaacaagctgcccttcgacggctctcctaagatcacttacagcacccctcctgtggccaacctgtacacatgtatcaacaacatccaacacacaggcgagtgcgccgtgggcctgctaggccctaggggcatcggagttacaatctacaacaccacccagcctcggcagaagcgggccctgggcctgattctggccggcatgggagctgctatcggcatgatcgccccatggggcggtttcacctaccacgacgtgaccctgcggaacctgtctagacagatcgacaacatcgccaagtccaccagagatagcattagcaagctgaaggccagcatcgatagcctggccaacgtggtgatgaacaaccggctggccttggattatctgctggctgaacagggcggcgtgtgcgccgtcatcagcaagtcctgctgcatctacgtgaacaatagcggcgccatcgaggaagatatcaagaagatctacgacgaggtgacctggctgcataattttggcaagggcgactctgctggcagcatctgggaggccgtgaagagcgccctgcctagcctgacatggttcgtgcccctcctgggccccgcagccctgaactctctgctgagccctctgtggcctctgtccctgtga

SEQ ID NO: 187 - DNA, 1617 bp, synthetic construct
  source 1..1617: mol_type = other DNA
atgggtcttctgctgctcgtgctgatcctgacacctagcctggccgcatatagacaccccgacttccccttactggaaaaggcccagcagctgctccagagcaccggatctccatacagcactaactgctggctgtgcacaagctccagcacggaaacccccggcaccgcctatcctgccagccctcgggaatggacatccatcgaggccgagctgcacatctcttacagatgggaccccaacctgaaaggcctgatgcggcctgctaatagccttctgtccaccgtgaagcaggattttccagatattagacagaagccccccatcttcggccccatcttcaccaatatcaacctgatgggcattgcccctatatgcgtgatggctaagagaaaaaacggcaccaacgtgggcaccctgccttctacagtgtgcaacgtgacattcacagtggacagcaaccaacagacctaccagacctacacccacaaccagttcagacaccaacctcgcttcccaaagcctccaaacatcaccttccctcagggcaccctgctggacaagagcagcagattctgccagggcagacctagcagctgcagcacaagaaatttctggttccggcccgccgattacaaccagtgtctgcagatcagcaacctgagcagcaccgccgagtgggtgctgctggaccagacccggaacagcctgttctgggaaaacaagacaaagggcgccaaccagagccagacaccttgtgtgcaagtgcttgccggaatgaccatcgctacaagctacctgggcatcagcgccgttagcgagttcttcggcacctctctgacccctctgttccacttccacatcagcacctgcctgaagacccagggagccttctacatctgcggccagagcatccaccagtgtctgccatctaattggaccggcacatgtaccatcggctacgtgacccctgatatctttatcgcccctggaaacctgtctctgccgatccctatctacggcaatagccccctgcctagagtgcggcgggccatccactttatccccctgctggccgggctgggcatcctggccggcaccggcacaggcatcgccggcatcaccaaggcctctctgacatacagccagctgagcaaggaaatcgccaacaacatcgacaccatggctaaagccctgaccaccatgcaggagcagatcgacagcctggctgccgtggtgctgcagaacagaaggggcctggacatgctgaccgccgctcagggcggaatctgtctggccctcgacgagaagtgctgcttctgggtcaatcagagcggcaaggtgcaggacaacatccggcagctgctgaaccaggcctcctctcttagagagagagccacacagggatggctgaactgggagggcacatggaagtggttcagctgggtcctgcctctgaccggacctctggtgtctctgctgctgctgctgctgtttggcccttgcctcctgaatctgatcacccaatttgtgtcctccagactgcaagctatcaaactgcaaaccaacctgtctgccggaagacatcctagaaacatccaggagagccctttctga

SEQ ID NO: 188 - DNA, 1692 bp, synthetic construct
  source 1..1692: mol_type = other DNA
atgggcagcctgagcaattacgcccttctgcagctgacgctgactgccttcctgaccatcctggtgcagccccagcacctgctggcccctgtgttccggacactgagtatcctgactaaccagagcaactgctggctgtgcgagcacctggataacgccgagcagcctgagctggtctttgtgccagcctctgcctcgacctggtggacctacagcggccagtggatgtacgagagagtgtggtacccccaggccgaagtgcagaaccacagcaccagcagttatagaaaggtgacatggcattgggaggctagcatggaagctcagggcctgagctttgcccaggtgcggctgctcgagggcaacttcagcctgtgcgtggaaaacaagaacggctctggacctttcctgggcaatatccctaagcagtactgcaaccagatcctgtggttcgacagcaccgatgggaccttcatgcctagcatcgacgtgaccaacgagagcagaaatgatgatgacgacacatctgtgtgcctgggcaccagacagtgtagctggttcgccggctgcacaaacaggacctggaacagcagcgccgtgcctctgatcggcttgcctaacacccaggactacaagtgggtcgacagaaacagcggactgacctggtccggcaacgacacatgcctgtactcttgtcagaatcaaaccaagggcctgctgtaccagctgttccggaacctgttctgcagctacggcctgacagaggctcatggaaaatggcggtgcgccgacgccagcattaccaacgacaagggacacgacggccacagaacccctacctggtggctgaccggcagcaatctgaccctgtctgtgaacaacagcggcctgttcttcctgtgtggtaacggcgtgtacaagggcttcccccccaagtggagcggcagatgtggccttggatatctggttccaagcctgacacgctacctgaccctgaatgccagccaaatcaccaacctgagaagcttcatccacaaggtgacccctcaccggtgcacccaaggcgacaccgacaacccccccctgtactgtaacccaaaagataacagcaccatcagagccctgttcccatccctgggaacctacgacctggaaaaggccatcctgaatatcagcaaggccatggaacaggagttcagcgctacaaagcagaccctggaagcccaccagtctaaggtttccagcctggcctccgctagcagaaaggaccacgtgctggacatccctacaacgcaacggcagacagcctgtggcacagtgggcaaacagtgctgcctgtacatcaactacagcgaggaaatcaagagcaacattcagagactgcacgaggcttccgagaacctgaaaaacgtgcctctgctggattggcagggcatcttcgccaaggtgggcgattggtttagatcttggggctacgtgctcctgatcgtgctgttttgcctgtttatcttcgtgctgatctacgtgcgggtgttcagaaagtctcgcagatccctgaactctcagcctctgaatctggcactgagccctcagcagagcgcccaactgctcgtatctgaaaccagctgccaggtgtccaaccgggccatgaaaggcctgacaacccaccagtacgacacctccctgctctga

SEQ ID NO: 189 - DNA, 2100 bp, synthetic construct
  source 1..2100: mol_type = other DNA
atgaacccatcggagatgcaaagaaaagcacctccgcggagacggagacaccgcaatcgagcaccgttgactcacaagatgaacaaaatggtgacgtcagaagaacagatgaagttgccatccaccaagaaggcagagccgccgacttgggcacaactaaagaagctgacgcagttagctacaaaatatctagagaacacaaaggtgacacaaaccccagagagtatgctgcttgcagccttgatgattgtatcaatggtggtaagtctccctatgcctgcaggagcagctgcagctaactatacctactgggcctatgtgcctttcccgcccttaattcgggcagtcacatggatggataatcctatagaagtatatgttaatgatagtgtatgggtacctggccccatagatgatcgctgccctgccaaacctgaggaagaagggatgatgataaatatttccattgggtatcgttatcctcctatttgcctagggagagcaccaggatgtttaatgcctgcagtccaaaattggttggtagaagtacctactgtcagtcccatcagtagattcacttatcacatggtaagcgggatgtcactcaggccacgggtaaattatttacaagacttttcttatcaaagatcattaaaatttagacctaaagggaaaccttgccccaaggaaattcccaaagaatcaaaaaatacagaagttttagtttgggaagaatgtgtggccaatagtgcggtgatattacaaaacaatgaatttggaactattatagattgggcacctcgaggtcaattctaccacaattgctcaggacaaactcagtcgtgtccaagtgcacaagtgagtccagctgttgatagcgacttaacagaaagtttagacaaacataagcataaaaaattgcagtctttctacccttgggaatggggagaaaaaggaatctctaccccaagaccaaaaatagtaagtcctgtttctggtcctgaacatccagaattatggaggcttactgtggcctcacaccacattagaatttggtctggaaatcaaactttagaaacaagagatcgtaagccattttatactgtcgacctaaattccagtctaacagttcctttacaaagttgcgtaaagcccccttatatgctagttgtaggaaatatagttattaaaccagactcccagactataacctgtgaaaattgtagattgcttacttgcattgattcaacttttaattggcaacaccgtattctgctggtgagagcaagagagggcgtgtggatccctgtgtccatggaccgaccgtgggaggcctcaccatccgtccatattttgactgaagtattaaaaggtgttttaaatagatccaaaagattcatttttactttaattgcagtgattatgggattaattgcagtcacagctacggctgctgtagcaggagttgcattgcactcttctgttcagtcagtaaactttgttaatgattggcaaaaaaattctacaagattgtggaattcacaatctagtattgatcaaaaattggcaaatcaaattaatgatcttagacaaactgtcatttggatgggagacagactcatgagcttagaacatcgtttccagttacaatgtgactggaatacgtcagatttttgtattacaccccaaatttataatgagtctgagcatcactgggacatggttagacgccatctacagggaagagaagataatctcactttagacatttccaaattaaaagaacaaattttcgaagcatcaaaagcccatttaaatttggtgccaggaactgaggcaattgcaggagttgctgatggcctcgcaaatcttaaccctgtcacttgggttaagaccattggaagtactacgattataaatctcatattaatccttgtgtgcctgttttgtctgttgttagtctgcaggtgtacccaacagctccgaagagacagcgaccatcgagaacgggccatgatgacgatggcggttttgtcgaaaagaaaagggggaaatgtggggaaaagcaagagagatcagattgttactgtgtctgtgtag

SEQ ID NO: 190 - DNA, 1617 bp, synthetic construct
  source 1..1617: mol_type = other DNA
atggccctgccttaccacatcttcctgttcaccgtgctgctgccttcttttacactgaccgcccctccaccttgcagatgcatgaccagctcctccccttatcaggagttcctgtggcggatgcagagacctggaaatatcgacgcccctagctaccggagcctcagcaagggcacacctacatttaccgcccacacgcatatgcctagaaactgctaccacagcgccaccctctgtatgcacgccaacacacactactggacaggaaagatgatcaacccctcctgccccggcggactgggcgtgaccgtgtgttggacctacttcacacagaccggcatgagcgatggcggcggtgttcaggaccaggcccgggagaagcacgtgaaagaggtgatctctcaactgacccgggtgcacggcaccagcagcccctacaagggcctggatctgagcaaactgcacgagacactgcggacccacaccagactggtgtctcttttcaacaccaccctgaccggcctgcatgaagtgagcgcccagaatcccacaaactgctggatctgcctgcctctgaatttcagaccctacgtgagcatccccgtgcctgagcagtggaacaacttcagcacagagattaacaccaccagcgtgctggtgggccctctcgtgagcaacctggaaatcacacacaccagcaacctgacctgtgtgaagttcagcaacaccacatacacaaccaacagccagtgcatcagatgggtcacccctcctacccagatcgtgtgcctgccatctggcatctttttcgtgtgcggcacctctgcatataggtgtctgaacggatctagtgagagcatgtgcttcctgagcttcctggtgccccccatgaccatctacaccgagcaggacctgtacagctacgtaatttctaaacctagaaacaagcgggtgcccatcctgccttttgtgatcggcgccggcgtgctgggagccctgggaaccggcatcggaggcatcaccacatctacccagttctactacaagctgtctcaggagctgaacggcgacatggaacgggtggccgacagcctggtcacactgcaagatcagctgaactctctggctgccgtggtgctgcagaacagaagagccctggacctgctgaccgccgagagaggcggcacatgtctgtttctgggcgaggaatgctgctactacgtgaaccagtccggcatcgtcacagagaaagtgaaggaaatccgggacagaatccagcggagagccgaagagctgagaaatactggaccttggggcctgctgtcccaatggatgccctggatcctgccattcctgggccctctggccgctatcatcctgctgctcctgttcggcccttgtatcttcaacctgctggttaatttcgtgtccagcagaatcgaggccgtgaagctgcagatggaacccaagatgcagagcaagaccaagatctaccgccggcctttggatagacccgccagccctagatccgacgtgaacgacatcaagggcacacctccagaagaaattagcgctgctcagcctctactgagacctaacagcgctggcagctcctga

SEQ ID NO: 191 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQNKAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 192 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVKNKAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 193 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSNGGGVQRQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 194 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSNGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 195 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQRQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 196 - protein, 699 aa, Homo sapiens
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDACPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 197 - protein, 699 aa, Homo sapiens
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDCCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 198 - DNA, 21 bp, synthetic construct
  source 1..21: mol_type = other DNA
gtttatcacaggctccaggaa

SEQ ID NO: 199 - DNA, 20 bp, synthetic construct
  source 1..20: mol_type = other DNA
ggtgagtgagtgtgtgcgtg

SEQ ID NO: 200 - protein, 180 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVADQGAEKHVKEVISQLTRVHGTSSPYKGLDLSKLHETLRTHTRLVSLFNTTLTGLHEVSA

SEQ ID NO: 201 - protein, 180 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQGAEKHVKEVISQLTRVHGTSSPYKGLDLSKLHETLRTHTRLVSLFNTTLTGLHEVSA

SEQ ID NO: 202 - protein, 180 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQGREKHVKEVISQLTRVHGTSSPYKGLDLSKLHETLRTHTRLVSLFNTTLTGLHEVSA

SEQ ID NO: 203 - protein, 180 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGGQDQAREKHVKEVISQLTRVHGTSSPYKGLDLSKLHETLRTHTRLVSLFNTTLTGLHEVSA

SEQ ID NO: 204 - protein, 180 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGGQAAAAEKHVKEVISQLTRVHGTSSPYKGLDLSKLHETLRTHTRLVSLFNTTLTGLHEVSA

SEQ ID NO: 205 - protein, 180 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGGQAQAAEKHVKEVISQLTRVHGTSSPYKGLDLSKLHETLRTHTRLVSLFNTTLTGLHEVSA

SEQ ID NO: 206 - protein, 180 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGGQDQAAEKHVKEVISQLTRVHGTSSPYKGLDLSKLHETLRTHTRLVSLFNTTLTGLHEVSA

SEQ ID NO: 207 - protein, 180 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAAEKHVKEVISQLTRVHGTSSPYKGLDLSKLHETLRTHTRLVSLFNTTLTGLHEVSA

SEQ ID NO: 208 - protein, 180 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGGQDAAAEKHVKEVISQLTRVHGTSSPYKGLDLSKLHETLRTHTRLVSLFNTTLTGLHEVSA

SEQ ID NO: 209 - protein, 180 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGGQDAAREKHVKEVISQLTRVHGTSSPYKGLDLSKLHETLRTHTRLVSLFNTTLTGLHEVSA

SEQ ID NO: 210 - protein, 180 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDAAREKHVKEVISQLTRVHGTSSPYKGLDLSKLHETLRTHTRLVSLFNTTLTGLHEVSA

SEQ ID NO: 211 - protein, 180 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVAAAAGEKHVKEVISQLTRVHGTSSPYKGLDLSKLHETLRTHTRLVSLFNTTLTGLHEVSA

SEQ ID NO: 212 - protein, 180 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVAAAAREKHVKEVISQLTRVHGTSSPYKGLDLSKLHETLRTHTRLVSLFNTTLTGLHEVSA

SEQ ID NO: 213 - protein, 180 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVAAQAREKHVKEVISQLTRVHGTSSPYKGLDLSKLHETLRTHTRLVSLFNTTLTGLHEVSA

SEQ ID NO: 214 - protein, 180 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQAQAREKHVKEVISQLTRVHGTSSPYKGLDLSKLHETLRTHTRLVSLFNTTLTGLHEVSA

SEQ ID NO: 215 - protein, 180 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVADAAAEKHVKEVISQLTRVHGTSSPYKGLDLSKLHETLRTHTRLVSLFNTTLTGLHEVSA

SEQ ID NO: 216 - protein, 180 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVADAAREKHVKEVISQLTRVHGTSSPYKGLDLSKLHETLRTHTRLVSLFNTTLTGLHEVSA

SEQ ID NO: 217 - protein, 180 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSA

SEQ ID NO: 218 - protein, 180 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVADQAREKHVKEVISQLTRVHGTSSPYKGLDLSKLHETLRTHTRLVSLFNTTLTGLHEVSA

SEQ ID NO: 219 - protein, 419 aa, synthetic construct
  VARIANT 22: note = Any amino acid
  VARIANT 23: note = Any amino acid
  VARIANT 24: note = Any amino acid
  VARIANT 25: note = Any amino acid
MALPYHIFLFTVLLPSFTLTAXXXXPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 220 - protein, 378 aa, synthetic construct
  VARIANT 22: note = Any amino acid
  VARIANT 23: note = Any amino acid
  VARIANT 24: note = Any amino acid
  VARIANT 25: note = Any amino acid
MALPYHIFLFTVLLPSFTLTAXXXXCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 221 - protein, 531 aa, synthetic construct
  VARIANT 115: note = Any amino acid
  VARIANT 116: note = Any amino acid
  VARIANT 117: note = Any amino acid
  VARIANT 118: note = Any amino acid
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMXXXXEKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 222 - protein, 538 aa, synthetic construct; identical to SEQ ID NO: 128

SEQ ID NO: 223 - protein, 538 aa, Homo sapiens; identical to SEQ ID NO: 129

SEQ ID NO: 224 - protein, 604 aa, Homo sapiens; identical to SEQ ID NO: 130

SEQ ID NO: 225 - protein, 514 aa, Homo sapiens; identical to SEQ ID NO: 131

SEQ ID NO: 226 - protein, 41 aa, synthetic construct
MALPVTALLLPLALLLHAARPEQKLISEEDLGSSGSGSAVS

SEQ ID NO: 227 - protein, 65 aa, synthetic construct
MLTRTLAVRSFAATMSPVKGGTKCIKYLLFGFNFIFWLAGIAVLAIGLWLRFDSQTKSIFEQETN

SEQ ID NO: 228 - protein, 45 aa, synthetic construct
MLTRTLAVRSFAATMALPVTALLLPLALLLHAARPEQKLISEEDL

SEQ ID NO: 229 - protein, 45 aa, synthetic construct; identical to SEQ ID NO: 228

SEQ ID NO: 230 - protein, 45 aa, synthetic construct; identical to SEQ ID NO: 228

SEQ ID NO: 231 - protein, 50 aa, synthetic construct
MLTRTLAVRSFAATMAVEGGMKCVKFLLYVLLLAFCACAVGLIAVGVGAQ

SEQ ID NO: 232 - protein, 63 aa, synthetic construct
MLTRTLAVRSFAATMGVEGCTKCIKYLLFVFNFVFWLAGGVILGVALWLRHDPQTTNLLYLEL

SEQ ID NO: 233 - protein, 45 aa, synthetic construct; identical to SEQ ID NO: 228

SEQ ID NO: 234 - protein, 45 aa, synthetic construct; identical to SEQ ID NO: 228

SEQ ID NO: 235 - protein, 21 aa, synthetic construct
MALPYHIFLFTVLLPSFTLTA

SEQ ID NO: 236 - protein, 114 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGM

SEQ ID NO: 237 - protein, 21 aa, synthetic construct
MALPYHIFLFTVLLPSFTLTA

SEQ ID NO: 238 - protein, 4 aa, unidentified
CFFC

(19) United States
(12) Patent Application Publication
(10) Pub. No.: US 2024/0189247 A1
(43) Pub. Date: Jun. 13, 2024

(54) Minimal Human-Derived Virus-Like Particles and Methods of Use Thereof for Delivery of Biomolecules

(71) Applicant: The General Hospital Corporation, Boston, MA (US)

(72) Inventors: J. Keith Joung, Winchester, MA (US); Peter Cabeceiras, Boston, MA (US)

(21) Appl. No.: 18/511,486

(22) Filed: Nov. 16, 2023

Related U.S. Application Data
(60) Provisional application No. 63/425,900, filed on Nov. 16, 2022.

(51) Int. Cl. A61K 9/51; A61K 48/00; C07K 14/005; C07K 14/47; C12N 9/22; C12N 15/11; C12N 15/86

(52) U.S. Cl. CPC A61K 9/5184; A61K 9/5192; A61K 48/0033; C07K 14/005; C07K 14/47; C12N 9/22; C12N 15/11; C12N 15/86; C07K 2317/569; C07K 2317/622; C07K 2319/30; C12N 2310/20; C12N 2740/10022; C12N 2740/10023; C12N 2740/10043; C12N 2740/10052; C12N 2800/80

(57) ABSTRACT

Described herein are virus-like particles (VLPs) and minimal human-derived virus-like particles (mhVLPs), comprising a membrane comprising a phospholipid bilayer with one or more human-derived envelope glycoproteins (env) on the external side. Optionally, a biomolecule cargo is disposed in the core of the VLP or mhVLP on the inside of the membrane. Preferably, the mhVLPs do not comprise any exogenous virally derived proteins, e.g., proteins from viral gag, pro, or pol, or other viral proteins that reside inside of enveloped particles (unless the cargo comprises the viral protein(s)). In some embodiments, the mhVLPs do not comprise any human endogenous retroviral (HERV) proteins other than the env (hENV), e.g., do not comprise gag, pol, or pro that was exogenously introduced into producer cells. In some embodiments, the VLPs include a targeting domain, either fused at the N or C terminus or internally into the hENV, or as a separate membrane-anchored targeting domain. Also described are methods of use of the VLPs or mhVLPs for delivery of the biomolecule cargo to cells.

### CLAIM OF PRIORITY

This application claims the benefit of U.S. Provisional Patent Application Ser. No. 63/425,900, filed on Nov. 16, 2022. The entire contents of the foregoing are hereby incorporated by reference.

### FEDERALLY SPONSORED RESEARCH OR DEVELOPMENT

This invention was made with Government support under Grant No. GM118158 awarded by the National Institutes of Health. The Government has certain rights in the invention.

### SEQUENCE LISTING

The instant application contains a Sequence Listing which has been submitted electronically in XML file format and is hereby incorporated by reference in its entirety. Said XML copy, created on Feb. 22, 2024, is named 29539-0678001_SL.xml and is 297,563 bytes in size.

### TECHNICAL FIELD

Described herein are virus-like particles (VLPs) and minimal human-derived virus-like particles (mhVLPs), including targeted human endogenous virus-like particles (theVLPs), comprising a membrane comprising a phospholipid bilayer with one or more human-derived envelope glycoproteins (env) on the external side. Optionally, a biomolecule cargo is disposed in the core of the VLPs and mhVLPs on the inside of the membrane. Preferably, the VLPs and mhVLPs do not comprise any exogenous virally derived proteins, e.g., proteins from viral gag, pro, or pol, or other viral proteins that reside inside of enveloped particles (unless the cargo comprises the viral protein(s)). In some embodiments, the VLPs and mhVLPs do not comprise any human endogenous retroviral (HERV) proteins other than the env, e.g., do not comprise gag, pol, or pro. Also described are methods of use of the VLPs and mhVLPs for delivery of the biomolecule cargo to cells.

### BACKGROUND

Delivery of cargo such as proteins, nucleic acids, and/or chemicals into the cytosol of living cells has been a significant hurdle in the development of biological therapeutics.

### SUMMARY

Described herein are virus-like particles (VLPs) and minimal human-derived virus-like particles (mhVLPs), including targeted human endogenous virus-like particles (theVLPs) that are capable of packaging and delivering a wide variety of payloads, e.g., biomolecules including nucleic acids (DNA, RNA) or proteins, chemical compounds including small molecules, and/or other molecules, and any combination thereof, into eukaryotic cells. The non-viral mhVLP systems described herein have the potential to be simpler, more efficient, safer, and/or less immunogenic than conventional, artificially-derived lipid/gold nanoparticles and viral particle-based delivery systems, because mhVLPs are comprised of human-derived components that are expressed in healthy human tissues in their unmodified forms, mhVLPs can also utilize but do not require chemical-based dimerizers, and mhVLPs have the ability to package and deliver cargo including, but not limited to, biomolecules including nucleic acids, e.g., specialty single and/or double-stranded DNA molecules (e.g., plasmid, mini circle, closed-ended linear DNA, AAV DNA, episomes, bacteriophage DNA, homology directed repair templates, etc.), single and/or double-stranded RNA molecules (e.g., single guide RNA, prime editing guide RNA, crRNA, tracrRNA, messenger RNA, transfer RNA, long non-coding RNA, circular RNA, RNA replicon, circular or linear splicing RNA, micro RNA, small interfering RNA, short hairpin RNA, piwi-interacting RNA, toehold switch RNA, RNAs that can be bound by RNA binding proteins, bacteriophage RNA, or internal ribosomal entry site containing RNA); proteins; chemical compounds and/or molecules, and combinations of the above listed cargos (e.g., AAV particles and/or ribonucleoprotein (RNP) complexes comprising RNA and protein, e.g., guide RNA/CRISPR Cas protein complexes). The mhVLPs described herein are different from conventional retroviral particles, virus-like particles (VLPs), exosomes and other previously described extracellular vesicles that can be loaded with cargo, at least because of the membrane configuration, HERV-derived envelope glycoprotein, vast diversity of possible cargos that are enabled by novel, innovative loading strategies, the lack of a limiting DNA/RNA length constraint, the lack of proteins derived from any viral gag, pro, or pol, and/or the mechanism of cellular entry.

Provided herein are virus-like particles (VLPs) that comprise mutant and truncated HERV glycoproteins/envelope proteins (hENV). In some embodiments, the hENV comprise a sequence that is at least 95% identical to a sequence as set forth herein, e.g., in Tables 1A, 1B, and/or 1C, optionally comprising a mutation as identified in Table 1B, and/or a truncation and/or targeting domain insertion as shown in Table 1C, or a combination thereof. In some embodiments, the truncated hENV comprises a C-terminal deletion of between 1 and 60 amino acids, or a C-terminal deletion that partially or entirely removes the intracellular domain. In some embodiments, the hENV comprises a mutation in the receptor binding domain, e.g., in an amino acid corresponding to amino acids 120-125 of the wild type HERV-W sequence set forth herein (e.g., a mutation as shown in Table 1B). In some embodiments, the hENV comprise a targeting domain that alters tropism of the particles, e.g., inserted into or fused to the N or C terminus of the hENV, e.g., as shown in Table 1C (e.g., a programmable tropism HERV env (pthENV). Optionally, linkers can be present between any or all of the parts of the fusion proteins.

In some embodiments, the targeting domain comprises a targeting peptide, e.g., as shown in Table A. In some embodiments, the targeting domain comprises a single chain variable fragment (scFv), nanobody, fibronectin type 3 domain (FN3), arginylglycylaspartic acid motif (RGD), single variable domain on a heavy chain/nanobody (VHH), variable domain of new antigen receptor (VNAR), or darpin.

In some embodiments, the Targeting Domain binds to human CD19, CD4, CD34, ASGR1, TfR1, HER2, CD25, CTLA-4, HB-EGF, ACE2, Aryl hydrocarbon receptor (AhR), keratin 5 (KRT5), KRT13, Fibronectin (FN1), Amyloid precursor protein (APP), neurotrophin receptor (p75NTR), Thy-1/CD90, EpCAM, and/or CFTR.

In some embodiments, the targeting domain is inserted internally into the hENV, optionally after an amino acid corresponding to amino acid 18 or amino acid 114 of wild type HERV W. In some embodiments, the hENV further comprises one or more of: a deletion of 123 to 163 amino acids following amino acid 18; truncation of one to 50 amino acids from the C RBD mutations. In some embodiments, the signal sequence comprises MKCLLYLAFLFIGVNCK (SEQ ID NO:1) or a secretion signal sequence that is derived from VSVG (e.g., MKCLLYLAFLFIGVNC, SEQ ID NO:2), or another signal sequence as known in the art or described herein.

Also provided herein are nucleic acids encoding the hENVs, and vectors comprising the nucleic acids, optionally operably linked to a promoter for expression of the hENV, as well as host (production) cells comprising the nucleic acids, and optionally expressing the hENV. In some embodiments, the nucleic acids are codon-optimized for expression in humans.

Further, provided herein are virus-like particles (VLPs) comprising a hENV as described herein. Additionally provided are targeted human endogenous virus-like particle (theVLP) comprising a human endogenous retroviral (HERV) envelope protein (hENV) and a targeting domain, wherein the hENV is at least 95% identical to a sequence as set forth in Tables 1A-C, and optionally comprises a truncation of one to 50 amino acids from the C terminus and/or one or more RBD mutations, wherein the targeting domain is (i) fused at the N or C terminus, or inserted internally into the of the hENV, or (ii) is a membrane-anchored targeting domain comprising a targeting domain fused to a transmembrane domain, with optional linkers therebetween.

Optionally, a cargo is disposed in the core of the theVLP; optionally the cargo is fused to a phospholipid bilayer recruitment domain.

Also provided are minimal human-derived virus-like particles (mhVLPs), comprising a membrane comprising a phospholipid bilayer and a human endogenous retroviral (HERV) envelope protein (hENV), wherein the hENV optionally comprises one, two, or all three of: a targeting domain at the N or C terminus, or inserted internally into the protein; a truncation of one to 50 amino acids from the C terminus; and/or one or more RBD mutations; and optionally, a cargo disposed in the core of the mhVLP, wherein the cargo is optionally fused to a phospholipid bilayer recruitment domain; preferably wherein the mhVLP does not comprise an exogenous gag, pro and/or pol protein, and optionally wherein the mhVLP further comprises a separate targeting domain. In some embodiments, the mhVLPs do or do not comprise any human endogenous retroviral (HERV) proteins other than the env, e.g., do not comprise gag, pol, or pro (Grandi and Tramontano, Front Microbiol. 2018 Mar. 14; 9:462). Exogenous virally-derived gag, pol, or pro refers to any gag, pro, pol, gag-pol, gag-pro-pol, and/or pol protein, or any other protein expressed from gag, pro, or pol, from any virus introduced into the cell.

In some embodiments, the targeting domain comprises an single chain variable fragment (scFv), nanobody, fibronectin type 3 domain (FN3), arginylglycylaspartic acid motif (RGD), single variable domain on a heavy chain/nanobody (VHH), variable domain of new antigen receptor (VNAR), or darpin. In some embodiments, the targeting domain is inserted internally into the hENV, optionally after an amino acid corresponding to amino acid 18 or amino acid 114 of wild type HERV W env. In some embodiments, the hENV further comprises one or more of: a deletion of 123 to 163 amino acids following amino acid 18; truncation of one to 50 amino acids from the C RBD mutations.

In some embodiments, the cargo is a therapeutic or diagnostic protein, and/or nucleic acid encoding a therapeutic or diagnostic protein, and/or a chemical, optionally a small molecule therapeutic or diagnostic. In some embodiments, the cargo is a gene editing or epigenetic modulating reagent. In some embodiments, the gene editing or epigenetic modulating reagent comprises a zinc finger (ZF), transcription activator-like effector (TALE), and/or CRISPR-Cas protein, variant, or fusion thereof; a nucleic acid encoding a zinc finger (ZF), transcription activator-like effector (TALE), and/or CRISPR-Cas protein, variant, or fusion thereof; a guide RNA and/or crRNA; or a ribonucleoprotein complex (RNP) comprising a CRISPR-Cas protein, variant, or fusion thereof and optionally a guide RNA and/or crRNA. In some embodiments, the cargo is selected from the proteins listed in Tables 2, 3, 4 & 5, or that is at least 95% identical to a sequence set forth herein, e.g., in Tables 2, 3, 4, and 5. In some embodiments, the cargo comprises a CRISPR-Cas protein, and the theVLP or VLP further comprises one or more guide RNAs and/or crRNAs that bind to and direct the CRISPR-Cas protein to a target nucleic acid sequence. In some embodiments, the cargo comprises a fusion to a phospholipid bilayer recruitment domain, preferably as shown in Table 6, or that is at least 95% identical to a sequence set forth herein in Table 6.

Also provided are methods of delivering a cargo to a target cell, optionally a cell in vivo or in vitro, the method comprising contacting the cell with a theVLP or mhVLP as described herein comprising the cargo.

Additionally provided herein are methods of producing a theVLP or an mhVLP, optionally comprising a cargo. The methods comprise providing a cell expressing a hENV as described herein and optionally a cargo, optionally wherein the cell does not express a human endogenous and/or exogenous any exogenous virally derived proteins, e.g., proteins from viral gag, pro, or pol, or other viral proteins that reside inside of enveloped particles (unless the cargo comprises the viral protein(s)); and maintaining the cell under conditions such that the cells produce the VLPs or mhVLPs. In some embodiments, the mhVLPs do or do not comprise any human endogenous retroviral (HERV) proteins other than the env, e.g., do not comprise gag, pol, or pro. Optionally the hENV optionally comprises one, two, or all three of: a targeting domain at the N or C terminus, or inserted internally into the protein; a truncation of one to 50 amino acids from the C terminus; and/or one or more RBD mutations, (ii) a cargo, and (iii) optionally a separate targeting domain.

In some embodiments, the methods further comprise harvesting and optionally purifying and/or concentrating the produced VLPs or mhVLPs. In some embodiments, the cargo is a therapeutic or diagnostic protein, and/or nucleic acid encoding a therapeutic or diagnostic protein, and/or a small molecule, optionally a therapeutic or diagnostic small molecule. In some embodiments, the cargo is a gene editing or epigenetic modulating reagent. In some embodiments, the gene editing or epigenetic modulating reagent comprises a zinc finger (ZF), transcription activator-like effector (TALE), and/or CRISPR-Cas protein, variant, or fusion thereof; a guide RNA and/or crRNA; a nucleic acid encoding a zinc finger (ZF), transcription activator-like effector (TALE), and/or CRISPR-Cas protein, variant, or fusion thereof; or a ribonucleoprotein complex (RNP) comprising a CRISPR-Cas protein, variant, or fusion thereof and optionally a guide RNA and/or crRNA.

In some embodiments, the cargo reagent is selected from the proteins listed in Tables 2, 3, 4 & 5, or that is at least 95% identical to a sequence set forth herein, e.g., in Tables 2, 3, 4, and 5. In some embodiments, the cargo reagent comprises a CRISPR-Cas protein, variant, or fusion thereof and the mhVLP further comprises one or more guide RNAs and/or crRNAs that bind to and direct the CRISPR-based genome editing or modulating protein to a target sequence. In some embodiments, the cargo comprises a fusion to a phospholipid bilayer recruitment domain, preferably as shown in Table 6, or that is at least 95% identical to a sequence set forth herein in Table 6.

Also provided herein are cells expressing a hENV as described herein, and a cargo, optionally wherein the cell does not express any exogenous virally derived proteins, e.g., proteins from viral gag, pro, or pol, or other viral proteins that reside inside of enveloped particles (unless the cargo comprises the viral protein(s)). Also provided are cells expressing (i) a human endogenous retroviral (HERV) envelope protein (hENV), wherein the hENV optionally comprises one, two, or all three of: a targeting domain at the N or C terminus, or inserted internally into the protein; a truncation of one to 50 amino acids from the C terminus; and/or one or more RBD mutations, (ii) a cargo, (iii) optionally a separate targeting domain. In some embodiments, the cells do or do not express any human endogenous retroviral (HERV) proteins other than the env, e.g., do not comprise gag, pol, or pro.

In some embodiments, the cargo is a therapeutic or diagnostic protein, and/or nucleic acid encoding a therapeutic or diagnostic protein, and/or a small molecule, optionally a therapeutic or diagnostic small molecule. In some embodiments, the cargo is a gene editing or epigenetic modulating reagent.

In some embodiments, the gene editing or epigenetic modulating reagent comprises a zinc finger (ZF), transcription activator-like effector (TALE), and/or CRISPR-Cas protein, variant, or fusion thereof; a guide RNA or crRNA; a nucleic acid encoding a zinc finger (ZF), transcription activator-like effector (TALE), and/or CRISPR-Cas protein, variant, or fusion thereof; or a ribonucleoprotein complex (RNP) comprising a CRISPR-Cas protein, variant, or fusion thereof and optionally a guide RNA or crRNA. In some embodiments, the cargo reagent is selected from the proteins listed in Tables 2, 3, 4, & 5, or that is at least 95% identical to a sequence set forth herein, e.g., in Tables 2, 3, 4, and 5. In some embodiments, the gene editing or epigenetic modulating reagent comprises a CRISPR-Cas protein, and the mhVLP further comprises one or more guide RNAs and/or crRNAs that bind to and direct the CRISPR-Cas protein to a target sequence. In some embodiments, the cargo comprises a fusion to a phospholipid bilayer recruitment domain, preferably as shown in Table 6, or that is at least 95% identical to a sequence set forth herein in Table 6. In some embodiments, the cells are primary or stable human cell lines. In some embodiments, the cells are Human Embryonic Kidney (HEK) 293 cells or HEK293 T cells.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Exemplary diagrams of DNA expression constructs that would be transfected into a producer cell to produce mhVLPs. Cas9 is shown as an exemplary cargo fused to a PH domain but could be replaced by any protein of interest.

FIGS. 2A-B. A. Diagram of HERV intracellular domain truncation strategy. Putative ancestral protease sites are found or predicted based on amino acid sequence alignments and truncations made to mimic the site in the envelope intracellular domain that would be cleaved by an ancestral protease (e.g., Trunc hENV W). B. Exemplary delivery of SpCas9-gRNA RNP by mhVLPs. K562 cells were transduced with mhVLPs containing human AKT Pleckstrin homology domain fused to SpCas9 and a gRNA targeted to the previously described VEGF site 3.1 genomic sequence (spacer sequence GAGCAGCGTCTTCGAGAGTG (SEQ ID NO:3)). mhVLPs were pseudotyped with a different HERV-derived envelope protein which either did not harbor (WT) or did harbor a truncation of its intracellular domain (Trunc). Gene modification frequencies of the intended VEGFs3.1 target site were measured by performing targeted amplicon sequencing using Illumina MiSeq NGS.

FIGS. 3A-D. Exemplary diagrams of mhVLPs with altered targeting tropisms. A & C. Exemplary diagrams of DNA expression constructs (including an scFv against a target cell receptor of interest) that would be transfected into a producer cell to produce mhVLPs with altered targeting tropisms. Note that in the constructs depicted in 3C the HERV-derived envelope harbors mutation(s) (starbursts) designed to reduce binding affinity for the cognate receptor of the HERV envelope. B & D. Schematics of mhVLP particles that would be produced by producer cells transfected with the DNA constructs shown in A. and C., respectively.

FIGS. 4A-D. Exemplary diagrams of mhVLPs with altered targeting tropism. A and C. Exemplary diagrams of DNA expression constructs that would be transfected into a producer cell to produce mhVLPs with altered targeting tropism. In both A and C, an scFv is fused to the HERV-derived envelope protein. Note that in the constructs depicted in C that the HERV-derived envelope harbors mutation(s) (starbursts) designed to reduce binding affinity for the cognate receptor of the HERV envelope. B and D. Schematics of mhVLP particles that would be produced by producer cells transfected with the DNA constructs shown in A and C, respectively.

FIG. 5. Exemplary editing efficiencies of eVLPs that package various PH-Cas9/sgRNA (VEGFs3.1-targeted) RNPs. HEK293T cells were treated with eVLPs pseudotyped with VSVG and editing efficiencies (x-axis) were determined by targeted amplicon sequencing using Illumina MiSeq NGS. Key for the various PH domains fused to Cas9:
PKD: protein kinase D1 (PRKD1)
DAPP: dual-adaptor for phosphotyrosine and 3-phosphoinositides-1 (DAPP-1)
FAPP: four-phosphate-adaptor protein (FAPP)
OSBP: oxysterol-binding protein (OSBP)
SWAP70: switch-associated protein 70 (SWAP70)
GRP: cytohesin 3 (CYTH3, formerly GRP1)
BTK: Bruton's tyrosine kinase (Btk)
PHLPP: Pleckstrin Homology Domain Leucine-rich Repeat Protein Phosphatase (PHLPP)
AKT: AKT serine/threonine kinase 1 (AKT1)
PLC: phospholipase C delta 1 (PLCδ1)

FIGS. 6A-C provides an alignment of the first 180 amino acids of HERV W, showing exemplary mutations in the RBD. Figure discloses SEQ ID NOS 200-208, 208-209, and 209-218, respectively, in order of appearance.

FIGS. 7A-B provides an alignment of sequences showing exemplary positions for insertion of targeting domains, with optional deletions. Figure discloses SEQ ID NOS 219-220, 222, and 221, respectively, in order of appearance.

FIG. 8 Depiction of exemplary mhVLP architecture for RNP/protein delivery. As shown in the figure, the mhVLP consists of an extracellular vesicle containing a cargo with or without a fusion to a pleckstrin homology (PH) domain (or other phospholipid bilayer recruitment domain), a human-derived envelope protein (hENV), and an optional guide RNA. All mhVLP expression DNA/RNA constructs could be transiently transfected into the producer cells and/or stably integrated in the genome of the producer cells. The human-derived envelope protein is expressed as a transmembrane protein on the plasma membrane. These particles are purified and are able to fuse with target cells and deliver cargo by interacting with surface receptors at the target cell surface.

FIG. 9 Exemplary mhVLP-delivered spCas9 ABE8e genome editing in vitro. Primary human hepatocytes transduced with mhVLPs containing AKT (E17K) PH fused to ABE8e, targeted to Bc11a. Two examples of transductions and resulting editing efficiencies from 400×-PEG purified mhVLPs pseudotyped with hENVs are shown. In this case, the hENVs were: “W483” (HERV W ENV 483 C-Terminal Truncation from Table 1C) or “HEMO” (HERV HEMO ENV from Table 1A). One example of a transduction and resulting editing efficiency from a 100×-PEG purified VSVG-pseudotyped eVLP (see WO 2022/020800) is also shown for reference.

FIG. 10 Exemplary mhVLP-delivered spCas9 ABE8e genome editing in vitro. Primary human hepatocytes transduced with mhVLPs having (+PH) or lacking (−PH) AKT (E17K) PH fused to ABE8e, targeted to VEGF Site #3. Two examples of transductions and resulting editing efficiencies from mhVLPs pseudotyped with hENVs are shown. In this case, the hENVs were: “W483” (HERV W ENV 483 C-Terminal Truncation from Table 1C) or “W FL” (HERV W ENV from Table 1A). Cas9 and sgRNA quantifications are shown for each mhVLP.

FIG. 11 Exemplary mhVLP-delivered spCas9 ABE8e genome editing in vitro. Primary human hepatocytes transduced with mhVLPs having AKT (E17K) PH fused to ABE8e, targeted to VEGF Site #3. Two examples of transductions and resulting editing efficiencies from mhVLPs pseudotyped with hENVs are shown. In this case, the hENVs were: “W483” (HERV W ENV 483 C-Terminal Truncation from Table 1C) or “W483 (D122N) (Q123K)” (HERV W ENV 483 C-Terminal Truncation from Table 1C with D122N and Q123K mutations), and “W483 (Q121K) (D122N) (Q123K)” (HERV W ENV 483 C-Terminal Truncation from Table 1C with Q121K, D122N and Q123K mutations).

FIG. 12 Exemplary mhVLP-delivered spCas9 ABE8e genome editing in vitro. Primary human skeletal muscle (phMUS) and primary human hematopoietic stem cells (phHSC) transduced with mhVLPs having AKT (E17K) PH (E17K PH) or nothing (no PH) fused to ABE8e, targeted to VEGF Site #3. Two examples of transductions and resulting editing efficiencies from mhVLPs pseudotyped with an hENV are shown. In this case, the hENV was: “W483 (D122N) (Q123K)” (HERV W ENV 483 C-Terminal Truncation from Table 1C with D122N and Q123K mutations).

FIG. 13 Exemplary “de-targeted” mhVLP-delivered spCas9 ABE8e genome editing in vitro. Primary human hepatocytes transduced with mhVLPs having AKT (E17K) PH fused to ABE8e, targeted to VEGF Site #3. Three de-targeted hENV-pseudotyped mhVLPs and 1 unmodified hENV-pseudotyped mhVLPs examples of transductions and resulting editing efficiencies from a 400×-PEG purification process are shown. In this case, the hENVs were: W483 (D116N, D122R), W483 (D116N), W483 (D122R), and W483 Cas9 and sgRNA quantifications are shown for each mhVLP.

FIG. 14 Depiction of exemplary stages of W hENV designs. Designs and rationales for improving activity and utility of W hENV are listed for different embodiments of the HERV W envelope (1-4a). In addition, the design for a de-targeted W hENV (pthENV) is listed (4b).

### DETAILED DESCRIPTION

Therapeutic proteins and nucleic acids hold great promise, but for many of these large biomolecules delivery into cells is a hurdle to clinical development. For example, genome editing reagents such as zinc finger nucleases (ZFNs) or RNA-guided, enzymatically active/inactive DNA binding proteins such as Cas9 have undergone rapid advancements in terms of specificity and the types of edits that can be executed, but the hurdle of safe in vivo delivery still remains an important challenge for gene editing and epigenetic editing therapies.

Virus-like particles (VLPs) have been utilized to deliver mRNA and protein cargo into the cytosol of cells.^{2,3,25-30} VLPs have emerged as an alternative delivery modality to retroviral or lentiviral particles. VLPs can be designed to lack the ability to integrate retroviral DNA, and to package and deliver combinations of protein/RNP/DNA. However, most VLPs, including recently conceived VLPs that deliver genome editing reagents known to date, utilize HIV or other virally-derived gag or gag-pol protein fusions and viral proteases to generate retroviral-like particles.^{25-27,29,30} Some VLPs containing RNA-guided nucleases (RGNs) also must package and express guide RNAs from a lentiviral DNA transcript,²⁷ and some VLPs require a viral protease in order to form functional particles and release genome editing cargo.^{25-27,29} Because this viral protease recognizes and cleaves at multiple amino acid motifs, it can cause damage to the protein cargo or potentially to other endogenous proteins in target recipient cells, which could be hazardous or create challenges for therapeutic applications. Most published VLP modalities that deliver genome editing proteins or RNPs to date exhibit low in vitro and in vivo gene modification efficiencies due to low packaging and transduction efficiency.²⁵⁻²⁷ The complex viral genomes utilized for these VLP components possess multiple reading frames and employ RNA splicing that could result in spurious fusion protein products being delivered.^{25-27,29,30} The presence of reverse transcriptase, integrase, capsid and a virally-derived envelope protein in these VLPs is not ideal for many therapeutic applications because of immunogenicity and off target concerns. In addition, most retroviral particles, such as lentiviral particles, are pseudotyped with VSVG and nearly all described VLPs that deliver genome editing reagents hitherto possess and rely upon VSVG.^{2,3,25-30}

Lentivirus and standard VLPs commonly require GAG and ENV proteins to drive particle formation via budding off of the plasma membrane of producer cells into the cell culture medium. In addition, the majority of retroviral ENV proteins require post-translational modifications in the form of proteolytic cleavage of the intracellular domain (ICD) of the ENV protein in order to fully activate the fusogenicity of the ENV protein; this is believed to be essential for infectivity of viral particles and for VLPs. Without wishing to be bound by theory, it is believed that the hENV protein alone is responsible for VLP/mhVLP particle generation and the ability of VLPs/mhVLPs as described herein to efficiently deliver cargo into cells.

Human Endogenous Retroviral (HERV) Envelope Proteins (hENVs)

A number of human endogenous retroviral proteins have been described (de Parseval et al., Journal of Virology 77, 10414-10422, (2003); Heidmann et al., Proc Natl Acad Sci USA. 2017 Aug. 8; 114(32):E6642-E6651). These sequences were used to reconstruct an ancestral proviral clone termed HERV-KcoN (Lee and Bieniasz, PLoS Pathog. 2007 January; 3(1):e10). Retroviral envelope proteins are glycoprotein composed of a surface unit (SU) and a transmembrane unit (TM). In the case of HERV W env, the SU contains a receptor binding domain (RBD), a furin cleavage site (RNKR at amino acids 314-317 (SEQ ID NO: 4)), six N-glycosylation sites, and a CFFC (SEQ ID NO: 286) (CX2C at amino acids 186-189) motif (Bastida-Ruiz D, et al. Int J Mol Sci. 2016 Apr. 28; 17(5):638). The HERV W env proteins bind to receptors including the monocarboxylate transporter-1 (MCT-1, Blanco-Melo et al., eLife 6:e22519 (2017)) and Major Facilitator Superfamily Domain Containing 2 (MFSD2, Esnault et al., Proc Natl Acad Sci USA. 2008 Nov. 11; 105(45):17532-7), and can be used to pseudotype lentiviruses to infect B cells (Coquin et al., bioRxiv 816223; doi.org/10.1101/816223) and to make recombinant vesicular stomatitis virus encoding HERV-K Env as its sole attachment and fusion protein (VSV-HERVK, Robinson-McCarthy et al., PLoS Pathog. 2018 Aug. 6; 14(8):e1007123).

The N-terminal 124 amino acids of the mature HERV W env glycoprotein have been identified as the minimal receptor-binding domain (RBD, Cheynet et al., Retrovirology. 2006 Jul. 4; 3:41). Chang et al., Biol Reprod. 2004 December; 71(6):1956-62, showed that hERV W env proteins (also referred to as syncytin-1) with C terminal deletions up to amino acid 480 retained cell fusion activity. Drewlo et al., Biol. Chem., 387:1113-1120 (2006) showed that variants of hERV W env truncated after residues 483 and 515 were hyperfusogenic compared to wild-type.

Provided herein are modified human endogenous retroviral (HERV) envelope proteins (hENV), wherein the hENV comprises one, two, or all three of: a targeting domain at the N or C terminus, or inserted internally into the protein; a truncation of one to 50 amino acids from the C terminus; and one or more RBD mutations. See, e.g., FIGS. 6A-C and 7A-B. These hENV proteins can be used, e.g., in VLPs and minimal human VLPs, e.g., comprising one, two, or all three of: (i) human endogenous retroviral (HERV) envelope protein (hENV), optionally wherein the hENV comprises one, two, or all three of: a targeting domain at the N or C terminus, or inserted internally into the protein; a truncation of one to 50 amino acids from the C terminus; and/or one or more RBD mutations, (ii) a cargo with or without fusion to a plasma membrane recruitment domain, (iii) optionally a membrane-anchored separate targeting domain (e.g., if not fused to the hENV, or in addition to a targeting domain fused to the hENV) for targeted delivery to a recipient cell, and (iv) optionally wherein the cell does not express, or does not overexpress, any other exogenous virally derived proteins, e.g., proteins from viral gag, pro, or pol, or other viral proteins that reside inside of enveloped particles, such as int(unless the cargo intentionally comprises the viral protein(s)).

Exemplary modified and unmodified hENVs are provided in Tables 1A-C. Combinations of the modifications shown in Tables 1B and 1C can be included.

Tables 1A-C. Exemplary hENVs

| # | Table 1A - HERV envelope proteins |
|---|---|
| | >AJ289709.1 Human endogenous retrovirus H HERV-H/env62 |
| | HERV_H/ENV_62 - hENVH1 |
| | >AJ289710.2 Human endogenous retrovirus H HERV-H/env60 - |
| | HERV_H_ENV_60 - hENVH2 |
| | >AJ289711.1 Human endogenous retrovirus H HERV-H/env59 - |
| | HERV_H_ENV_59 - hENVH3 |
| | >AC074261.3 Homo sapiens chromosome 12 clone RP11-55F19 |
| | envK1 - ENVK1 |
| | >AC072054.10 Homo sapiens BAC clone RP11-33P21 - ENVK2 |
| | >Y17833.1 Human endogenous retrovirus K (HERV-K) envK3 - |
| | ENVK3 |
| | >AF164615.1 Homo sapiens endogenous retrovirus HERV-K109 |
| | envK4 - ENVK4 |
| | >AY037928.1 Human endogenous retrovirus K113 envK5 - ENVK5 |
| | >AY037929.1 Human endogenous retrovirus K115 envK6 - ENVK6 |
| | >AC078899.1 Homo sapiens chromosome 19, BAC BC371065 |
| | envT - ENVT |
| | >AC000064.1 Human BAC clone RG083M05 from 7q21-7q22 envW |
| | (Syncytin-1) - ENVW (Syncytin-1) |
| | >AL136139.6 Human DNA sequence from clone RP4-76112 |
| | envFRD - ENVFRD (Syncytin-2) |
| | >AC073210.8 Homo sapiens BAC clone RP11-460N20 envR - |
| | ENVR |
| | >AC093488.1 Homo sapiens chromosome 3 clone RP11-10O8 |
| | envR(b) - ENVR(b) |
| | hENVF(>AC016222.4 Homo sapiens clone RP11-26J6 envF(c)2 - |
| | ENVF(c)2 |
| | >AL354685.17 Human DNA sequence from clone RP13-75G22 |
| | envF(c)1 - ENVF(c)1 |
| | HERV-Kcon ENV - hENVKcon |
| | HERV T ENV |
| | HERV W ENV |
| | HERV H ENV |
| | HERV Pb ENV |
| | HERV Rb ENV |
| | HERV 3-1 ENV |
| | HERV V1 ENV |
| | HERV HEMO ENV |
| | HERV FRD ENV |

TABLE 1B
Modified HERV envelope proteins - RBD mutations

HERV W ENV RBD MUT 1-1 (Q121A)
HERV W ENV RBD MUT 1-2 (Q121A) (Q123A)
HERV W ENV RBD MUT 1-3 (Q121A) (Q123A) (R125A)
HERV W ENV RBD MUT 2-1 (D122A)
HERV W ENV RBD MUT 2-2 (Q121A) (D122A)
HERV W ENV RBD MUT 2-3 (Q121A) (D122A) (Q123A)
HERV W ENV RBD MUT 2-4 (Q121A) (D122A) (Q123A) (R125G)
HERV W ENV RBD MUT 3.0 (Q123A)
HERV W ENV RBD MUT 3-1 (V120G) (Q123A)
HERV W ENV RBD MUT 3-2 (V120G) (Q123A) (R125A)
HERV W ENV RBD MUT 4.0 (R125A)
HERV W ENV RBD MUT 4-1 (V120G) (R125A)
HERV W ENV RBD MUT 4-2 (V120G) (D122A) (R125A)
HERV W ENV RBD MUT 4-3 (V120G) (D122A) (Q123A) (R125A)
HERV W ENV RBD MUT 5.0 (V120G)
HERV W ENV RBD MUT 5-1 (V120G) (Q123A)
HERV W ENV RBD MUT 5-2 (V120G) (Q123A) (R125A)
HERV W ENV RBD MUT 6.0 (A124G)
HERV W ENV RBD MUT 6-1 (A124G) (R125A)
HERV W ENV RBD MUT 6-2 (Q121A) (A124G) (R125A)
HERV W ENV RBD MUT (D122N) (Q123K)
HERV W ENV RBD MUT (Q121K) (D122N) (Q123K)
HERV W ENV RBD MUT (D116N) (D122R)
HERV W ENV RBD MUT (D116N)
HERV W ENV RBD MUT (D122R)
HERV Kcon ENV MUT 1 (R140A)
HERV Kcon ENV MUT 2 (R140C)

TABLE 1C
Modified HERV envelope proteins - Truncations
and Targeting Domain Insertions

HERV W ENV with targeting domain fusion site v1
HERV W ENV with targeting domain fusion site v2
HERV W ENV with targeting domain fusion site v3
HERV W ENV 480 C-Terminal Truncation
HERV W ENV 483 C-Terminal Truncation
HERV Kcon ENV 658 C-Terminal Truncation
HERV T ENV 611 C-Terminal Truncation
HEMO ENV 518 C-Terminal Truncation
HEMO ENV 521 C-Terminal Truncation
HERV Pb ENV 626 C-Terminal Truncation
HERV FRD ENV 515 C-Terminal Truncation
HERV H ENV 555 C-Terminal Truncation
HERV Rb 476 ENV C-Terminal Truncation
HERV 3-1 ENV 586 C-Terminal Truncation
HERV V-1 ENV 448 C-Terminal Truncation
*hENVK_{con} is a consensus sequence derived from ten proviral ENV sequences. The ENV sequences used to derive this consensus ENV sequence are from the following HERVs: HERV-K113, HERV-K101, HERV-K102, HERV-K104, HERV-K107, HERV-K108, HERV-K109, HERV-K115, HERV- K11p22, and HERV-K12q13.
#, SEQ ID NO:

In some embodiments, an hENV is at least 80%, 85%, 90%, 95%, 97%, 98%, or 99% identical to a protein identified in Table 1A, and retains the ability of the reference protein to generate VLP/mhVLP particles and to efficiently promote cargo delivery into cells. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

The hENV can also include one or more mutations in the receptor binding domain (RBD) described herein, e.g., in an amino acid corresponding to amino acids 115-125 of the hERV W env protein sequence, e.g., a MUT sequence as shown in Table 1B. Other mutations (e.g., R140C in HERV K ENV) that can be included have been described, e.g., in Hanke et al., J Virol. 2009 December; 83(24):12790-800.

The hENV can include targeting domains as described herein.

In some embodiments, the hENV is used in place of an ENV protein in a standard VLP, e.g., those VLPs described in previous publications.^{29,39,40} In some embodiments, the VLPs or mhVLPs or theVLPs can be composed of a mixture of ectosomes and exosomes that can be separated by purification, if desired. In part because of the above mentioned design simplifications and optimizations, VLPs/mhVLPs/theVLPs as described herein are particularly suited for delivery of cargo including but not limited to DNA, RNA, protein, and/or combinations of biomolecules and/or chemicals, such as DNA-encoded or RNP-based genome editing reagents.

### Targeting Domains

Provided herein are VLPs/mhVLPs/theVLPs that include targeting domains that bind to antigens on target cells to alter tropism of the VLPs/mhVLPs/theVLPs. A number of such antigens are known in the art. Exemplary antigens include CD19,⁷³ asialoglycoprotein receptor 1 (ASGR1),⁷⁴ Transferrin receptor (TfR),⁷⁵ HER2,⁷⁶ CD34,⁷⁷ CD4,⁷⁸ CD25,⁷⁹ CTLA-4,⁸⁰ HB-EGF,⁸¹ ACE2,⁸² Aryl hydrocarbon receptor (AhR),⁸³ keratin 5 (KRT5),⁸⁴ keratin 17 (KRT17),⁸⁵ keratin 14 (KRT14),⁸⁶ keratin 13 (KRT13),⁸⁷ Neural cell adhesion molecule L1,⁸⁸ Fibronectin (FN1),^{89,90,91} Amyloid precursor protein (APP),⁹² Programmed cell death protein 1 (PD-1),^{93,94} neurotrophin receptor (p75NTR),⁹⁵ Thy-1/CD90,⁹⁶ EpCAM,⁹⁷ and/or CFTR.⁹⁸

Targeting domains can include single chain variable fragment (scFv), nanobody, fibronectin type 3 domain (FN3), arginylglycylaspartic acid motif (RGD), single variable domain on a heavy chain/nanobody (VHH), variable domain of new antigen receptor (VNAR), darpin, or other targeting ligand that binds to an antigen on a target cell.⁶³⁻⁶⁹

Targeting domains can also include peptides, e.g., as shown in Table A. The targeting domains can be inserted into the sequence of an hENV protein such that it will be displayed on the surface of the VLP/mhVLP membrane, as described herein, or can be present as a separate molecule anchored on the outside of the VLP/mhVLP/theVLP membrane. Thus, fusion proteins comprising (i) a targeting domain and an envelope glycoprotein (programmable tropism HERV envelope protein (e.g., also referred to as a pthENV)), or (ii) a targeting domain and a membrane anchor are provided herein, as well as nucleic acids encoding the fusion proteins. In some embodiments, the targeting domain is inserted into an hENV protein between the signal sequence and the transmembrane domain, optionally replacing some or most of the N terminus of the ENV, including the RBD.

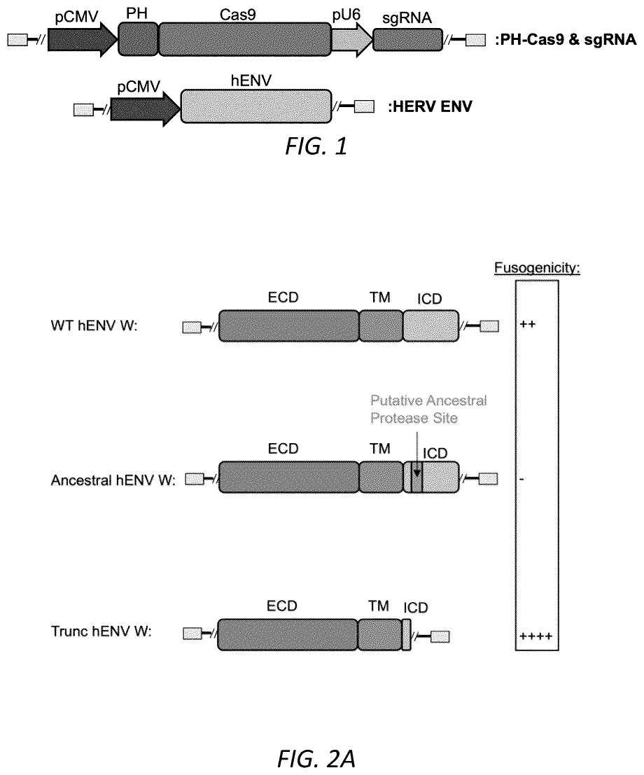

Membrane anchors can be any transmembrane (TM) domain, such as a TM from Platelet-derived growth factor receptor (PDGFR),⁹⁹ CD9,¹⁰⁰ CD63,¹⁰⁰ CD81,¹⁰⁰ CD86, Notch,⁷³ CD28.¹⁰¹ CD8,¹⁰² or CD4.¹⁰³ In general, the membrane anchored targeting domain fusion proteins will comprise, from N terminus to C terminus, the following: a secretion signal sequence-optional linker-targeting domain-optional linker-transmembrane domain (see, e.g., FIG. 1). Preferably, the optional linker between the three domains is a polypeptide linker that is 5-20, e.g., 8-12, e.g., 10, amino acids in length primarily composed of glycines and serines.

TABLE A
Exemplary Targeting Peptide Sequences

| | | SEQ |
|---|---|---|
| Targeting | | ID |
| Peptide | Sequence | NO: |
| CSP peptide of | CKNEKKNKIERNNKLKQPP | 5 |
| plasmodium | | |
| falciparum | | |
| CSP peptide of | DNEKLRKPKHKKLKQPADG | 6 |
| plasmodium | | |
| falciparum | | |
| peptide in ApoB- | RLTRKRGLK | 7 |
| 100 | | |
| RGD Peptide | RGD | |
| repeating peptide | CGRGDSPC | 8 |
| cyclic peptide 1 | RGDYK | 9 |
| cyclic peptide 2 | RGDFK | 10 |
| cyclic peptide 3 | PHSCNK | 11 |
| cyclic peptide 4 | CSRNLIDC | 12 |
| peptide 431 | VHWDFRQWWQPS | 13 |
| Pep1 | CHPREVDVELYSTVFGH | 14 |
| Pep2 | CEPEAEADAEAGPAGIGAVLKVLTTGLPALISWI | 15 |
| | KRKRQQ | |
| CendR | RPARPAR | 16 |
| IRGD | CRGDKGPDC | 17 |
| LinTT1 | AKRGARSTA | 18 |
| TT1 | CKRGARSTC | 19 |
| Lyp-1 | CGNKRTRGC | 20 |
| GLP-1 | HAEGTFTSDVSSYLEGQAAKEFIAWLVRGRG | 21 |
| HTPP | KNSRSLGENDDGNNEDNEKLR | 22 |
| M27-39 | AQQAANVAATLK | 23 |
| M27-39-HTPP | AQQAANVAATLKKNSRSLGENDDGNNEDNEKL | 24 |
| | R | |
| HSTP1 | CDGRPDRAC | 25 |
| GNSTM-HSTP1 | GNSTMCDGRPDRAC | 26 |

### Signal Sequences

Preferably, the membrane anchored targeting domains and the hENV comprise an N-terminal signal sequence; the original signal sequence can be used or can be replaced with a heterologous signal sequence. Exemplary signal sequences include the one from the VSV-G protein, e.g., MKCLLYLAFLFIGVNCK (SEQ ID NO: 1) and/or any other secretion signal sequence that is derived from VSVG (e.g., MKCLLYLAFLFIGVNC, SEQ ID NO:2) or a homolog thereof, or from a transmembrane protein and/or a synthetic/engineered signal sequence. A number of secretory signal peptide sequences are known in the art, including human signal sequences, examples of which are shown in Table B (Table adapted from novoprolabs.com/support/articles/commonly-used-leader-peptide-sequences-forefficient-secretion-of-a-recombinant-protein-expressed-in-mammalian-cells-201804211337.html).

TABLE B
Exemplary Human Secretory Signal Peptide Sequences

| Human Signal | | |
|---|---|---|
| sequence | Sequence | SEQ ID NO |
| Oncostatin M | MGVLLTQRTLLSLVLALLFPSMASM | 3. |
| IgG2 H | MGWSCIILFLVATATGVHS | 4. |
| Secrecon* | MWWRLWWLLLLLLLLWPMVWA | 5. |
| IgKVIII | MDMRVPAQLLGLLLLWLRGARC | 6. |
| CD33 | MPLLLLLPLLWAGALA | 7. |
| tPA | MDAMKRGLCCVLLLCGAVFVSPS | 8. |
| Chymotrypsinogen | MAFLWLLSCWALLGTTFG | 9. |
| trypsinogen-2 | MNLLLILTFVAAAVA | 10. |
| Interleukin 2 (IL-2) | MYRMQLLSCIALSLALVTNS | 11. |
| Albumin (HSA) | MKWVTFISLLFSSAYS | 12. |
| insulin | MALWMRLLPLLALLALWGPDPAAA | 13. |
| alpha 1-antitrypsin | MPSSVSWGILLLAGLCCLVPVSLA | 14. |

*, Barash et al., Biochem Biophys Res Commun. 2002 Jun. 21;294(4)835-42.

In some embodiments, another signal sequence that promotes secretion is used, e.g., as described in Table 5 of U.S. Ser. No. 10/993,967; von Heijne, J Mol Biol. 1985 Jul. 5; 184(1):99-105; Kober et al., Biotechnol. Bioeng. 2013; 110: 1164-1173; Tsuchiya et al., Nucleic Acids Research Supplement No. 3 261-262 (2003).

In general, the signal peptide is cleaved by a signal peptidase after the nascent protein is inserted into the membrane, as part of the secretory pathway processing inherent to cells.

### Phospholipid Bilayer Recruitment Domains

Conventional VLPs that have been engineered to encapsulate and deliver protein-based cargo commonly fuse the cargo to the INT, GAG or GAG-PRO-POL polyprotein.^{25-27,29,30,39,40} After transient transfection of production plasmid DNA constructs encoding the GAG/GAG-PRO-POL-fused cargo proteins and a viral envelope (ENV) protein, the protein fusions are translated in the cytosol of conventional VLP production cell lines, the gag matrix is acetylated and recruited to the cell membrane, and the gag fusions are encapsulated within VLPs as they bud off of the membrane into extracellular space.

In contrast, in some embodiments, proteins can be packaged into the mhVLPs/theVLPs by fusing select phospholipid bilayer recruitment domains, preferably human protein-derived phospholipid bilayer recruitment domains to protein-based cargo (e.g., as shown in Table 6).

One such human protein-derived phospholipid bilayer recruitment domain used for this purpose is a human pleckstrin homology (PH) domain. PH domains interact with phosphatidylinositol lipids and proteins within biological membranes, such as PIP2, PIP3, βγ-subunits of GPCRs, and PKC.^{41,42} Alternatively, the human Arc protein can be fused to protein-based cargo to recruit cargo to the cytosolic side of the phospholipid bilayer.⁴³ These human protein-derived phospholipid bilayer recruitment domains, or variants thereof (e.g., as shown in Table 6) can be fused to the N-terminus or C-terminus of protein-based cargo via polypeptide linkers of variable length regardless of the location or locations of one or more nuclear localization sequence(s) (NLS) within the cargo. Preferably, the linker between protein-based cargo and the phospholipid bilayer recruitment domain is a polypeptide linker 5-20, e.g., 8-12, e.g., 10, amino acids in length primarily composed of glycines and serines. The human protein-derived phospholipid bilayer recruitment domain localizes the cargo to the cytosolic face of the phospholipid bilayer and this protein cargo is packaged within mhVLPs/theVLPs that also contain and use an envelope glycoprotein to trigger budding-off of particles from the producer cell into extracellular space. These human protein-derived domains and human proteins can facilitate for localization of cargo to the cytosolic face of the plasma membrane within the mhVLP/theVLP production cells, and they also allow for the cargo to localize to the nucleus of mhVLP/theVLP-transduced cells without the utilization of exogenous retroviral gag/pol or chemical and/or light-based dimerization systems. The delivery of Cas9, for example, may be significantly more efficiently loaded as cargo into particles with fusion to a phospholipid bilayer recruitment domain compared to without fusion to a phospholipid bilayer recruitment domain.

mhVLP-Mediated Delivery of DNAs, Proteins and RNAs

The VLPs, mhVLPs, and theVLPs described herein (e.g., comprising hENV proteins) can package and deliver biomolecule cargo. “Cargo” refers to any payload that can be delivered, including chemicals, e.g., small molecule compounds, and biomolecules, including DNA, RNA, peptide nucleic acid (PNA), RNP, proteins, and combinations thereof, including combinations of DNA and RNP, DNA and RNA, RNP, combinations of DNA and protein(s), or protein(s), as well as viruses and portions thereof, e.g., for therapeutic or diagnostic use, or for the applications of genome editing, epigenome modulating, and/or transcriptome modulation. RNA in this context can include, for example, single guide RNA (sgRNA), Clustered Regularly Interspaced Palindromic Repeat (CRISPR) RNA (crRNA), and/or mRNA coding for cargo. Other exemplary nucleic acids can include specialty single and/or double-stranded DNA molecules (e.g., plasmid, mini circle, closed-ended linear DNA, AAV DNA, episomes, bacteriophage DNA, homology directed repair templates, etc.), single and/or double-stranded RNA molecules (e.g., single guide RNA, prime editing guide RNA, crRNA, tracrRNA, messenger RNA, transfer RNA, long non-coding RNA, circular RNA, RNA replicon, circular or linear splicing RNA, micro RNA, small interfering RNA, short hairpin RNA, piwi-interacting RNA, toehold switch RNA, RNAs that can be bound by RNA binding proteins, bacteriophage RNA, or internal ribosomal entry site containing RNA). Combinations of the above cargos (e.g., AAV particles and/or ribonucleoprotein (RNP) complexes comprising RNA and protein, e.g., guide RNA/CRISPR Cas protein complexes) can also be included.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, the cargo is limited by the diameter of the particles, e.g., which in some embodiments can range from 30 nm to 500 nm.

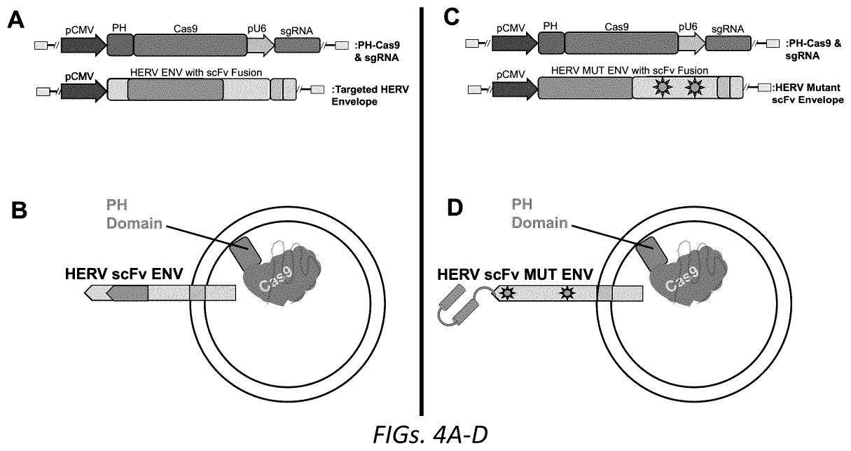

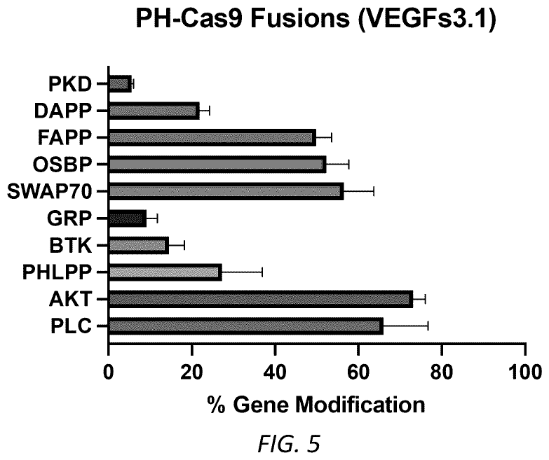

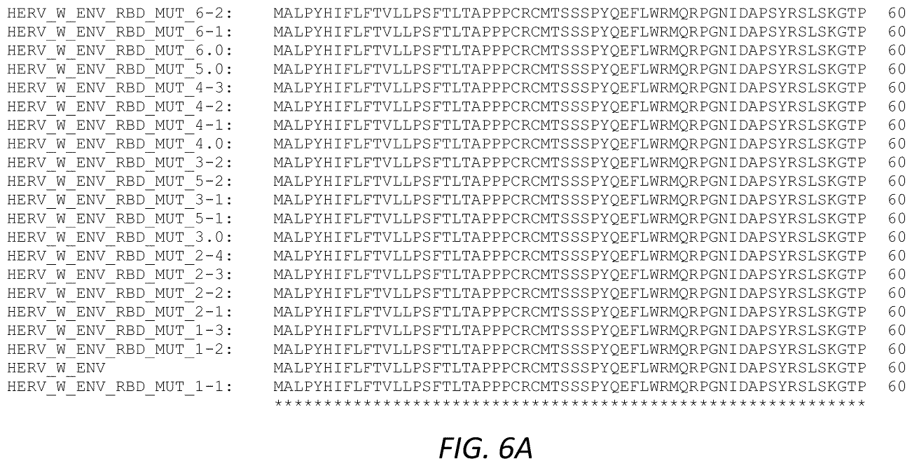

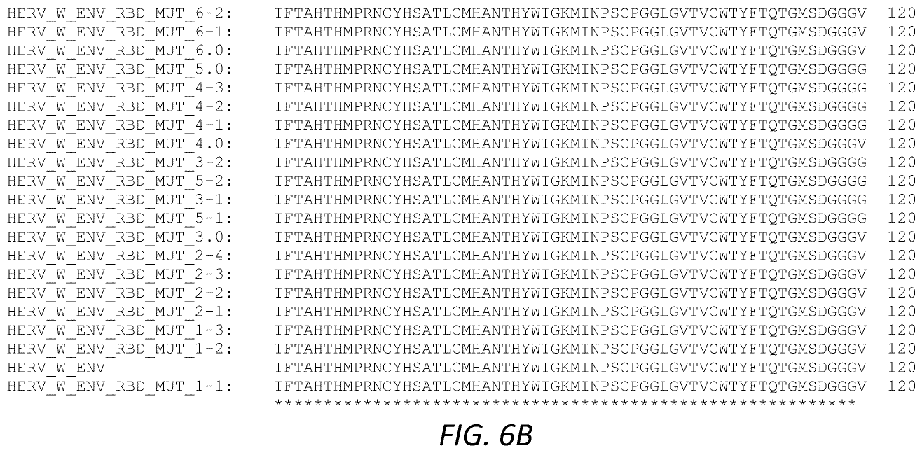

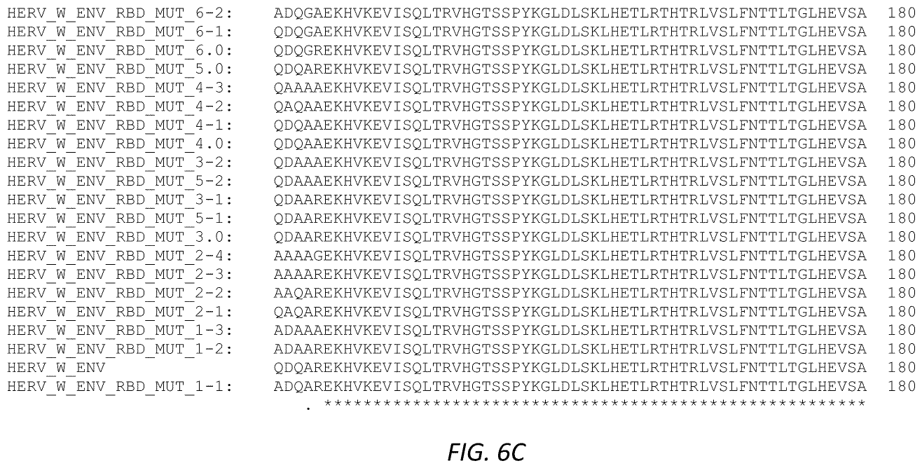

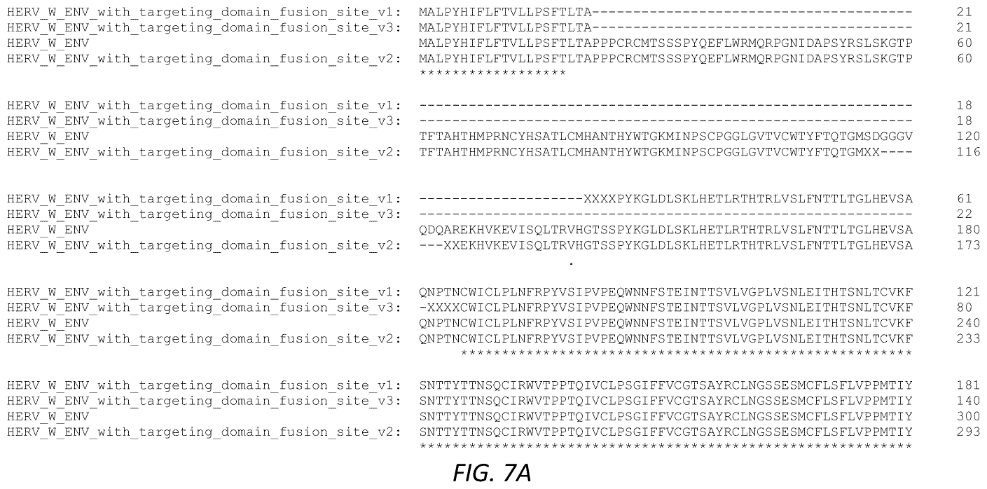

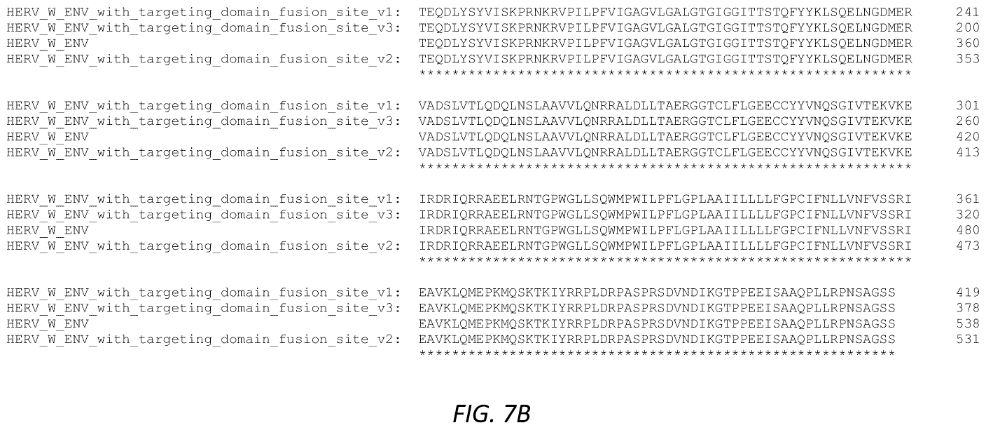

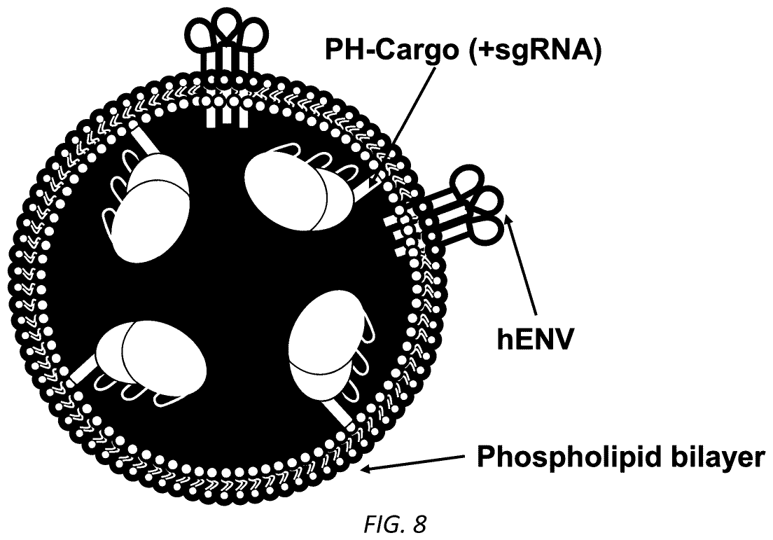

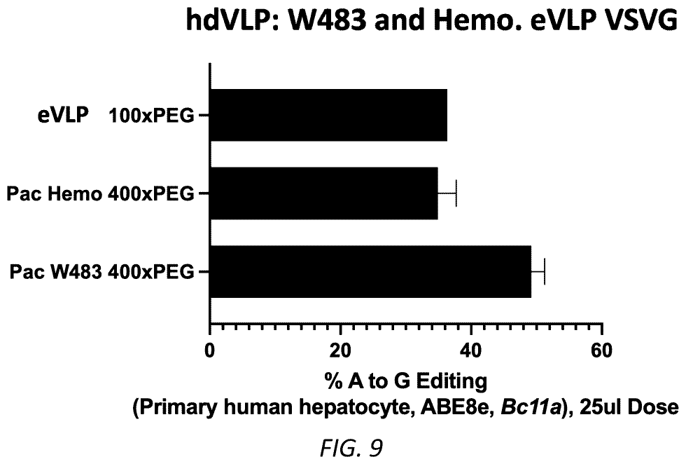

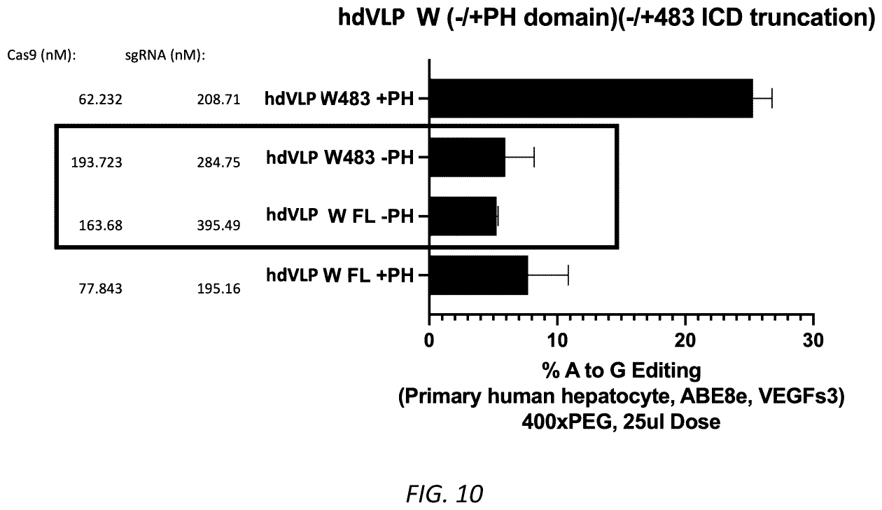

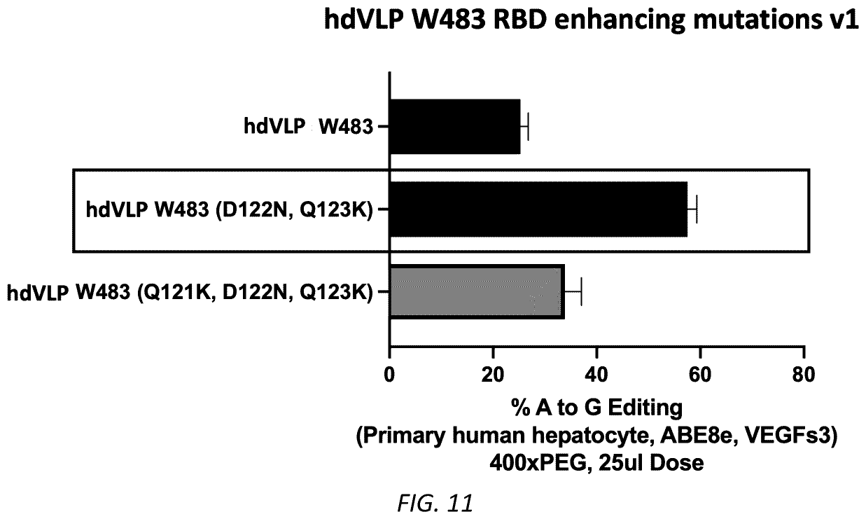

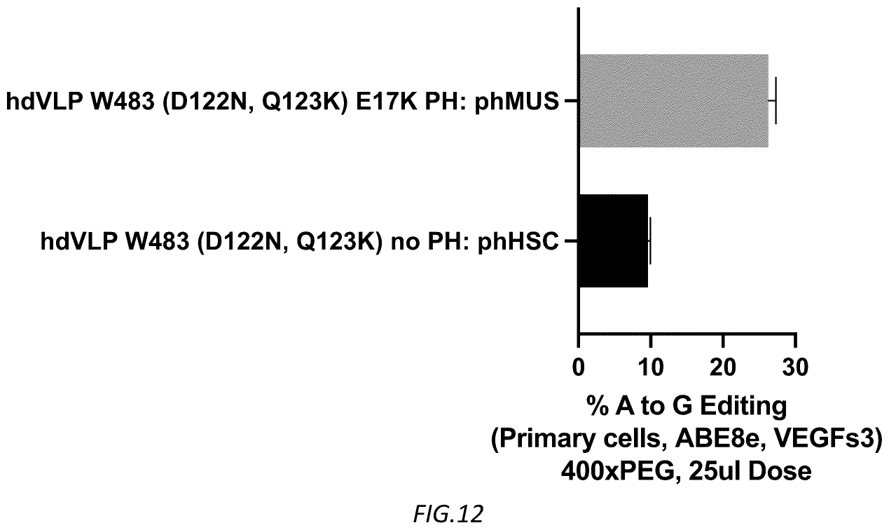

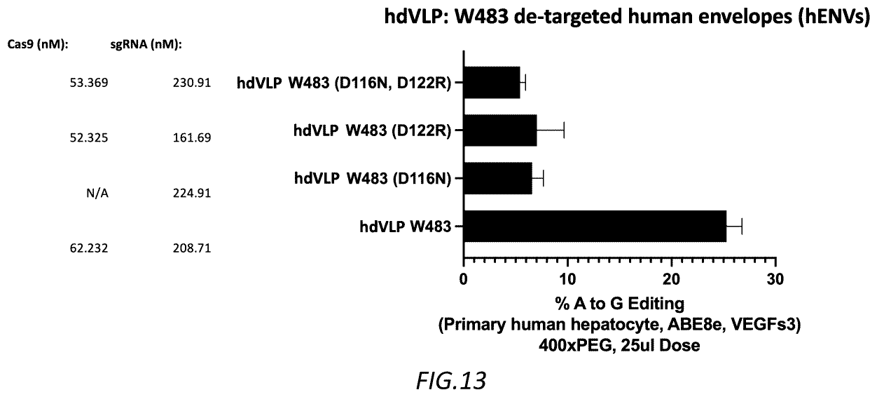

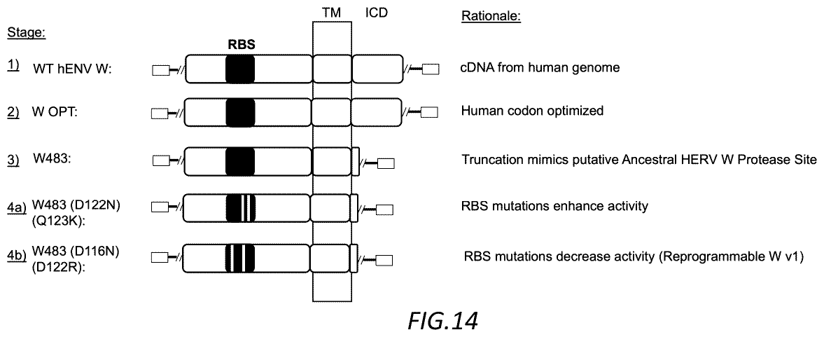

In some embodiments, VLPs/mhVLPs/theVLPs can also package and deliver a combination of DNA and RNA, e.g., when VLPs/mhVLPs/theVLPs are produced via transient transfection of a production cell line. DNA that is transfected into cells will possess size-dependent mobility such that a fraction of the transfected DNA will remain in the cytosol while another fraction of the transfected DNA will localize to the nucleus.⁴⁴⁻⁴⁶ A fraction of the transfected DNA in the nucleus will express components encoded on these plasmids needed to create mhVLPs/theVLPs and another fraction in the cytosol/near the plasma membrane could be encapsulated and delivered in mhVLPs. See, e.g., FIGS. 1-4 of WO 2022/020800.

Cargo developed for applications of genome or gene editing also includes CRISPR-Cas nucleases and fusions and variants thereof, e.g., prime editors, and base editors. Nucleases include ZFNs and Transcription activator-like effector nucleases (TALENs) that comprise a FokI or AcuI nuclease domain; and CRISPR Cas proteins or a functional derivative thereof (e.g., as shown in Table 2) (ZFNs are described, for example, in United States Patent Publications 20030232410; 20050208489; 20050026157; 20050064474; 20060188987; 20060063231; and International Publication WO 07/014275) (TALENs are described, for example, in U.S. Pat. No. 9,393,257B2; and International Publication WO2014134412A1) (CRISPR Cas proteins are described, for example, in U.S. Pat. No. 8,697,359B1; US20180208976A1; and International Publications WO2014093661A2; WO2017184786A8).³⁴⁻³⁶ Base editors can include any CRISPR based nuclease orthologs (wt, nickase, or catalytically inactive (CI)), e.g., as shown in Table 2, fused at the N-terminus to a nucleotide deaminase or nucleoside deaminase or a functional derivative thereof (e.g., as shown in Table 3), or comprising a deaminase domain inlaid internally, with or without a fusion at the C-terminus to one or multiple uracil glycosylase inhibitors (UGIs) using polypeptide linkers of variable length (Base editors are described, for example, in United States Patent Publications US20150166982A1; US20180312825A1; U.S. Ser. No. 10/113,163B2; and International Publications WO2015089406A1; WO2018218188A2; WO2017070632A2; WO2018027078A8; WO2018165629A1; WO 2018/218166).^{37,38,70-72} In addition, prime editors are also compatible with mVLP delivery modalities (Prime editors are described, for example, in Anzalone et al., Nature. 2019 December; 576(7785):149-157). Prime editors can be delivered, e.g., as fusions of Cas nickase to a reverse transcriptase or as separate components (see, e.g., Grunewald et al., Nat Biotechnol. 2022 Sep. 26. doi: 10.1038/s41587-022-01473-1; and Liu et al., Nat Biotechnol. 2022 September; 40(9):1388-1393).

Cargo designed for the purposes of epigenome modulating includes CRISPR Cas proteins, zinc fingers (ZFs) and TALEs fused to an epigenome/epigenetic modulating agent or combination of epigenome/epigenetic modulating agent or a functional derivative thereof connected together by one or more variable length polypeptide linkers. Exemplary epigenetic modulating agents include CRISPR-Cas proteins (e.g., nickases or catalytically inactive Cas) fused to DNA methylases, histone acetyltransferases, and deacetylases, as well as transcriptional activators or repressors (see, e.g., Tables 2 & 4). Examples include, e.g., transcriptional repressors (e.g., KRAB, ERD, SID, and others, e.g., amino acids 473-530 of the ets2 repressor factor (ERF) repressor domain (ERD), amino acids 1-97 of the KRAB domain of KOX1, or amino acids 1-36 of the Mad mSIN3 interaction domain (SID); see Beerli et al., PNAS USA 95:14628-14633 (1998)) or silencers such as Heterochromatin Protein 1 (HP1, also known as swi6), e.g., HP1α or HP1β; proteins or peptides that could recruit long non-coding RNAs (lncRNAs) fused to a fixed RNA binding sequence such as those bound by the MS2 coat protein, endoribonuclease Csy4, or the lambda N protein; enzymes that modify the methylation state of DNA (e.g., DNA methyltransferase (DNMT) or TET proteins); or enzymes that modify histone subunits (e.g., histone acetyltransferases (HAT), histone deacetylases (HDAC), histone methyltransferases (e.g., for methylation of lysine or arginine residues) or histone demethylases (e.g., for demethylation of lysine or arginine residues)) In some embodiments, the sequence of the cargo is at least 95% identical to a sequence set forth herein.

sgRNAs can complex with genome editing reagents during the packaging process to be co-delivered within VLPs/mhVLPs/theVLPs. Also, linear or circular RNAs encoding cargo or edits that are to be installed by a prime editor could be co-packaged with genome editing reagents that are fused to RNA binding proteins, such as MS2, PP7, COM, or TAR hairpin binding protein (TBP) or human SLBP. Cargo designed for the purposes of transcriptome editing includes CRISPR Cas proteins or any functional derivatives thereof (e.g., as shown in Table 5) or CRISPR Cas proteins or any functional derivatives thereof (e.g., as shown in Table 5) fused to nucleotide deaminases or nucleoside deaminases (e.g., as shown in Table 3) by one or more variable length polypeptide linkers.

The cargo can also include any therapeutically or diagnostically useful protein, DNA, RNP, or combination of DNA, protein and/or RNP. See, e.g., WO2014005219; U.S. Ser. No. 10/137,206; US20180339166; U.S. Pat. No. 5,892,020A; EP2134841B1; WO2007020965A1. For example, cargo encoding or composed of nuclease or base editor proteins or RNPs or derivatives thereof can be delivered to retinal cells for the purposes of correcting a splice site defect responsible for Leber Congenital Amaurosis type 10. In the mammalian inner ear, mhVLP delivery of base editing reagents or HDR promoting cargo to sensory cells such as cochlear supporting cells and hair cells for the purposes of editing β-catenin (β-catenin Ser 33 edited to Tyr, Pro, or Cys) in order to better stabilize β-catenin could help reverse hearing loss.

In another application, mhVLP/VLP/theVLP delivery of RNA editing reagents or proteome perturbing reagents could cause a transitory reduction in cellular levels of one or more specific proteins of interest (potentially at a systemic level, in a specific organ or a specific subset of cells, such as a tumor), and this could create a therapeutically actionable window when secondary drug(s) could be administered (this secondary drug is more effective in the absence of the protein of interest or in the presence of lower levels of the protein of interest). For example, mhVLP/VLP/theVLP delivery of RNA editing reagents or proteome perturbing reagents could trigger targeted degradation of MAPK and PI3K/AKT proteins and related mRNAs in vemurafenib/dabrafenib-resistant BRAF-driven tumor cells, and this could open a window for the administration of vemurafenib/dabrafenib because BRAF inhibitor resistance is temporarily abolished (resistance mechanisms based in the MAPK/PI3K/AKT pathways are temporarily downregulated by mhVLP/VLP/theVLP cargo). This example is especially pertinent when combined with mhVLPs that are antigen inducible and therefore specific for tumor cells. Alternatively, the transitory reduction in cellular levels of a specific protein of interest may itself have therapeutic benefit.

In some embodiments, mhVLPs/VLPs/theVLPs as described herein could be used deliver factors, e.g., including the Yamanaka factors Oct3/4, Sox2, Klf4, and c-Myc, to cells such as human or mouse fibroblasts, in order to generate induced pluripotent stem cells or to deliver factors that induce forward differentiation or trans-differentiation into a specific cell-type. This can be accomplished by overexpressing the Yamanaka factors and a hENV(s) in the producer cell. In addition, mhVLPs/VLPs/theVLPs can be used to deliver mitochondria. This can be accomplished by overexpressing a hENV in the producer cell. Particle populations that are within a certain size range will be enriched with mitochondria.

In some embodiments, mhVLPs/VLPs/theVLPs as described herein could deliver dominant-negative forms of proteins in order to elicit a therapeutic effect.

mhVLPs/VLPs/theVLPs as described herein that are antigen-specific (e.g., tumor-antigen specific) could be targeted to cancer cells in order to deliver proapoptotic proteins BIM, BID, PUMA, NOXA, BAD, BIK, BAX, BAK and/or HRK in order to trigger apoptosis of cancer cells. Tumor antigens are known in the art.

90% of pancreatic cancer patients present with unresectable disease. Around 30% of patients with unresectable pancreatic tumors will die from local disease progression, so it is desirable to treat locally advanced pancreatic tumors with ablative radiation, but the intestinal tract cannot tolerate high doses of radiation needed to cause tumor ablation. Selective radioprotection of the intestinal tract enables ablative radiation therapy of pancreatic tumors while minimizing damage done to the surrounding gastrointestinal tract. To this end, mhVLPs/VLPs/theVLPs as described herein could be loaded with dCas9 fused to the transcriptional repressor KRAB and guide RNA targeting EGLN. EGLN inhibition has been shown to significantly reduce gastrointestinal toxicity from ablative radiation treatments because it causes selective radioprotection of the gastrointestinal tract but not the pancreatic tumor.⁴⁷ Such fusion proteins, mhVLPs, and methods of making and using the same are provided herein.

Unbound steroid receptors reside in the cytosol. After binding to ligands, these receptors will translocate to the nucleus and initiate transcription of response genes. mhVLPs/VLPs/theVLPs as described herein could deliver single chain variable fragment (scFv) antibodies to the cytosol of cells that bind to and disrupt cytosolic steroid receptors. For example, the scFv could bind to the glucocorticoid receptor and prevent it from binding dexamethasone, and this would prevent transcription of response genes, such as metallothionein 1E which has been linked to tumorigenesis.⁴⁸

mhVLPs/VLPs/theVLPs as described herein can be indicated for treatments that involve targeted disruption of proteins. For example, mhVLPs/VLPs/theVLPs as described herein can be utilized for targeting and disrupting proteins in the cytosol of cells by delivering antibodies/scFvs to the cytosol of cells. Classically, delivery of antibodies through the plasma membrane to the cytosol of cells has been notoriously difficult and inefficient. This mode of protein inhibition is similar to how a targeted small molecule binds to and disrupts proteins in the cytosol and could be useful for the treatment of a diverse array of diseases.⁴⁹⁻⁵¹ Such fusion proteins, mhVLPs/VLPs/theVLPs as described herein, and methods of making and using the same are provided herein.

In addition, the targeting of targeted small molecules is limited to proteins of a certain size that contain binding pockets which are relevant to catalytic function or protein-protein interactions. scFvs are not hampered by these limitations because scFvs can be generated that bind to many different moieties of a protein in order to disrupt catalysis and interactions with other proteins. For example, RAS oncoproteins are implicated across a multitude of cancer subtypes, and RAS is one of the most frequently observed oncogenes in cancer. For instance, the International Cancer Genome Consortium found KRAS to be mutated in 95% of their Pancreatic Adenocarcinoma samples. RAS isoforms are known to activate a variety of pathways that are dysregulated in human cancers, like the PI3K and MAPK pathways. Despite the aberrant roles RAS plays in cancer, no efficacious pharmacologic direct or indirect small molecule inhibitors of RAS have been developed and approved for clinical use. One strategy for targeting RAS could be mhVLPs that can deliver specifically to cancer cells scFvs that bind to and disrupt the function of multiple RAS isoforms.⁴⁹⁻⁵¹ VLP/mhVLP/theVLP composition, production, purification and applications mhVLPs/VLPs/theVLPs as described herein can be produced from producer cell lines that are either transiently transfected with at least one plasmid/polynucleic acid construct or stably expressing construct(s) that have been integrated into the producer cell line genomic DNA. In some embodiments, for mhVLPs/theVLPs, e.g., if a single plasmid is used in the transfection, it should comprise sequences encoding one or more transmembrane HERV envelope glycoproteins (with or without specified mutation(s)/truncations and/or targeting domain fusions) (e.g., unmodified HERV envelopes are shown in Table 1A) or a transmembrane HERV envelope glycoprotein with or without specified mutation(s)/truncations with a membrane-anchored targeting domain in trans, cargo (e.g., a therapeutic protein or a gene editing reagent such as a zinc finger, transcription activator-like effector (TALE), and/or CRISPR-based genome editing/modulating protein and/or RNP such as those found in Tables 2, 3, 4 & 5), with or without fusion to a plasma membrane recruitment domain (e.g., as shown in Table 6), and at least one guide RNA, if necessary. Preferably, two to three plasmids are used in the transient transfection. These two to three plasmids can include the following (any two or more components listed here can also be combined in a single plasmid):
1. A plasmid comprising sequences encoding a therapeutic protein or a genome editing reagent, with or without a fusion to a plasma membrane recruitment domain.
2. A plasmid comprising one or more HERV envelope glycoproteins with or without specified mutation(s)/truncation(s) and/or targeting domain fusions (e.g., unmodified HERV envelopes are listed in Table TA).
3. If the genome editing reagent from plasmid 1 requires one or more guide RNAs, a plasmid comprising one or more guide RNAs apposite for the genome editing reagent in plasmid 1.

In addition, four or more plasmids could be used in the transient transfection. These four or more plasmids can include the following (any two or more components listed here can also be combined in a single plasmid):
1. A plasmid comprising sequences encoding a therapeutic protein or a genome editing reagent, with or without a fusion to a plasma membrane recruitment domain.
2. A plasmid comprising one or more HERV envelope glycoproteins with or without specified mutation(s)/truncation(s) (e.g., as listed in Tables 1A-C).
3. A plasmid comprising one or more membrane anchored targeting domains(s) (e.g., scFv, FN3, RGD, VHH, VNAR, nanobody, darpin, or other targeting ligands).
4. If the genome editing reagent from plasmid 1 requires one or more guide RNAs, a plasmid comprising one or more guide RNAs apposite for the genome editing reagent in plasmid 1.

If it is desired to deliver a type of DNA molecule other than plasmid(s), the above-mentioned transfection can be performed with double-stranded closed-end linear DNA, episome, mini circle, double-stranded oligonucleotide and/or other specialty DNA molecules. Alternatively, for mhVLPs/theVLPs, the producer cell line can be made to stably express the constructs (1 through 3) described in the transfection above.

As stated earlier, in some embodiments, the methods include using cells that have or have not been manipulated to express any exogenous proteins except for a targeted HERV envelope with or without targeting domain fusion or HERV envelope with associated targeting domain in trans with or without specified mutation(s)/truncation(s) (e.g., as shown in Tables 1A-C), and, if desired, a plasma membrane recruitment domain (e.g., as shown in Table 6). In this embodiment, the “empty” particles that are produced can be loaded with cargo and/or small molecules by utilizing incubation, nucleofection, lipid, polymer, or CaCl₂) transfection, sonication, freeze thaw, and/or heat shock of purified particles mixed with cargo. In some embodiments, producer cells do not express any exogenous gag protein. This type of loading allows for cargo to be unmodified by fusions to plasma membrane recruitment domains and represents a significant advancement from previous VLP technologies.

The plasmids, or other types of specialty DNA molecules known in the art or described above, can also preferably include other elements to drive expression or translation of the encoded sequences, e.g., a promoter sequence; an enhancer sequence, e.g., 5′ untranslated region (UTR) or a 3′ UTR; a polyadenylation site; IRES; 2A peptide; an insulator sequence; or another sequence that increases or controls expression (e.g., an inducible promoter element).

Preferably, appropriate producer cell lines are primary or stable human cell lines refractory to the effects of transfection reagents and fusogenic effects due to virally-derived glycoproteins. Examples of appropriate cell lines include Human Embryonic Kidney (HEK) 293 cells, HEK293 T/17 SF cells kidney-derived Phoenix-AMPHO cells, and placenta-derived BeWo cells. For example, such cells could be selected for their ability to grow as adherent cells, or suspension cells. In some embodiments, the producer cells can be cultured in classical DMEM under serum conditions, serum-free conditions, or exosome-free serum conditions. mhVLPs/theVLPs can be produced from cells that have been derived from patients (autologous mhVLPs/theVLPs) and other FDA-approved cell lines (allogenic mhVLPs/theVLPs) as long as these cells can be transfected with DNA constructs that encode the aforementioned mhVLP/theVLP production components by various techniques known in the art.

In addition, if it is desirable, more than one genome editing reagent encoded in polynucleic acid construct(s) can be included in the transfection. The DNA constructs can be designed to overexpress proteins in the producer cell lines. The plasmid backbones, for example, used in the transfection can be familiar to those skilled in the art, such as the pCDNA3 backbone that employs the CMV promoter for RNA polymerase II transcripts or the U6 promoter for RNA polymerase III transcripts. Various techniques known in the art may be employed for introducing polynucleic acid molecules into producer cells. Such techniques include chemical-facilitated transfection using compounds such as calcium phosphate, cationic lipids, cationic polymers, liposome-mediated transfection, such as cationic liposome like LIPOFECTAMINE (LIPOFECTAMINE 2000 or 3000 and TransIT-X2), polyethyleneimine, non-chemical methods such as electroporation, particle bombardment, or microinjection.

A human producer cell line that stably expresses the necessary mhVLP/theVLP components in a constitutive and/or inducible fashion can be used for production of mhVLPs/theVLPs. mhVLPs/theVLPs can be produced from cells that have been derived from patients (autologous mhVLPs/theVLPs) and other FDA-approved cell lines (allogenic mhVLPs/theVLPs) if these cells have been converted into stable cell lines that express the aforementioned mhVLP/theVLP components.

Also provided herein are the producer cells themselves.

Production of Cargo-Loaded mhVLPs theVLPs and Compositions

Preferably mhVLPs/theVLPs are harvested from cell culture medium supernatant 36-48 hours post-transfection, or when mhVLPs/theVLPs are at the maximum concentration in the medium of the producer cells (the producer cells are expelling particles into the media and at some point in time, the particle concentration in the media will be optimal for harvesting the particles). Supernatant can be purified by any known methods in the art, such as centrifugation, ultracentrifugation, precipitation, ultrafiltration, tangential flow filtration, and/or chromatography. In some embodiments, the supernatant is first filtered, e.g., to remove particles larger than 1 m, e.g., through 0.45 pore size polyvinylidene fluoride hydrophilic membrane (Millipore Millex-HV) or 0.8 m pore size mixed cellulose esters hydrophilic membrane (Millipore Millex-AA). After filtration, the supernatant can be further purified and concentrated, e.g., using ultracentrifugation, e.g., at a speed of 80,000 to 100,000×g at a temperature between 1° C. and 5° C. for 1 to 2 hours, or at a speed of 8,000 to 15,000 g at a temperature between 1° C. and 5° C. for 10 to 16 hours. After this centrifugation step, the mhVLPs/theVLPs are concentrated in the form of a centrifugate (pellet), which can be resuspended to a desired concentration, mixed with transduction-enhancing reagents, subjected to a buffer exchange, or used as is. In some embodiments, mhVLP/theVLP-containing supernatant can be filtered, precipitated, centrifuged, and resuspended to a concentrated solution. For example, polyethylene glycol (PEG), e.g., PEG 8000, or antibody-bead conjugates that bind to mhVLP/theVLP surface proteins or membrane components can be used to precipitate particles. Purified particles are stable and can be stored at 4° C. for up to a week or −80° C. for years without losing appreciable activity.

Preferably, mhVLPs/theVLPs are resuspended or undergo buffer exchange so that particles are suspended in an appropriate carrier. In some embodiments, buffer exchange can be performed by ultrafiltration (e.g., Sartorius Vivaspin 500 MWCO 100,000). An exemplary appropriate carrier for mhVLPs/theVLPs to be used for in vitro applications would preferably be a cell culture medium that is suitable for the cells that are to be transduced by mhVLPs/theVLPs. Transduction-enhancing reagents that can be mixed into the purified and concentrated mhVLP/theVLP solution for in vitro applications include reagents known by those familiar with the art (e.g., Miltenyi Biotec Vectofusin-1, Millipore Polybrene, Takara Retronectin, Sigma Protamine Sulfate, and the like). After mhVLPs/theVLPs in an appropriate carrier are applied to the cells to be transduced, transduction efficiency can be further increased by centrifugation. Preferably, the plate containing mhVLPs/theVLPs applied to cells can be centrifuged at a speed of 1,150 g at room temperature for 30 minutes. After centrifugation, cells are returned into the appropriate cell culture incubator (e.g., humidified incubator at 37° C. with 5% CO2).

An appropriate carrier for mhVLPs/theVLPs to be administered to a mammal, especially a human, would preferably be a pharmaceutically acceptable composition. A “pharmaceutically acceptable composition” refers to a non-toxic semisolid, liquid, or aerosolized filler, diluent, encapsulating material, colloidal suspension or formulation auxiliary of any type. Preferably, this composition is suitable for injection. These may be in particular isotonic, sterile, saline solutions (monosodium or disodium phosphate, sodium, potassium, calcium or magnesium chloride and similar solutions or mixtures of such salts), or dry, especially freeze-dried compositions which upon addition, depending on the case, of sterilized water or physiological saline, permit the constitution of injectable solutions. Another appropriate pharmaceutical form would be aerosolized particles for administration by intranasal inhalation or intratracheal intubation.

The pharmaceutical forms suitable for injectable use include sterile aqueous solutions or suspensions. The solution or suspension may comprise additives which are compatible with mhVLPs/theVLPs and do not prevent mhVLP/theVLP entry into target cells. In all cases, the form must be sterile and must be fluid to the extent that the form can be administered with a syringe. It must be stable under the conditions of manufacture and storage and must be preserved against the contaminating action of microorganisms, such as bacteria and fungi. An example of an appropriate solution is a buffer, such as phosphate buffered saline.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization.

Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

The compositions comprising cargo-loaded mhVLPs/theVLPs can be included in a container, pack, or dispenser together with instructions for administration.

TABLE 2
Exemplary Potential Cas9 and Cas12a orthologs

| DNA-binding Cas | | | |
|---|---|---|---|
| ortholog | Enzyme class | Nickase mutation | CI mutations |
| SpCas9 | Type II-A | D10A | D10A, H840A |
| SaCas9 | Type II-A | D10A | D10A, |
| CjCas9 | Type II-C | D8A | D8A, |
| NmeCas9 | Type II-C | D16A | D16A, H588A |
| AsCas12a | Type II-C | | D908A, E993A |
| LbCas12a | Type II-C | | D832A, E925A |

Nickase mutation residues represent a position of the enzyme either known to be required for catalytic activity of the conserved RuvC nuclease domain or predicted to be required for this catalytic activity based on sequence alignment to CjCas9 where structural information is lacking (* indicates which proteins lack sufficient structural information). All positional information refers to the wild-type protein sequences acquired from uniprot.org.

TABLE 3
Exemplary Deaminase domains and their
substrate sequence preferences.

| Deaminase | Nucleotide sequence preference |
|---|---|
| hAID | 5′-WRC |
| rAPOBEC1* | 5′-TC ≥ CC ≥ AC > GC |
| mAPOBEC3 | 5′-TYC |
| hAPOBEC3A | 5′-TCG |
| hAPOBEC3B | 5′-TCR > TCT |
| hAPOBEC3C | 5′-WYC |
| hAPOBEC3F | 5′-TTC |
| hAPOBEC3G | 5′-CCC |
| hAPOBEC3H | 5′-TTCA ~ TTCT ~ TTCG > ACCCA > TGCA |
| E. coli TadA | A |
| hAdar1 | A |
| hAdar2 | A |

Nucleotide positions that are poorly specified or are permissive of two or more nucleotides are annotated according to IUPAC codes, where W = A or T, R = A or G, and Y = C or T. “h” before the deaminase name indicates Homo sapiens origin. “m” before the deaminase name indicates Mus musculus origin. “r” before the deaminase name indicates Rattus origin.

TABLE 4
Exemplary Epigenetic modulating domains.

| Epigenetic modulator | Epigenetic modulation |
|---|---|
| VP16 | transcriptional activation |
| VP64 | transcriptional activation |
| P65 | transcriptional activation |
| RTA | transcriptional activation |
| KRAB | transcriptional repression |
| MeCP2 | transcriptional repression |
| TET1 | Methylation |
| DNMT3a | Methylation |

TABLE 5
Exemplary CRISPR based RNA-guided RNA binding enzymes

| RNA-binding Cas ortholog | Enzyme class |
|---|---|
| LshCas13a | Type-VI |
| LwaCas13a | Type-VI |

TABLE 6
Exemplary Plasma membrane recruitment domains

| Plasma membrane recruitment domain | Substitution(s) |
|---|---|
| Pleckstrin homology domain of human | |
| phospholipase Cδ1 (hPLCδ1) | |
| Pleckstrin homology domain of human | R40L⁵² |
| phospholipase Cδ1 (hPLCδ1) | |
| Pleckstrin homology domain of human | |
| Akt1 (hAkt1) | |
| Mutant Pleckstrin homology domain of | E17K⁵³ |
| human Akt1 | |
| Pleckstrin homology domain of human 3- | |
| phosphoinositide-dependent protein | |
| kinase 1 (hPDPK1) | |
| Mutant Pleckstrin homology domain of | K14R⁵⁶ |
| human Akt1 | |
| Mutant Pleckstrin homology domain of | K8R⁵⁷ |
| human Akt1 | |
| Mutant Pleckstrin homology domain of | T72A⁵⁸ |
| human Akt1 | |
| Mutant Pleckstrin homology domain of | T92A⁵⁹ |
| human Akt1 | |
| Mutant Pleckstrin homology domain of | R25C⁵² |
| human Akt1 | |
| Mutant Pleckstrin homology domain of | T34D⁵⁴ |
| human Akt1 | |
| Mutant Pleckstrin homology domain of | T34F⁵⁴ |
| human Akt1 | |
| Mutant Pleckstrin homology domain of | T34L⁵⁴ |
| human Akt1 | |
| Mutant Pleckstrin homology domain of | T81Y⁵⁵ |
| human Akt1 | |
| Mutant Pleckstrin homology domain of | K142A, H143A, |
| human Akt1 | R144A⁶⁰ |
| Mutant Pleckstrin homology domain of | T101C⁶¹ |
| human Akt1 | |
| Pleckstrin homology domain of Human | |
| Dapp1 | |
| Pleckstrin homology domain of Human | |
| GRP1 | |
| Pleckstrin homology domain of Human | R284C⁵² |
| GRP1 | |
| Pleckstrin homology domain of Human | |
| OSBP1 | |
| Pleckstrin homology domain of Human | R108E⁵² |
| OSBP1 | |
| Pleckstrin homology domain of Human | |
| ARNO (CYTH2) | |
| Pleckstrin homology domain of Human | R279C⁵² |
| ARNO (CYTH2) | |
| Pleckstrin homology domain of Human | |
| Btk1 | |
| Pleckstrin homology domain of Human | R28C⁵² |
| Btk1 | |
| FYVE domain of Human EEA1 | |
| FYVE domain of Human EEA1 | R1375L⁵² |
| PX domain of p40^{phox} (NCF4) | |
| PX domain of p40^{phox} (NCF4) | R58L⁵² |
| Pleckstrin homology domain of Human | |
| FAPP1 | |
| Pleckstrin homology domain of Human | |
| CERT | |
| Pleckstrin homology domain of Human | |
| PHLPP1 | |
| Pleckstrin homology domain of Human | |
| SWAP70 | |
| Pleckstrin homology domain of Human | R223E and R224E⁶² |
| SWAP70 | |
| Pleckstrin Homology Domain of Human | |
| PKD | |
| Pleckstrin homology domain of Human | |
| MAPKAP1 | |
| Pleckstrin homology domain of Human | |
| Son Of Sevenless Homolog 2 | |
| Pleckstrin homology domain of Human | |
| Dynamin | |
| Pleckstrin homology domain of Human | |
| BCR | |
| Pleckstrin homology domain of Human | |
| DBS | |

### Exemplary Sequences

In some embodiments, the sequence of a protein or nucleic acid used in a composition or method described herein is at least 80%, 85%, 90%, 95%, 97%, 98%, or 99% identical to a sequence set forth herein. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

Prime Editor: spCas9 H840A-MMLV Reverse Transcriptase (delta RNase H
domain):
(SEQ ID NO: 39)
MKRTADGSEFESPKKKRKVDKKYSIGLDIGTNSVGWAVITDEYKVPSKKFKVLGNTDRHSIKKNLIGAL
LEDSGETAEATRLKRTARRRYTRRKNRICYLQEIFSNEMAKVDDSFFHRLEESELVEEDKKHERHPIFG
NIVDEVAYHEKYPTIYHLRKKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLEGNLIALSLGLTPNFKSNEDL
AEDAKLQLSKDTYDDDLDNLLAQIGDQYADLFLAAKNLSDAILLSDILRVNTEITKAPLSASMIKRYDE
HHQDLTLLKALVRQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPLARGNSRFAWM
TRKSEETITPWNFEEVVDKGASAQSFIERMTNEDKNLPNEKVLPKHSLLYEYFTVYNELTKVKYVTEGM
RKPAFLSGEQKKAIVDLLFKTNRKVTVKQLKEDYFKKIECFDSVEISGVEDRENASLGTYHDLLKIIKD
KDELDNEENEDILEDIVLTLTLFEDREMIEERLKTYAHLEDDKVMKQLKRRRYTGWGRLSRKLINGIRD
KQSGKTILDELKSDGFANRNEMQLIHDDSLTFKEDIQKAQVSGQGDSLHEHIANLAGSPAIKKGILQTV
KVVDELVKVMGRHKPENIVIEMARENQTTQKGQKNSRERMKRIEEGIKELGSQILKEHPVENTQLQNEK
LYLYYLQNGRDMYVDQELDINRLSDYDVDAIVPQSFLKDDSIDNKVLTRSDKNRGKSDNVPSEEVVKKM
KNYWRQLLNAKLITQRKEDNLTKAERGGLSELDKAGFIKRQLVETRQITKHVAQILDSRMNTKYDENDK
LIREVKVITLKSKLVSDERKDFQFYKVREINNYHHAHDAYLNAVVGTALIKKYPKLESEFVYGDYKVYD
VRKMIAKSEQEIGKATAKYFFYSNIMNFFKTEITLANGEIRKRPLIETNGETGEIVWDKGRDFATVRKV
LSMPQVNIVKKTEVQTGGFSKESILPKRNSDKLIARKKDWDPKKYGGEDSPTVAYSVLVVAKVEKGKSK
KLKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLSA
YNKHRDKPIREQAENIIHLFTLTNLGAPAAFKYEDTTIDRKRYTSTKEVLDATLIHQSITGLYETRIDL
SQLGGDSGGSSGGSSGSETPGTSESATPESSGGSSGGSSTLNIEDEYRLHETSKEPDVSLGSTWLSDFP
QAWAETGGMGLAVRQAPLIIPLKATSTPVSIKQYPMSQEARLGIKPHIQRLLDQGILVPCQSPWNTPLL
PVKKPGTNDYRPVQDLREVNKRVEDIHPTVPNPYNLLSGLPPSHQWYTVLDLKDAFFCLRLHPTSQPLE
AFEWRDPEMGISGQLTWTRLPQGEKNSPTLENEALHRDLADFRIQHPDLILLQYVDDLLLAATSELDCQ
QGTRALLQTLGNLGYRASAKKAQICQKQVKYLGYLLKEGQRWLTEARKETVMGQPTPKTPRQLREFLGK
AGFCRLFIPGFAEMAAPLYPLTKPGTLENWGPDQQKAYQEIKQALLTAPALGLPDLTKPFELFVDEKQG
YAKGVLTQKLGPWRRPVAYLSKKLDPVAAGWPPCLRMVAAIAVLTKDAGKLTMGQPLVILAPHAVEALV
KQPPDRWLSNARMTHYQALLLDTDRVQFGPVVALNPATLLPLPEEGLQHNCLSGGSKRTADGSEPKKKR
KVGS
Rattus norvegicus & synthetic: APOBEC1-XTEN L8-nspCas9-UGI-SV40 NLS
(SEQ ID NO: 40)
MSSETGPVAVDPTLRRRIEPHEFEVFFDPRELRKETCLLYEINWGGRHSIWRHTSQNTNKHVEVNFIEK
FTTERYFCPNTRCSITWFLSWSPCGECSRAITEFLSRYPHVTLFIYIARLYHHADPRNRQGLRDLISSG
VTIQIMTEQESGYCWRNFVNYSPSNEAHWPRYPHLWVRLYVLELYCIILGLPPCLNILRRKQPQLTEFT
IALQSCHYQRLPPHILWATGLKSGSETPGTSESATPESDKKYSIGLAIGTNSVGWAVITDEYKVPSKKE
KVLGNTDRHSIKKNLIGALLFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADLRLIYLALAHMIKERGHFLI
EGDLNPDNSDVDKLFIQLVQTYNQLFEENPINASGVDAKAILSARLSKSRRLENLIAQLPGEKKNGLEG
NLIALSLGLTPNEKSNFDLAEDAKLQLSKDTYDDDLDNLLAQIGDQYADLFLAAKNLSDAILLSDILRV
NTEITKAPLSASMIKRYDEHHQDLTLLKALVRQQLPEKYKEIFFDQSKNGYAGYIDGGASQEEFYKFIK
PILEKMDGTEELLVKLNREDLLRKQRTFDNGSIPHQIHLGELHAILRRQEDFYPFLKDNREKIEKILTF
RIPYYVGPLARGNSRFAWMTRKSEETITPWNFEEVVDKGASAQSFIERMTNEDKNLPNEKVLPKHSLLY
EYFTVYNELTKVKYVTEGMRKPAFLSGEQKKAIVDLLEKTNRKVTVKQLKEDYFKKIECFDSVEISGVE
DRFNASLGTYHDLLKIIKDKDELDNEENEDILEDIVLTLTLFEDREMIEERLKTYAHLEDDKVMKQLKR
RRYTGWGRLSRKLINGIRDKQSGKTILDFLKSDGFANRNFMQLIHDDSLTFKEDIQKAQVSGQGDSLHE
HIANLAGSPAIKKGILQTVKVVDELVKVMGRHKPENIVIEMARENQTTQKGQKNSRERMKRIEEGIKEL
GSQILKEHPVENTQLQNEKLYLYYLQNGRDMYVDQELDINRLSDYDVDHIVPQSFLKDDSIDNKVLTRS
DKNRGKSDNVPSEEVVKKMKNYWRQLLNAKLITQRKEDNLTKAERGGLSELDKAGFIKRQLVETRQITK
HVAQILDSRMNTKYDENDKLIREVKVITLKSKLVSDERKDFQFYKVREINNYHHAHDAYLNAVVGTALI
KKYPKLESEFVYGDYKVYDVRKMIAKSEQEIGKATAKYFFYSNIMNFFKTEITLANGEIRKRPLIETNG
ETGEIVWDKGRDFATVRKVLSMPQVNIVKKTEVQTGGFSKESILPKRNSDKLIARKKDWDPKKYGGEDS
PTVAYSVLVVAKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAAFKYEDTTIDRKRYTSTKEVL
DATLIHQSITGLYETRIDLSQLGGDSGGSTNLSDIIEKETGKQLVIQESILMLPEEVEEVIGNKPESDI
LVHTAYDESTDENVMLLTSDAPEYKPWALVIQDSNGENKIKMLSGGSPKKKRKV
Homo sapiens: AID
(SEQ ID NO: 41)
MDSLLMNRRKFLYQFKNVRWAKGRRETYLCYVVKRRDSATSFSLDEGYLRNKNGCHVELLFLRYISDWD
LDPGRCYRVTWFTSWSPCYDCARHVADELRGNPNLSLRIFTARLYFCEDRKAEPEGLRRLHRAGVQIAI
MTFKDYFYCWNTEVENHERTFKAWEGLHENSVRLSRQLRRILLPLYEVDDLRDAFRTLGL
Homo sapiens: AIDv solubility variant lacking N-terminal RNA-binding
region
(SEQ ID NO: 42)
LMDPHIFTSNENNGIGRHKTYLCYEVERLDSATSFSLDFGYLRNKNGCHVELLFLRYISDWDLDPGRCY
RVTWFTSWSPCYDCARHVADFLRGNPNLSLRIFTARLYFCEDRKAEPEGLRRLHRAGVQIAIMTFKDYF
YCWNTEVENHERTFKAWEGLHENSVRLSRQLRRILLPLYEVDDLRDAFRTLGL
Homo sapiens: AIDv solubility variant lacking N-terminal RNA-binding
region and the C-terminal poorly structured region
(SEQ ID NO: 43)
MDPHIFTSNENNGIGRHKTYLCYEVERLDSATSESLDFGYLRNKNGCHVELLFLRYISDWDLDPGRCYR
VTWFTSWSPCYDCARHVADFLRGNPNLSLRIFTARLYFCEDRKAEPEGLRRLHRAGVQIAIMTFKDYFY
CWNTEVENHERTFKAWEGLHENSVRLSRQLRRILLPL
Rattus norvegicus: APOBEC1
(SEQ ID NO: 44)
MSSETGPVAVDPTLRRRIEPHEFEVFFDPRELRKETCLLYEINWGGRHSIWRHTSQNTNKHVEVNFIEK
FTTERYFCPNTRCSITWELSWSPCGECSRAITEFLSRYPHVTLFIYIARLYHHADPRNRQGLRDLISSG
VTIQIMTEQESGYCWRNFVNYSPSNEAHWPRYPHLWVRLYVLELYCIILGLPPCLNILRRKQPQLTFFT
IALQSCHYQRLPPHILWATGLK
Mus musculus: APOBEC3
(SEQ ID NO: 45)
MGPFCLGCSHRKCYSPIRNLISQETFKFHFKNLGYAKGRKDTFLCYEVTRKDCDSPVSLHHGVFKNKDN
IHAEICFLYWFHDKVLKVLSPREEFKITWYMSWSPCFECAEQIVRFLATHHNLSLDIFSSRLYNVQDPE
TQQNLCRLVQEGAQVAAMDLYEFKKCWKKFVDNGGRRFRPWKRLLTNFRYQDSKLQEILRRMDPLSEEE
FYSQFYNQRVKHLCYYHRMKPYLCYQLEQFNGQAPLKGCLLSEKGKQHAEILELDKIRSMELSQVTITC
YLTWSPCPNCAWQLAAFKRDRPDLILHIYTSRLYFHWKRPFQKGLCSLWQSGILVDVMDLPQFTDCWTN
FVNPKRPFRPWKGLEIISRRTQRRLRRIKESWGLQDLVNDEGNLQLGPPMSN
Mus musculus: APOBEC3 catalytic domain
(SEQ ID NO: 46)
MGPFCLGCSHRKCYSPIRNLISQETFKFHFKNLGYAKGRKDTFLCYEVTRKDCDSPVSLHHGVEKNKDN
IHAEICFLYWFHDKVLKVLSPREEFKITWYMSWSPCFECAEQIVRFLATHHNLSLDIFSSRLYNVQDPE
TQQNLCRLVQEGAQVAAMDLYEFKKCWKKFVDNGGRRFRPWKRLLTNFRYQDSKLQEILRR
Homo sapiens: APOBEC3A
(SEQ ID NO: 47)
MEASPASGPRHLMDPHIFTSNENNGIGRHKTYLCYEVERLDNGTSVKMDQHRGFLHNQAKNLLCGFYGR
HAELRELDLVPSLQLDPAQIYRVTWFISWSPCESWGCAGEVRAFLQENTHVRLRIFAARIYDYDPLYKE
ALQMLRDAGAQVSIMTYDEFKHCWDTFVDHQGCPFQPWDGLDEHSQALSGRLRAILQNQGN
Homo sapiens: APOBEC3G
(SEQ ID NO: 48)
MKPHFRNTVERMYRDTESYNFYNRPILSRRNTVWLCYEVKTKGPSRPPLDAKIFRGQVYSELKYHPEMR
FFHWFSKWRKLHRDQEYEVTWYISWSPCTKCTRDMATFLAEDPKVTLTIFVARLYYFWDPDYQEALRSL
CQKRDGPRATMKIMNYDEFQHCWSKFVYSQRELFEPWNNLPKYYILLHIMLGEILRHSMDPPTFTENEN
NEPWVRGRHETYLCYEVERMHNDTWVLLNQRRGFLCNQAPHKHGFLEGRHAELCELDVIPFWKLDLDQD
YRVTCFTSWSPCFSCAQEMAKFISKNKHVSLCIFTARIYDDQGRCQEGLRTLAEAGAKISIMTYSEFKH
CWDTFVDHQGCPFQPWDGLDEHSQDLSGRLRAILQNQEN
Homo sapiens: APOBEC3G catalytic domain
(SEQ ID NO: 49)
PPTFTFNENNEPWVRGRHETYLCYEVERMHNDTWVLLNQRRGFLCNQAPHKHGFLEGRHAELCELDVIP
FWKLDLDQDYRVTCFTSWSPCFSCAQEMAKFISKNKHVSLCIFTARIYDDQGRCQEGLRTLAEAGAKIS
IMTYSEFKHCWDTFVDHQGCPFQPWDGLDEHSQDLSGRLRAILQNQEN
Homo sapiens: APOBEC3H
(SEQ ID NO: 50)
MALLTAETFRLQFNNKRRLRRPYYPRKALLCYQLTPQNGSTPTRGYFENKKKCHAEICFINEIKSMGLD
ETQCYQVTCYLTWSPCSSCAWELVDFIKAHDHLNLGIFASRLYYHWCKPQQKGLRLLCGSQVPVEVMGE
PKFADCWENFVDHEKPLSENPYKMLEELDKNSRAIKRRLERIKIPGVRAQGRYMDILCDAEV
Homo sapiens: APOBEC3F
(SEQ ID NO: 51)
MKPHFRNTVERMYRDTFSYNFYNRPILSRRNTVWLCYEVKTKGPSRPRLDAKIFRGQVYSQPEHHAEMC
FLSWFCGNQLPAYKCFQITWFVSWTPCPDCVAKLAEFLAEHPNVTLTISAARLYYYWERDYRRALCRLS
QAGARVKIMDDEEFAYCWENFVYSEGQPFMPWYKEDDNYAFLHRTLKEILRNPMEAMYPHIFYFHEKNL
RKAYGRNESWLCFTMEVVKHHSPVSWKRGVERNQVDPETHCHAERCELSWFCDDILSPNTNYEVTWYTS
WSPCPECAGEVAEFLARHSNVNLTIFTARLYYFWDTDYQEGLRSLSQEGASVEIMGYKDEKYCWENFVY
NDDEPFKPWKGLKYNFLFLDSKLQEILE
Homo sapiens: APOBEC3F catalytic domain
(SEQ ID NO: 52)
KEILRNPMEAMYPHIFYFHFKNLRKAYGRNESWLCFTMEVVKHHSPVSWKRGVERNQVDPETHCHAERC
FLSWFCDDILSPNTNYEVTWYTSWSPCPECAGEVAEFLARHSNVNLTIFTARLYYFWDTDYQEGLRSLS
QEGASVEIMGYKDEKYCWENFVYNDDEPFKPWKGLKYNFLFLDSKLQEILE
Escherichia coli: TadA
(SEQ ID NO: 53)
MKRTADGSEFESPKKKRKVSEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGRVVEGARDAKTGAAGSLMDV
LHHPGMNHRVEITEGILADECAALLSDFFRMRRQEIKAQKKAQSSTDSGGSSGGSSGSETPGTSESATP
ESSGGSSGGSSEVEFSHEYWMRHALTLAKRARDEREVPVGAVLVLNNRVIGEGWNRAIGLHDPTAHAEI
MALRQGGLVMQNYRLIDATLYVTFEPCVMCAGAMIHSRIGRVVFGVRNAKTGAAGSLMDVLHYPGMNHR
VEITEGILADECAALLCYFFRMPROVENAQKKAQSSTD
Homo sapiens: Adar1
(SEQ ID NO: 54)
MNPRQGYSLSGYYTHPFQGYEHRQLRYQQPGPGSSPSSFLLKQIEFLKGQLPEAPVIGKQTPSLPPSLP
GLRPRFPVLLASSTRGRQVDIRGVPRGVHLGSQGLQRGFQHPSPRGRSLPQRGVDCLSSHEQELSIYQD
QEQRILKFLEELGEGKATTAHDLSGKLGTPKKEINRVLYSLAKKGKLQKEAGTPPLWKIAVSTQAWNQH
SGVVRPDGHSQGAPNSDPSLEPEDRNSTSVSEDLLEPFIAVSAQAWNQHSGVVRPDSHSQGSPNSDPGL
EPEDSNSTSALEDPLEFLDMAEIKEKICDYLENVSDSSALNLAKNIGLTKARDINAVLIDMERQGDVYR
QGTTPPIWHLTDKKRERMQIKRNTNSVPETAPAAIPETKRNAEFLTCNIPTSNASNNMVTTEKVENGQE
PVIKLENRQEARPEPARLKPPVHYNGPSKAGYVDFENGQWATDDIPDDLNSIRAAPGEFRAIMEMPSFY
SHGLPRCSPYKKLTECQLKNPISGLLEYAQFASQTCEENMIEQSGPPHEPREKFQVVINGREFPPAEAG
SKKVAKQDAAMKAMTILLEEAKAKDSGKSEESSHYSTEKESEKTAESQTPTPSATSFFSGKSPVTTLLE
CMHKLGNSCEFRLLSKEGPAHEPKFQYCVAVGAQTFPSVSAPSKKVAKQMAAEEAMKALHGEATNSMAS
DNQPEGMISESLDNLESMMPNKVRKIGELVRYLNTNPVGGLLEYARSHGFAAEFKLVDQSGPPHEPKFV
YQAKVGGRWFPAVCAHSKKQGKQEAADAALRVLIGENEKAERMGFTEVTPVTGASLRRTMLLLSRSPEA
QPKTLPLTGSTFHDQIAMLSHRCENTLTNSFQPSLLGRKILAAIIMKKDSEDMGVVVSLGTGNRCVKGD
SLSLKGETVNDCHAEIISRRGFIRFLYSELMKYNSQTAKDSIFEPAKGGEKLQIKKTVSFHLYISTAPC
GDGALFDKSCSDRAMESTESRHYPVFENPKQGKLRTKVENGEGTIPVESSDIVPTWDGIRLGERLRTMS
CSDKILRWNVLGLQGALLTHFLQPIYLKSVTLGYLFSQGHLTRAICCRVTRDGSAFEDGLRHPFIVNHP
KVGRVSIYDSKRQSGKTKETSVNWCLADGYDLEILDGTRGTVDGPRNELSRVSKKNIFLLEKKLCSFRY
RRDLLRLSYGEAKKAARDYETAKNYFKKGLKDMGYGNWISKPQEEKNFYLCPV
Homo sapiens: Adar2
MDIEDEENMSSSSTDVKENRNLDNVSPKDGSTPGPGEGSQLSNGGGGGPGRKRPLEEGSNGHSKYRLKK
RRKTPGPVLPKNALMQLNEIKPGLQYTLLSQTGPVHAPLFVMSVEVNGQVFEGSGPTKKKAKLHAAEKA
LRSFVQFPNASEAHLAMGRTLSVNTDFTSDQADEPDTLENGFETPDKAEPPFYVGSNGDDSFSSSGDLS
LSASPVPASLAQPPLPVLPPFPPPSGKNPVMILNELRPGLKYDELSESGESHAKSFVMSVVVDGQFFEG
SGRNKKLAKARAAQSALAAIFNLHLDQTPSRQPIPSEGLQLHLPQVLADAVSRLVLGKFGDLTDNESSP
HARRKVLAGVVMTTGTDVKDAKVISVSTGTKCINGEYMSDRGLALNDCHAEIISRRSLLRFLYTQLELY
LNNKDDQKRSIFQKSERGGFRLKENVQFHLYISTSPCGDARIFSPHEPILEEPADRHPNRKARGQLRTK
IESGQGTIPVRSNASIQTWDGVLQGERLLTMSCSDKIARWNVVGIQGSLLSIFVEPIYESSIILGSLYH
GDHLSRAMYQRISNIEDLPPLYTLNKPLLSGISNAEARQPGKAPNFSVNWTVGDSAIEVINATTGKDEL
GRASRLCKHALYCRWMRVHGKVPSHLLRSKITKPNVYHESKLAAKEYQAAKARLFTAFIKAGLGAWVEK
PTEQDQFSLTP (SEQ ID NO: 55)
Streptococcus pyogenes: Cas9 Bipartite NLS
(SEQ ID NO: 56)
MDKKYSIGLDIGTNSVGWAVITDEYKVPSKKFKVLGNTDRHSIKKNLIGALLEDSGETAEATRLKRTAR
RRYTRRKNRICYLQEIFSNEMAKVDDSFFHRLEESELVEEDKKHERHPIFGNIVDEVAYHEKYPTIYHL
RKKLVDSTDKADLRLIYLALAHMIKERGHFLIEGDLNPDNSDVDKLFIQLVQTYNQLFEENPINASGVD
AKAILSARLSKSRRLENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTEDNGSIPHQI
HLGELHAILRRQEDFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRENASLGTYHDLLKIIKDKDELDNEENEDILEDIVL
TLTLFEDREMIEERLKTYAHLFDDKVMKQLKRRRYTGWGRLSRKLINGIRDKQSGKTILDELKSDGFAN
RNFMQLIHDDSLTFKEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQLLNAKLITQRKE
DNLTKAERGGLSELDKAGFIKRQLVETRQITKHVAQILDSRMNTKYDENDKLIREVKVITLKSKLVSDE
RKDFQFYKVREINNYHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMPQVNIVKKTEVQTGG
FSKESILPKRNSDKLIARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGDGSGGGGSGKRTA
DGSEFEPKKKRKVSSGGDYKDHDGDYKDHDIDYKDDDDK
Staphylococcus aureus: Cas9
(SEQ ID NO: 57)
MKRNYILGLDIGITSVGYGIIDYETRDVIDAGVRLEKEANVENNEGRRSKRGARRLKRRRRHRIQRVKK
LLEDYNLLTDHSELSGINPYEARVKGLSQKLSEEEFSAALLHLAKRRGVHNVNEVEEDTGNELSTKEQI
SRNSKALEEKYVAELQLERLKKDGEVRGSINRFKTSDYVKEAKQLLKVQKAYHQLDQSFIDTYIDLLET
RRTYYEGPGEGSPFGWKDIKEWYEMLMGHCTYFPEELRSVKYAYNADLYNALNDLNNLVITRDENEKLE
YYEKFQIIENVFKQKKKPTLKQIAKEILVNEEDIKGYRVTSTGKPEFTNLKVYHDIKDITARKEIIENA
ELLDQIAKILTIYQSSEDIQEELTNLNSELTQEEIEQISNLKGYTGTHNLSLKAINLILDELWHTNDNQ
IAIFNRLKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPLEDLLNNPEN
YEVDHIIPRSVSFDNSFNNKVLVKQEENSKKGNRTPFQYLSSSDSKISYETFKKHILNLAKGKGRISKT
KKEYLLEERDINRESVQKDFINRNLVDTRYATRGLMNLLRSYERVNNLDVKVKSINGGFTSFLRRKWKF
KKERNKGYKHHAEDALIIANADFIFKEWKKLDKAKKVMENQMFEEKQAESMPEIETEQEYKEIFITPHQ
IKHIKDFKDYKYSHRVDKKPNRELINDTLYSTRKDDKGNTLIVNNLNGLYDKDNDKLKKLINKSPEKLL
MYHHDPQTYQKLKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLKKISNQAEFIASF
YNNDLIKINGELYRVIGVNNDLLNRIEVNMIDITYREYLENMNDKRPPRIIKTIASKTQSIKKYSTDIL
GNLYEVKSKKHPQIIKKG
Campylobacter jejuni: Cas9
(SEQ ID NO: 58)
MARILAFDIGISSIGWAFSENDELKDCGVRIFTKVENPKTGESLALPRRLARSARKRLARRKARLNHLK
HLIANEFKLNYEDYQSFDESLAKAYKGSLISPYELRFRALNELLSKQDFARVILHIAKRRGYDDIKNSD
DKEKGAILKAIKQNEEKLANYQSVGEYLYKEYFQKFKENSKEFTNVRNKKESYERCIAQSFLKDELKLI
FKKQREFGFSFSKKFEEEVLSVAFYKRALKDESHLVGNCSFFTDEKRAPKNSPLAFMFVALTRIINLLN
NLKNTEGILYTKDDLNALLNEVLKNGTLTYKQTKKLLGLSDDYEFKGEKGTYFIEFKKYKEFIKALGEH
NLSQDDLNEIAKDITLIKDEIKLKKALAKYDLNQNQIDSLSKLEFKDHLNISFKALKLVTPLMLEGKKY
DEACNELNLKVAINEDKKDELPAFNETYYKDEVTNPVVLRAIKEYRKVLNALLKKYGKVHKINIELARE
VGKNHSQRAKIEKEQNENYKAKKDAELECEKLGLKINSKNILKLRLFKEQKEFCAYSGEKIKISDLQDE
KMLEIDHIYPYSRSFDDSYMNKVLVFTKQNQEKLNQTPFEAFGNDSAKWQKIEVLAKNLPTKKQKRILD
KNYKDKEQKNFKDRNLNDTRYIARLVLNYTKDYLDFLPLSDDENTKLNDTQKGSKVHVEAKSGMLTSAL
RHTWGFSAKDRNNHLHHAIDAVIIAYANNSIVKAFSDEKKEQESNSAELYAKKISELDYKNKRKFFEPF
SGFRQKVLDKIDEIFVSKPERKKPSGALHEETFRKEEEFYQSYGGKEGVLKALELGKIRKVNGKIVKNG
DMFRVDIFKHKKTNKFYAVPIYTMDFALKVLPNKAVARSKKGEIKDWILMDENYEFCFSLYKDSLILIQ
TKDMQEPEFVYYNAFTSSTVSLIVSKHDNKFETLSKNQKILFKNANEKEVIAKSIGIQNLKVFEKYIVS
ALGEVTKAEFRQREDEKK
Neisseria meningitidis: Cas9
(SEQ ID NO: 59)
MAAFKPNSINYILGLDIGIASVGWAMVEIDEEENPIRLIDLGVRVFERAEVPKTGDSLAMARRLARSVR
RLTRRRAHRLLRTRRLLKREGVLQAANEDENGLIKSLPNTPWQLRAAALDRKLTPLEWSAVLLHLIKHR
GYLSQRKNEGETADKELGALLKGVAGNAHALQTGDFRTPAELALNKFEKESGHIRNQRSDYSHTFSRKD
LQAELILLFEKQKEFGNPHVSGGLKEGIETLLMTQRPALSGDAVQKMLGHCTFEPAEPKAAKNTYTAER
FIWLTKLNNLRILEQGSERPLTDTERATLMDEPYRKSKLTYAQARKLLGLEDTAFFKGLRYGKDNAEAS
TLMEMKAYHAISRALEKEGLKDKKSPLNLSPELQDEIGTAFSLFKTDEDITGRLKDRIQPEILEALLKH
ISFDKFVQISLKALRRIVPLMEQGKRYDEACAEIYGDHYGKKNTEEKIYLPPIPADEIRNPVVLRALSQ
ARKVINGVVRRYGSPARIHIETAREVGKSFKDRKEIEKRQEENRKDREKAAAKFREYFPNFVGEPKSKD
ILKLRLYEQQHGKCLYSGKEINLGRLNEKGYVEIDHALPFSRTWDDSENNKVLVLGSENQNKGNQTPYE
YFNGKDNSREWQEFKARVETSRFPRSKKQRILLQKFDEDGEKERNLNDTRYVNRFLCQFVADRMRLTGK
GKKRVFASNGQITNLLRGFWGLRKVRAENDRHHALDAVVVACSTVAMQQKITRFVRYKEMNAFDGKTID
KETGEVLHQKTHFPQPWEFFAQEVMIRVFGKPDGKPEFEEADTLEKLRTLLAEKLSSRPEAVHEYVTPL
FVSRAPNRKMSGQGHMETVKSAKRLDEGVSVLRVPLTQLKLKDLEKMVNREREPKLYEALKARLEAHKD
DPAKAFAEPFYKYDKAGNRTQQVKAVRVEQVQKTGVWVRNHNGIADNATMVRVDVFEKGDKYYLVPIYS
WQVAKGILPDRAVVQGKDEEDWQLIDDSENFKESLHPNDLVEVITKKARMFGYFASCHRGTGNINIRIH
DLDHKIGKNGILEGIGVKTALSFQKYQIDELGKEIRPCRLKKRPPVR
Acidaminococcus sp. Cas12a
(SEQ ID NO: 60)
MTQFEGFTNLYQVSKTLRFELIPQGKTLKHIQEQGFIEEDKARNDHYKELKPIIDRIYKTYADQCLQLV
QLDWENLSAAIDSYRKEKTEETRNALIEEQATYRNAIHDYFIGRTDNLTDAINKRHAEIYKGLFKAELF
NGKVLKQLGTVTTTEHENALLRSFDKFTTYFSGFYENRKNVESAEDISTAIPHRIVQDNFPKFKENCHI
FTRLITAVPSLREHFENVKKAIGIFVSTSIEEVESFPFYNQLLTQTQIDLYNQLLGGISREAGTEKIKG
LNEVLNLAIQKNDETAHIIASLPHRFIPLFKQILSDRNTLSFILEEFKSDEEVIQSFCKYKTLLRNENV
LETAEALFNELNSIDLTHIFISHKKLETISSALCDHWDTLRNALYERRISELTGKITKSAKEKVQRSLK
HEDINLQEIISAAGKELSEAFKQKTSEILSHAHAALDQPLPTTLKKQEEKEILKSQLDSLLGLYHLLDW
FAVDESNEVDPEFSARLTGIKLEMEPSLSFYNKARNYATKKPYSVEKFKLNFQMPTLASGWDVNKEKNN
GAILFVKNGLYYLGIMPKQKGRYKALSFEPTEKTSEGFDKMYYDYFPDAAKMIPKCSTQLKAVTAHFQT
HTTPILLSNNFIEPLEITKEIYDLNNPEKEPKKFQTAYAKKTGDQKGYREALCKWIDFTRDELSKYTKT
TSIDLSSLRPSSQYKDLGEYYAELNPLLYHISFQRIAEKEIMDAVETGKLYLFQIYNKDFAKGHHGKPN
LHTLYWTGLESPENLAKTSIKLNGQAELFYRPKSRMKRMAHRLGEKMLNKKLKDQKTPIPDTLYQELYD
YVNHRLSHDLSDEARALLPNVITKEVSHEIIKDRRFTSDKFFFHVPITLNYQAANSPSKENQRVNAYLK
EHPETPIIGIDRGERNLIYITVIDSTGKILEQRSLNTIQQFDYQKKLDNREKERVAARQAWSVVGTIKD
LKQGYLSQVIHEIVDLMIHYQAVVVLENLNFGFKSKRTGIAEKAVYQQFEKMLIDKLNCLVLKDYPAEK
VGGVLNPYQLTDQFTSFAKMGTQSGFLFYVPAPYTSKIDPLTGFVDPFVWKTIKNHESRKHFLEGEDEL
HYDVKTGDFILHFKMNRNLSFQRGLPGFMPAWDIVFEKNETQFDAKGTPFIAGKRIVPVIENHRFTGRY
RDLYPANELIALLEEKGIVERDGSNILPKLLENDDSHAIDTMVALIRSVLQMRNSNAATGEDYINSPVR
DLNGVCFDSRFQNPEWPMDADANGAYHIALKGQLLLNHLKESKDLKLQNGISNQDWLAYIQELRN
Lachnospiraceae bacterium Cas12a:
(SEQ ID NO: 61)
MSKLEKFTNCYSLSKTLRFKAIPVGKTQENIDNKRLLVEDEKRAEDYKGVKKLLDRYYLSFINDVLHSI
KLKNLNNYISLFRKKTRTEKENKELENLEINLRKEIAKAFKGNEGYKSLFKKDIIETILPEFLDDKDEI
ALVNSFNGFTTAFTGFFDNRENMFSEEAKSTSIAFRCINENLTRYISNMDIFEKVDAIFDKHEVQEIKE
KILNSDYDVEDFFEGEFFNFVLTQEGIDVYNAIIGGFVTESGEKIKGLNEYINLYNQKTKQKLPKEKPL
YKQVLSDRESLSFYGEGYTSDEEVLEVERNTLNKNSEIFSSIKKLEKLEKNEDEYSSAGIFVKNGPAIS
TISKDIFGEWNVIRDKWNAEYDDIHLKKKAVVTEKYEDDRRKSFKKIGSESLEQLQEYADADLSVVEKL
KEIIIQKVDEIYKVYGSSEKLFDADFVLEKSLKKNDAVVAIMKDLLDSVKSFENYIKAFFGEGKETNRD
ESFYGDFVLAYDILLKVDHIYDAIRNYVTQKPYSKDKFKLYFQNPQFMGGWDKDKETDYRATILRYGSK
YYLAIMDKKYAKCLQKIDKDDVNGNYEKINYKLLPGPNKMLPKVFFSKKWMAYYNPSEDIQKIYKNGTE
KKGDMENLNDCHKLIDFFKDSISRYPKWSNAYDENESETEKYKDIAGFYREVEEQGYKVSFESASKKEV
DKLVEEGKLYMFQIYNKDFSDKSHGTPNLHTMYFKLLEDENNHGQIRLSGGAELFMRRASLKKEELVVH
PANSPIANKNPDNPKKTTTLSYDVYKDKRFSEDQYELHIPIAINKCPKNIFKINTEVRVLLKHDDNPYV
IGIDRGERNLLYIVVVDGKGNIVEQYSLNEIINNENGIRIKTDYHSLLDKKEKERFEARQNWTSIENIK
ELKAGYISQVVHKICELVEKYDAVIALEDLNSGFKNSRVKVEKQVYQKFEKMLIDKLNYMVDKKSNPCA
TGGALKGYQITNKFESFKSMSTQNGFIFYIPAWLTSKIDPSTGFVNLLKTKYTSIADSKKFISSFDRIM
YVPEEDLFEFALDYKNFSRTDADYIKKWKLYSYGNRIRIFRNPKKNNVEDWEEVCLTSAYKELENKYGI
NYQQGDIRALLCEQSDKAFYSSFMALMSLMLQMRNSITGRTDVDELISPVKNSDGIFYDSRNYEAQENA
ILPKNADANGAYNIARKVLWAIGQFKKAEDEKLDKVKIAISNKEWLEYAQTSVKH
Leptotrichia shahii Cas13a
(SEQ ID NO: 62)
MGNLFGHKRWYEVRDKKDFKIKRKVKVKRNYDGNKYILNINENNNKEKIDNNKFIRKYINYKKNDNILK
EFTRKFHAGNILFKLKGKEGIIRIENNDDFLETEEVVLYIEAYGKSEKLKALGITKKKIIDEAIRQGIT
KDDKKIEIKRQENEEEIEIDIRDEYTNKTLNDCSIILRIIENDELETKKSIYEIFKNINMSLYKIIEKI
IENETEKVFENRYYEEHLREKLLKDDKIDVILTNFMEIREKIKSNLEILGFVKFYLNVGGDKKKSKNKK
MLVEKILNINVDLTVEDIADFVIKELEFWNITKRIEKVKKVNNEFLEKRRNRTYIKSYVLLDKHEKFKI
ERENKKDKIVKFFVENIKNNSIKEKIEKILAEFKIDELIKKLEKELKKGNCDTEIFGIFKKHYKVNEDS
KKFSKKSDEEKELYKIIYRYLKGRIEKILVNEQKVRLKKMEKIEIEKILNESILSEKILKRVKQYTLEH
IMYLGKLRHNDIDMTTVNTDDESRLHAKEELDLELITFFASTNMELNKIFSRENINNDENIDFFGGDRE
KNYVLDKKILNSKIKIIRDLDFIDNKNNITNNFIRKFTKIGTNERNRILHAISKERDLQGTQDDYNKVI
NIIQNLKISDEEVSKALNLDVVEKDKKNIITKINDIKISEENNNDIKYLPSFSKVLPEILNLYRNNPKN
EPFDTIETEKIVLNALIYVNKELYKKLILEDDLEENESKNIFLQELKKTLGNIDEIDENIIENYYKNAQ
ISASKGNNKAIKKYQKKVIECYIGYLRKNYEELFDFSDFKMNIQEIKKQIKDINDNKTYERITVKTSDK
TIVINDDFEYIISIFALLNSNAVINKIRNRFFATSVWLNTSEYQNIIDILDEIMQLNTLRNECITENWN
LNLEEFIQKMKEIEKDEDDEKIQTKKEIFNNYYEDIKNNILTEFKDDINGCDVLEKKLEKIVIEDDETK
FEIDKKSNILQDEQRKLSNINKKDLKKKVDQYIKDKDQEIKSKILCRIIFNSDELKKYKKEIDNLIEDM
ESENENKFQEIYYPKERKNELYIYKKNLFLNIGNPNFDKIYGLISNDIKMADAKFLENIDGKNIRKNKI
SEIDAILKNLNDKLNGYSKEYKEKYIKKLKENDDEFAKNIQNKNYKSFEKDYNRVSEYKKIRDLVEFNY
LNKIESYLIDINWKLAIQMARFERDMHYIVNGLRELGIIKLSGYNTGISRAYPKRNGSDGFYTTTAYYK
FFDEESYKKFEKICYGFGIDLSENSEINKPENESIRNYISHFYIVRNPFADYSIAEQIDRVSNLLSYST
RYNNSTYASVFEVFKKDVNLDYDELKKKFKLIGNNDILERLMKPKKVSVLELESYNSDYIKNLIIELLT
KIENTNDTL
Leptotrichia wadei Cas13a
(SEQ ID NO: 63)
MKVTKVDGISHKKYIEEGKLVKSTSEENRTSERLSELLSIRLDIYIKNPDNASEEENRIRRENLKKFFS
NKVLHLKDSVLYLKNRKEKNAVQDKNYSEEDISEYDLKNKNSESVLKKILLNEDVNSEELEIFRKDVEA
KLNKINSLKYSFEENKANYQKINENNVEKVGGKSKRNIIYDYYRESAKRNDYINNVQEAFDKLYKKEDI
EKLFFLIENSKKHEKYKIREYYHKIIGRKNDKENFAKIIYEEIQNVNNIKELIEKIPDMSELKKSQVFY
KYYLDKEELNDKNIKYAFCHFVEIEMSQLLKNYVYKRLSNISNDKIKRIFEYQNLKKLIENKLLNKLDT
YVRNCGKYNYYLQVGEIATSDFIARNRQNEAFLRNIIGVSSVAYESLRNILETENENGITGRMRGKTVK
NNKGEEKYVSGEVDKIYNENKQNEVKENLKMFYSYDENMDNKNEIEDFFANIDEAISSIRHGIVHENLE
LEGKDIFAFKNIAPSEISKKMFQNEINEKKLKLKIFKQLNSANVENYYEKDVIIKYLKNTKENFVNKNI
PFVPSFTKLYNKIEDLRNTLKFFWSVPKDKEEKDAQIYLLKNIYYGEFLNKFVKNSKVFFKITNEVIKI
NKQRNQKTGHYKYQKFENIEKTVPVEYLAIIQSREMINNQDKEEKNTYIDFIQQIFLKGFIDYLNKNNL
KYIESNNNNDNNDIFSKIKIKKDNKEKYDKILKNYEKHNRNKEIPHEINEFVREIKLGKILKYTENLNM
FYLILKLLNHKELTNLKGSLEKYQSANKEETFSDELELINLLNLDNNRVTEDFELEANEIGKELDENEN
KIKDRKELKKFDTNKIYEDGENIIKHRAFYNIKKYGMLNLLEKIADKAKYKISLKELKEYSNKKNEIEK
NYTMQQNLHRKYARPKKDEKENDEDYKEYEKAIGNIQKYTHLKNKVEFNELNLLQGLLLKILHRLVGYT
SIWERDLRFRLKGEFPENHYIEEIFNFDNSKNVKYKSGQIVEKYINFYKELYKDNVEKRSIYSDKKVKK
LKQEKKDLYIRNYIAHFNYIPHAEISLLEVLENLRKLLSYDRKLKNAIMKSIVDILKEYGFVATFKIGA
DKKIEIQTLESEKIVHLKNLKKKKLMTDRNSEELCELVKVMFEYKALE
Pleckstrin homology domain of Human ARNO:
(SEQ ID NO: 64)
NPDREGWLLKLGGGRVKTWKRRWFILTDNCLYYFEYTTDKEPRGIIPLENLSIREVDDPRKPNCFELYI
PNNKGQLIKACKTEADGRVVEGNHMVYRISAPTQEEKDEWIKSIQAAVS
Pleckstrin homology domain of Human ARNO R279C:
(SEQ ID NO: 65)
NPDREGWLLKLGGGRVKTWKCRWFILTDNCLYYFEYTTDKEPRGIIPLENLSIREVDDPRKPNCFELYI
PNNKGQLIKACKTEADGRVVEGNHMVYRISAPTQEEKDEWIKSIQAAVS
FYVE domain of Human EEA1:
(SEQ ID NO: 66)
DNEVQNCMACGKGFSVTVRRHHCRQCGNIFCAECSAKNALTPSSKKPVRVCDACENDLQ
FYVE domain of Human EEA1 R1375L:
(SEQ ID NO: 67)
DNEVQNCMACGKGFSVTVRRHHCLQCGNIFCAECSAKNALTPSSKKPVRVCDACENDLQ
PX domain of p40^{phox} (NCF4):
(SEQ ID NO: 68)
DVAISANIADIEEKRGFTSHFVFVIEVKTKGGSKYLIYRRYRQFHALQSKLEERFGPDSKSSALACTLP
TLPAKVYVGVKQEIAEMRIPALNAYMKSLLSLPVWVLMDEDVRIFFYQSPYDS
PX domain of p40^{phox} (NCF4) R58L:
(SEQ ID NO: 69)
DVAISANIADIEEKRGFTSHFVFVIEVKTKGGSKYLIYLRYRQFHALQSKLEERFGPDSKSSALACTLP
TLPAKVYVGVKQEIAEMRIPALNAYMKSLLSLPVWVLMDEDVRIFFYQSPYDS
Pleckstrin homology domain of Homo sapiens DAPP1
(SEQ ID NO: 70)
MQTGRTEDDLVPTAPSLGTKEGYLTKQGGLVKTWKTRWFTLHRNELKYFKDQMSPEPIRILDLTECSAV
QFDYSQERVNCFCLVFPFRTFYLCAKTGVEADEWIKILRWKLSQIRKOLNQGEGTIR
Pleckstrin homology domain of Homo sapiens GRP1 (CYTH3)
(SEQ ID NO: 71)
PFKIPEDDGNDLTHTFFNPDREGWLLKLGGRVKTWKRRWFILTDNCLYYFEYTTDKEPRGIIPLENLSI
REVEDPRKPNCFELYNPSHKGQVIKACKTEADGRVVEGNHVVYRISAPSPEEKEEWMKSIKASISRDPE
YDMLATRKRRIANKK
Pleckstrin homology domain of Homo sapiens GRP1 (CYTH3) R284C:
(SEQ ID NO: 72)
MPFKIPEDDGNDLTHTFFNPDREGWLLKLGGRVKTWKCRWFILTDNCLYYFEYTTDKEPRGIIPLENLS
IREVEDPRKPNCFELYNPSHKGQVIKACKTEADGRVVEGNHVVYRISAPSPEEKEEWMKSIKASISRDP
FYDMLATRKRRIANKK
Pleckstrin homology domain of Human OSBP1
(SEQ ID NO: 73)
MGSGSAREGWLFKWTNYIKGYQRRWFVLSNGLLSYYRSKAEMRHTCRGTINLATANITVEDSCNFIISN
GGAQTYHLKASSEVERQRWVTALELAKAKAVK
Pleckstrin homology domain of Human OSBP1 R108E:
(SEQ ID NO: 74)
MGSGSAREGWLFKWTNYIKGYQERWFVLSNGLLSYYRSKAEMRHTCRGTINLATANITVEDSCNFIISN
GGAQTYHLKASSEVERQRWVTALELAKAKAVK
Pleckstrin homology domain of Human Btk1
(SEQ ID NO: 75)
MAAVILESIFLKRSQQKKKTSPLNFKKRLFLLTVHKLSYYEYDFERGRRGSKKGSIDVEKITCVETVVP
EKNPPPERQIPRRGEESSEMEQISIIERFPYPFQVVYDEGPLYVESPTEELRKRWIHQLKNVIRYNSDL
VQKYHPCFWIDGQYLCCSQTAKNAMGCQILENRNGSLKP
Pleckstrin homology domain of Human Btk1 R28C:
(SEQ ID NO: 76)
MAAVILESIFLKRSQQKKKTSPLNFKKCLFLLTVHKLSYYEYDFERGRRGSKKGSIDVEKITCVETVVP
EKNPPPERQIPRRGEESSEMEQISIIERFPYPFQVVYDEGPLYVESPTEELRKRWIHQLKNVIRYNSDL
VQKYHPCFWIDGQYLCCSQTAKNAMGCQILENRNGSLKP
Pleckstrin homology domain of Human FAPP1
(SEQ ID NO: 77)
MEGVLYKWTNYLTGWQPRWFVLDNGILSYYDSQDDVCKGSKGSIKMAVCEIKVHSADNTRMELIIPGEQ
HFYMKAVNAAERQRWLVALGSSKACLTDT
Pleckstrin homology domain of Human CERT
(SEQ ID NO: 78)
PVERCGVLSKWTNYIHGWQDRWVVLKNNALSYYKSEDETEYGCRGSICLSKAVITPHDFDECREDISVN
DSVWYLRAQDPDHRQQWIDAIEQHKT
Pleckstrin homology domain of Human PHLPP1
(SEQ ID NO: 79)
MRIQLSGMYNVRKGKMQLPVNRWTRRQVILCGTCLIVSSVKDSLTGKMHVLPLIGGKVEEVKKHQHCLA
FSSSGPQSQTYYICFDTFTEYLRWLRQVSKVAS
Pleckstrin homology domain of Human SWAP70
(SEQ ID NO: 80)
MDVLKQGYMMKKGHRRKNWTERWFVLKPNIISYYVSEDLKDKKGDILLDENCCVESLPDKDGKKCLFLV
KCFDKTFEISASDKKKKQEWIQAIHSTIH
Pleckstrin homology domain of Human SWAP70 R223E, R224E:
(SEQ ID NO: 81)
MDVLKQGYMMKKGHEEKNWTERWFVLKPNIISYYVSEDLKDKKGDILLDENCCVESLPDKDGKKCLFLV
KCFDKTFEISASDKKKKQEWIQAIHSTIH
Pleckstrin homology domain of Human MAPKAP1
(SEQ ID NO: 82)
MDMLSSHHYKSFKVSMIHRLRFTTDVQLGISGDKVEIDPVTNQKASTKFWIKQKPISIDSDLLCACDLA
EEKSPSHAIFKLTYLSNHDYKHLYFESDAATVNEIVLKVNYILES
Pleckstrin Homology Domain of Human PKD
(SEQ ID NO: 83)
MGTVMKEGWMVHYTSKDTLRKRHYWRLDSKCITLFQNDTGSRYYKEIPLSEILSLEPVKTSALIPNGAN
PHCFEITTANVVYYVGENVVNPSSPSPNNSVLTSGVGADVARMWEIAIQHALM
Pleckstrin homology domain of Human Son Of Sevenless Homolog 2
(SEQ ID NO: 84)
FIMEGPLTRIGAKHERHIFLFDGLMISCKPNHGQTRLPGYSSAEYRLKEKFVMRKIQICDKEDTCEHKH
AFELVSKDENSIIFAAKSAEEKNNWMAALISLHYRS
Pleckstrin homology domain of Human Dynamin
QGTNLPPSRQIVIRKGWLTISNIGIMKGGSKGYWFVLTAESLSWYKDDEEKEKKYMLPLDNLKVRDVEK
(SEQ ID NO: 85)
SEMSSKHIFALENTEQRNVYKDYRFLELACDSQEDVDS
Pleckstrin homology domain of Human BCR
(SEQ ID NO: 86)
QLLKDSFMVELVEGARKLRHVFLFTDLLLCTKLKKQSGGKTQQYDCKWYIPLTDLSFQMVDELEAVPNI
PLVPDEELDALKIKISQIKNDIQREKRANKGSKATERLKKKLSEQESLLLLMSPSMAFRVHSRNGKSYT
FLISSDYERAEWRENIREQQK
Pleckstrin homology domain of Human DBS
(SEQ ID NO: 87)
KLLMQGSFSVWTDHKRGHTKVKELARFKPMQRHLFLHEKAVLFCKKREENGEGYEKAPSYSYKQSLNMA
AVGITENVKGDAKKFEIWYNAREEVYIVQAPTPEIKAAWVNEIRKVLT
Pleckstrin homology domain of Homo sapiens phospholipase Cδ1 (hPLC81)
(SEQ ID NO: 88)
MDSGRDFLTLHGLQDDEDLQALLKGSQLLKVKSSSWRRERFYKLQEDCKTIWQESRKVMRTPESQLESI
EDIQEVRMGHRTEGLEKFARDVPEDRCFSIVEKDQRNTLDLIAPSPADAQHWVLGLHKIIHHSGSMDQR
QKLQHWIHSCLRKADKNKDNKMSFKELQNELKELNIQ
Pleckstrin homology domain of Homo sapiens phospholipase Cδ1 (hPLC81)
R40L:
(SEQ ID NO: 89)
MDSGRDFLTLHGLQDDEDLQALLKGSQLLKVKSTSWRRELFYKLQEDCKTIWQESRKVMRTPESQLFSI
EDIQEVRMGHRTEGLEKFARDVPEDRCFSIVFKDQRNTLDLIAPSPADAQHWVLGLHKIIHHSGSMDQR
QKLQHWIHSCLRKADKNKDNKMSFKELQNFLK
Pleckstrin homology domain of Homo sapiens Akt1 (hAkt)
(SEQ ID NO: 90)
MSDVAIVKEGWLHKRGEYIKTWRPRYFLLKNDGTFIGYKERPQDVDQREAPLNNESVAQCQLMKTERPR
PNTFIIRCLQWTTVIERTFHVETPEEREEWTTAIQTVADGLKKQEEEEMDFRSGSPSDNSGAEEMEVSL
AKPKHRVTMNEFEYLKLLGKGTFGKVDPPV
Pleckstrin homology domain of Homo sapiens Akt1 (hAkt) E17K:
(SEQ ID NO: 91)
MSDVAIVKEGWLHKRGKYIKTWRPRYFLLKNDGTFIGYKERPQDVDQREAPLNNFSVAQCQLMKTERPR
PNTFIIRCLQWTTVIERTFHVETPEEREEWTTAIQTVADGLKKQEEEEMDERSGSPSDNSGAEEMEVSL
AKPKHRVTMNEFEYLKLLGKGTFGKVDPPV
Pleckstrin homology domain of Homo sapiens Akt1 (hAkt) K14R:
(SEQ ID NO: 92)
MSDVAIVKEGWLHRRGEYIKTWRPRYFLLKNDGTFIGYKERPQDVDQREAPLNNFSVAQCQLMKTERPR
PNTFIIRCLQWTTVIERTFHVETPEEREEWTTAIQTVADGLKKQEEEEMDERSGSPSDNSGAEEMEVSL
AKPKHRVTMNEFEYLKLLGKGTFGKVDPPV
Pleckstrin homology domain of Homo sapiens Akt1 (hAkt) K8R:
(SEQ ID NO: 93)
MSDVAIVREGWLHKRGEYIKTWRPRYFLLKNDGTFIGYKERPQDVDQREAPLNNFSVAQCQLMKTERPR
PNTFIIRCLQWTTVIERTFHVETPEEREEWTTAIQTVADGLKKQEEEEMDFRSGSPSDNSGAEEMEVSL
AKPKHRVTMNEFEYLKLLGKGTFGKVDPPV
Pleckstrin homology domain of Homo sapiens Akt1 (hAkt) T72A:
(SEQ ID NO: 94)
MSDVAIVKEGWLHKRGEYIKTWRPRYFLLKNDGTFIGYKERPQDVDQREAPLNNESVAQCQLMKTERPR
PNAFIIRCLQWTTVIERTFHVETPEEREEWTTAIQTVADGLKKQEEEEMDFRSGSPSDNSGAEEMEVSL
AKPKHRVTMNEFEYLKLLGKGTFGKVDPPV
Pleckstrin homology domain of Homo sapiens Akt1 (hAkt) T92A:
(SEQ ID NO: 95)
MSDVAIVKEGWLHKRGEYIKTWRPRYFLLKNDGTFIGYKERPQDVDQREAPLNNFSVAQCQLMKTERPR
PNTFIIRCLQWTTVIERTFHVEAPEEREEWTTAIQTVADGLKKQEEEEMDERSGSPSDNSGAEEMEVSL
AKPKHRVTMNEFEYLKLLGKGTFGKVDPPV
Pleckstrin homology domain of Homo sapiens Akt1 (hAkt) R25C:
(SEQ ID NO: 96)
MSDVAIVKEGWLHKRGEYIKTWRPCYFLLKNDGTFIGYKERPQDVDQREAPLNNFSVAQCQLMKTERPR
PNTFIIRCLQWTTVIERTFHVETPEEREEWTTAIQTVADGLKKQEEEEMDFRSGSPSDNSGAEEMEVSL
AKPKHRVTMNEFEYLKLLGKGTFGKVDPPV
Pleckstrin homology domain of Homo sapiens Akt1 (hAkt) T34D:
(SEQ ID NO: 97)
MSDVAIVKEGWLHKRGEYIKTWRPRYFLLKNDGDFIGYKERPQDVDQREAPLNNFSVAQCQLMKTERPR
PNTFIIRCLQWTTVIERTFHVETPEEREEWTTAIQTVADGLKKQEEEEMDERSGSPSDNSGAEEMEVSL
AKPKHRVTMNEFEYLKLLGKGTFGKVDPPV
Pleckstrin homology domain of Homo sapiens Akt1 (hAkt) T34F:
(SEQ ID NO: 98)
MSDVAIVKEGWLHKRGEYIKTWRPRYFLLKNDGFFIGYKERPQDVDQREAPLNNESVAQCQLMKTERPR
PNTFIIRCLQWTTVIERTFHVETPEEREEWTTAIQTVADGLKKQEEEEMDFRSGSPSDNSGAEEMEVSL
AKPKHRVTMNEFEYLKLLGKGTFGKVDPPV
Pleckstrin homology domain of Homo sapiens Akt1 (hAkt) T34L:
(SEQ ID NO: 99)
MSDVAIVKEGWLHKRGEYIKTWRPRYFLLKNDGLFIGYKERPQDVDQREAPLNNESVAQCQLMKTERPR
PNTFIIRCLQWTTVIERTFHVETPEEREEWTTAIQTVADGLKKQEEEEMDERSGSPSDNSGAEEMEVSL
AKPKHRVTMNEFEYLKLLGKGTFGKVDPPV
Pleckstrin homology domain of Homo sapiens Akt1 (hAkt) T81Y:
(SEQ ID NO: 100)
MSDVAIVKEGWLHKRGEYIKTWRPRYFLLKNDGTFIGYKERPQDVDQREAPLNNESVAQCQLMKTERPR
PNTFIIRCLQWYTVIERTFHVETPEEREEWTTAIQTVADGLKKQEEEEMDERSGSPSDNSGAEEMEVSL
AKPKHRVTMNEFEYLKLLGKGTFGKVDPPV
Pleckstrin homology domain of Homo sapiens Akt1 (hAkt) K142A, H143A,
R144A:
(SEQ ID NO: 101)
MSDVAIVKEGWLHKRGEYIKTWRPRYFLLKNDGTFIGYKERPQDVDQREAPLNNESVAQCQLMKTERPR
PNTFIIRCLQWTTVIERTFHVETPEEREEWTTAIQTVADGLKKQEEEEMDERSGSPSDNSGAEEMEVSL
AKPAAAVTMNEFEYLKLLGKGTFGKVDPPV
Pleckstrin homology domain of Homo sapiens Akt1 (hAkt) T101C:
(SEQ ID NO: 102)
MSDVAIVKEGWLHKRGEYIKTWRPRYFLLKNDGTFIGYKERPQDVDQREAPLNNESVAQCQLMKTERPR
PNTFIIRCLQWTTVIERTFHVETPEEREEWTCAIQTVADGLKKQEEEEMDERSGSPSDNSGAEEMEVSL
AKPKHRVTMNEFEYLKLLGKGTFGKVDPPV
Pleckstrin homology domain of Homo sapiens PDPK1 (hPDPK1)
(SEQ ID NO: 103)
KMGPVDKRKGLFARRRQLLLTEGPHLYYVDPVNKVLKGEIPWSQELRPEAKNFKTFFVHTPNRTYYLMD
PSGNAHKWCRKIQEVWRQRYQSH
MS2 (RNA Binding protein);
(SEQ ID NO: 104)
MASNFTQFVLVDNGGTGDVTVAPSNFANGIAEWISSNSRSQAYKVTCSVRQSSAQKRKYTIKVEVPKVA
TQTVGGVELPVAAWRSYLNMELTIPIFATNSDCELIVKAMQGLIKDGNPIPSAIAANSGIY
COM (RNA Binding protein):
(SEQ ID NO: 105)
MKSIRCKNCNKLLFKADSFDHIEIRCPRCKRHIIMLNACEHPTEKHCGKREKITHSDETVRY
PP7 (RNA Binding protein):
(SEQ ID NO: 106)
MAKTIVLAVGEATRTLTEIQSTADRQIFEEKVGPLVGRLRLTASLRQNGAKTAYRVNLKLDQADVVDAS
TSVAGELPKVRYTQVWSHDVTIVANSTEASRKSLYDLTKSLVATSQVEDLVVNLVPLGRSLE
TBP (RNA Binding protein):
(SEQ ID NO: 107)
MAVPETRPNHTIYINNLNSKIKKDELKKSLYAIFSQFGQILDILVPRQRTPRGQAFVIFKEVSSATNAL
RSMQGFPFYDKPMRIQYAKTDKRIPAKMKGTEV
Human SLBP (RNA Binding protein):
(SEQ ID NO: 108)
MADFETDESVLMRRQKQINYGKNTIAYDRYIKEVPRHLRQPGIHPKTPNKFKKYSRRSWDQQIKLWKVA
LHEWD
Herpes simplex virus (HSV) type 1: VP16 Transcription Activation
Domain
(SEQ ID NO: 109)
PTDALDDEDLDMLPADALDDEDLDMLPADALDDEDLDM
Herpes simplex virus (HSV) type 1 & Synthetic: VP64
(SEQ ID NO: 110)
GRADALDDFDLDMLGSDALDDEDLDMLGSDALDDEDLDMLGSDALDDEDLDML
Homo sapiens: P65
(SEQ ID NO: 111)
SQYLPDTDDRHRIEEKRKRTYETFKSIMKKSPFSGPTDPRPPPRRIAVPSRSSASVPKPAPQPYPFTSS
LSTINYDEFPTMVFPSGQISQASALAPAPPQVLPQAPAPAPAPAMVSALAQAPAPVPVLAPGPPQAVAP
PAPKPTQAGEGTLSEALLQLQFDDEDLGALLGNSTDPAVFTDLASVDNSEFQQLLNQGIPVAPHTTEPM
LMEYPEAITRLVTGAQRPPDPAPAPLGAPGLPNGLLSGDEDESSIADMDESALL
Kaposi's Sarcoma-Associated Herpesvirus Transactivator: RTA
(SEQ ID NO: 112)
RDSREGMFLPKPEAGSAISDVFEGREVCQPKRIRPFHPPGSPWANRPLPASLAPTPTGPVHEPVGSLTP
APVPQPLDPAPAVTPEASHLLEDPDEETSQAVKALREMADTVIPQKEEAAICGQMDLSHPPPRGHLDEL
TTTLESMTEDLNLDSPLTPELNEILDTFLNDECLLHAMHISTGLSIFDTSLE
Homo sapiens: KRAB
(SEQ ID NO: 113)
MDAKSLTAWSRTLVTFKDVFVDFTREEWKLLDTAQQIVYRNVMLENYKNLVSLGYQLTKPDVILRLEKG
EEP
Homo sapiens: MeCP2
(SEQ ID NO: 114)
EASVQVKRVLEKSPGKLLVKMPFQASPGGKGEGGGATTSAQVMVIKRPGRKRKAEADPQAIPKKRGRKP
GSVVAAAAAEAKKKAVKESSIRSVQETVLPIKKRKTRETVSIEVKEVVKPLLVSTLGEKSGKGLKTCKS
PGRKSKESSPKGRSSSASSPPKKEHHHHHHHAESPKAPMPLLPPPPPPEPQSSEDPISPPEPQDLSSSI
CKEEKMPRAGSLESDGCPKEPAKTQPMVAAAATTTTTTTTTVAEKYKHRGEGERKDIVSSSMPRPNREE
PVDSRTPVTERVS
Homo sapiens: Tet
(SEQ ID NO: 115)
LPTCSCLDRVIQKDKGPYYTHLGAGPSVAAVREIMENRYGQKGNAIRIEIVVYTGKEGKSSHGCPIAKW
VLRRSSDEEKVLCLVRQRTGHHCPTAVMVVLIMVWDGIPLPMADRLYTELTENLKSYNGHPTDRRCTLN
ENRTCTCQGIDPETCGASESFGCSWSMYENGCKFGRSPSPRRFRIDPSSPLHEKNLEDNLQSLATRLAP
IYKQYAPVAYQNQVEYENVARECRLGSKEGRPFSGVTACLDFCAHPHRDIHNMNNGSTVVCTLTREDNR
SLGVIPQDEQLHVLPLYKLSDTDEFGSKEGMEAKIKSGAIEVLAPRRKKRTCFTQPVPRSGKKRAAMMT
EVLAHKIRAVEKKPIPRIKRKNNSTTTNNSKPSSLPTLGSNTETVQPEVKSETEPHFILKSSDNTKTYS
LMPSAPHPVKEASPGESWSPKTASATPAPLKNDATASCGESERSSTPHCTMPSGRLSGANAAAADGPGI
SQLGEVAPLPTLSAPVMEPLINSEPSTGVTEPLTPHQPNHQPSFLTSPQDLASSPMEEDEQHSEADEPP
SDEPLSDDPLSPAEEKLPHIDEYWSDSEHIFLDANIGGVAIAPAHGSVLIECARRELHATTPVEHPNRN
HPTRLSLVFYQHKNLNKPQHGFELNKIKFEAKEAKNKKMKASEQKDQAANEGPEQSSEVNELNQIPSHK
ALTLTHDNVVTVSPYALTHVAGPYNHWV
Homo sapiens: Dnmt3a
(SEQ ID NO: 116)
MPAMPSSGPGDTSSSAAEREEDRKDGEEQEEPRGKEERQEPSTTARKVGRPGRKRKHPPVESGDTPKDP
AVISKSPSMAQDSGASELLPNGDLEKRSEPQPEEGSPAGGQKGGAPAEGEGAAETLPEASRAVENGCCT
PKEGRGAPAEAGKEQKETNIESMKMEGSRGRLRGGLGWESSLRQRPMPRLTFQAGDPYYISKRKRDEWL
ARWKREAEKKAKVIAGMNAVEENQGPGESQKVEEASPPAVQQPTDPASPTVATTPEPVGSDAGDKNATK
AGDDEPEYEDGRGFGIGELVWGKLRGFSWWPGRIVSWWMTGRSRAAEGTRWVMWFGDGKFSVVCVEKLM
PLSSFCSAFHQATYNKQPMYRKAIYEVLQVASSRAGKLFPVCHDSDESDTAKAVEVQNKPMIEWALGGF
QPSGPKGLEPPEEEKNPYKEVYTDMWVEPEAAAYAPPPPAKKPRKSTAEKPKVKEIIDERTRERLVYEV
RQKCRNIEDICISCGSLNVTLEHPLFVGGMCQNCKNCFLECAYQYDDDGYQSYCTICCGGREVLMCGNN
NCCRCFCVECVDLLVGPGAAQAAIKEDPWNCYMCGHKGTYGLLRRREDWPSRLQMFFANNHDQEFDPPK
VYPPVPAEKRKPIRVLSLEDGIATGLLVLKDLGIQVDRYIASEVCEDSITVGMVRHQGKIMYVGDVRSV
TQKHIQEWGPFDLVIGGSPCNDLSIVNPARKGLYEGTGRLFFEFYRLLHDARPKEGDDRPFFWLFENVV
AMGVSDKRDISRFLESNPVMIDAKEVSAAHRARYFWGNLPGMNRPLASTVNDKLELQECLEHGRIAKES
KVRTITTRSNSIKQGKDQHFPVEMNEKEDILWCTEMERVEGFPVHYTDVSNMSRLARQRLLGRSWSVPV
IRHLFAPLKEYFACV
BaEVTRless
(SEQ ID NO: 117)
MGFTTKIIFLYNLVLVYAGEDDPRKAIELVQKRYGRPCDCSGGQVSEPPSDRVSQVTCSGKTAYLMPDQ
RWKCKSIPKDTSPSGPLQECPCNSYQSSVHSSCYTSYQQCRSGNKTYYTATLLKTQTGGTSDVQVLGST
NKLIQSPCNGIKGQSICWSTTAPIHVSDGGGPLDTTRIKSVQRKLEEIHKALYPELQYHPLAIPKVRDN
LMVDAQTLNILNATYNLLLMSNTSLVDDCWLCLKLGPPTPLAIPNFLLSYVTRSSDNISCLIIPPLLVQ
PMQFSNSSCLFSPSYNSTEEIDLGHVAFSNCTSITNVTGPICAVNGSVELCGNNMAYTYLPTNWTGLCV
LATLLPDIDIIPGDEPVPIPAIDHFIYRPKRAIQFIPLLAGLGITAAFTTGATGLGVSVTQYTKLSNQL
ISDVQILSSTIQDLQDQVDSLAEVVLQNRRGLDLLTAEQGGICLALQEKCCFYVNKSGIVRDKIKTLQE
ELERRRKDLASNPLWTGLQGLLPYLLPFLGPLLTLLLLLTIGPCIENRLTAFINDKLNIIHAM
>AJ289709.1 Human endogenous retrovirus H HERV-H/env62 HERV_H/ENV_62
hENVH1:
(SEQ ID NO: 118)
MIFAGKAPSNTSTLMKFYSLLLYSLLFSFPFLCHPLPLPSYLHHTINLTHSLLAASNPSLVNNCWLCIS
LSSSAYTAVPAVQTDWATSPISLHLRTSENSPHLYPPEELIYFLDRSSKTSPDISHQQAAALLRTYLKN
LSPYINSTPPIFGPLTTQTTIPVAAPLCISWQRPTGIPLGNLSPSRCSFTLHLRSPTTNINETIGAFQL
HITDKPSINTDKLKNISSNYCLGRHLPCISLHPWLSSPCSSDSPPRPSSCLLIPSPENNSERLLVDTRR
FLIHHENRTFPSTQLPHQSPLQPLTAAALAGSLGVWVQDTPFSTPSHLFTLHLQFCLAQGLFFLCGSST
YMCLPANWTGTCTLVFLTPKIQFANGTEELPVPLMTPTQQKRVIPLIPLMVGLGLSASTVALGTGIAGI
STSVMTFRSLSNDFSASITDISQTLSVLQAQVDSLAAVVLQNRRGLDLLTAEKGGLCIFLNEECCFYLN
QSGLVYDNIKKLKDRAQKLANQASNYAEPPWALSNWMSWVLPIVSPLIPIFLLLLFGPCIFRLVSQFIQ
NRIQAITNHSIRQMFLLTSPQYHPLPQDLPSA
>AJ289710.2 Human endogenous retrovirus H HERV-H/env60-
HERV_H_ENV_60-hENVH2:
(SEQ ID NO: 119)
MIFAGRASSNTSTLMKFYSLLLYSLLESFPILCHPLPLPSYLHHTINLTHSLLAVSNPSLAKNCWLCIS
LPSSAYPAVPALQTDWGTSPVSPHLRTSENSPHLYPPEKLIYFLDRSSKTSPDISHQQAAALLCTYLKN
LSPYINSTPPTFGPLTTQTTIPVAAPLCISRQRPTGIPLGNLSPSRCSFTLHLRSPTTHITETNGAFQL
HITDKPSINTDKLKNVSSNYCLGRHLSCISLHPWLFSPCSSDSPPRPSSCLLIPSPKNNSESLLVDAQR
FLIYHENRTSPSTQLPHQSPLQPLTAAPLGGSLRVWVQDTPFSTPSHLFTLHLQFCLVQSLFFLCGSST
YMCLPANWTGTCTLVFLTSKIQFANGTEELPVPLMTPTRQKRVIPLIPLMVGLGLSASTVALGTGIAGI
STSVTTFRILSNDESASITDISQTLSGLQAQVDSSAAVVLQNRQGLDLLTAEKGGLCIFLNEESYFYLN
QSGLVYDNIKKLKDKAQNLANQASNYAEPPWPLSNWMSWVLPILSPLIPIFLLLFFRPCIFHLVSQFIQ
NHIQAITDHSI
>AJ289711.1 Human endogenous retrovirus H HERV-H/env59-
HERV_H_ENV_59-hENVH3:
(SEQ ID NO: 120)
MILAGRAPSNTSTLMKFYSLLLYSLLFSFPFLYHPLPLPSYLHHTINLTHSLPAASNPSLANNCWLCIS
LSSSAYIAVPTLQTDRATSPVSLHLRTSENSPHLYPPEELIYFLDRSSKTSPDISHQPAAALLHIYLKN
LSPYINSTPPIFGPLTTQTTIPVAAPLCISRQRPTGIPLGNISPSRCSFTLHLQSPTTHVTETIGVEQL
HIIDKPSINTDKLKNVSSNYCLGRHLPYISLHPWLPSPCSSDSPPRPSSCLLTPSPQNNSERLLVDTQR
FLIHHENRTSSSMQLAHQSPLQPLTAAALAGSLGVWVQDTPESTPSHPFSLHLQFCLTQGLFFLCGSST
YMCLPANWTGTCTLVFLTPKIQFANGTKELPVPLMTLTPQKRVIPLIPLMVGLGLSASTIALSTGIAGI
STSVTTERSPSNDFSASITDISQTLSVLQAQVDSLAAVVLQNRRGLGLSILLNEECCFYLNQSGLVYEN
IKKLKDRAQKLANQASNYAESPWALSNWMSWVLPILSPLIPIFLLLLFGPCIFHLVSQFIQNRIQAITN
HSI
>AC074261.3 Homo sapiens chromosome 12 clone RP11-55F19 envk1-
ENVK1:
(SEQ ID NO: 121)
MHPSEMQRKAPPRRRRHRNRAPLTHKMNKMVTSEQMKLPSTKKAEPPTWAQLKKLTQLATKYLENTKVT
QTPESMLLAALMIVSMVVSLPMPAGAAAANYTNWAYVPFPPLIRAVTWMDNPIEVYVNDSVWVHGPIDD
RCPAKPEEEGMMINISIGYHYPPICLGRAPGCLMPAVQNWLVEVPTVSPISRFTYNMVSGMSLRPRVNY
LQDESYQRSLKFRPKGKPCPKEIPKESKNTEVLVWEECVANSVVILQNNEFGTIIDWAPRGQFYHNCSG
QTQSCPSAQVSPAVDSDLTESLDKHKHKKLQSFYPWEWGEKGISTPRPKIISPVSGPEHPELWRLTVAS
HHIRIWSGNQTLETRDRKPFYTVDLNSSLTVPLQSCVKPPYMLVVGNIVIKPDSQTITCENCRLLTCID
STFNWQHRILLVRAREGVWIPVSMDRPWEASPSIHILTEVLKGVLNRSKRFIFTLIAVIMGLIAVTAMA
AVAGVALHSFVQSVNFVNDWQKNSTRLWNSQSSIDQKLANQINDLRQTVIWMGDRLMSLEHRFQLQCDW
NTSDFCITPQIYNESEHHWDMVRRHLQGREDNLTLDISKLKEQIFEASKAHLNLVPGTEAIAGVADGLA
NLNPVTWVKTIGSTTIINLILILVCLFCLLLVCRFTQQLRRDSYHRERAMMTMVVLSKRKGGNVGKSKR
DQIVTVSV
>AC072054.10 Homo sapiens BAC clone RP11-33P21-ENVK2:
(SEQ ID NO: 122)
MNPSEMQRKAPPRRRRHRNRAPLTHKMNKMVTSEEQMKLPSTKKAEPPTWAQLKKLTQLATKYLENTKV
TQTPESMLLAALMIVSMVVSLPMPAGAAAANYTYWAYVPFPPLIRAVTWMDNPTEVYVNDSVWVPGPID
DRCPAKPEEEGMMINISIGYHYPPICLGRAPGCLMPAVQNWLVEVPTVSPICRFTYHMVSGMSLRPRVN
YLQDESYQRSLKFRPKGKPCPKEIPKESKNTEVLVWEECVANSAVILQNNEFGTIIDWAPRGQFYHNCS
GQTQSCPSAQVSPAVDSDLTESLDKHKHKKLQSFYPWEWGEKGISTPRPKIVSPVSGPEHPELWRLTVA
SHHIRIWSGNQTLETRDRKPFYTIDLNSSLTVPLQSCVKPPYMLVVGNIVIKPDSQTITCENCRLLTCI
DSTFNWQHRILLVRAREGVWIPVSMDRPWEASPSVHILTEVLKGVLNRSKRFIFTLIAVIMGLIAVTAT
AAVAGVALHSSVQSVNFVNDWQKNSTRLWNSQSSIDQKLANQINDLRQTVIWMGDRLMSLEHRFQLQCD
WNTSDFCITPQIYNESEHHWDMVRRHLQGREDNLTLDISKLKEQIFEASKAHLNLVPGTEAIAGVADGL
ANLNPVTWVKTIGSTTIINLILILVCLFCLLLVCRCTQQLRRDSDHRERAMMTMAVLSKRKGGNVGKSK
RDQIVTVSV
>Y17833.1 Human endogenous retrovirus K (HERV-K) envK3-ENVK3:
(SEQ ID NO: 123)
MNPSEMQRKAPPRRRRHRNRAPLTHKMNKMVTSEEQMKLPSTKKAEPPTWAQLKKLTQLATKYLENTKV
TQTPESMLLAALMIVSMVVSLPMPAGAAAANYTYWAYVPFPPLIRAVTWMDNPIEVYVNDSVWVPGPTD
DHCPAKPEEEGMMINISIGYRYPPICLGRAPGCLMPAVQNWLVEVPTVSPISRFTYHMVSGMSLRPRVN
YLQDESYQRSFKFRPKGKPCPKEIPKESKNTEVLVWEECVANSAVILQNNEFGTIIDWAPRGQFYHNCS
GQTQSCPSAQVSPAVDSDLTESLDKHKHKKLQSFYPWEWGEKGISTPRPKIISPVSGPEHPELWRLTVA
SHHIRIWSGNQTLETRDRKPFYTVDLNSSVTVPLQSCIKPPYMLVVGNIVIKPDSQTITCENCRLLTCI
DSTENWQHRILLVRAREGVWIPVSMDRPWETSPSIHTLTEVLKGVLNRSKRFIFTLIAVIMGLIAVTAT
AAVAGVALHSSVQSVNFVNDWQKNSTRLWNSQSSIDQKLANQINDLRQTVIWMGDRLMSLEHRFQLQCD
WNTSDFSITPQIYNESEHHWDMVRRHLQGREDNLTLDISKLKEQIFEASKAHLNLVPGTEAIAGVADGL
ANLNPVTWVKTIGSTTIINLILILVCLFCLLLVCRCTQQLRRDSDHRERAMMTMAVLSKRKGGNVGKSK
RDQIVTVSV
>AF164615.1 Homo sapiens endogenous retrovirus HERV-K109 envk4-
ENVK4:
(SEQ ID NO: 124)
MNPSEMQRKAPPRRRRHRNRAPLTHKMNKMVTSEEQMKLPSTKKAEPPTWAQLKKLTQLATKYLENTKV
TQTPESMLLAALMIVSMVVSLPMPAGAAAANYTNWAYVPFPPLIRAVTWMDNPIEVYVNDSVWVPGPID
DRCPAKPEEEGMMINISIGYRYPICLGRAPGCLMPAVQNWLVEVPIVSPICRFTYHMVSGMSLRPRVNY
LQDESYQRSLKFRPKGKPCPKEIPKESKNTEVLVWEECVANSAVILQNNEFGTIIDWTPQGQFYHNCSG
QTQSCPSAQVSPAVDSDLTESLDKHKHKKLQSFYPWEWGEKGISTPRPKIISPVSGPEHPELWRLTVAS
HHIRIWSGNQTLETRDRKPFYTVDLNSSLTLPLQSCVKPPYMLVVGNIVIKPDSQTITCENCRLLTCID
STFNWQHRILLVRAREGVWIPVSMDRPWEASPSIHILTEVLKGVLNRSKRFIFTLIAVIMGLIAVTATA
AVAGVALHSSVQSVNFVNDGQKNSTRLWNSQSSIDQKLANQINDLRQTVIWMGDRLMSLEHREQLQCDW
NTSDFCITPQIYNESEHHWDMVRRHLQGREDNLTLDISKLKEQIFEASKAHLNLVPGTEAIAGVADGLA
NLNPVTWVKTIGSTTIINLILILVCLFCLLLVCRCTQQLRRDSDHRERAMMTMAVLSKRKGGNVGKSKR
DQIVTVSV
>AY037928.1 Human endogenous retrovirus K113 envK5-ENVK5:
(SEQ ID NO: 125)
MNPSEMQRKAPPRRRRHRNRAPLTHKMNKMVTSEEQMKLPSTKKAEPPTWAQLKKLTQLATKYLENTKV
TQTPESMLLAALMIVSMVVSLPMPAGAAAANYTYWAYVPFPPLIRAVTWMDNPIEIYVNDSVWVPGPTD
DCCPAKPEEEGMMINISIGYRYPPICLGRAPGCLMPAVQNWLVEVPTVSPISRFTYHMVSGMSLRPRVN
YLQDESYQRSLKFRPKGKPCPKEIPKESKNTEVLVWEECVANSAVILQNNEFGTLIDWAPRGQFYHNCS
GQTQSCPSAQVSPAVDSDLTESLDKHKHKKLQSFYPWEWGEKGISTARPKIISPVSGPEHPELWRLTVA
SHHIRIWSGNQTLETRDRKPFYTIDLNSSLTVPLQSCVKPPYMLVVGNIVIKPDSQTITCENCRLLTCI
DSTFNWQHRILLVRAREGVWIPVSMDRPWEASPSVHILTEVLKGVLNRSKRFIFTLIAVIMGLIAVTAT
AAVAGVALHSSVQSVNFVNDWQNNSTRLWNSQSSIDQKLANQINDLRQTVIWMGDRLMSLEHRFQLQCD
WNTSDFCITPQIYNESEHHWDMVRCHLQGREDNLTLDISKLKEQIFEASKAHLNLVPGTEAIAGVADGL
ANLNTVTWVKTIGSTTIINLILILVCLFCLLLVYRCTQQLRRDSDHRERAMMTMVVLSKRKGGNVGKSK
RDQIVTVSV
>AY037929.1 Human endogenous retrovirus K115 envK6-ENVK6:
(SEQ ID NO: 126)
MNPSEMQRKAPPRRRRHRNRAPLTHKMNKMVTSEEQMKLPSTKKAEPPTWAQLKKLTQLATKYLENTKV
TQTPESMLLAALMIVSMVVSLPMPAGAAVANYTNWAYVPFPPLIRAVTWMDNPIEVYVNDSVWVPGPID
DRCPAKPEEEGMMINISIGYRYPPICLGRAPGCLMPAVQNWLVEVPTVSPISRFTYHMVSGMSLRPRVN
YLQDFSYQRSLKFRPKGKPCPKEIPKESKNTEVLVWEECVANSAVILQNNEFGTIIDWAPRGQFYHNCS
GQTQSCPSAQVSPAVDSDLTESLDKHKHKKLQSFYPWEWGEKRISTPRPKIVSPVSGPEHPELWRLTVA
SHHIRIWSGNQTLETRDRKPFYTVDLNSSLTLPLQSCVKPPYMLVVGNIVIKPDSQTITCENCRLLTCI
DSTENWQHRILLVRAREGVWIPVSMDRPWEASPSVHILTEVLKGVLNRSKRFIFTLIAVIMGLIAVTAT
AAVAGVALHSSVQSVNFVNDGQKNSTRLWNSQSSIDQKLANQINDLRQTVIWMGDRLMSLEHRFQLQCD
WNTSDFCITPQIYNDSEHHWDMVRRHLQGREDNLTLDISKLKEQIFEASKAHLNLVPGTEAIAGVADGL
ANLNPVTWVKTIGSTTIINLILILVCLFCLLLVCRCTQQLRRDSDHRERAMMTMAVLSKRKGGNVGKSK
RDQIVTVSV
>AC078899.1 Homo sapiens chromosome 19, BAC BC371065 envT-ENVT:
(SEQ ID NO: 127)
MGPEAWVRPLKTAPKPGEAIRLILFIYLSCFFLPVMSSEPSYSELLTSFTTGRVFANTTWRAGTSKEVS
FAVDLCVLFPEPARTHEEQHNLPVIGAGSVDLAAGFGHSGSQTGCGSSKGAEKGLQNVDFYLCPGNHPD
ASCRDTYQFFCPDWTCVTLATYSGGSTRSSTLSISRVPHPKLCTRKNCNPLTITVHDPNAAQWYYGMSW
GLRLYIPGFDVGTMFTIQKKILVSWSSPKPIGPLTDLGDPIFQKHPDKVDLTVPLPFLVPRPQLQQQHL
QPSLMSILGGVHHLLNLTQPKLAQDCWLCLKAKPPYYVGLGVEATLKRGPLSCHTRPRALTIGDVSGNA
SCLISTGYNLSASPFQATCNQSLLTSISTSVSYQAPNNTWLACTSGLTRCINGTEPGPLLCVLVHVLPQ
VYVYSGPEGRQLIAPPELHPRLHQAVPLLVPLLAGLSIAGSAAIGTAALVQGETGLISLSQQVDADESN
LQSAIDILHSQVESLAEVVLQNCRCLDLLFLSQGGLCAALGESCCFYANQSGVIKGTVKKVRENLDRHQ
QERENNIPWYQSMFNWNPWLTTLITGLAGPLLILLLSLIFGPCILNSFLNFIKQRIASVKLTYLKTQYD
TLVNN
>AC000064.1 Human BAC clone RG083M05 from 7q21-7q22 envW (Syncytin-1)-
ENVW (Syncytin-1):
(SEQ ID NO: 128)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGVQDQAREKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKFSNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPELGPLAAIILLLLFGPCIFNLLVNFVSSRIEAV
KLQMEPKMQSKTKIYRRPLDRPASPRSDVNDIKGTPPEEISAAQPLLRPNSAGSS
>AL136139.6 Human DNA sequence from clone RP4-76112 envFRD-ENVERD
(Syncytin-2):
(SEQ ID NO: 129)
MGLLLLVLILTPSLAAYRHPDEPLLEKAQQLLQSTGSPYSTNCWLCTSSSTETPGTAYPASPREWTSIE
AELHISYRWDPNLKGLMRPANSLLSTVKQDFPDIRQKPPIFGPIFTNINLMGIAPICVMAKRKNGTNVG
TLPSTVCNVTFTVDSNQQTYQTYTHNQFRHQPRFPKPPNITFPQGTLLDKSSRFCQGRPSSCSTRNFWF
RPADYNQCLQISNLSSTAEWVLLDQTRNSLFWENKTKGANQSQTPCVQVLAGMTIATSYLGISAVSEFF
GTSLTPLFHFHISTCLKTQGAFYICGQSIHQCLPSNWTGTCTIGYVTPDIFIAPGNLSLPIPIYGNSPL
PRVRRAIHFIPLLAGLGILAGTGTGIAGITKASLTYSQLSKEIANNIDTMAKALTTMQEQIDSLAAVVL
QNRRGLDMLTAAQGGICLALDEKCCFWVNQSGKVQDNIRQLLNQASSLRERATQGWLNWEGTWKWESWV
LPLTGPLVSLLLLLLFGPCLLNLITQFVSSRLQAIKLQTNLSAGRHPRNIQESPF
>AC073210.8 Homo sapiens BAC clone RP11-460N20 envR-ENVR:
(SEQ ID NO: 130)
MLGMNMLLITLFLLLPLSMLKGEPWEGCLHCTHTTWSGNIMTKTLLYHTYYECAGTCLGTCTHNQTTYS
VCDPGRGQPYVCYDPKSSPGTWEEIHVGSKEGDLLNQTKVFPSGKDVVSLYFDVCQIVSMGSLFPVIFS
SMEYYSSCHKNRYAHPACSTDSPVTTCWDCTTWSTNQQSLGPIMLTKIPLEPDCKTSTCNSVNLTILEP
DQPIWTTGLKAPLGARVSGEEIGPGAYVYLYIIKKTRTRSTQQFRVFESFYEHVNQKLPEPPPLASNLF
AQLAENIASSLHVASCYVCGGMNMGDQWPWEARELMPQDNFTLTASSLEPAPSSQSIWELKTSIIGKFC
IARWGKAFTDPVGELTCLGQQYYNETLGKTLWRGKSNNSESPHPSPESRFPSLNHSWYQLEAPNTWQAP
SGLYWICGPQAYRQLPAKWSGACVLGTIRPSFFLMPLKQGEALGYPIYDETKRKSKRGITIGDWKDNEW
PPERIIQYYGPATWAEDGMWGYRTPVYMLNRIIRLQAVLEIITNETAGALNLLAQQATKMRNVIYQNRL
ALDYLLAQEEGVCGKENLTNCCLELDDEGKVIKEITAKIQKLAHIPVQTWKG
>AC093488.1 Homo sapiens chromosome 3 clone RP11-1008 envR(b)-ENVR(b):
(SEQ ID NO: 131)
MDPLHTIEKVPARRNIHDRGHQGHRMGDGTPGRPKISVQQMTRFSLIIFFLSAPFVVNASTSNVFLQWA
HSYADGLQQGDPCWVCGSLPVTNTMELPWWVSPLQGKDWVFFQSFIGDLKQWTGAQMTGVTRKNISEWP
INKTLNEPGHDKPFSVNETRDKVIAFAIPLLDTKVFVQTSRPQNTQYRNGELQIWDGFIWLTATKGHLS
QIAPLCWEQRNHSLDNWPNTTRVMGWIPPGQCRHTILLQQRDLFATDWSQQPGLNWYAPNGTQWLCSPN
LWPWLPSGWLGCCTLGIPWAQGRWVKTMEVYPYLPHVVNQGTRAIVHRNDHLPTIFMPSVGLGTVIQHI
EALANFTQRALNDSLQSISLMNAEVYYMHEDILQNRMALDILTAAEGGTCALIKTECCVYIPNNSRNIS
LALEDTCRQIQVISSSALSLHDWIASQFSGRPSWWQKILIVLATLWSVGIALCCGLYFCRMESQHIPQT
HSIIFQQELPLSPPSQEHYQSQRDIFHSNAP
>AC016222.4 Homo sapiens clone RP11-26J6 envF(c)2-ENVF(c)2:
(SEQ ID NO: 132)
MNSPCDRLQQFIQVLLEESWSFPSFANTLHWPENLLSYIDELVWQGSLQNFHQHEVREDKPPLRLPLTG
FSSLTENWSSRQAVSSRLVATAASPPAGCQAPIAFLGLKESSLGPARKNPALCFLYDQSNSKCNTSWVK
ENVGCPWHWCNIHEALIRTEKGSDPMFYVNTSTGGRDGENGENLQISDPWDPRWASGVDGGLYEHKTFM
YPVAKIRIARTLKTTVTGLSDLASSIQSAEKELTSQLQPAADQAKSSRFSWLTLISEGAQLLQSTGVQN
LSHCFLCAALRRPPLVAVPLPTPENYTINSSTPIPPVPKGQVPLESDPIRHKFPFCYSTPNASWCNQTR
MLTSTPAPPRGYFWCNSTLTKVLNSTGNHTLCLPISLIPGLTLYSQDELSHLLAWTEPRPQNKSKWAIF
LPLVLGISLASSLVASGLGKGALTHSIQTSQDLSTHLQLAIEASAESLDSLQRQITTVAQVAAQNRQAL
DLLMAEKGRTCLFLQEECCYYLNESGVVENSLQTLKKKKSSKRS
>AL354685.17 Human DNA sequence from clone RP13-75G22 envF(c)1-ENVE(c)1:
(SEQ ID NO: 133)
MARPSPLCLLLLLTLLTPIVPSNSLLTEPPFRWRFYLHETWTQGNRLSTVTLATVDCQPHGCQAQVTEN
FTSFKSVLRGWSNPTICFVYDQTHSNCRDYWVDTNGGCPYAYCRMHVTQLHTAKKLQHTYRLTSDGRTT
YFLTIPDPWDSRWVSGVTGRLYRWPTDSYPVGKLRIFLTYIRVIPQVLSNLKDQADNIKHQEEVINTLV
QSHPKADMVTYDDKAEAGPFSWITLVRHGARLVNMAGLVNLSHCFLCTALSQPPLVAVPLPQAENTSGN
HTAHPSGVFSEQVPLERDPLQPQFPFCYTTPNSSWCNQTYSGSLSNLSAPAGGYFWCNFTLTKHLNISS
NNTLSRNLCLPISLVPRLTLYSEAELSSLVNPPMRQKRAVFPPLVIGVSLTSSLVASGLGTGAIVHFIS
SSQDLSIKLQMAIEASAESLASLQRQITSVAKVAMQNRRALDLLTADKGGTCMELGEECCYYINESGLV
ETSLLTLDKIRDGLHRPSSTPNYGGGWWQSPLTTWIIPFISPILIICLLLLIAPCVLKFIKNRISEVSR
VTVNQMLLHPYSRLPTSEDHYDDALTQQEAAR
HERV-Kcon ENV-hENVKcon:
(SEQ ID NO: 134)
MNPSEMQRKAPPRRRRHRNRAPLTHKMNKMVTSEEQMKLPSTKKAEPPTWAQLKKLTQLATKYLENTKV
TQTPESMLLAALMIVSMVVSLPMPAGAAAANYTYWAYVPFPPLIRAVTWMDNPIEVYVNDSVWVPGPID
DRCPAKPEEEGMMINISIGYRYPPICLGRAPGCLMPAVQNWLVEVPTVSPISRFTYHMVSGMSLRPRVN
YLQDFSYQRSLKFRPKGKPCPKEIPKESKNTEVLVWEECVANSAVILQNNEFGTIIDWAPRGQFYHNCS
GQTQSCPSAQVSPAVDSDLTESLDKHKHKKLQSFYPWEWGEKGISTPRPKIVSPVSGPEHPELWRLTVA
SHHIRIWSGNQTLETRDRKPFYTVDLNSSLTVPLQSCVKPPYMLVVGNIVIKPDSQTITCENCRLLTCI
DSTFNWQHRILLVRAREGVWIPVSMDRPWEASPSVHILTEVLKGVLNRSKRFIFTLIAVIMGLIAVTAT
AAVAGVALHSSVQSVNFVNDWQKNSTRLWNSQSSIDQKLANQINDLRQTVIWMGDRLMSLEHRFQLQCD
WNTSDFCITPQIYNESEHHWDMVRRHLQGREDNLTLDISKLKEQIFEASKAHLNLVPGTEAIAGVADGL
ANLNPVTWVKTIGSTTIINLILILVCLFCLLLVCRCTQQLRRDSDHRERAMMTMAVLSKRKGGNVGKSK
RDQIVTVSV
HERV T ENV:
(SEQ ID NO: 135)
MGPQAWARPLKTAPKSSEAIKLILFIYLFCLEPPITPSAPSYSELLTSETTGRVFANTTWKAGTSKEVS
FAVDLCALFPEPARTHEEQCNLPVMGAGNVDLAAGFGHTGSRTGCGSSKGAEKGLQSVDFYLCPGNHPD
SSCRDSYQFFCPHWSCVTLATYSGGSTRSSTLSITRTSRPRPCTIRNCNPLTITVRNPNSAQWYYGMSW
GLRLYISGFDVGTMFTIQKKVLVPWSPPKPIGPLTDLGDPMFQKHPDRVDLTVPPPLLVPKSQLQRQHL
QPSLMSILDGVHHLLNLTQPKLAQDCWLCLKAKPPYYVGLGVEAMFKVNSLSCHTRPHALTLGDVSGNA
SCLISAGYNLSASPFQATCNQSLLTSLNASVSYQAPNNTWLACTSGLTRCINGTEPGPLLCVLVHVLPQ
VYVYSGPEGQLLIAPPELHSRFRRAAPLLVPLLAGLSIAGSAAIGTAALVQGETGLMSLSQQVDADLSN
LQSAINMLHTQVESLAEVVLQNRRGLDLLFLSQGGLCAALGESCCFYANQSGVIKDTLQRVRENLDRRQ
QERENNTPWYQSMFNWNPWLTTLITGLAGPLLLLLLGLVFGPCILNWELNFVKQRIASVKLTYLRTQYN
PLVITEESMI
HERV W ENV
(SEQ ID NO: 222)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGVQDQAREKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKESNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPELGPLAAIILLLLFGPCIFNLLVNFVSSRIEAV
KLQMEPKMQSKTKIYRRPLDRPASPRSDVNDIKGTPPEEISAAQPLLRPNSAGSS
HERV H ENV:
(SEQ ID NO: 118)
MIFAGKAPSNTSTLMKFYSLLLYSLLFSFPFLCHPLPLPSYLHHTINLTHSLLAASNPSLVNNCWLCIS
LSSSAYTAVPAVQTDWATSPISLHLRTSENSPHLYPPEELIYFLDRSSKTSPDISHQQAAALLRTYLKN
LSPYINSTPPIFGPLTTQTTIPVAAPLCISWQRPTGIPLGNLSPSRCSFTLHLRSPTTNINETIGAFQL
HITDKPSINTDKLKNISSNYCLGRHLPCISLHPWLSSPCSSDSPPRPSSCLLIPSPENNSERLLVDTRR
FLIHHENRTFPSTQLPHQSPLQPLTAAALAGSLGVWVQDTPFSTPSHLFTLHLQFCLAQGLFFLCGSST
YMCLPANWTGTCTLVFLTPKIQFANGTEELPVPLMTPTQQKRVIPLIPLMVGLGLSASTVALGTGIAGI
STSVMTFRSLSNDFSASITDISQTLSVLQAQVDSLAAVVLQNRRGLDLLTAEKGGLCIFLNEECCFYLN
QSGLVYDNIKKLKDRAQKLANQASNYAEPPWALSNWMSWVLPIVSPLIPIFLLLLFGPCIFRLVSQFIQ
NRIQAITNHSIRQMELLTSPQYHPLPQDLPSA
HERV Pb ENV:
(SEQ ID NO: 136)
MDPSHPSQESTAPSSVMGHSPRGKSYQTKAKESLILFHLFCYSFFFPCALASHLIINVTRSDSPQTITE
DACLVIPCGDLQSQRQLAAAEKYLCPSEADASTLFSFPFCHTWEYVVWTTQRQDWVPSQDEPLAVLKPY
IHFTKGIAPPNCRYNQCNPVQISITIPTLQDSSPTLNRFYGMGADVRGKDPIGFFELHLSTSPSLISPR
LSSSTPANQTIVSSSNDKSKVAIVEVKNLKQTLTIETGYKETNAWMEWIEYSVRSLNKSDCYACAQGRP
EAQVVPFPLGWSSDQPGMGCMVALFQHPTAWDSEFCRTLSVLFPETQHLEGEPPRAIQPPSPDAKFTSC
LSRQGKNLEFLGDLKGCSELKSFQELTNQSALVHARADVWWYCGGHLLDTLPSNWSGTCALIQLAIPFT
LAFQQPEKKKPQRRKTREAPQGSFDSHVYVDEIGVPQGVPDREKARDPVAAGFESLFPMVAINKNVAWI
NYIYYNQQRFINYTRDAIQGIAEQLGPTSQMAWENRMALDMILAEKGGVCVMIGTQCCTYIPNNTAPDG
TITKALQGLTSLSDELATNSGITDPFTGWLGQWFGKWKGLMASIVTSLAIAIAVLILVGCCIMPCIRGL
VQRLIETASNKTFPSSSQSYSNKFFPVNEHEIRIILDREKAEHV
HERV Rb ENV:
(SEQ ID NO: 225)
MDPLHTIEKVPARRNIHDRGHQGHRMGDGTPGRPKISVQQMTRFSLIIFFLSAPFVVNASTSNVELQWA
HSYADGLQQGDPCWVCGSLPVTNTMELPWWVSPLQGKDWVFFQSFIGDLKQWTGAQMTGVTRKNISEWP
INKTLNEPGHDKPFSVNETRDKVIAFAIPLLDTKVFVQTSRPQNTQYRNGFLQIWDGFIWLTATKGHLS
QIAPLCWEQRNHSLDNWPNTTRVMGWIPPGQCRHTILLQQRDLFATDWSQQPGLNWYAPNGTQWLCSPN
LWPWLPSGWLGCCTLGIPWAQGRWVKTMEVYPYLPHVVNQGTRAIVHRNDHLPTIFMPSVGLGTVIQHI
EALANFTQRALNDSLQSISLMNAEVYYMHEDILQNRMALDILTAAEGGTCALIKTECCVYIPNNSRNIS
LALEDTCRQIQVISSSALSLHDWIASQFSGRPSWWQKILIVLATLWSVGIALCCGLYFCRMESQHIPQT
HSIIFQQELPLSPPSQEHYQSORDIFHSNAP
HERV 3-1 ENV:
(SEQ ID NO: 224)
MLGMNMLLITLFLLLPLSMLKGEPWEGCLHCTHTTWSGNIMTKTLLYHTYYECAGTCLGTCTHNQTTYS
VCDPGRGQPYVCYDPKSSPGTWFEIHVGSKEGDLLNQTKVFPSGKDVVSLYFDVCQIVSMGSLFPVIFS
SMEYYSSCHKNRYAHPACSTDSPVTTCWDCTTWSTNQQSLGPIMLTKIPLEPDCKTSTCNSVNLTILEP
DQPIWTTGLKAPLGARVSGEEIGPGAYVYLYIIKKTRTRSTQQFRVFESFYEHVNQKLPEPPPLASNLF
AQLAENIASSLHVASCYVCGGMNMGDQWPWEARELMPQDNFTLTASSLEPAPSSQSIWFLKTSIIGKFC
IARWGKAFTDPVGELTCLGQQYYNETLGKTLWRGKSNNSESPHPSPFSREPSLNHSWYQLEAPNTWQAP
SGLYWICGPQAYRQLPAKWSGACVLGTIRPSFFLMPLKQGEALGYPIYDETKRKSKRGITIGDWKDNEW
PPERIIQYYGPATWAEDGMWGYRTPVYMLNRIIRLQAVLEIITNETAGALNLLAQQATKMRNVIYQNRL
ALDYLLAQEEGVCGKENLTNCCLELDDEGKVIKEITAKIQKLAHIPVQTWKG
HERV V1 ENV:
(SEQ ID NO: 137)
MTEKFLFLYLSLLPMPLLSQAQWNENSLVSFSKIIASGNHLSNCWICHNFITRSSSYQYILVRNESLNL
TFGSGIPEGQHKSVPLQVSLANSAHQVPCLDLTPPFNQSSKTSFYFYNCSSLNQTCCPCPEGHCDRKNT
SEEGFPSPTIHPMSFSPAGCHPNLTHWCPAKQMNDYRDKSPQNRCAAWEGKELITWRVLYLLPKAHTVP
TWPKSTVPLGGPLSPACNQTIPAGWKSQLHKWFDSHIPRWACTPPGYVFLCGPQKNKLPEDGSPKITYS
TPPVANLYTCINNIQHTGECAVGLLGPRGIGVTIYNTTQPRQKRALGLILAGMGAAIGMIAPWGGFTYH
DVTLRNLSRQIDNIAKSTRDSISKLKASIDSLANVVMNNRLALDYLLAEQGGVCAVISKSCCIYVNNSG
AIEEDIKKIYDEVTWLHNEGKGDSAGSIWEAVKSALPSLTWFVPLLGPAALNSLLSPLWPLSL
HERV HEMO ENV:
(SEQ ID NO: 138)
MGSLSNYALLQLTLTAFLTILVQPQHLLAPVERTLSILTNQSNCWLCEHLDNAEQPELVFVPASASTWW
TYSGQWMYERVWYPQAEVQNHSTSSYRKVTWHWEASMEAQGLSFAQVRLLEGNFSLCVENKNGSGPFLG
NIPKQYCNQILWFDSTDGTEMPSIDVTNESRNDDDDTSVCLGTRQCSWFAGCTNRTWNSSAVPLIGLPN
TQDYKWVDRNSGLTWSGNDTCLYSCQNQTKGLLYQLFRNLFCSYGLTEAHGKWRCADASITNDKGHDGH
RTPTWWLTGSNLTLSVNNSGLFFLCGNGVYKGFPPKWSGRCGLGYLVPSLTRYLTLNASQITNLRSFIH
KVTPHRCTQGDTDNPPLYCNPKDNSTIRALFPSLGTYDLEKAILNISKAMEQEFSATKQTLEAHQSKVS
SLASASRKDHVLDIPTTQRQTACGTVGKQCCLYINYSEEIKSNIQRLHEASENLKNVPLLDWQGIFAKV
GDWFRSWGYVLLIVLFCLFIFVLIYVRVERKSRRSLNSQPLNLALSPQQSAQLLVSETSCQVSNRAMKG
LTTHQYDTSLL
HERV FRD ENV:
(SEQ ID NO: 223)
MGLLLLVLILTPSLAAYRHPDFPLLEKAQQLLQSTGSPYSTNCWLCTSSSTETPGTAYPASPREWTSIE
AELHISYRWDPNLKGLMRPANSLLSTVKQDFPDIRQKPPIFGPIFTNINLMGIAPICVMAKRKNGTNVG
TLPSTVCNVTFTVDSNQQTYQTYTHNQFRHQPRFPKPPNITFPQGTLLDKSSRFCQGRPSSCSTRNEWE
RPADYNQCLQISNLSSTAEWVLLDQTRNSLFWENKTKGANQSQTPCVQVLAGMTIATSYLGISAVSEFF
GTSLTPLFHFHISTCLKTQGAFYICGQSIHQCLPSNWTGTCTIGYVTPDIFIAPGNLSLPIPIYGNSPL
PRVRRAIHFIPLLAGLGILAGTGTGIAGITKASLTYSQLSKEIANNIDTMAKALTTMQEQIDSLAAVVL
QNRRGLDMLTAAQGGICLALDEKCCFWVNQSGKVQDNIRQLLNQASSLRERATQGWLNWEGTWKWESWV
LPLTGPLVSLLLLLLFGPCLLNLITQFVSSRLQAIKLOTNLSAGRHPRNIQESPF
HERV Kcon ENV 658 C-Terminal Truncation:
(SEQ ID NO: 139)
MNPSEMQRKAPPRRRRHRNRAPLTHKMNKMVTSEEQMKLPSTKKAEPPTWAQLKKLTQLATKYLENTKV
TQTPESMLLAALMIVSMVVSLPMPAGAAAANYTYWAYVPFPPLIRAVTWMDNPIEVYVNDSVWVPGPID
DRCPAKPEEEGMMINISIGYRYPPICLGRAPGCLMPAVQNWLVEVPTVSPISRFTYHMVSGMSLRPRVN
YLQDFSYQRSLKFRPKGKPCPKEIPKESKNTEVLVWEECVANSAVILQNNEFGTIIDWAPRGQFYHNCS
GQTQSCPSAQVSPAVDSDLTESLDKHKHKKLQSFYPWEWGEKGISTPRPKIVSPVSGPEHPELWRLTVA
SHHIRIWSGNQTLETRDRKPFYTVDLNSSLTVPLQSCVKPPYMLVVGNIVIKPDSQTITCENCRLLTCI
DSTFNWQHRILLVRAREGVWIPVSMDRPWEASPSVHILTEVLKGVLNRSKRFIFTLIAVIMGLIAVTAT
AAVAGVALHSSVQSVNFVNDWQKNSTRLWNSQSSIDQKLANQINDLRQTVIWMGDRLMSLEHRFQLQCD
WNTSDFCITPQIYNESEHHWDMVRRHLQGREDNLTLDISKLKEQIFEASKAHLNLVPGTEAIAGVADGL
ANLNPVTWVKTIGSTTIINLILILVCLFCLLLVCRCT
HERV T ENV 611 C-Terminal Truncation:
(SEQ ID NO: 140)
MGPQAWARPLKTAPKSSEAIKLILFIYLFCLFPPITPSAPSYSELLTSETTGRVFANTTWKAGTSKEVS
FAVDLCALFPEPARTHEEQCNLPVMGAGNVDLAAGFGHTGSRTGCGSSKGAEKGLQSVDFYLCPGNHPD
SSCRDSYQFFCPHWSCVTLATYSGGSTRSSTLSITRTSRPRPCTIRNCNPLTITVRNPNSAQWYYGMSW
GLRLYISGFDVGTMFTIQKKVLVPWSPPKPIGPLTDLGDPMFQKHPDRVDLTVPPPLLVPKSQLQRQHL
QPSLMSILDGVHHLLNLTQPKLAQDCWLCLKAKPPYYVGLGVEAMFKVNSLSCHTRPHALTLGDVSGNA
SCLISAGYNLSASPFQATCNQSLLTSLNASVSYQAPNNTWLACTSGLTRCINGTEPGPLLCVLVHVLPQ
VYVYSGPEGQLLIAPPELHSRFRRAAPLLVPLLAGLSIAGSAAIGTAALVQGETGLMSLSQQVDADLSN
LQSAINMLHTQVESLAEVVLQNRRGLDLLELSQGGLCAALGESCCFYANQSGVIKDTLQRVRENLDRRQ
QERENNTPWYQSMFNWNPWLTTLITGLAGPLLLLLLGLVFGPCILNWELNFVKQRIASV
HERV W ENV 480 C-Terminal Truncation:
(SEQ ID NO: 141)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGVQDQAREKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKFSNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIFNLLVNFVSSRI
HERV W ENV 483 C-Terminal Truncation:
(SEQ ID NO: 142)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGVQDQAREKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKESNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIFNLLVNFVSSRIEAV
HEMO ENV 518 C-Terminal Truncation:
(SEQ ID NO: 143)
MGSLSNYALLQLTLTAFLTILVQPQHLLAPVERTLSILTNQSNCWLCEHLDNAEQPELVFVPASASTWW
TYSGQWMYERVWYPQAEVQNHSTSSYRKVTWHWEASMEAQGLSFAQVRLLEGNESLCVENKNGSGPFLG
NIPKQYCNQILWEDSTDGTEMPSIDVTNESRNDDDDTSVCLGTRQCSWFAGCTNRTWNSSAVPLIGLPN
TQDYKWVDRNSGLTWSGNDTCLYSCQNQTKGLLYQLFRNLFCSYGLTEAHGKWRCADASITNDKGHDGH
RTPTWWLTGSNLTLSVNNSGLFFLCGNGVYKGFPPKWSGRCGLGYLVPSLTRYLTLNASQITNLRSFIH
KVTPHRCTQGDTDNPPLYCNPKDNSTIRALFPSLGTYDLEKAILNISKAMEQEFSATKQTLEAHQSKVS
SLASASRKDHVLDIPTTQRQTACGTVGKQCCLYINYSEEIKSNIQRLHEASENLKNVPLLDWQGIFAKV
GDWFRSWGYVLLIVLFCLFIFVLIYVRVERKSRRS
HEMO ENV 521 C-Terminal Truncation:
(SEQ ID NO: 144)
MGSLSNYALLQLTLTAFLTILVQPQHLLAPVERTLSILTNQSNCWLCEHLDNAEQPELVFVPASASTWW
TYSGQWMYERVWYPQAEVQNHSTSSYRKVTWHWEASMEAQGLSFAQVRLLEGNESLCVENKNGSGPFLG
NIPKQYCNQILWEDSTDGTEMPSIDVTNESRNDDDDTSVCLGTRQCSWFAGCTNRTWNSSAVPLIGLPN
TQDYKWVDRNSGLTWSGNDTCLYSCQNQTKGLLYQLFRNLFCSYGLTEAHGKWRCADASITNDKGHDGH
RTPTWWLTGSNLTLSVNNSGLFFLCGNGVYKGFPPKWSGRCGLGYLVPSLTRYLTLNASQITNLRSFIH
KVTPHRCTQGDTDNPPLYCNPKDNSTIRALFPSLGTYDLEKAILNISKAMEQEFSATKQTLEAHQSKVS
SLASASRKDHVLDIPTTQRQTACGTVGKQCCLYINYSEEIKSNIQRLHEASENLKNVPLLDWQGIFAKV
GDWFRSWGYVLLIVLFCLFIFVLIYVRVERKSRRSLNS
HERV Pb ENV 626 C-Terminal Truncation:
(SEQ ID NO: 145)
MDPSHPSQESTAPSSVMGHSPRGKSYQTKAKESLILFHLFCYSFFFPCALASHLIINVTRSDSPQTITE
DACLVIPCGDLQSQRQLAAAEKYLCPSEADASTLESFPFCHTWEYVVWTTQRQDWVPSQDEPLAVLKPY
IHFTKGIAPPNCRYNQCNPVQISITIPTLQDSSPTLNRFYGMGADVRGKDPIGFFELHLSTSPSLISPR
LSSSTPANQTIVSSSNDKSKVAIVEVKNLKQTLTIETGYKETNAWMEWIEYSVRSLNKSDCYACAQGRP
EAQVVPFPLGWSSDQPGMGCMVALFQHPTAWDSEFCRTLSVLFPETQHLEGEPPRAIQPPSPDAKFTSC
LSRQGKNLEFLGDLKGCSELKSFQELTNQSALVHARADVWWYCGGHLLDTLPSNWSGTCALIQLAIPFT
LAFQQPEKKKPQRRKTREAPQGSFDSHVYVDEIGVPQGVPDREKARDPVAAGFESLEPMVAINKNVAWI
NYIYYNQQRFINYTRDAIQGIAEQLGPTSQMAWENRMALDMILAEKGGVCVMIGTQCCTYIPNNTAPDG
TITKALQGLTSLSDELATNSGITDPFTGWLGQWFGKWKGLMASIVTSLAIAIAVLILVGCCIMPCIRGL
VQRLI
HERV FRD ENV 515 C-Terminal Truncation:
(SEQ ID NO: 146)
MGLLLLVLILTPSLAAYRHPDFPLLEKAQQLLQSTGSPYSTNCWLCTSSSTETPGTAYPASPREWTSIE
AELHISYRWDPNLKGLMRPANSLLSTVKQDFPDIRQKPPIFGPIFTNINLMGIAPICVMAKRKNGTNVG
TLPSTVCNVTFTVDSNQQTYQTYTHNQFRHQPRFPKPPNITFPQGTLLDKSSRFCQGRPSSCSTRNEWF
RPADYNQCLQISNLSSTAEWVLLDQTRNSLFWENKTKGANQSQTPCVQVLAGMTIATSYLGISAVSEFF
GTSLTPLFHFHISTCLKTQGAFYICGQSIHQCLPSNWTGTCTIGYVTPDIFIAPGNLSLPIPIYGNSPL
PRVRRAIHFIPLLAGLGILAGTGTGIAGITKASLTYSQLSKEIANNIDTMAKALTTMQEQIDSLAAVVL
QNRRGLDMLTAAQGGICLALDEKCCFWVNQSGKVQDNIRQLLNQASSLRERATQGWLNWEGTWKWFSWV
LPLTGPLVSLLLLLLFGPCLLNLITQFVSSRL
HERV H ENV 555 C-Terminal Truncation:
(SEQ ID NO: 147)
MIFAGKAPSNTSTLMKFYSLLLYSLLESFPFLCHPLPLPSYLHHTINLTHSLLAASNPSLVNNCWLCIS
LSSSAYTAVPAVQTDWATSPISLHLRTSENSPHLYPPEELIYFLDRSSKTSPDISHQQAAALLRTYLKN
LSPYINSTPPIFGPLTTQTTIPVAAPLCISWQRPTGIPLGNLSPSRCSFTLHLRSPTTNINETIGAFQL
HITDKPSINTDKLKNISSNYCLGRHLPCISLHPWLSSPCSSDSPPRPSSCLLIPSPENNSERLLVDTRR
FLIHHENRTFPSTQLPHQSPLQPLTAAALAGSLGVWVQDTPFSTPSHLFTLHLQFCLAQGLFFLCGSST
YMCLPANWTGTCTLVFLTPKIQFANGTEELPVPLMTPTQQKRVIPLIPLMVGLGLSASTVALGTGIAGI
STSVMTFRSLSNDESASITDISQTLSVLQAQVDSLAAVVLQNRRGLDLLTAEKGGLCIFLNEECCFYLN
QSGLVYDNIKKLKDRAQKLANQASNYAEPPWALSNWMSWVLPIVSPLIPIFLLLLFGPCIFRLVSQFIQ
NRI
HERV Rb 476 ENV C-Terminal Truncation:
(SEQ ID NO: 148)
MDPLHTIEKVPARRNIHDRGHQGHRMGDGTPGRPKISVQQMTRFSLIIFFLSAPFVVNASTSNVFLQWA
HSYADGLQQGDPCWVCGSLPVTNTMELPWWVSPLQGKDWVFFQSFIGDLKQWTGAQMTGVTRKNISEWP
INKTLNEPGHDKPFSVNETRDKVIAFAIPLLDTKVFVQTSRPQNTQYRNGFLQIWDGFIWLTATKGHLS
QIAPLCWEQRNHSLDNWPNTTRVMGWIPPGQCRHTILLQQRDLFATDWSQQPGLNWYAPNGTQWLCSPN
LWPWLPSGWLGCCTLGIPWAQGRWVKTMEVYPYLPHVVNQGTRAIVHRNDHLPTIFMPSVGLGTVIQHI
EALANFTQRALNDSLQSISLMNAEVYYMHEDILQNRMALDILTAAEGGTCALIKTECCVYIPNNSRNIS
LALEDTCRQIQVISSSALSLHDWIASQFSGRPSWWQKILIVLATLWSVGIALCCGLYFCRMF
HERV 3-1 ENV 586 C-Terminal Truncation:
(SEQ ID NO: 149)
MLGMNMLLITLFLLLPLSMLKGEPWEGCLHCTHTTWSGNIMTKTLLYHTYYECAGTCLGTCTHNQTTYS
VCDPGRGQPYVCYDPKSSPGTWFEIHVGSKEGDLLNQTKVFPSGKDVVSLYFDVCQIVSMGSLFPVIFS
SMEYYSSCHKNRYAHPACSTDSPVTTCWDCTTWSTNQQSLGPIMLTKIPLEPDCKTSTCNSVNLTILEP
DQPIWTTGLKAPLGARVSGEEIGPGAYVYLYIIKKTRTRSTQQFRVFESFYEHVNQKLPEPPPLASNLF
AQLAENIASSLHVASCYVCGGMNMGDQWPWEARELMPQDNFTLTASSLEPAPSSQSIWFLKTSIIGKFC
IARWGKAFTDPVGELTCLGQQYYNETLGKTLWRGKSNNSESPHPSPFSRFPSLNHSWYQLEAPNTWQAP
SGLYWICGPQAYRQLPAKWSGACVLGTIRPSFFLMPLKQGEALGYPIYDETKRKSKRGITIGDWKDNEW
PPERIIQYYGPATWAEDGMWGYRTPVYMLNRIIRLQAVLEIITNETAGALNLLAQQATKMRNVIYQNRL
ALDYLLAQEEGVCGKENLTNCCLELDDEGKVIK
HERV V-1 ENV 448 C-Terminal Truncation:
(SEQ ID NO: 150)
MTEKFLFLYLSLLPMPLLSQAQWNENSLVSFSKIIASGNHLSNCWICHNFITRSSSYQYILVRNESLNL
TFGSGIPEGQHKSVPLQVSLANSAHQVPCLDLTPPENQSSKTSFYFYNCSSLNQTCCPCPEGHCDRKNT
SEEGFPSPTIHPMSFSPAGCHPNLTHWCPAKQMNDYRDKSPQNRCAAWEGKELITWRVLYLLPKAHTVP
TWPKSTVPLGGPLSPACNQTIPAGWKSQLHKWFDSHIPRWACTPPGYVFLCGPQKNKLPFDGSPKITYS
TPPVANLYTCINNIQHTGECAVGLLGPRGIGVTIYNTTQPRQKRALGLILAGMGAAIGMIAPWGGFTYH
DVTLRNLSRQIDNIAKSTRDSISKLKASIDSLANVVMNNRLALDYLLAEQGGVCAVISKSCCIYVNNSG
AIEEDIKKIYDEVTWLHNFGKGDSAGSIWEAVKS
>HERV W ENV RBD MUT 1-1 (Q121A):
(SEQ ID NO: 151)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGVADQAREKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKFSNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPELGPLAAIILLLLFGPCIFNLLVNFVSSRIEAV
KLQMEPKMQSKTKIYRRPLDRPASPRSDVNDIKGTPPEEISAAQPLLRPNSAGSS
>HERV W ENV RBD MUT 1-2 (Q121A) (Q123A):
(SEQ ID NO: 152)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGVADAAREKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKFSNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLELGEECCYYVNQSGIV
TEKVKEIRDRIQRRAEELRNTGPWGLLSQWMPWILPFLGPLAAIILLLLFGPCIFNLLVNFVSSRIEAV
KLQMEPKMQSKTKIYRRPLDRPASPRSDVNDIKGTPPEEISAAQPLLRPNSAGSS
>HERV W ENV RBD MUT 1-3 (Q121A) (Q123A) (R125A):
(SEQ ID NO: 153)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGVADAAAEKHVKEVISQLTR
VHGTSSPYKGLDLSKLHETLRTHTRLVSLENTTLTGLHEVSAQNPTNCWICLPLNFRPYVSIPVPEQWN
NFSTEINTTSVLVGPLVSNLEITHTSNLTCVKESNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIFNLLVNFVSSRIEAV
KLQMEPKMQSKTKIYRRPLDRPASPRSDVNDIKGTPPEEISAAQPLLRPNSAGSS
>HERV W ENV RBD MUT 2-1 (D122A):
(SEQ ID NO: 154)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGVQAQAREKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKESNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIFNLLVNFVSSRIEAV
KLQMEPKMQSKTKIYRRPLDRPASPRSDVNDIKGTPPEEISAAQPLLRPNSAGSS
>HERV W ENV RBD MUT 2-2 (Q121A) (D122A):
(SEQ ID NO: 155)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGVAAQAREKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKFSNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPELGPLAAIILLLLFGPCIFNLLVNFVSSRIEAV
KLQMEPKMQSKTKIYRRPLDRPASPRSDVNDIKGTPPEEISAAQPLLRPNSAGSS
>HERV W ENV RBD MUT 2-3 (Q121A) (D122A) (Q123A):
(SEQ ID NO: 156)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGVAAAAREKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKESNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIENLLVNFVSSRIEAV
KLQMEPKMQSKTKIYRRPLDRPASPRSDVNDIKGTPPEEISAAQPLLRPNSAGSS
>HERV W ENV RBD MUT 2-4 (Q121A) (D122A) (Q123A) (R125G):
(SEQ ID NO: 157)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGVAAAAGEKHVKEVISQLTR
VHGTSSPYKGLDLSKLHETLRTHTRIVSLENTTLTGLHEVSAQNPTNCWICLPLNFRPYVSIPVPEQWN
NESTEINTTSVLVGPLVSNLEITHTSNLTCVKESNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIFNLLVNFVSSRIEAV
KLQMEPKMQSKTKIYRRPLDRPASPRSDVNDIKGTPPEEISAAQPLLRPNSAGSS
>HERV W ENV RBD MUT 3.0 (Q123A):
(SEQ ID NO: 158)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGVQDAAREKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKESNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIFNLLVNFVSSRIEAV
KLQMEPKMQSKTKIYRRPLDRPASPRSDVNDIKGTPPEEISAAQPLLRPNSAGSS
>HERV W ENV RBD MUT 3-1 (V120G) (Q123A):
(SEQ ID NO: 159)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGGQDAAREKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKESNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIFNLLVNFVSSRIEAV
KLQMEPKMQSKTKIYRRPLDRPASPRSDVNDIKGTPPEEISAAQPLLRPNSAGSS
>HERV W ENV RBD MUT 3-2 (V120G) (Q123A) (R125A):
(SEQ ID NO: 160)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGGQDAAAEKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKFSNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIFNLLVNFVSSRIEAV
KLQMEPKMQSKTKIYRRPLDRPASPRSDVNDIKGTPPEEISAAQPLLRPNSAGSS
>HERV W ENV RBD MUT 4.0 (R125A):
(SEQ ID NO: 161)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGVQDQAAEKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKFSNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIENLLVNFVSSRIEAV
KLQMEPKMQSKTKIYRRPLDRPASPRSDVNDIKGTPPEEISAAQPLLRPNSAGSS
>HERV W ENV RBD MUT 4-1 (V120G) (R125A):
(SEQ ID NO: 162)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGGQDQAAEKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKESNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIFNLLVNFVSSRIEAV
KLQMEPKMQSKTKIYRRPLDRPASPRSDVNDIKGTPPEEISAAQPLLRPNSAGSS
>HERV W ENV RBD MUT 4-2 (V120G) (D122A) (R125A):
(SEQ ID NO: 163)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGGQAQAAEKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKESNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIENLLVNFVSSRIEAV
KLQMEPKMQSKTKIYRRPLDRPASPRSDVNDIKGTPPEEISAAQPLLRPNSAGSS
>HERV W ENV RBD MUT 4-3 (V120G) (D122A) (Q123A) (R125A):
(SEQ ID NO: 164)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGGQAAAAEKHVKEVISQLTR
VHGTSSPYKGLDLSKLHETLRTHTRLVSLENTTLTGLHEVSAQNPTNCWICLPLNFRPYVSIPVPEQWN
NFSTEINTTSVLVGPLVSNLEITHTSNLTCVKFSNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIFNLLVNFVSSRIEAV
KLQMEPKMQSKTKIYRRPLDRPASPRSDVNDIKGTPPEEISAAQPLLRPNSAGSS
>HERV W ENV RBD MUT 5.0 (V120G):
(SEQ ID NO: 165)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGGQDQAREKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKFSNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIFNLLVNFVSSRIEAV
KLQMEPKMQSKTKIYRRPLDRPASPRSDVNDIKGTPPEEISAAQPLLRPNSAGSS
>HERV W ENV RBD MUT 5-1 (V120G) (Q123A):
(SEQ ID NO: 159)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGGQDAAREKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKESNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIFNLLVNFVSSRIEAV
KLQMEPKMQSKTKIYRRPLDRPASPRSDVNDIKGTPPEEISAAQPLLRPNSAGSS
>HERV W ENV RBD MUT 5-2 (V120G) (Q123A) (R125A):
(SEQ ID NO: 160)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGGQDAAAEKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKESNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIFNLLVNFVSSRIEAV
KLQMEPKMQSKTKIYRRPLDRPASPRSDVNDIKGTPPEEISAAQPLLRPNSAGSS
>HERV W ENV RBD MUT 6.0 (A124G):
(SEQ ID NO: 166)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGVQDQGREKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKFSNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIFNLLVNFVSSRIEAV
KLQMEPKMQSKTKIYRRPLDRPASPRSDVNDIKGTPPEEISAAQPLLRPNSAGSS
>HERV W ENV RBD MUT 6-1 (A124G) (R125A):
(SEQ ID NO: 167)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGVQDQGAEKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKFSNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIFNLLVNFVSSRIEAV
KLQMEPKMQSKTKIYRRPLDRPASPRSDVNDIKGTPPEEISAAQPLLRPNSAGSS
>HERV W ENV RBD MUT 6-2 (Q121A) (A124G) (R125A):
(SEQ ID NO: 168)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGVADQGAEKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKFSNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIENLLVNFVSSRIEAV
KLQMEPKMQSKTKIYRRPLDRPASPRSDVNDIKGTPPEEISAAQPLLRPNSAGSS
Targeting domain fusion site to transmembrane PDGFR anchor:
(SEQ ID NO: 226)
MALPVTALLLPLALLLHAARPEQKLISEEDLGSSGSGSAVS-(TARGETING DOMAIN)-
(SEQ ID NO: 169)
NAVGODTQEVIVVPHSLPFKVVVISAILALVVLTIISLIILIMLWQKKPR
Targeting domain fusion site to transmembrane CD9 anchor:
(SEQ ID NO: 227)
MLTRTLAVRSFAATMSPVKGGTKCIKYLLFGENFIFWLAGIAVLAIGLWLREDSQTKSIFEQETN-
(TARGETING DOMAIN)-
(SEQ ID NO: 170)
NNNSSFYTGVYILIGAGALMMLVGFLGCCGAVQESQCMLGLFFGELLVIFAIEIAAAIWGYSHKDEVIK
EVQEFYKDTYNKLKTKDEPQRETLKAIHYALNCCGLAGGVEQFISDICPKKDVLETFTVKSCPDAIKEV
FDNKFHIIGAVGIGIAVVMIFGMIFSMILCCAIRRNREMV
Targeting domain fusion site to transmembrane CD28 anchor:
(SEQ ID NO: 228)
MLTRTLAVRSFAATMALPVTALLLPLALLLHAARPEQKLISEEDL-(TARGETING DOMAIN)-
(SEQ ID NO: 171)
TGKLFWALVVVAGVLFCYGLLVTVALCVIWVRSG
Targeting domain fusion site to transmembrane CD8 anchor:
(SEQ ID NO: 229)
MLTRTLAVRSFAATMALPVTALLLPLALLLHAARPEQKLISEED-(TARGETING DOMAIN)-
(SEQ ID NO: 172)
IYIWAPLAGTCGVLLLSLVITLYCNHRNRRRVCKCPRPVVKSGDKPSLSARYV
Targeting domain fusion site to transmembrane CD4 anchor:
(SEQ ID NO: 230)
MLTRTLAVRSFAATMALPVTALLLPLALLLHAARPEQKLISEEDL-(TARGETING DOMAIN)-
(SEQ ID NO: 173)
MALIVLGGVAGLLLFIGLGIFFCVRCRHRRRQAERMSQIKRLLSEKKTCQCPHRFQKTCSPI
Targeting domain fusion site to transmembrane CD63 anchor:
(SEQ ID NO: 231)
MLTRTLAVRSFAATMAVEGGMKCVKELLYVLLLAFCACAVGLIAVGVGAQ-(TARGETING DOMAIN)-
(SEQ ID NO: 174)
LVLSQTIIQGATPGSLLPVVIIAVGVFLFLVAFVGCCGACKENYCLMITFAIFLSLIMLVEVAAAIAGY
VFRDKVMSEFNNNFRQQMENYPKNNHTASILDRMQADFKCCGAANYTDWEKIPSMSKNRVPDSCCINVT
VGCGINFNEKAIHKEGCVEKIGGWLRKNVLVVAAAALGIAFVEVLGIVFACCLVKSIRSGYEVM
Targeting domain fusion site to transmembrane CD81 anchor:
(SEQ ID NO: 232)
MLTRTLAVRSFAATMGVEGCTKCIKYLLFVENFVFWLAGGVILGVALWLRHDPQTTNLLYLEL-
(TARGETING DOMAIN)-
(SEQ ID NO: 175)
GDKPAPNTFYVGIYILIAVGAVMMFVGFLGCYGAIQESQCLLGTFFTCLVILFACEVAAGIWGEVNKDQ
IAKDVKQFYDQALQQAVVDDDANNAKAVVKTFHETLDCCGSSTLTALTTSVLKNNLCPSGSNIISNLEK
EDCHQKIDDLFSGKLYLIGIAAIVVAVIMIFEMILSMVLCCGIRNSSVY
Targeting domain fusion site to transmembrane CD86 anchor:
(SEQ ID NO: 233)
MLTRTLAVRSFAATMALPVTALLLPLALLLHAARPEQKLISEEDL-(TARGETING DOMAIN)-
(SEQ ID NO: 176)
PPDHIPWITAVLPTVIICVMVFCLILWKWKKKKRPRS
Targeting domain fusion site to transmembrane Notch anchor:
(SEQ ID NO: 234)
MLTRTLAVRSFAATMALPVTALLLPLALLLHAARPEQKLISEEDL-(TARGETING DOMAIN)-
(SEQ ID NO: 177)
ILDYSFTGGAGRDIPPPQIEEACELPECQVDAGNKVCNLQCNNHACGWDGGDCSLNENDPWKNCTQSLQ
CWKYFSDGHCDSQCNSAGCLFDGFDCQLTEGQCNPLYDQYCKDHESDGHCDQGCNSAECEWDGLDCAEH
VPERLAAGTLVLVVLLPPDQLRNNSFHFLRELSHVLHTNVVFKRDAQGQQMIFPYYGHEEELRKHPIKR
STVGWATSSLLPGTSGGRQRRELDPMDIRGSIVYLEIDNRQCVQSSSQCFQSATDVAAFLGALASLGSL
NIPYKIEAVKSEPVEPPLPSQLHLMYVAAAAFVLLFFVGCGVLLSRKRRRQLCIQKL
>HERV W ENV with targeting domain fusion site v1:
(SEQ ID NO: 178)
MALPYHIFLFTVLLPSFTLTA (SEQ ID NO: 235)-(TARGETING DOMAIN)-
PYKGLDLSKLHETLRTHTRIVSLENTTLTGLHEVSAQNPTNCWICLPLNFRPYVSIPVPEQWNNESTEI
NTTSVLVGPLVSNLEITHTSNLTCVKESNTTYTTNSQCIRWVTPPTQIVCLPSGIFFVCGTSAYRCLNG
SSESMCFLSFLVPPMTIYTEQDLYSYVISKPRNKRVPILPFVIGAGVLGALGTGIGGITTSTQFYYKLS
QELNGDMERVADSLVTLQDQLNSLAAVVLQNRRALDLLTAERGGTCLFLGEECCYYVNQSGIVTEKVKE
IRDRIQRRAEELRNTGPWGLLSQWMPWILPFLGPLAAIILLLLFGPCIENLLVNFVSSRIEAVKLQMEP
KMQSKTKIYRRPLDRPASPRSDVNDIKGTPPEEISAAQPLLRPNSAGSS
>HERV W ENV with targeting domain fusion site v2:
(SEQ ID NO: 236)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGM-(TARGETING DOMAIN)-
(SEQ ID NO: 179)
EKHVKEVISQLTRVHGTSSPYKGLDLSKLHETLRTHTRIVSLENTTLTGLHEVSAQNPTNCWICLPLNE
RPYVSIPVPEQWNNESTEINTTSVLVGPLVSNLEITHTSNLTCVKESNTTYTTNSQCIRWVTPPTQIVC
LPSGIFFVCGTSAYRCLNGSSESMCFLSFLVPPMTIYTEQDLYSYVISKPRNKRVPILPFVIGAGVLGA
LGTGIGGITTSTQFYYKLSQELNGDMERVADSLVTLQDQLNSLAAVVLQNRRALDLLTAERGGTCLFLG
EECCYYVNQSGIVTEKVKEIRDRIQRRAEELRNTGPWGLLSQWMPWILPFLGPLAAIILLLLEGPCIEN
LLVNFVSSRIEAVKLQMEPKMQSKTKIYRRPLDRPASPRSDVNDIKGTPPEEISAAQPLLRPNSAGSS
>HERV W ENV with targeting domain fusion site v3:
(SEQ ID NO: 237)
MALPYHIFLFTVLLPSFTLTA -(TARGETING DOMAIN)-
(SEQ ID NO: 180)
CWICLPLNFRPYVSIPVPEQWNNESTEINTTSVLVGPLVSNLEITHTSNLTCVKFSNTTYTTNSQCIRW
VTPPTQIVCLPSGIFFVCGTSAYRCLNGSSESMCFLSELVPPMTIYTEQDLYSYVISKPRNKRVPILPF
VIGAGVLGALGTGIGGITTSTQFYYKLSQELNGDMERVADSLVTLQDQLNSLAAVVLQNRRALDLLTAE
RGGTCLFLGEECCYYVNQSGIVTEKVKEIRDRIQRRAEELRNTGPWGLLSQWMPWILPFLGPLAAIILL
LLFGPCIFNLLVNFVSSRIEAVKLQMEPKMQSKTKIYRRPLDRPASPRSDVNDIKGTPPEEISAAQPLL
RPNSAGSS
Codon Optimized HERV T ENV:
(SEQ ID NO: 181)
ATGGGCCCACAGGCTTGGGCCAGACCCCTTAAAACCGCCCCTAAGTCCAGCGAGGCCATCAAGCTGATC
CTGTTCATCTACCTGTTTTGCCTGTTTCCTCCAATCACACCTAGCGCTCCATCCTACAGCTTTCTGCTG
ACAAGCTTCACAACCGGCAGAGTGTTTGCCAATACCACCTGGAAGGCCGGCACCTCCAAGGAAGTGTCC
TTCGCCGTGGACCTGTGCGCCCTGTTCCCCGAGCCTGCCAGAACACACGAGGAACAGTGCAACCTGCCT
GTGATGGGAGCCGGCAACGTGGATCTGGCCGCTGGCTTCGGCCACACCGGCTCACGGACCGGTTGTGGC
AGCTCTAAGGGCGCCGAGAAGGGCCTGCAAAGCGTCGACTTCTACCTGTGCCCCGGCAATCACCCCGAC
AGCTCTTGTAGAGATAGCTACCAGTTCTTCTGCCCTCATTGGAGCTGTGTGACCCTGGCTACATACAGC
GGCGGCAGCACCAGAAGCAGCACCCTGAGCATCACCAGAACCAGCAGACCTAGACCTTGCACTATCAGA
AACTGCAACCCTCTGACAATCACCGTGCGGAATCCGAATTCTGCCCAGTGGTACTACGGCATGAGCTGG
GGCCTGAGACTGTACATCAGCGGCTTCGACGTGGGCACCATGTTCACAATCCAGAAAAAGGTGCTGGTG
CCATGGAGCCCTCCTAAACCCATCGGCCCCCTGACCGACCTCGGAGATCCTATGTTCCAGAAGCACCCT
GACAGAGTGGACCTGACCGTGCCTCCCCCACTGCTGGTCCCTAAGAGCCAGCTGCAGAGACAGCACCTG
CAGCCTAGCCTCATGAGCATCCTGGATGGCGTTCATCACCTGCTCAACCTGACCCAACCTAAGCTGGCT
CAGGACTGCTGGCTGTGCCTGAAGGCTAAGCCCCCCTACTACGTCGGCCTGGGCGTGGAAGCCATGTTC
AAGGTGAACAGCCTGAGCTGCCACACCCGCCCCCACGCCCTGACCCTGGGCGACGTGTCCGGCAACGCC
TCTTGTCTGATTAGCGCCGGTTATAACCTGAGCGCCTCTCCTTTCCAAGCCACATGCAATCAATCTCTC
CTGACAAGCCTGAATGCCTCTGTGTCTTATCAGGCCCCTAACAACACCTGGCTGGCCTGCACAAGCGGA
CTGACCCGGTGCATCAACGGCACAGAACCTGGCCCTCTGCTGTGTGTGCTGGTGCACGTGCTGCCTCAG
GTATACGTGTACTCTGGACCTGAGGGCCAGCTGCTGATTGCCCCTCCTGAGCTGCACAGCAGATTCAGA
CGGGCCGCTCCACTGCTGGTGCCCTTGCTCGCCGGACTGAGCATCGCCGGATCAGCTGCTATCGGCACC
GCCGCCCTGGTGCAAGGCGAGACCGGCCTGATGAGCCTGAGCCAGCAGGTGGATGCCGACCTGTCCAAC
CTGCAGAGCGCCATCAACATGCTGCACACACAGGTGGAAAGTCTGGCCGAGGTTGTGCTGCAGAACCGG
CGGGGCCTGGATCTGCTGTTTCTGTCTCAGGGAGGACTGTGTGCCGCCCTGGGGGAGAGCTGCTGCTTC
TACGCCAACCAGAGCGGAGTTATCAAGGACACCCTGCAAAGGGTGCGGGAAAACCTGGACCGGAGACAG
CAGGAGAGAGAGAACAACACACCCTGGTACCAGAGCATGTTTAACTGGAACCCCTGGCTGACCACACTG
ATCACCGGCCTCGCAGGCCCCCTCCTGCTGCTGCTGCTGGGCCTCGTGTTCGGCCCTTGTATCCTGAAC
TGGTTCCTGAACTTCGTGAAGCAGCGGATCGCCAGCGTAAAACTTACATACCTGAGAACTCAGTACAAC
CCTCTGGTCATCACCGAGGAATCCATGATCTGA
Codon Optimized HERV H ENV:
(SEQ ID NO: 182)
ATGATCTTTGCCGGCAAGGCCCCTAGTAATACCAGCACCCTGATGAAATTCTACAGCCTGTTGCTGTAC
AGCCTGCTGTTTAGCTTCCCCTTCCTGTGCCACCCCCTTCCGCTGCCCTCCTACCTGCATCACACCATC
AACCTGACACACAGCCTGCTGGCTGCTAGCAACCCTAGCCTCGTTAACAACTGCTGGCTGTGCATAAGC
CTGAGCTCTAGCGCCTATACAGCCGTGCCTGCCGTGCAGACAGACTGGGCCACATCTCCCATCAGCCTG
CACCTCAGAACTTCTTTCAACAGCCCACACCTGTATCCTCCAGAAGAGCTGATCTACTTCCTCGACAGA
TCTAGCAAGACCTCCCCTGACATCTCTCACCAGCAGGCCGCTGCCCTGCTGAGAACCTACCTGAAAAAC
CTGAGCCCCTACATCAACAGCACCCCTCCAATCTTCGGACCTCTGACCACCCAGACCACCATCCCTGTG
GCCGCTCCTCTGTGCATCAGCTGGCAGAGACCTACCGGCATCCCTCTGGGAAACCTGTCTCCCAGCAGA
TGCAGCTTTACGCTGCACCTGCGGAGCCCCACAACCAATATCAACGAGACAATCGGCGCCTTCCAGCTG
CACATCACAGATAAGCCTTCTATCAACACCGATAAGCTGAAGAACATCTCAAGCAACTACTGCCTGGGA
AGACACCTGCCTTGTATCAGCCTGCATCCTTGGCTGTCTTCTCCATGTAGCAGTGATAGCCCACCTAGA
CCCTCTAGCTGCCTGCTGATTCCTAGCCCTGAAAACAACAGCGAGCGGCTGCTGGTCGACACAAGGAGA
TTCCTGATCCACCACGAGAACCGGACATTCCCTAGCACCCAGCTGCCACACCAGAGCCCTCTGCAACCT
CTGACAGCCGCTGCCCTGGCCGGAAGCCTGGGAGTGTGGGTCCAGGACACCCCTTTCAGCACACCTAGC
CACCTGTTCACCCTGCACCTCCAGTTTTGCCTGGCCCAGGGCCTGTTCTTCCTGTGTGGCAGCTCCACG
TACATGTGCCTGCCTGCTAATTGGACCGGCACCTGCACCCTGGTGTTCCTGACCCCCAAGATCCAGTTC
GCCAACGGCACAGAGGAACTGCCCGTGCCCCTGATGACCCCAACCCAACAGAAGCGGGTGATCCCCCTG
ATCCCTCTGATGGTGGGCCTGGGCCTGTCTGCCTCTACAGTGGCCTTAGGCACCGGCATCGCCGGCATC
AGCACAAGCGTGATGACCTTCCGGAGCCTGTCTAACGACTTCAGCGCCAGCATCACCGACATCTCTCAG
ACTCTGTCCGTGCTGCAGGCTCAGGTGGACTCTCTGGCCGCCGTGGTGCTGCAGAACCGCAGAGGCCTC
GATCTGCTGACCGCCGAGAAGGGCGGCCTCTGTATTTTTCTGAACGAAGAGTGCTGCTTCTACCTGAAC
CAGTCAGGCCTGGTGTACGACAACATCAAGAAGCTGAAGGACCGGGCCCAGAAACTGGCCAATCAAGCC
TCCAATTACGCCGAACCTCCCTGGGCTCTGTCCAATTGGATGAGCTGGGTGCTCCCTATCGTGTCCCCC
CTGATCCCCATCTTCCTCCTGCTGCTGTTTGGCCCTTGTATCTTCAGACTGGTGTCCCAGTTCATCCAA
AACAGAATTCAGGCCATCACCAACCACAGCATCAGACAGATGTTCCTGCTGACCAGCCCTCAGTACCAC
CCTCTTCCTCAGGATTTGCCTAGCGCCTGA
Codon Optimized HERV Pb ENV:
(SEQ ID NO: 183)
ATGGACCCGAGCCACCCCTCCCAGGAGTCTACAGCTCCCAGCAGCGTGATGGGCCACAGCCCCAGAGGA
AAGAGTTATCAGACTAAGGCTAAGGAAAGCCTGATTCTGTTCCACCTGTTCTGCTACAGCTTCTTCTTC
CCATGTGCCCTGGCTTCCCATCTGATCATCAACGTGACCAGAAGCGACTCTCCTCAAACCATCACCTTC
GATGCATGCCTGGTCATCCCCTGTGGCGACCTGCAGTCTCAGAGACAGCTGGCCGCTGCTGAAAAGTAC
CTGTGCCCTTCCGAAGCTGATGCCAGCACACTGTTCAGCTTCCCCTTCTGTCACACCTGGGAGTACGTG
GTTTGGACCACCCAGAGACAGGACTGGGTCCCCAGCCAGGACTTCCCACTGGCCGTGCTGAAGCCCTAC
ATCCACTTCACCAAGGGCATCGCCCCACCTAACTGCAGATATAACCAGTGTAATCCTGTGCAGATCTCT
ATCACAATTCCTACCCTGCAAGATAGCAGCCCCACCCTGAATAGATTCTACGGCATGGGCGCCGACGTG
CGGGGCAAGGACCCTATCGGCTTCTTCGAGCTGCACCTGAGCACAAGCCCTTCACTGATCAGCCCTAGA
CTGAGCAGCAGTACCCCTGCCAACCAGACCATCGTGTCCTCTAGCAACGACAAGTCAAAGGTGGCGATC
GTGGAAGTGAAGAACCTGAAACAGACACTCACAATCGAGACAGGCTACAAGGAAACAAACGCCTGGATG
GAGTGGATCGAATACTCCGTGCGGAGCCTGAACAAAAGCGACTGCTACGCCTGCGCCCAGGGCAGACCT
GAGGCCCAGGTGGTGCCTTTCCCACTGGGCTGGTCATCTGATCAGCCCGGCATGGGCTGCATGGTGGCC
CTCTTTCAGCACCCTACAGCCTGGGACAGCGAGTTCTGTAGAACCCTGAGCGTGCTGTTCCCTGAGACC
CAGCACCTTGAGGGCGAGCCTCCTCGGGCCATTCAACCTCCTAGCCCCGACGCCAAGTTTACCAGCTGC
CTGAGTAGACAGGGAAAGAATCTGGAATTCCTGGGAGATCTGAAGGGCTGCAGCGAACTCAAGAGCTTC
CAGGAGCTGACCAACCAGAGCGCCCTGGTGCACGCCCGGGCCGACGTGTGGTGGTACTGCGGCGGACAC
CTGCTGGACACCCTGCCTTCCAATTGGAGCGGAACCTGCGCCCTGATCCAGCTGGCCATCCCCTTCACC
CTGGCCTTCCAACAACCTGAAAAGAAGAAACCTCAGAGGCGGAAGACCAGAGAGGCCCCTCAGGGCAGC
TTTGACTCCCATGTGTACGTGGACGAGATCGGCGTGCCCCAGGGAGTGCCTGACCGGTTTAAGGCCAGA
GATCCCGTGGCCGCCGGATTTGAGAGCCTGTTTCCCATGGTCGCTATCAACAAGAACGTGGCCTGGATC
AACTACATCTACTACAACCAACAGAGATTCATCAACTACACCCGGGACGCCATCCAGGGCATCGCCGAA
CAGCTGGGCCCTACAAGCCAGATGGCCTGGGAAAACCGGATGGCTCTGGACATGATCCTGGCAGAGAAA
GGCGGCGTGTGCGTGATGATCGGAACACAGTGCTGCACCTACATCCCTAACAATACCGCCCCTGATGGC
ACAATAACCAAGGCCCTGCAGGGCCTGACCTCTCTGTCTGATGAGCTGGCTACCAACAGCGGCATCACC
GACCCCTTTACCGGCTGGCTGGGCCAGTGGTTCGGCAAATGGAAGGGCCTGATGGCTTCTATCGTTACA
AGCCTGGCCATTGCCATCGCCGTGCTGATCCTGGTTGGTTGTTGTATCATGCCATGCATCAGAGGGCTG
GTGCAGCGACTGATCGAGACAGCCTCTAACAAGACATTCCCTAGCTCTAGCCAGTCCTACTCCAACAAG
TTCTTTCCAGTGAACGAGCACGAGATCCGGATCATCCTGGATAGATTCAAGGCCGAGCACGTGTGA
Codon Optimized HERV Rb ENV:
(SEQ ID NO: 184)
ATGGACCCCCTGCATACCATCGAAAAGGTGCCCGCCAGAAGAAACATCCACGATAGAGGCCACCAGGGA
CATAGAATGGGCGACGGCACCCCTGGCAGGCCTAAGATCAGCGTCCAGCAGATGACCAGGTTCAGCCTG
ATCATCTTCTTCCTGTCTGCCCCTTTCGTGGTGAATGCCTCTACAAGCAACGTGTTCCTGCAGTGGGCC
CACTCCTACGCCGATGGCCTGCAGCAAGGCGATCCTTGTTGGGTGTGCGGCAGCCTGCCCGTGACCAAC
ACCATGGAACTGCCCTGGTGGGTGTCTCCACTGCAGGGCAAGGACTGGGTCTTTTTCCAGAGCTTTATC
GGAGATCTGAAGCAGTGGACCGGTGCCCAGATGACAGGCGTGACAAGAAAGAACATCTCCGAGTGGCCT
ATCAACAAGACCCTGAACGAGCCTGGCCACGACAAACCTTTTAGCGTGAACGAGACACGGGACAAGGTG
ATCGCCTTCGCCATCCCTCTGCTGGACACCAAGGTGTTCGTGCAGACCAGCAGACCTCAGAACACCCAG
TACCGGAATGGCTTCCTGCAGATCTGGGACGGATTCATCTGGCTGACCGCCACAAAGGGCCACCTGAGC
CAGATTGCCCCACTGTGTTGGGAACAGAGAAACCACAGCCTGGACAACTGGCCTAACACCACAAGAGTG
ATGGGCTGGATCCCGCCAGGACAGTGCAGACACACCATCCTGCTGCAGCAGCGGGACCTGTTCGCCACC
GACTGGTCTCAGCAGCCTGGCCTGAACTGGTACGCCCCTAACGGCACACAGTGGCTGTGCAGCCCTAAC
CTGTGGCCCTGGCTCCCCAGCGGCTGGCTGGGCTGCTGCACACTGGGAATACCTTGGGCTCAAGGAAGA
TGGGTTAAAACAATGGAAGTGTATCCTTACCTGCCCCACGTGGTCAACCAGGGAACGCGGGCCATAGTG
CACCGGAACGACCACCTGCCCACCATCTTTATGCCTAGCGTGGGCCTGGGCACCGTGATCCAGCACATC
GAGGCCCTGGCTAATTTCACCCAGAGAGCCCTGAATGACTCCCTGCAATCTATTTCTCTTATGAACGCC
GAGGTGTACTACATGCACGAGGACATCCTGCAAAACCGGATGGCCCTGGATATTCTGACAGCCGCTGAA
GGCGGCACCTGCGCCCTGATCAAGACAGAGTGCTGCGTGTACATCCCTAACAACAGCCGGAATATCAGC
CTGGCCCTGGAAGATACCTGTCGACAGATCCAGGTGATCTCCAGCAGCGCCCTCAGCCTTCACGACTGG
ATCGCCAGCCAATTCTCTGGCAGACCAAGCTGGTGGCAGAAAATCCTGATCGTGCTGGCAACCCTGTGG
TCCGTGGGCATCGCTCTCTGTTGCGGCCTGTACTTCTGCAGAATGTTCAGCCAACACATCCCCCAGACC
CACAGTATCATCTTTCAGCAGGAGCTGCCTCTGAGCCCCCCTTCTCAGGAGCACTACCAGAGCCAAAGA
GATATCTTCCACAGCAATGCCCCTTGA
Codon Optimized HERV 3-1 ENV:
(SEQ ID NO: 185)
ATGCTGGGCATGAACATGCTCCTGATCACCCTGTTCCTGCTGCTGCCTCTGAGCATGCTGAAAGGCGAA
CCTTGGGAGGGCTGCCTGCACTGCACCCACACCACCTGGAGCGGCAACATCATGACCAAGACACTGTTG
TACCACACCTACTACGAGTGCGCCGGTACATGTCTGGGCACCTGTACACACAACCAGACAACATACTCT
GTCTGCGACCCTGGCAGAGGCCAACCTTACGTGTGCTACGACCCCAAGAGCAGCCCCGGCACCTGGTTC
GAGATCCACGTCGGCAGCAAGGAAGGCGATCTGCTGAATCAGACCAAGGTGTTCCCCTCCGGCAAAGAT
GTGGTGTCTCTGTACTTCGACGTGTGCCAGATCGTGTCCATGGGCTCTCTGTTTCCAGTGATCTTCAGC
AGCATGGAATACTATAGCAGCTGCCACAAGAACAGATACGCCCATCCTGCCTGCAGCACAGACAGCCCC
GTGACCACCTGTTGGGACTGCACCACATGGTCCACAAATCAGCAATCTCTGGGCCCTATCATGCTGACC
AAGATCCCCCTGGAGCCTGATTGCAAGACCAGCACCTGCAACAGCGTGAACCTGACCATCCTGGAGCCT
GACCAGCCTATCTGGACCACAGGCCTGAAGGCCCCTCTGGGCGCCCGGGTGTCCGGAGAAGAAATCGGC
CCAGGAGCCTACGTGTACCTGTATATCATCAAGAAGACTAGAACCAGAAGCACCCAGCAATTTAGAGTG
TTCGAGTCTTTCTATGAGCACGTTAACCAGAAGCTGCCTGAGCCCCCCCCCCTGGCCTCCAATCTGTTC
GCCCAGCTGGCAGAAAACATCGCTAGCTCTCTGCACGTGGCCAGTTGTTACGTGTGTGGCGGCATGAAC
ATGGGAGATCAGTGGCCTTGGGAAGCTAGAGAACTGATGCCCCAGGACAACTTCACCCTCACCGCCTCC
TCTCTGGAGCCTGCTCCTAGCAGCCAGAGCATCTGGTTTCTGAAGACCAGCATCATTGGCAAGTTCTGC
ATCGCCAGATGGGGCAAGGCCTTTACCGATCCTGTGGGCGAACTGACATGTCTGGGCCAGCAGTACTAC
AACGAGACACTGGGAAAAACACTTTGGCGGGGAAAAAGCAACAACAGCGAGAGCCCCCATCCGAGCCCT
TTTTCAAGATTCCCCAGCCTGAACCACTCTTGGTACCAGCTCGAGGCCCCAAACACCTGGCAGGCCCCA
AGCGGACTGTACTGGATCTGCGGCCCTCAGGCCTACAGACAGCTGCCCGCCAAGTGGAGCGGCGCTTGT
GTGCTGGGAACGATCCGGCCTAGCTTCTTCCTGATGCCTCTGAAGCAGGGCGAGGCCCTGGGCTACCCT
ATCTACGATGAGACAAAGAGGAAGAGTAAGCGGGGAATCACGATTGGCGACTGGAAGGACAATGAGTGG
CCTCCTGAAAGAATCATCCAATACTACGGCCCAGCCACCTGGGCCGAGGACGGCATGTGGGGCTACCGG
ACCCCTGTCTACATGCTGAACCGGATCATCAGACTGCAGGCCGTGCTGGAAATCATCACCAACGAGACA
GCCGGGGCCCTGAACCTGCTGGCTCAGCAGGCCACAAAGATGCGGAACGTAATCTATCAGAACAGACTG
GCTCTGGACTACCTGCTGGCCCAGGAGGAAGGCGTGTGCGGCAAGTTCAATCTGACCAATTGCTGCCTC
GAACTGGACGACGAGGGCAAAGTGATCAAAGAGATTACCGCTAAGATCCAGAAACTGGCCCACATCCCT
GTGCAAACCTGGAAGGGCTGA
Codon Optimized HERV V-1 ENV:
(SEQ ID NO: 186)
ATGACCGAGAAGTTCCTGTTCCTGTACCTCTCTCTCCTGCCTATGCCTCTGCTGTCTCAGGCCCAGTGG
AACGAGAACAGCCTGGTTTCTTTTTCCAAAATCATCGCCAGCGGCAACCACCTGTCTAATTGCTGGATC
TGCCACAACTTTATCACCAGAAGCAGCAGCTACCAGTACATCCTGGTCAGAAATTTCAGCCTGAATCTG
ACCTTCGGCTCTGGAATCCCTGAGGGCCAGCACAAAAGCGTGCCCCTGCAAGTGTCCCTGGCTAATAGC
GCCCACCAGGTGCCGTGCCTGGACCTGACCCCTCCTTTCAACCAGAGCTCCAAAACCAGCTTTTACTTC
TACAACTGCAGCTCCCTGAACCAGACCTGTTGTCCTTGTCCTGAAGGACACTGCGACAGAAAGAACACA
AGCGAGGAAGGCTTCCCCTCCCCTACCATCCACCCTATGAGCTTCAGCCCCGCCGGTTGTCACCCCAAC
CTGACCCACTGGTGCCCCGCCAAACAGATGAATGACTACAGAGATAAGTCCCCACAGAACAGATGCGCC
GCTTGGGAGGGAAAGGAACTGATCACATGGCGCGTGCTGTATCTGCTTCCTAAGGCCCATACAGTGCCT
ACATGGCCCAAGTCTACCGTGCCACTGGGAGGACCTCTGAGCCCCGCCTGCAACCAAACAATCCCTGCC
GGCTGGAAGAGCCAGCTGCACAAGTGGTTCGACAGCCACATCCCCAGATGGGCCTGTACCCCACCAGGC
TACGTGTTCCTGTGCGGCCCTCAGAAAAACAAGCTGCCCTTCGACGGCTCTCCTAAGATCACTTACAGC
ACCCCTCCTGTGGCCAACCTGTACACATGTATCAACAACATCCAACACACAGGCGAGTGCGCCGTGGGC
CTGCTAGGCCCTAGGGGCATCGGAGTTACAATCTACAACACCACCCAGCCTCGGCAGAAGCGGGCCCTG
GGCCTGATTCTGGCCGGCATGGGAGCTGCTATCGGCATGATCGCCCCATGGGGCGGTTTCACCTACCAC
GACGTGACCCTGCGGAACCTGTCTAGACAGATCGACAACATCGCCAAGTCCACCAGAGATAGCATTAGC
AAGCTGAAGGCCAGCATCGATAGCCTGGCCAACGTGGTGATGAACAACCGGCTGGCCTTGGATTATCTG
CTGGCTGAACAGGGCGGCGTGTGCGCCGTCATCAGCAAGTCCTGCTGCATCTACGTGAACAATAGCGGC
GCCATCGAGGAAGATATCAAGAAGATCTACGACGAGGTGACCTGGCTGCATAATTTTGGCAAGGGCGAC
TCTGCTGGCAGCATCTGGGAGGCCGTGAAGAGCGCCCTGCCTAGCCTGACATGGTTCGTGCCCCTCCTG
GGCCCCGCAGCCCTGAACTCTCTGCTGAGCCCTCTGTGGCCTCTGTCCCTGTGA
Codon Optimized HERV FRD ENV:
(SEQ ID NO: 187)
ATGGGTCTTCTGCTGCTCGTGCTGATCCTGACACCTAGCCTGGCCGCATATAGACACCCCGACTTCCCC
TTACTGGAAAAGGCCCAGCAGCTGCTCCAGAGCACCGGATCTCCATACAGCACTAACTGCTGGCTGTGC
ACAAGCTCCAGCACGGAAACCCCCGGCACCGCCTATCCTGCCAGCCCTCGGGAATGGACATCCATCGAG
GCCGAGCTGCACATCTCTTACAGATGGGACCCCAACCTGAAAGGCCTGATGCGGCCTGCTAATAGCCTT
CTGTCCACCGTGAAGCAGGATTTTCCAGATATTAGACAGAAGCCCCCCATCTTCGGCCCCATCTTCACC
AATATCAACCTGATGGGCATTGCCCCTATATGCGTGATGGCTAAGAGAAAAAACGGCACCAACGTGGGC
ACCCTGCCTTCTACAGTGTGCAACGTGACATTCACAGTGGACAGCAACCAACAGACCTACCAGACCTAC
ACCCACAACCAGTTCAGACACCAACCTCGCTTCCCAAAGCCTCCAAACATCACCTTCCCTCAGGGCACC
CTGCTGGACAAGAGCAGCAGATTCTGCCAGGGCAGACCTAGCAGCTGCAGCACAAGAAATTTCTGGTTC
CGGCCCGCCGATTACAACCAGTGTCTGCAGATCAGCAACCTGAGCAGCACCGCCGAGTGGGTGCTGCTG
GACCAGACCCGGAACAGCCTGTTCTGGGAAAACAAGACAAAGGGCGCCAACCAGAGCCAGACACCTTGT
GTGCAAGTGCTTGCCGGAATGACCATCGCTACAAGCTACCTGGGCATCAGCGCCGTTAGCGAGTTCTTC
GGCACCTCTCTGACCCCTCTGTTCCACTTCCACATCAGCACCTGCCTGAAGACCCAGGGAGCCTTCTAC
ATCTGCGGCCAGAGCATCCACCAGTGTCTGCCATCTAATTGGACCGGCACATGTACCATCGGCTACGTG
ACCCCTGATATCTTTATCGCCCCTGGAAACCTGTCTCTGCCGATCCCTATCTACGGCAATAGCCCCCTG
CCTAGAGTGCGGCGGGCCATCCACTTTATCCCCCTGCTGGCCGGGCTGGGCATCCTGGCCGGCACCGGC
ACAGGCATCGCCGGCATCACCAAGGCCTCTCTGACATACAGCCAGCTGAGCAAGGAAATCGCCAACAAC
ATCGACACCATGGCTAAAGCCCTGACCACCATGCAGGAGCAGATCGACAGCCTGGCTGCCGTGGTGCTG
CAGAACAGAAGGGGCCTGGACATGCTGACCGCCGCTCAGGGCGGAATCTGTCTGGCCCTCGACGAGAAG
TGCTGCTTCTGGGTCAATCAGAGCGGCAAGGTGCAGGACAACATCCGGCAGCTGCTGAACCAGGCCTCC
TCTCTTAGAGAGAGAGCCACACAGGGATGGCTGAACTGGGAGGGCACATGGAAGTGGTTCAGCTGGGTC
CTGCCTCTGACCGGACCTCTGGTGTCTCTGCTGCTGCTGCTGCTGTTTGGCCCTTGCCTCCTGAATCTG
ATCACCCAATTTGTGTCCTCCAGACTGCAAGCTATCAAACTGCAAACCAACCTGTCTGCCGGAAGACAT
CCTAGAAACATCCAGGAGAGCCCTTTCTGA
Codon Optimized HERV HEMO ENV:
(SEQ ID NO: 188)
ATGGGCAGCCTGAGCAATTACGCCCTTCTGCAGCTGACGCTGACTGCCTTCCTGACCATCCTGGTGCAG
CCCCAGCACCTGCTGGCCCCTGTGTTCCGGACACTGAGTATCCTGACTAACCAGAGCAACTGCTGGCTG
TGCGAGCACCTGGATAACGCCGAGCAGCCTGAGCTGGTCTTTGTGCCAGCCTCTGCCTCGACCTGGTGG
ACCTACAGCGGCCAGTGGATGTACGAGAGAGTGTGGTACCCCCAGGCCGAAGTGCAGAACCACAGCACC
AGCAGTTATAGAAAGGTGACATGGCATTGGGAGGCTAGCATGGAAGCTCAGGGCCTGAGCTTTGCCCAG
GTGCGGCTGCTCGAGGGCAACTTCAGCCTGTGCGTGGAAAACAAGAACGGCTCTGGACCTTTCCTGGGC
AATATCCCTAAGCAGTACTGCAACCAGATCCTGTGGTTCGACAGCACCGATGGGACCTTCATGCCTAGC
ATCGACGTGACCAACGAGAGCAGAAATGATGATGACGACACATCTGTGTGCCTGGGCACCAGACAGTGT
AGCTGGTTCGCCGGCTGCACAAACAGGACCTGGAACAGCAGCGCCGTGCCTCTGATCGGCTTGCCTAAC
ACCCAGGACTACAAGTGGGTCGACAGAAACAGCGGACTGACCTGGTCCGGCAACGACACATGCCTGTAC
TCTTGTCAGAATCAAACCAAGGGCCTGCTGTACCAGCTGTTCCGGAACCTGTTCTGCAGCTACGGCCTG
ACAGAGGCTCATGGAAAATGGCGGTGCGCCGACGCCAGCATTACCAACGACAAGGGACACGACGGCCAC
AGAACCCCTACCTGGTGGCTGACCGGCAGCAATCTGACCCTGTCTGTGAACAACAGCGGCCTGTTCTTC
CTGTGTGGTAACGGCGTGTACAAGGGCTTCCCCCCCAAGTGGAGCGGCAGATGTGGCCTTGGATATCTG
GTTCCAAGCCTGACACGCTACCTGACCCTGAATGCCAGCCAAATCACCAACCTGAGAAGCTTCATCCAC
AAGGTGACCCCTCACCGGTGCACCCAAGGCGACACCGACAACCCCCCCCTGTACTGTAACCCAAAAGAT
AACAGCACCATCAGAGCCCTGTTCCCATCCCTGGGAACCTACGACCTGGAAAAGGCCATCCTGAATATC
AGCAAGGCCATGGAACAGGAGTTCAGCGCTACAAAGCAGACCCTGGAAGCCCACCAGTCTAAGGTTTCC
AGCCTGGCCTCCGCTAGCAGAAAGGACCACGTGCTGGACATCCCTACAACGCAACGGCAGACAGCCTGT
GGCACAGTGGGCAAACAGTGCTGCCTGTACATCAACTACAGCGAGGAAATCAAGAGCAACATTCAGAGA
CTGCACGAGGCTTCCGAGAACCTGAAAAACGTGCCTCTGCTGGATTGGCAGGGCATCTTCGCCAAGGTG
GGCGATTGGTTTAGATCTTGGGGCTACGTGCTCCTGATCGTGCTGTTTTGCCTGTTTATCTTCGTGCTG
ATCTACGTGCGGGTGTTCAGAAAGTCTCGCAGATCCCTGAACTCTCAGCCTCTGAATCTGGCACTGAGC
CCTCAGCAGAGCGCCCAACTGCTCGTATCTGAAACCAGCTGCCAGGTGTCCAACCGGGCCATGAAAGGC
CTGACAACCCACCAGTACGACACCTCCCTGCTCTGA
Codon Optimized HERV Kcon ENV:
(SEQ ID NO: 189)
ATGAACCCATCGGAGATGCAAAGAAAAGCACCTCCGCGGAGACGGAGACACCGCAATCGAGCACCGTTG
ACTCACAAGATGAACAAAATGGTGACGTCAGAAGAACAGATGAAGTTGCCATCCACCAAGAAGGCAGAG
CCGCCGACTTGGGCACAACTAAAGAAGCTGACGCAGTTAGCTACAAAATATCTAGAGAACACAAAGGTG
ACACAAACCCCAGAGAGTATGCTGCTTGCAGCCTTGATGATTGTATCAATGGTGGTAAGTCTCCCTATG
CCTGCAGGAGCAGCTGCAGCTAACTATACCTACTGGGCCTATGTGCCTTTCCCGCCCTTAATTCGGGCA
GTCACATGGATGGATAATCCTATAGAAGTATATGTTAATGATAGTGTATGGGTACCTGGCCCCATAGAT
GATCGCTGCCCTGCCAAACCTGAGGAAGAAGGGATGATGATAAATATTTCCATTGGGTATCGTTATCCT
CCTATTTGCCTAGGGAGAGCACCAGGATGTTTAATGCCTGCAGTCCAAAATTGGTTGGTAGAAGTACCT
ACTGTCAGTCCCATCAGTAGATTCACTTATCACATGGTAAGCGGGATGTCACTCAGGCCACGGGTAAAT
TATTTACAAGACTTTTCTTATCAAAGATCATTAAAATTTAGACCTAAAGGGAAACCTTGCCCCAAGGAA
ATTCCCAAAGAATCAAAAAATACAGAAGTTTTAGTTTGGGAAGAATGTGTGGCCAATAGTGCGGTGATA
TTACAAAACAATGAATTTGGAACTATTATAGATTGGGCACCTCGAGGTCAATTCTACCACAATTGCTCA
GGACAAACTCAGTCGTGTCCAAGTGCACAAGTGAGTCCAGCTGTTGATAGCGACTTAACAGAAAGTTTA
GACAAACATAAGCATAAAAAATTGCAGTCTTTCTACCCTTGGGAATGGGGAGAAAAAGGAATCTCTACC
CCAAGACCAAAAATAGTAAGTCCTGTTTCTGGTCCTGAACATCCAGAATTATGGAGGCTTACTGTGGCC
TCACACCACATTAGAATTTGGTCTGGAAATCAAACTTTAGAAACAAGAGATCGTAAGCCATTTTATACT
GTCGACCTAAATTCCAGTCTAACAGTTCCTTTACAAAGTTGCGTAAAGCCCCCTTATATGCTAGTTGTA
GGAAATATAGTTATTAAACCAGACTCCCAGACTATAACCTGTGAAAATTGTAGATTGCTTACTTGCATT
GATTCAACTTTTAATTGGCAACACCGTATTCTGCTGGTGAGAGCAAGAGAGGGCGTGTGGATCCCTGTG
TCCATGGACCGACCGTGGGAGGCCTCACCATCCGTCCATATTTTGACTGAAGTATTAAAAGGTGTTTTA
AATAGATCCAAAAGATTCATTTTTACTTTAATTGCAGTGATTATGGGATTAATTGCAGTCACAGCTACG
GCTGCTGTAGCAGGAGTTGCATTGCACTCTTCTGTTCAGTCAGTAAACTTTGTTAATGATTGGCAAAAA
AATTCTACAAGATTGTGGAATTCACAATCTAGTATTGATCAAAAATTGGCAAATCAAATTAATGATCTT
AGACAAACTGTCATTTGGATGGGAGACAGACTCATGAGCTTAGAACATCGTTTCCAGTTACAATGTGAC
TGGAATACGTCAGATTTTTGTATTACACCCCAAATTTATAATGAGTCTGAGCATCACTGGGACATGGTT
AGACGCCATCTACAGGGAAGAGAAGATAATCTCACTTTAGACATTTCCAAATTAAAAGAACAAATTTTC
GAAGCATCAAAAGCCCATTTAAATTTGGTGCCAGGAACTGAGGCAATTGCAGGAGTTGCTGATGGCCTC
GCAAATCTTAACCCTGTCACTTGGGTTAAGACCATTGGAAGTACTACGATTATAAATCTCATATTAATC
CTTGTGTGCCTGTTTTGTCTGTTGTTAGTCTGCAGGTGTACCCAACAGCTCCGAAGAGACAGCGACCAT
CGAGAACGGGCCATGATGACGATGGCGGTTTTGTCGAAAAGAAAAGGGGGAAATGTGGGGAAAAGCAAG
AGAGATCAGATTGTTACTGTGTCTGTGTAG
Codon Optimized HERV W ENV:
(SEQ ID NO: 190)
ATGGCCCTGCCTTACCACATCTTCCTGTTCACCGTGCTGCTGCCTTCTTTTACACTGACCGCCCCTCCA
CCTTGCAGATGCATGACCAGCTCCTCCCCTTATCAGGAGTTCCTGTGGCGGATGCAGAGACCTGGAAAT
ATCGACGCCCCTAGCTACCGGAGCCTCAGCAAGGGCACACCTACATTTACCGCCCACACGCATATGCCT
AGAAACTGCTACCACAGCGCCACCCTCTGTATGCACGCCAACACACACTACTGGACAGGAAAGATGATC
AACCCCTCCTGCCCCGGCGGACTGGGCGTGACCGTGTGTTGGACCTACTTCACACAGACCGGCATGAGC
GATGGCGGCGGTGTTCAGGACCAGGCCCGGGAGAAGCACGTGAAAGAGGTGATCTCTCAACTGACCCGG
GTGCACGGCACCAGCAGCCCCTACAAGGGCCTGGATCTGAGCAAACTGCACGAGACACTGCGGACCCAC
ACCAGACTGGTGTCTCTTTTCAACACCACCCTGACCGGCCTGCATGAAGTGAGCGCCCAGAATCCCACA
AACTGCTGGATCTGCCTGCCTCTGAATTTCAGACCCTACGTGAGCATCCCCGTGCCTGAGCAGTGGAAC
AACTTCAGCACAGAGATTAACACCACCAGCGTGCTGGTGGGCCCTCTCGTGAGCAACCTGGAAATCACA
CACACCAGCAACCTGACCTGTGTGAAGTTCAGCAACACCACATACACAACCAACAGCCAGTGCATCAGA
TGGGTCACCCCTCCTACCCAGATCGTGTGCCTGCCATCTGGCATCTTTTTCGTGTGCGGCACCTCTGCA
TATAGGTGTCTGAACGGATCTAGTGAGAGCATGTGCTTCCTGAGCTTCCTGGTGCCCCCCATGACCATC
TACACCGAGCAGGACCTGTACAGCTACGTAATTTCTAAACCTAGAAACAAGCGGGTGCCCATCCTGCCT
TTTGTGATCGGCGCCGGCGTGCTGGGAGCCCTGGGAACCGGCATCGGAGGCATCACCACATCTACCCAG
TTCTACTACAAGCTGTCTCAGGAGCTGAACGGCGACATGGAACGGGTGGCCGACAGCCTGGTCACACTG
CAAGATCAGCTGAACTCTCTGGCTGCCGTGGTGCTGCAGAACAGAAGAGCCCTGGACCTGCTGACCGCC
GAGAGAGGCGGCACATGTCTGTTTCTGGGCGAGGAATGCTGCTACTACGTGAACCAGTCCGGCATCGTC
ACAGAGAAAGTGAAGGAAATCCGGGACAGAATCCAGCGGAGAGCCGAAGAGCTGAGAAATACTGGACCT
TGGGGCCTGCTGTCCCAATGGATGCCCTGGATCCTGCCATTCCTGGGCCCTCTGGCCGCTATCATCCTG
CTGCTCCTGTTCGGCCCTTGTATCTTCAACCTGCTGGTTAATTTCGTGTCCAGCAGAATCGAGGCCGTG
AAGCTGCAGATGGAACCCAAGATGCAGAGCAAGACCAAGATCTACCGCCGGCCTTTGGATAGACCCGCC
AGCCCTAGATCCGACGTGAACGACATCAAGGGCACACCTCCAGAAGAAATTAGCGCTGCTCAGCCTCTA
CTGAGACCTAACAGCGCTGGCAGCTCCTGA
HERV W ENV RBD MUT (D122N) (Q123K):
(SEQ ID NO: 191)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGVQNKAREKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKFSNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIFNLLVNFVSSRIEAV
KLQMEPKMQSKTKIYRRPLDRPASPRSDVNDIKGTPPEEISAAQPLLRPNSAGSS
HERV W ENV RBD MUT (Q121K) (D122N) (Q123K):
(SEQ ID NO: 192)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGVKNKAREKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKFSNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIENLLVNFVSSRIEAV
KLQMEPKMQSKTKIYRRPLDRPASPRSDVNDIKGTPPEEISAAQPLLRPNSAGSS
HERV W ENV RBD MUT (D116N) (D122R):
(SEQ ID NO: 193)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSNGGGVQRQAREKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKFSNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIFNLLVNFVSSRIEAV
KLQMEPKMQSKTKIYRRPLDRPASPRSDVNDIKGTPPEEISAAQPLLRPNSAGSS
HERV W ENV RBD MUT (D116N):
(SEQ ID NO: 194)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSNGGGVQDQAREKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKFSNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIFNLLVNFVSSRIEAV
KLQMEPKMQSKTKIYRRPLDRPASPRSDVNDIKGTPPEEISAAQPLLRPNSAGSS
HERV W ENV RBD MUT (D122R):
(SEQ ID NO: 195)
MALPYHIFLFTVLLPSFTLTAPPPCRCMTSSSPYQEFLWRMQRPGNIDAPSYRSLSKGTPTFTAHTHMP
RNCYHSATLCMHANTHYWTGKMINPSCPGGLGVTVCWTYFTQTGMSDGGGVQRQAREKHVKEVISQLTR
VHGTSSPYKGLDLSKLHETLRTHTRIVSLENTTLTGLHEVSAQNPTNCWICLPLNFRPYVSIPVPEQWN
NFSTEINTTSVLVGPLVSNLEITHTSNLTCVKFSNTTYTTNSQCIRWVTPPTQIVCLPSGIFFVCGTSA
YRCLNGSSESMCFLSFLVPPMTIYTEQDLYSYVISKPRNKRVPILPFVIGAGVLGALGTGIGGITTSTQ
FYYKLSQELNGDMERVADSLVTLQDQLNSLAAVVLQNRRALDLLTAERGGTCLFLGEECCYYVNQSGIV
TEKVKEIRDRIQRRAEELRNTGPWGLLSQWMPWILPFLGPLAAIILLLLFGPCIFNLLVNFVSSRIEAV
KLQMEPKMQSKTKIYRRPLDRPASPRSDVNDIKGTPPEEISAAQPLLRPNSAGSS
HERV Kcon ENV MUT 1 (R140A):
(SEQ ID NO: 196)
MNPSEMQRKAPPRRRRHRNRAPLTHKMNKMVTSEEQMKLPSTKKAEPPTWAQLKKLTQLATKYLENTKV
TQTPESMLLAALMIVSMVVSLPMPAGAAAANYTYWAYVPFPPLIRAVTWMDNPIEVYVNDSVWVPGPID
DACPAKPEEEGMMINISIGYRYPPICLGRAPGCLMPAVQNWLVEVPTVSPISRFTYHMVSGMSLRPRVN
YLQDESYQRSLKFRPKGKPCPKEIPKESKNTEVLVWEECVANSAVILQNNEFGTIIDWAPRGQFYHNCS
GQTQSCPSAQVSPAVDSDLTESLDKHKHKKLQSFYPWEWGEKGISTPRPKIVSPVSGPEHPELWRLTVA
SHHIRIWSGNQTLETRDRKPFYTVDLNSSLTVPLQSCVKPPYMLVVGNIVIKPDSQTITCENCRLLTCI
DSTFNWQHRILLVRAREGVWIPVSMDRPWEASPSVHILTEVLKGVLNRSKRFIFTLIAVIMGLIAVTAT
AAVAGVALHSSVQSVNFVNDWQKNSTRLWNSQSSIDQKLANQINDLRQTVIWMGDRLMSLEHRFQLQCD
WNTSDFCITPQIYNESEHHWDMVRRHLQGREDNLTLDISKLKEQIFEASKAHLNLVPGTEAIAGVADGL
ANLNPVTWVKTIGSTTIINLILILVCLFCLLLVCRCTQQLRRDSDHRERAMMTMAVLSKRKGGNVGKSK
RDQIVTVSV
HERV Kcon ENV MUT 2 (R140C):
(SEQ ID NO: 197)
MNPSEMQRKAPPRRRRHRNRAPLTHKMNKMVTSEEQMKLPSTKKAEPPTWAQLKKLTQLATKYLENTKV
TQTPESMLLAALMIVSMVVSLPMPAGAAAANYTYWAYVPFPPLIRAVTWMDNPIEVYVNDSVWVPGPID
DCCPAKPEEEGMMINISIGYRYPPICLGRAPGCLMPAVQNWLVEVPTVSPISRFTYHMVSGMSLRPRVN
YLQDFSYQRSLKFRPKGKPCPKEIPKESKNTEVLVWEECVANSAVILQNNEFGTIIDWAPRGQFYHNCS
GQTQSCPSAQVSPAVDSDLTESLDKHKHKKLQSFYPWEWGEKGISTPRPKIVSPVSGPEHPELWRLTVA
SHHIRIWSGNQTLETRDRKPFYTVDLNSSLTVPLQSCVKPPYMLVVGNIVIKPDSQTITCENCRLLTCI
DSTFNWQHRILLVRAREGVWIPVSMDRPWEASPSVHILTEVLKGVLNRSKRFIFTLIAVIMGLIAVTAT
AAVAGVALHSSVQSVNFVNDWQKNSTRLWNSQSSIDQKLANQINDLRQTVIWMGDRLMSLEHRFQLQCD
WNTSDFCITPQIYNESEHHWDMVRRHLQGREDNLTLDISKLKEQIFEASKAHLNLVPGTEAIAGVADGL
ANLNPVTWVKTIGSTTIINLILILVCLFCLLLVCRCTQQLRRDSDHRERAMMTMAVLSKRKGGNVGKSK
RDQIVTVSV
Bc11a sgRNA spacer sequence DNA 5′ to 3′:
(SEQ ID NO: 198)
GTTTATCACAGGCTCCAGGAA
VEGF Site #3 (VEGFs3) sgRNA spacer sequence DNA 5′ to 3′:
(SEQ ID NO: 199)
GGTGAGTGAGTGTGTGCGTG

### EXAMPLES

The invention is further described in the following examples, which do not limit the scope of the invention described in the claims.

### Example 1. Minimal Virus-Like Particles Deliver Gene Editing Cargo to Target Cells

### Methods

The following methods were used in the Examples below. mhVLP or theVLP particles were produced in HEK293T cells by using jetPRIME® (Polyplus) or polyethylenimine (PEI) to transfect plasmids into these cells. PEI is Polyethylenimine 25 kD linear (Polysciences #23966-2). To make a stock ‘PEI MAX’ solution, Ig of PEI was added to 1 L endotoxin-free dH₂O that was previously heated to −80° C. and cooled to room temperature. This mixture was neutralized to pH 7.1 by addition of TON NaOH and filter sterilized with 0.22 μm polyethersulfone (PES). PEI MAX is stored at −20° C.

HEK293T cells were split to reach a confluency of 70%-90% at time of transfection and cultured in 2% FBS DMEM media. Plasmid vectors encoding cargo, e.g., encoding a CMV promoter driving expression of a hPLCδ1 or hAKT (E17K) PH domain fused to codon optimized Cas9-based ABE8e were co-transfected with plasmids encoding a U6 promoter driving expression of a sgRNA and another expressing the VSV-G envelope or hENV from a CMV promoter. Transfection reactions were assembled in JETPRIME buffer. For mhVLP particle production on 300 cm² T300 flasks, 19 μg PH-ABE8e expressing plasmid, 30 μg sgRNA-expression plasmid and 15 μg hENV expressing plasmid were mixed in 2 mL of JetPrime buffer, followed by addition of 4.6 μl/μg JetPrime. After 10 min incubation at room temperature, the transfection reactions were dispersed dropwise over the HEK293T cells.

Alternatively, if PEI was used for mhVLP particle production on 10 cm plates, 7.5 μg PH-ABE8e expressing plasmid, 7.5 μg sgRNA-expression plasmid and 5 μg hENV expressing plasmid were mixed in 1 mL Opti-MEM reduced serum media (Opti-MEM; GIBCO #31985-070), followed by addition of 27.5 μl PEI MAX. After 20-30 min incubation at room temperature, the transfection reactions were dispersed dropwise over the HEK293T cells. In the following examples, JETPRIME was used for transfection.

mhVLPs were harvested at 48 hours post-transfection and a media swap of 36 ml 2% DMEM is performed 18 hours-post transfection. 48-hours-post transfection, mhVLP supernatants were filtered using 0.45 m PVDF membrane filters and PEG precipitated (PEG-it, System Biosciences). The following examples employ PEG precipitation.

Alternatively, 0.45 um PVDF clarified harvest was transferred to polypropylene Beckman ultracentrifuge tubes that are used with the SW28 rotor (Beckman Coulter #326823). Each ultracentrifuge tube was filled with mhVLP-containing supernatant from three 10 cm plates or 1 T300 flask to reach an approximate final volume of 35-37.5 ml with a 1.5-3 ml 10% sucrose cushion. mhVLP supernatant underwent ultracentrifugation at approximately 100,000×g, or 25,000 rpm, at 4° C. for 2 hours.

After ultracentrifugation, supernatants were decanted and mhVLP pellets resuspended in DMEM 2% FBS media, or other media appropriate for the culturing of the target recipient cells, to be up to 2,000 times more concentrated than they were before ultracentrifugation. mhVLPs were added dropwise to cells that were seeded in a 24-well plate 24-hours prior to transduction. Polybrene (5-10 μg/mL in cell culture medium; Sigma-Aldrich #TR-1003-G) can be supplemented to enhance transduction efficiency, if necessary. Vectofusin-1 (10 μg/mL in cell culture medium, Miltenyi Biotec #130-111-163) can be supplemented to enhance transduction efficiency, if necessary. Immediately following the addition of mhVLPs, the 24-well plate can be centrifuged at 1,150×g for 30 min at room temperature to enhance transduction efficiency, if necessary (“Spinduction”). The following examples did not employ transduction enhancers or “spinduction.”

### Example 1.1

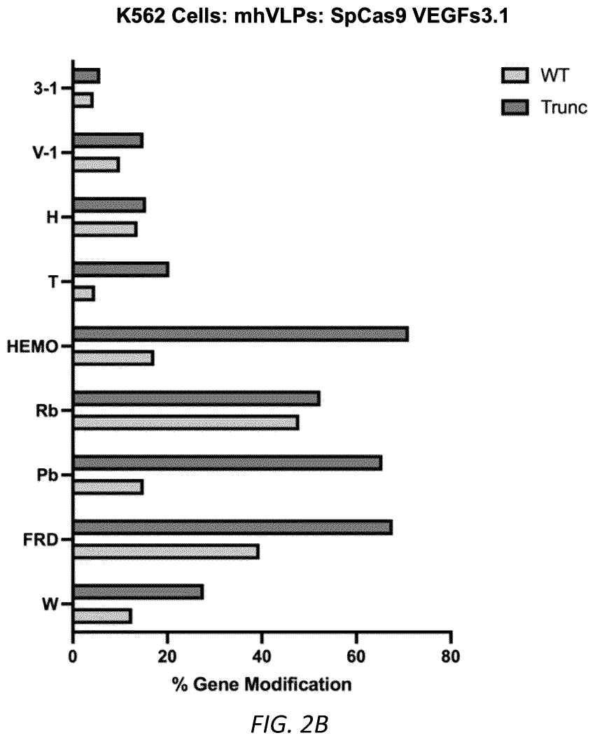

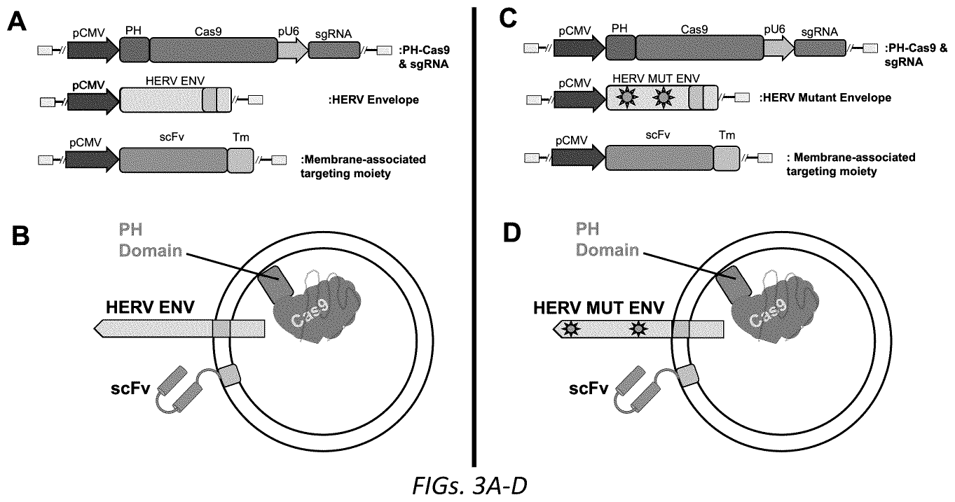

mhVLPs were produced by transient plasmid transfection of HEK293T cells (FIG. 1). mhVLPs were purified and concentrated 100-fold by filtration and PEG precipitation. These mhVLPs employed unmodified and truncated versions of the HERV envelopes (FIG. 2). mhVLPs were applied to K562 cells for an incubation period of 48 hours. K562 cells were harvested and genomic DNA was extracted. Targeted amplicon sequencing of extracted genomic DNA was used to quantify frequencies of gene edits (FIG. 2). FIGS. 3 & 4 show exemplary targeted mhVLPs that employ targeting domains on the mhVLP.

### Example 1.2

FIG. 5 shows that different phospholipid bilayer recruitment domains are capable of delivering cargo in previously described eVLPs (WO 2022/020800). eVLPs were produced by transient transfection of HEK293T cells, purified and concentrated 100-fold by filtration and PEG precipitation, and normalized based on Cas9 ELISA prior to transducing HEK293T cells so that the same pmol of Cas9 was applied in each well and comparisons could be made between different PH domains. Efficiencies of gene editing of endogenous VEGF target site were determined by targeted amplicon sequencing (FIG. 5). These eVLPs were pseudotyped with VSVG. The results showed that different PH domain and mutant PH domain fusions to cargos will result in different delivery efficiencies.

### Example 1.3

mhVLPs and one eVLP (WO 2022/020800) were produced by transient transfection of producer cells (FIG. 9). mhVLPs and eVLP were purified and concentrated 400-fold by 0.45 um PVDF filtration and PEG precipitation. These mhVLP and eVLP preparations packaged ABE8e targeting Bc11a and were applied to primary human hepatocytes for an incubation period of 48 hours. Primary human hepatocyte cells were harvested and genomic DNA was extracted. Targeted amplicon sequencing of extracted genomic DNA was used to quantify frequencies of gene edits (FIG. 9).

### Example 1.4

mhVLPs were produced by transient transfection of producer cells (FIG. 10). These mhVLPs were pseudotyped with a full-length W hENV, or a W hENV truncated at amino acid position 483. The cargo of these mhVLPs either possess or lack a fusion to the AKT (E17K) PH domain. mhVLPs were purified and concentrated 400-fold by 0.45 um PVDF filtration and PEG precipitation. These mhVLP preparations packaged ABE8e targeting VEGF Site #3 and were applied to primary human hepatocytes for an incubation period of 48 hours. Primary human hepatocyte cells were harvested and genomic DNA was extracted. Targeted amplicon sequencing of extracted genomic DNA was used to quantify frequencies of gene edits (FIG. 10). Cas9 ELISA and sgRNA qPCR was used to quantify ABE8e and sgRNA concentrations in the mhVLP preparations.

### Example 1.5

mhVLPs were produced by transient transfection of producer cells (FIG. 11). One mhVLP was pseudotyped with a W hENV truncated at amino acid position 483. Two mhVLPs are pseudotyped with W hENV truncated at amino acid position 483 that contain novel mutations. All cargos of these mhVLPs possess a fusion to the AKT (E17K) PH domain. mhVLPs were purified and concentrated 400-fold by 0.45 um PVDF filtration and PEG precipitation. These mhVLP preparations packaged ABE8e targeting VEGF Site #3 and were applied to primary human hepatocytes for an incubation period of 48 hours. Primary human hepatocyte cells were harvested and genomic DNA was extracted. Targeted amplicon sequencing of extracted genomic DNA was used to quantify frequencies of gene edits (FIG. 11).

### Example 1.6

mhVLPs were produced by transient transfection of producer cells (FIG. 12). One mhVLP was pseudotyped with a W hENV truncated at amino acid position 483 with novel enhancement mutations and the cargo possesses an AKT (E17K) PH domain. One mhVLP was pseudotyped with a W hENV truncated at amino acid position 483 with novel mutations and the cargo lacks a fusion to the AKT (E17K) PH domain. mhVLPs were purified and concentrated 400-fold by 0.45 um PVDF filtration and PEG precipitation. These mhVLP preparations packaged ABE8e targeting VEGF Site #3 and were applied to primary human skeletal muscle cells (phMUS) or primary human hematopoietic stem cells (phHSC) for an incubation period of 48 hours. Primary human cells were harvested and genomic DNA was extracted. Targeted amplicon sequencing of extracted genomic DNA was used to quantify frequencies of gene edits (FIG. 12).

### Example 2. Minimal Virus-Like Particles can be De-Targeted to Open the Possibility of Reprogrammed Tropism

### Methods

The following methods were used in the example below. mhVLP particles were produced in HEK293T cells by using jetPRIME® (Polyplus) or polyethylenimine (PEI) to transfect plasmids into these cells. PEI is Polyethylenimine 25 kD linear (Polysciences #23966-2). To make a stock ‘PEI MAX’ solution, Ig of PEI was added to 1 L endotoxin-free dH₂O that was previously heated to −80° C. and cooled to room temperature. This mixture was neutralized to pH 7.1 by addition of 10N NaOH and filter sterilized with 0.22 m polyethersulfone (PES). PEI MAX is stored at −20° C.

HEK293T cells were split to reach a confluency of 70%-90% at time of transfection and are cultured in 2% FBS DMEM media. Plasmid vectors encoding cargo, e.g., encoding a CMV promoter driving expression of a hPLCδ1 or hAKT (E17K) PH domain fused to codon optimized Cas9-based ABE8e were co-transfected with plasmids encoding a U6 promoter driving expression of a sgRNA and another expressing the the VSV-G envelope or hENV from a CMV promoter. Transfection reactions were assembled in JetPrime buffer. For mhVLP particle production on 300 cm² T300 flasks, 19 μg PH-ABE8e expressing plasmid, 30 μg sgRNA-expression plasmid and 15 μg hENV expressing plasmid were mixed in 2 mL of JetPrime buffer, followed by addition of 4.6 μl/μg JetPrime. After 10 min incubation at room temperature, the transfection reactions were dispersed dropwise over the HEK293T cells.

Alternatively, if PEI is used for mhVLP particle production on 10 cm plates, 7.5 μg PH-ABE8e expressing plasmid, 7.5 μg sgRNA-expression plasmid and 5 μg hENV expressing plasmid are mixed in 1 mL Opti-MEM reduced serum media (Opti-MEM; GIBCO #31985-070), followed by addition of 27.5 μl PEI MAX. After 20-30 min incubation at room temperature, the transfection reactions are dispersed dropwise over the HEK293T cells. In the following example, JetPrime was used for transfection.

mhVLPs were harvested at 48 hours post-transfection and a media swap of 36 ml 2% DMEM is performed 18 hours-post transfection. 48-hours-post transfection, mhVLP supernatants were filtered using 0.45 m PVDF membrane filters and PEG precipitated (PEG-it, System Biosciences). The following example employs PEG precipitation.

Alternatively, 0.45 um PVDF clarified harvest can be transferred to polypropylene Beckman ultracentrifuge tubes that are used with the SW28 rotor (Beckman Coulter #326823). Each ultracentrifuge tube is filled with mhVLP-containing supernatant from three 10 cm plates or 1 T300 flask to reach an approximate final volume of 35-37.5 ml with a 1.5-3 ml 10% sucrose cushion. mhVLP supernatant can undergo ultracentrifugation at approximately 100,000×g, or 25,000 rpm, at 4° C. for 2 hours.

After ultracentrifugation, supernatants were decanted and mhVLP pellets resuspended in DMEM 2% FBS media, or other media appropriate for the culturing of the target recipient cells, to be up to 2,000 times more concentrated than they were before ultracentrifugation. mhVLPs were added dropwise to cells that were seeded in a 24-well plate 24-hours prior to transduction. Polybrene (5-10 μg/mL in cell culture medium; Sigma-Aldrich #TR-1003-G) can be supplemented to enhance transduction efficiency, if necessary. Vectofusin-1 (10 μg/mL in cell culture medium, Miltenyi Biotec #130-111-163) can be supplemented to enhance transduction efficiency, if necessary. Immediately following the addition of mhVLPs, the 24-well plate can be centrifuged at 1,150×g for 30 min at room temperature to enhance transduction efficiency, if necessary (“Spinduction”). The following example do not employ transduction enhancers or “spinduction.”

### Example 2

mhVLPs were produced by transient transfection of producer cells (FIG. 13). One mhVLP was pseudotyped with a W hENV truncated at amino acid position 483 with de-targeting mutations that mimic the de-targeting mutation(s) in the envelope protein of the Spleen Necrosis Virus,¹⁰⁴ and the cargo possesses an AKT (E17K) PH domain. mhVLPs were purified and concentrated 400-fold by 0.45 um PVDF filtration and PEG precipitation. These mhVLP preparations packaged ABE8e targeting VEGF Site #3 and were applied to primary human hepatocytes for an incubation period of 48 hours. Primary human hepatocyte cells were harvested and genomic DNA was extracted. Targeted amplicon sequencing of extracted genomic DNA was used to quantify frequencies of gene edits (FIG. 13). Cas9 ELISA and sgRNA qPCR was used to quantify ABE8e and sgRNA concentrations in the mhVLP preparations.

### REFERENCES

1. Parseval, N. et al. Survey of human genes of retroviral origin: identification and transcriptome of the genes with coding capacity for complete envelope proteins. Journal of Virology 77, 10414-10422, (2003).
2. Okimoto, T. et al. VSV-G envelope glycoprotein forms complexes with plasmid DNA and MLV retrovirus-like particles in cell-free conditions and enhances DNA transfection. Molecular Therapy 4, 232-238, (2001).
3. Mangeot, P. et al. Protein transfer into human cells by VSV-G-induced nanovesicles. Molecular Therapy 19, 1656-1666, (2011).
4. Wagner, D. et al. High prevalence of Streptococcus pyogenes Cas9-reactive T cells within the adult human population. Nature Medicine 25, 242-248 (2019)
5. Kim, S. et al. CRISPR RNAs trigger innate immune responses in human cells. Genome Research 28, 1-7 (2018).
6. Charlesworth, C. et al. Identification of preexisting adaptive immunity to Cas9 proteins in humans. Nature Medicine 25, 249-254 (2019)
7. Ferdosi, S. et al. Multifunctional CRISPR-Cas9 with engineered immunosilenced human T cell epitopes. Nature Communications 10, Article number: 1842 (2019).
8. Wang, D. et al. Adenovirus-mediated somatic genome editing of Pten by CRISPR/Cas9 in mouse liver in spite of Cas9-specific immune responses. Human Gene Therapy 26, 432-442 (2015).
9. Devanabanda, M. et al. Immunotoxic effects of gold and silver nanoparticles: Inhibition of mitogen-induced proliferative responses and viability of human and murine lymphocytes in vitro. Journal of Immunotoxicology 13, 1547-6901 (2016).
10. Mout, R. et al. Direct cytosolic delivery of CRISPR/Cas9-ribonucleoprotein for efficient gene editing. ACS Nano 11, 2452-2458 (2017).
11. Yin, H. et al. structure-guided chemical modification of guide RNA enables potent non-viral in vivo genome editing. Nature Biotechnology 35, 1179-1187 (2017).
12. Qiao, J. et al. Cytosolic delivery of CRISPR/Cas9 ribonucleoproteins for genome editing using chitosan-coated red fluorescent protein. Chemical Communications 55, 4707-4710 (2019).
13. Li, L. et al. A rationally designed semiconducting polymer brush for NIR-II imaging guided light-triggered remote control of CRISPR/Cas9 genome editing. Advanced Materials 1901187, 1-9 (2019).
14. Gao, X. et al. Treatment of autosomal dominant hearing loss by in vivo delivery of genome editing agents. Nature 553, 217-221 (2018)
15. Lee, K. et al. Nanoparticle delivery of Cas9 ribonucleoprotein and donor DNA in vivo induces homology-directed DNA repair. Nature Biomedical Engineering 1, 889-901 (2017).
16. Staahl, B. et al. Efficient genome editing in the mouse brain by local delivery of engineered Cas9 ribonucleoprotein complexes. Nature Biotechnology 35, 431-433 (2017).
17. Zuris, J. et al. Cationic lipid-mediated delivery of proteins enables efficient protein-based genome editing in vitro and in vivo. Nature Biotechnology 33, 73-79 (2015).
18. Finn, J. et al. A single administration of CRISPR/Cas9 lipid nanoparticles achieves robust and persistent in vivo genome editing. Cell Reports 22, 2227-2235 (2018).
19. Wang, H. et al. Nonviral gene editing via CRISPR/Cas9 delivery by membrane-disruptive and endosomolytic helical polypeptide. PNAS 115, 4903-4908 (2018).
20. Del'Guidice, T. et al. Membrane permeabilizing amphiphilic peptide delivers recombinant transcription factor and CRISPR-Cas9/Cpf1 ribonucleoproteins in hard-to-modify cells. PLOS ONE 13, e0195558 (2018).
21. Colella, P. et al. Emerging Issues in AAV-Mediated In Vivo Gene Therapy. Molecular Therapy: Methods & Clinical Development 8, 87-104 (2018).
22. Naso, F. et al. Adeno-Associated Virus (AAV) as a Vector for Gene Therapy. BioDrugs 31, 317-334 (2017).
23. Handel, E. et al. Versatile and efficient genome editing in human cells by combining zinc-finger nucleases with adeno-associated viral vectors. Human Gene Therapy 23, 321-329 (2012).
24. Chadwick, A. et al. Reduced Blood Lipid Levels With In Vivo CRISPR-Cas9 Base Editing of ANGPTL3. Circulation 137, 975-977 (2018).
25. Schenkwein, D. et al. Production of HIV-1 Integrase Fusion Protein-Carrying Lentiviral Vectors for Gene Therapy and Protein Transduction. Human Gene Therapy 21, 589-602 (2010).
26. Cai, Y. et al. Targeted genome editing by lentiviral protein transduction of zinc-finger and TAL-effector nucleases. eLife 3, e01911 (2014).
27. Choi, J. et al. Lentivirus pre-packed with Cas9 protein for safer gene editing. Gene Therapy 23, 627-633 (2016).
28. Meyer, C. et al. Pseudotyping exosomes for enhanced protein delivery in mammalian cells. International Journal of Nanomedicine 12, 3153-3170 (2017).
29. Mangeot, P. et al. Genome editing in primary cells and in vivo using viral-derived Nanoblades loaded with Cas9-sgRNA ribonucleoproteins. Nature Communications 10, Article number: 45 (2019).
30. Lu, B. et al. Delivering SaCas9 mRNA by lentivirus-like bionanoparticles for transient expression and efficient genome editing. Nucleic Acids Research 47, e44 (2019).
31. Wang, Q. et al. ARMMs as a versatile platform for intracellular delivery of macromolecules. Nature Communications 9, 1-7 (2018).
32. Lainscek, D. et al. Delivery of an Artificial Transcription Regulator dCas9-VPR by Extracellular Vesicles for Therapeutic Gene Activation. ACS Synthetic Biology 7, 2715-2725 (2018).
33. Fuchs, J. et al. First-in-Human Evaluation of the Safety and Immunogenicity of a Recombinant Vesicular Stomatitis Virus Human Immunodeficiency Virus-1 gag Vaccine (HVTN 090). Open Forum Infectious Diseases 2, 1-9, (2015).
34. Cong, L. et al. Multiplex Genome Engineering Using CRISPR/Cas Systems. Science 339, 819-823, (2013).
35. Ran, F. et al. In vivo genome editing using Staphylococcus aureus Cas9. Nature 520, 186-191, (2015).
36. Zetsche, B. et al. Cpf1 Is a Single RNA-Guided Endonuclease of a Class 2 CRISPR-Cas System. Cell 163, 759-771, (2015).
37. Komor, A. et al. Programmable editing of a target base in genomic DNA without double-stranded DNA cleavage. Nature 533, 420-424, (2016).
38. Gaudelli, N. et al. Programmable base editing of A⋅T to G⋅C in genomic DNA without DNA cleavage. Nature 551, 464-471, (2017).
39. Voelkel, C. et al. Protein transduction from retroviral Gag precursors. Proc Natl Acad Sci USA 107, 7805-7810, (2010).
40. Kaczmarczyk, S. et al. Protein delivery using engineered virus-like particles. Proc Natl Acad Sci USA 108, 16998-17003, (2011).
41. Ebner, M. et al. PI(3,4,5)P3 Engagement Restricts Akt Activity to Cellular Membranes. Mol Cell 65, 416-431, (2017).
42. Urano, E. et al. Substitution of the myristoylation signal of human immunodeficiency virus type 1 Pr55Gag with the phospholipase C-d1 pleckstrin homology domain results in infectious pseudovirion production. J. Gen Virology 89, 3144-3149, (2008).
43. Pastuzyn, E. et al. The Neuronal Gene Arc Encodes a Repurposed Retrotransposon Gag Protein that Mediates Intercellular RNA Transfer. Cell 172, 275-288, (2018).
44. Lukacs, G. et al. Size-dependent DNA Mobility in Cytoplasm and Nucleus. Journal of Biological Chemistry 275, 1625-1629, (1999).
45. Kreiss, P. et al. Plasmid DNA size does not affect the physicochemical properties of lipoplexes but modulates gene transfer efficiency. Nucleic Acids Research 27, 3792-3798 (1999).
46. Nafissi, N. et al. DNA Ministrings: Highly Safe and Effective Gene Delivery Vectors. Molecular Therapy-Nucleic Acids 3, e165, (2014).
47. Fujimoto, T. et al. Selective EGLN Inhibition Enables Ablative Radiotherapy and Improves Survival in Unresectable Pancreatic Cancer. Cancer Research 79, 2327-2338 (2019).
48. Tai, S. et al. Differential Expression of Metallothionein 1 and 2 Isoforms in Breast Cancer Lines with Different Invasive Potential: Identification of a Novel Nonsilent Metallothionein-1H Mutant Variant. American Journal of Pathology 163, 2009-2019 (2003).
49. Caussinus, E. et al. Fluorescent fusion protein knockout mediated by anti-GFP nanobody. Nature Structural & Molecular Biology 19, 117-121, (2012).
50. Zhao, W. et al. Quantitatively Predictable Control of Cellular Protein Levels through Proteasomal Degradation. ACS Synthetic Biology 7, 540-552, (2018).
51. Clift, D. et al. A Method for the Acute and Rapid Degradation of Endogenous Proteins. Cell 171, 1692-1706, (2017).
52. Balla, T. & Vaimai, T. Visualizing Cellular Phosphoinositide Pools with GFP-Fused Protein-Modules. SCIENCE'S STKE 2002, p. p 13, DOI: 10.1126/stke.2002.125.p 13 (2002).
53. Carpten, J. et al. A transforming mutation in the pleckstrin homology domain of AKT1 in cancer. Nature 448, 439-445, (2007).
54. Virnai, P. et al. Selective cellular effects of overexpressed pleckstrin-homology domains that recognize PtdIns(3,4,5)P3 suggest their interaction with protein binding partners. Journal of Cell Science 118, 4879-4888, (2005).
55. Parikh, C. et al. Disruption of PH-kinase domain interactions leads to oncogenic activation of AKT in human cancers. PNAS 109, 19368-19373, (2012).
56. Jo, H. et al. Small molecule-induced cytosolic activation of protein kinase Akt rescues ischemia-elicited neuronal death. PNAS 109 (26) 10581-10586, (2012).
57. Han, F. et al. The critical role of AMPK in driving Akt activation under stress, tumorigenesis and drug resistance. Nat Commun 9, 4728 (2018).
58. Li, X. et al. Autophosphorylation of Akt at Threonine 72 and Serine 246: A POTENTIAL MECHANISM OF REGULATION OF Akt KINASE ACTIVITY*. Journal of Biological Chemistry 281, 13837-13843, (2006).
59. Liao, Y. et al. Peptidyl-prolyl cis/trans isomerase Pin1 is critical for the regulation of PKB/Akt stability and activation phosphorylation. Oncogene 28(26):2436-45 (2009).
60. Chu, N. et al. Akt Kinase Activation Mechanisms Revealed Using Protein Semisynthesis. Cell 174(4): 897-907.e14. (2018).
61. Lucid, I. et al. Conformational sampling of membranes by Akt controls its activation and inactivation. Proc Natl Acad Sci USA 115(17): E3940-E3949, (2018).
62. Baranov, M. et al SWAP70 Organizes the Actin Cytoskeleton and Is Essential for Phagocytosis. Cell Reports 17, p 1518-1531 (2016).
63. Munch, R. et al. Displaying High-affinity Ligands on Adeno-associated Viral Vectors Enables Tumor Cell-specific and Safe Gene Transfer. Molecular Therapy 21, 109-118 (2013).
64. Koide, S. et al. Chapter six—Target-Binding Proteins Based on the 10th Human Fibronectin Type III Domain (10Fn3). Methods in Enzymology 503, 5-156 (2012).
65. Leach, A. et al. Anti-DLL4 VNAR targeted nanoparticles for targeting of both tumour and tumour associated vasculature. Nanoscale 12, 14751-14763 (2020).
66. Hamann, M. et al. Improved targeting of human CD4+ T cells by nanobody-modified AAV2 gene therapy vectors. PLOS ONE 16(12): e0261269 (2021).
67. Dobson, C. et al. Antigen identification and high-throughput interaction mapping by reprogramming viral entry. Nature Methods 19, 449-460 (2022).
68. Sati, S. et al. RGD Peptide as a Targeting Moiety for Theranostic Purpose: An Update Study. International Journal of Peptide Research and Therapeutics 25, 49-65 (2019).
69. Bannas, P. et al. Nanobodies and Nanobody-Based Human Heavy Chain Antibodies As Antitumor Therapeutics. Front. Immunol. 8:1603. doi: 10.3389/fimmu.2017.01603 (2017).
70. Chu, S H et al., Rationally Designed Base Editors for Precise Editing of the Sickle Cell Disease Mutation. CRISPR J. 2021 April; 4(2):169-177.
71. Nguyen Tran M T et al., Engineering domain-inlaid SaCas9 adenine base editors with reduced RNA off-targets and increased on-target DNA editing. Nat Commun. 2020 Sep. 25; 11(1):4871.
72. Jiang L. et al., Internally inlaid SaCas9 base editors enable window specific base editing. Theranostics. 2022 Jun. 6; 12(10):4767-4778
73. Morsut L, Roybal K T, Xiong X, Gordley R M, Coyle S M, Thomson M, Lim W A. Engineering Customized Cell Sensing and Response Behaviors Using Synthetic Notch Receptors. Cell, 2016 Feb. 11; 164(4):780-91 doi: 10.1016/j.cell.2016.01.012. Epub 2016 Jan. 28. PMID: 26830878; PMCID: PMC4752866.
74. Trahtenherts A, Benhar I. An internalizing antibody specific for the human asialoglycoprotein receptor. Hybridoma (Larchmt). 2009 August; 28(4):225-33. doi: 10.1089/hyb.2009.0019. PMID: 19663694.
75. Tillotson B J, Goulatis L I, Parenti I, Duxburv E, Shusta E V. Engineering an Anti-Transferrin Receptor ScFv for p1-Sensitive Binding Leads to Increased Intracellular Accumulation. PLoS One. 2015 Dec. 29; 10(12):e0145820. doi: 10.1371/journal.pone.0145820. PMID: 26713870; PMCID: PMC4694649.
76. Wang J H, Forterre A V, Zhao J, Frimannsson D O, Delcayre A, Antes T J, Efron B, Jeffrey S S, Pegram M D, Matin A C. Anti-HER2 scFv-Directed Extracellular Vesicle-Mediated rnRNA-Based Gene Delivery Inhibits Growth of HER2-Positive Human Breast Tumor Xenografts by Prodrug Activation. Mol Cancer Ther. 2018 May; 17(5):1133-1142. doi: 10.1158/1535-7163.MCT-17-0827. Epub 2018 Feb. 26. PMID: 29483213; PMCID: PMC5932266.
77. Benedict C A, MacKrell A J, Anderson W F. Determination of the binding affinity of an anti-CD34 single-chain antibody using a novel, flow cytometry based assay. J Immunol Methods. 1997 Feb. 28; 201(2):223-31. doi: 10.1016/s0022-1759(96)00227-x. PMID: 9050944.
78. Babaei A, Zarkesh-Esfahani S H, Gharagozloo M. Production of a recombinant anti-human CD4 single-chain variable-fragment antibody using phage display technology and its expression in Escherichia coli. J Microbiol Biotechnol. 2011 Mav; 21(5):529-35. doi: 10.4014/jmb.1010.10022. PMID: 21617352.
79. Dehbashi M, Hojati Z, Motovali-Bashi M, Ganjalikhany M R, Cho W C, Shirnosaka A, Navabi P, Ganjialikhani-Hakerni M. A Novel CAR Expressing NK Cell Targeting CD25 With the Prospect of Overcoming Immune Escape Mechanism in Cancers. Front Oncol. 2021 May 14:11:649710. doi: 10.3389/fonc.2021.649710. PMID: 34055618; PMCID: PMC8160382,
80. Hosseinzadeh F, Moharnmnadi S, Nejatollahi F. Production and Evaluation of Specific Single-Chain Antibodies against CTLA-4 for Cancer-Targeted Therapy. Rep Biochem Mol Biol. 2017 October; 6(1):8-14, PMID: 29090224; PMCID: PMC5643449,
81. Lu P, Qiu S, Pan Y, Shi S, Yu Q, Yu F, Miao L, Wang H, Chen K. Discovery of an Heparin-Binding Epidermal Growth Factor Domain Antibody from a Phage Library and Analysis of Its Inhibitory Effects in SKOV3 Cells. Cancer Biother Radiopharm. 2021 Sep. 16. doi: 10.1089/cbr.2021.0123. Epub ahead of print. PMID: 34529926.
82. Chen Y, Zhang Y N, Yan R, Wang G, Zhang Y, Zhang Z R, Li Y, Ou J, Chu W, Liang Z, Wang Y, Chen Y L, Chen G. Wang Q, Zhou Q, Zhang B, Wang C. ACE2-targeting monoclonal antibody as potent and broad-spectrum coronavirus blocker. Signal Transduct Target Ther. 2021 Aug. 25; 6(1):315. doi: 10.1038/s41392-021-00740-y. PMID: 34433803; PMCID: PMC8385704.
83. Oesch-Bartlomowicz B, Huelster A, Wiss O, Antoniou-Lipfert P, Dietrich C, Arand M, Weiss C, Bockamp E, Oesch F. Aryl hydrocarbon receptor activation by cAMP vs. dioxin: divergent signaling pathways. Proc Natl Acad Sci USA. 2005 Jun. 28; 102(26):9218-23. doi: 10.1073/pnas.0503488102. Epub 2005 Jun. 21. PMID: 15972329: PMCID: PMC1154791.
84. Moll R, Dhouailly D, Sun T T. Expression of keratin 5 as a distinctive feature of epithelial and biphasic mesotheliomas. An inmnunohistochemical study using monoclonal antibody AE14. Virchows Arch B Cell Pathol Incl Mol Pathol. 1989; 58(2):129-45. doi: 10.1007/BF02890064. PMID: 2482572.
85. Fan J, Shen Z, Wang G, Yang H, Liu Y. Secretory expression of human ScFv against keratin in Pichia pastoris and its effects on cultured keratinocytes. Arch Dermatol Res. 2009 June; 301(5):367-72. doi: 10.1007/s00403-008-0908-4. Epub 2008 Oct. 21. Erratum in: Arch Dermatol Res. 2009 June; 301(5):395. PMID: 18936942.
86. Stausbol-Gron B, Jensen K B, Jensen Kit Jensen M Ø, Clark B F. De novo identification of cell-type specific antibody-antigen pairs by phage display subtraction. Isolation of a human single chain antibody fragment against human keratin 14. Eur J Biochem. 2001 May; 268(10):3099-107. doi: 10.1046/j.1432-1327.2001.02210.x, PMID: 11358530.
87. Malecha M J, Mietiinen M. Expression of keratin 13 in human epithelial neoplasms. Virchows Arch A Pathol Anat Histopathol. 1991; 418(3):249-54, doi: 10.1007/BF01606063. PMID: 1706547.
88. Wang Y, Loers G, Pan H C, Gouveia R, Zhao W J, Shen Y Q, Kleene R, Costa J, Schachner M. Antibody fragments directed against different portions of the human neural cell adhesion molecule L1 act as inhibitors or activators of L1 function. PLoS One. 2012; 7(12):e52404. doi: 10.1371/journal.pone.0052404. Epub 2012 Dec. 18. PMID: 23272240; PMCID: PMC3525558,
89. Martin-Otal C, Lasarte-Cia A, Serrano D, Casares N, Conde E, Navarro F, Sánchez-Moreno I, Gorraiz M, Sarrión P, Calvo A, De Andrea C E, Echeveste J, Vilas A, Rodriguez-Madoz J R. San Miguel J, Prosper F, Henas-Stubbs S. Lasarte J J, Lozano T. Targeting the extra domain A of fibronectin for cancer therapy with CAR-T cells. J Immunother Cancer. 2022 August; 10(8):e004479. doi: 10.1136/jitc-2021-004479. PMID: 35918123; PMCID: PMC9351345.
90. Kimizuka F, Taguchi Y, Ohdate Y. Kawase Y, Shinojo T, Hashino K, Kato I, Sekiguchi K, Titani K. Production and characterization of functional domains of human fibronectin expressed in Escherichia coli. J Biochem. 1991 August; 110(2):284-91. doi: 10.1093/oxfordjournals.jbchen.a123572. PMID: 1761524.
91. Tiwari A, Kumar R. Ram J. Sharma M, Luthra-Guptasarma M. Control of fibrotic changes through the synergistic effects of anti-fibronectin antibody and an RGDS-tagged form of the same antibody. Sci Rep. 2016 Aug. 3; 6:30872. doi: 10.1038/srep30872. PMID: 27484779; PMCID: PMC4971484.
92. Sebollela A, Cline E N, Popova I, Luo K, Sun X, Ahn J, Barcelos M A, Bezerra V N, Lyra E Silva N M, Patel J, Pinheiro N R, Qin L A, Kamel J M, Weng A, DiNunno N, Bebenek A M, Velasco P T, Viola K L, Lacor P N, Ferreira S T, Klein W L. A human scFv antibody that targets and neutralizes high molecular weight pathogenic amyloid-β oligomers. J Neurochem. 2017 September; 142(6):934-947. doi: 10.1111/jnc.14118. Epub 2017 Aug. 2. PMID: 28670737; PMCID: PMC5752625.
93. Rafiq S, Yeku O O, Jackson I-J, Purdon T J, van Leeuwen D G, Drakes D J, Song M, Miele M M, Li Z, Wang P, Yan S, Xiang J, Ma X, Seshan V E, Hendrickson R C, Liu C, Brentjens R J. Targeted delivery of a PD-1-blocking scFv by CAR-T cells enhances anti-tumor efficacy in vivo. Nat Biotechnol. 2018 October; 36(9):847-856. doi: 10.1038/nbt.4195. Epub 2018 Aug. 13. PMID: 30102295; PMCID: PMC6126939.
94. Harrasser M, Gohil S H, Lau H, Della Peruta M, Muczynski V, Patel D, Miranda E, Grigoriadis K, Grigoriadis A, Granger D, Evans R, Nathwani A C. Inducible localized delivery of an anti-PD-1 scFv enhances anti-tumor activity of ROR1 CAR-T cells in TNBC. Breast Cancer Res. 2022 Jun. 3; 24(1):39. doi: 10.1186/s13058-022-01531-1. PMCID: 35659040: PMCID: PMC9166313.
95. Zhang C, Helmsing S, Zagrebelsky M, Schirrmann T, Marschall A L, Schüngel M, Korte M, Hust M, Dübel S. Suppression of p75 neurotrophin receptor surface expression with intrabodies influences Bcl-xL mRRNA expression and neurite outgrowth in PC12 cells. PLoS One. 2012; 7(1):e30684. doi: 10.1371/journal.pone.0030684. Epub 2012 Jan. 24. PMID: 22292018; PMCID: PMC3265506.
96. Moazen B, Zarrinhaghighi A, Nejatollahi F. Selection and Evaluation of Specific Single Chain Antibodies against CD90, a Marker for Mesenchymal and Cancer Stem Cells. Rep Biochem Mol Biol. 2018 October; 7(1):45-51. PMID: 30324117; PMCID: PMC6175597.
97. Hao H, Zhen Y, Wang Z Chen F, Xie X. A novel therapeutic drug for colon cancer: EpCAM scFv-truncated protamine (tp)-siRNA. Cell Bio Int. 2013 August; 37(8):860-4. doi: 10.1002/cbin.10112. Epub 2013 Apr. 30. PMID: 23576466.
98. Sato Y, Mustafina K R, Luo Y, Martini C, Thomas D Y, Wisernan P W. Hanrahan J W. Nonspecific binding of common anti-CFTR antibodies in ciliated cells of human airway epithelium. Sci Rep. 2021 Dec. 1; 11(1):23256. doi: 10.1038/s41598-021-02420-x. PMID: 34853321; PMCID: PMC8636639.
99. Federica Toffalini, Jean-Baptiste Demoulin; The Transmembrane Domain of PDGFR-β Plays An Important Role in ETV6-PDGFR-β Activation. Blood 2008; 112 (11): 5320. doi: https://doi.org/10.1182/blood.V112.11.5320.5320
100. Verweij F J, Bebelman M P, Jimnenez C R, Garcia-Vallejo J J, Janssen H, Neefjes J, Knol J C, de Goeij-de Haas R, Piersma S R, Baglio S R, Verhage M, Middeldorp J M, Zomer A, van Rheenen J, Coppolino M G, Hurbain I, Raposo C, Smit M J, Toonen R F G, van Niel G, Pegtel D M. Quantifying exosome secretion from single cells reveals a modulatory role for GPCR signaling, J Cell Biol, 2018 Mar. 5; 217(3):1129-1142. doi: 10.1083/jcb.201703206. Epub 2018 Jan. 16. Erratum in: J Cell Biol. 2018 Jan. 23; PMID: 29339438; PMCID: PMC5839777.
101 Leddon S A, Fettis M M, Abramo K, Kelly R, Oleksyn D, Miller J. The CD28 Transmembrane Domain Contains an Essential Dimerization Motif Front Immunol. 2020 Jul. 16; 11:1519. doi: 10.3389/fimmu.2020.01519. PMID: 32765524; PMCID: PMC7378745.
102. Roselli E, Boucher J C, Li G. Kotani H, Spitler K. Reid K. Cervantes E V, Bulliard Y, Tu N, Lee S B, Yu B, Locke F L, Davila M L. 4-1BB and optimized CD28 co-stimulation enhances function of human mono-specific and bi-specific third-generation CART cells. J Immunother Cancer. 2021 October; 9(10):e003354. doi: 10.1136/jitc-2021-003354. PMID: 34706886; PMCID: PMC8552146.
103. Parrish H L, Glassman C R, Keenen M M, Deshpande N R, Bronnimann M P, Kuhns M S. A Transmembrane Domain GGxxG Motif in CD4 Contributes to Its Lck-Independent Function but Does Not Mediate CD4 Dimerization. PLoS One. 2015 Jul. 6; 10(7):e0132333. doi: 10.1371/journal.pone.0132333. Erratum in: PLoS One. 2016; 11(3):e0150876. PMID: 26147390; PMCID: PMC4493003.
104. Cheynet, V, Oriol, G, Mallet, F. Identification of the hASCT2-binding domain of the Env ERVWE1/syncytin-1 fusogenic glycoprotein. Retrovirology 2006, 3:41 doi:10.1186/1742-4690-3-41. PMID: 16820059; PMCID: PMC1524976.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A HERV envelope protein (hENV) comprising a sequence that is at least 95% identical to a sequence as set forth in Tables 1A-C, wherein the hENV comprises one, two, or all three of: a targeting domain at the N or C terminus, or inserted internally into the protein sequence; a truncation of one to 50 amino acids from the C terminus; and/or one or more RBD mutations.

2. The hENV of claim 1, wherein the targeting domain comprises a targeting peptide or a single chain variable fragment (scFv), nanobody, fibronectin type 3 domain (FN3), arginylglycylaspartic acid motif (RGD), single variable domain on a heavy chain/nanobody (VHH), variable domain of new antigen receptor (VNAR), or darpin.

3. The hENV of claim 1, wherein the targeting domain is inserted internally into the hENV, optionally after an amino acid corresponding to amino acid 18 or amino acid 114 of wild type HERV W.

4. The hENV of claim 3, further comprising one or more of: a deletion of 123 to 163 amino acids following amino acid 18; truncation of one to 50 amino acids from the C RBD mutations.

5. A nucleic acid sequence encoding the hENV of claim 1, optionally wherein the nucleic acid sequence is codon optimized for expression in human cells.

6. A vector comprising the nucleic acid sequence of claim 5, optionally operably linked to a promoter for expression of the hENV.

7. A host cell comprising the nucleic acid sequence of claim 5, and optionally expressing the hENV.

8. A targeted human endogenous virus-like particle (theVLP) comprising a human endogenous retroviral (HERV) envelope protein (hENV) and a targeting domain, wherein the hENV optionally comprises a truncation of one to 50 amino acids from the C terminus and/or one or more RBD mutations, wherein the targeting domain is (i) fused at the N or C terminus, or inserted internally into the of the hENV, or (ii) is a membrane-anchored targeting domain comprising a targeting domain fused to a transmembrane domain, and
optionally, a cargo disposed in the core of the theVLP, wherein the cargo is optionally fused to a phospholipid bilayer recruitment domain.

9. A minimal human virus-like particle (mhVLP), comprising:
a membrane comprising a phospholipid bilayer and a human endogenous retroviral (HERV) envelope protein (hENV), wherein the hENV optionally comprises one, two, or all three of: a targeting domain at the N or C terminus, or inserted internally into the protein; a truncation of one to 50 amino acids from the C terminus; and/or one or more RBD mutations; and
optionally, a cargo disposed in the core of the mhVLP, wherein the cargo is optionally fused to a phospholipid bilayer recruitment domain;
preferably wherein the mhVLP does not comprise an exogenous gag, pro and/or pol protein, and optionally wherein the mhVLP further comprises a separate targeting domain.

10. The theVLP of claim 8, wherein the targeting domain comprises a targeting peptide or an scFv, nanobody, FN3, RGD, VHH, VNAR, or darpin.

11. The theVLP of claim 8, wherein the targeting domain is inserted internally into the hENV, optionally after a signal sequence, optionally after an amino acid corresponding to amino acid 18 or amino acid 114 of wild type HERV W.

12. The theVLP of claim 8, further comprising one or more of: a deletion of 123 to 163 amino acids following amino acid 18; truncation of one to 50 amino acids from the C RBD mutations.

13. The theVLP of claim 8, wherein the cargo is a therapeutic or diagnostic protein and/or nucleic acid encoding a therapeutic or diagnostic protein, and/or a chemical, optionally a small molecule therapeutic or diagnostic.

14. The theVLP of claim 8, wherein the cargo is a gene editing and/or epigenetic modulating reagent.

15. The theVLP of claim 8, wherein the gene editing and/or epigenetic modulating reagent comprises a zinc finger (ZF), transcription activator-like effector (TALE), and/or CRISPR-Cas protein, variant, or fusion thereof; a nucleic acid encoding a zinc finger (ZF), transcription activator-like effector (TALE), and/or CRISPR-Cas protein, variant, or fusion thereof; a guide RNA and/or crRNA; and/or a ribonucleoprotein complex (RNP) comprising a CRISPR-Cas protein, variant, or fusion thereof and optionally a guide RNA and/or crRNA.

16. The theVLP of claim 15, wherein the cargo is selected from the proteins listed in Tables 2, 3, 4 & 5, or that is at least 95% identical to a sequence set forth herein, optionally in Tables 2, 3, 4, and 5.

17. The theVLP of claim 15, wherein the cargo comprises a CRISPR-Cas protein, and the mhVLP further comprises one or more guide RNAs that bind to and direct the CRISPR-Cas protein to a target nucleic acid sequence.

18. The theVLP of claim 8, wherein the cargo comprises a fusion to a phospholipid bilayer recruitment domain, preferably as shown in Table 6, or that is at least 95% identical to a sequence set forth herein in Table 6.

19. A method of delivering a cargo to a target cell, optionally a cell in vivo or in vitro, the method comprising contacting the cell with the theVLP of claim 8 comprising the cargo.

20. A method of producing a theVLP or an mhVLP comprising a cargo, the method comprising:
providing a cell expressing (i) a human endogenous retroviral (HERV) envelope protein (hENV), wherein the hENV optionally comprises one, two, or all three of: a targeting domain at the N or C terminus, or inserted internally into the protein; a truncation of one to 50 amino acids from the C terminus; and/or one or more RBD mutations, (ii) a cargo, and (iii) optionally a separate targeting domain, and
(iv) optionally wherein the cell does not express or overexpress an exogenous gag, pro, or pol, protein; and
maintaining the cell under conditions such that the cells produce the VLPs or mhVLPs.

21. The method of claim 20, further comprising harvesting and optionally purifying and/or concentrating the produced VLPs or mhVLPs.

22. The method of claim 20, wherein the cargo is a therapeutic and/or diagnostic protein and/or nucleic acid encoding a therapeutic and/or diagnostic protein, and/or a small molecule, optionally a therapeutic and/or diagnostic small molecule.

23. The method of claim 20, wherein the cargo is a gene editing and/or epigenetic modulating reagent.

24. The method of claim 20, wherein the gene editing or epigenetic modulating reagent comprises a zinc finger (ZF), transcription activator-like effector (TALE), and/or CRISPR-Cas protein, variant, or fusion thereof; a nucleic acid encoding a zinc finger (ZF), transcription activator-like effector (TALE), and/or CRISPR-Cas protein, variant, or fusion thereof; a guide RNA and/or crRNA; or a ribonucleoprotein complex (RNP) comprising a CRISPR-Cas protein, variant, or fusion thereof and optionally a guide RNA.

25. The method of claim 24, wherein the cargo reagent is selected from the proteins listed in Tables 2, 3, 4 & 5, or that is at least 95% identical to a sequence set forth herein, optionally in Tables 2, 3, 4, and 5.

26. The method of claim 24, wherein the cargo reagent comprises a CRISPR-Cas protein, variant, or fusion thereof and the VLP or mhVLP further comprises one or more guide RNAs that bind to and direct the CRISPR-based genome editing or modulating protein to a target sequence.

27. The method of claim 20, wherein the cargo comprises a fusion to a phospholipid bilayer recruitment domain, preferably as shown in Table 6, or that is at least 95% identical to a sequence set forth herein in Table 6.

28. A cell expressing (i) a human endogenous retroviral (HERV) envelope protein (hENV), wherein the hENV optionally comprises one, two, or all three of a targeting domain at the N or C terminus, or inserted internally into the protein; a truncation of one to 50 amino acids from the C terminus; and/or one or more RBD mutations, (ii) a cargo, (iii) optionally a separate targeting domain, and
(iv) optionally wherein the cell does not express or overexpress an exogenous gag, pro, or pol, protein.

29. The cell of claim 28, wherein the cargo is a therapeutic and/or diagnostic protein and/or nucleic acid encoding a therapeutic and/or diagnostic protein, and/or a small molecule, optionally a therapeutic and/or diagnostic small molecule.

30. The cell of claim 28, wherein the cargo is a gene editing and/or epigenetic modulating reagent.

31. The cell of claim 28, wherein the gene editing or epigenetic modulating reagent comprises a zinc finger (ZF), transcription activator-like effector (TALE), and/or CRISPR-Cas protein, variant, or fusion thereof; and/or a nucleic acid encoding a zinc finger (ZF), transcription activator-like effector (TALE), and/or CRISPR-Cas protein, variant, or fusion thereof; a guide RNA and/or crRNA; and/or a ribonucleoprotein complex (RNP) comprising a CRISPR-Cas protein, variant, or fusion thereof and optionally a guide RNA.

32. The cell of claim 31, wherein the cargo reagent is selected from the proteins listed in Tables 2, 3, 4, & 5, or that is at least 95% identical to a sequence set forth herein, optionally in Tables 2, 3, 4, and 5.

33. The cell of claim 31, wherein the gene editing and/or epigenetic modulating reagent comprises a CRISPR-Cas protein, and the mhVLP further comprises one or more guide RNAs that bind to and direct the CRISPR-Cas protein to a target sequence.

34. The cell of claim 28, wherein the cargo comprises a fusion to a phospholipid bilayer recruitment domain, preferably as shown in Table 6, or that is at least 95% identical to a sequence set forth herein in Table 6.

35. The cell of claim 28, wherein the cell is from a primary or stable human cell lines.

36. The cell of claim 28, which is a Human Embryonic Kidney (HEK) 293 cell or HEK293 T cell.
